(19) United States
(12) Patent Application Publication
(10) Pub. No.: US 2025/0017922 A1
(43) Pub. Date: Jan. 16, 2025

(54) BIFUNCTIONAL COMPOUNDS FOR DEGRADING BTK WITH ENHANCED IMID ACTIVITY

(71) Applicant: NURIX THERAPEUTICS, INC., San Francisco, CA (US)

(72) Inventors: Cristiana Guiducci, San Francisco, CA (US); Mark Noviski, San Francisco, CA (US)

(21) Appl. No.: 18/290,920

(22) PCT Filed: Jul. 22, 2022
(86) PCT No.: PCT/US2022/038084

§ 371 Date: Jan. 22, 2024

Related U.S. Application Data
(60) Provisional application No. 63/225,401, filed on Jul. 23, 2021.

(51) Int. Cl. A61K 31/497; A61P 35/00; A61P 35/02; C07D 401/14

(52) U.S. Cl. CPC A61K 31/497; A61P 35/00; A61P 35/02; C07D 401/14

(57) ABSTRACT

This disclosure relates to compounds useful for degrading BTK via a ubiquitin proteolytic pathway with enhanced IMiD activity. The description also provides pharmaceutically acceptable compositions comprising said compounds and methods of using the compositions in the treatment of various disease, conditions, or disorders.

### CROSS-REFERENCE TO RELATED APPLICATIONS

This application claims the benefit of U.S. Provisional Application No. 63/225,401, filed Jul. 23, 2021, the content of which is herein incorporated by reference in its entirety.

### FIELD

This disclosure provides novel bifunctional compounds for proteolytically degrading targeted Bruton's tyrosine kinases (BTK) and methods for treating diseases modulated by BTK. In particular embodiments, the compounds are capable of degrading Bruton's tyrosine kinase with enhanced IMiD activity. In particular embodiments, the compounds are useful for methods of treating diseases amenable to a combination of BTK and IMiD modulation.

### BACKGROUND

BTK is a member of the TEC family of kinases and is a crucial signaling hub in the B cell antigen receptor (BCR) pathway. Mutations in BTK result in X-linked agammaglobulinaemia (XLA), in which B cell maturation is impaired, resulting in reduced immunoglobulin production. Hendriks, et al., 2011, Expert Opin Ther Targets 15:1002-1021, 2011. The central role of BTK in B cell signaling and function makes BTK an attractive therapeutic target for B cell malignancies as well as autoimmune and inflammatory diseases. Ibrutinib, a covalent inhibitor of BTK, has been approved to treat chronic lymphocytic leukemia (CLL), mantle cell lymphoma (MCL) rand other B cell malignancies, as well as graft-versus-host disease (GvHD). Miklos, et al., 2017, Blood, 120(21):2243-2250. Currently, ibrutinib and second-generation BTK inhibitors are being investigated for oncology and immune-related indications such as rheumatoid arthritis. Akinleye, et al., 2013, J of Hematolo Oncol. 6:59; Liu, et al., 2011, J Pharm and Exper Ther. 338(1):154-163; Di Paolo, et al., 2011, Nat Chem Biol. 7(1): 41-50.

As an alternative to stoichiometric inhibition, proteolytic degradation of BTK could have dramatic consequences for B cell function by effectively blocking BCR signaling. Removal of BTK protein would eliminate BTK kinase activity as well as any protein interaction or scaffolding function of BTK. Specific degradation of BTK could be accomplished using heterobifunctional small molecules to recruit BTK to a ubiquitin ligase thus promoting ubiquitylation and proteasomal degradation of BTK. Thalidomide derivatives, such as lenalidomide or pomalidomide, can be used to recruit potential substrates to cereblon (CRBN), a component of a ubiquitin ligase complex. This unique therapeutic approach could present a mechanism of action for interfering with BTK activity and BCR signaling that is distinct from the mechanism of stoichiometric BTK inhibition. Furthermore, this degradative approach could effectively target the C481S mutated form of BTK, a mutation which has been clinically observed and confers resistance to inhibition by ibrutinib. Woyach, et al., 2012, Blood, 120(6): 1175-1184, 2012.

Using degrader compounds to destroy target proteins through CRBN has already led to candidate anti-cancer drugs. Okumura et al., 2020, Pharmaceuticals 13:95. These drugs not only target the cancer cell, but also trigger a strong immune response in part by degrading, for instance, Ikaros and Aiolos, and by increasing IL-2 secretion. The Immunomodulatory imide Drug (IMiD) portion of these compounds is believed to be responsible for the potent immune effect. Together these degrader compounds hinder tumor growth directly and through the immune system. Quach et al., 2010, Leukemia 24:22-32.

Certain of these compounds were discovered to have activity in addition to degrading BTK. In particular, certain BTK compounds possess activity similar to immunomodulatory imide (IMiD) drugs such as pomalidomide and lenalidomide. Through binding to CRBN, IMiDs alter the substrate repertoire of the CRBN ubiquitin ligase complex, in part, by leading to the degradation of non-physiologic substrates, or neosubstrates. Like other IMiDs, these compounds can promote formation of ternary complexes with CRBN leading to ubiquitylation and degradation of two transcription factors, Aiolos and Ikaros. Gandhi et al., 2014, Brit. J. Haematol. 164(6):8111-821; Kronke et al., 2014, Science 343:301-305; Lu et al., 2013, Science 343:305-309. Clinically, the IMiD lenalidomide is FDA approved for the treatment of multiple myeloma (MM), myelodysplastic syndromes with a 5q deletion (MDS), mantle cell lymphoma (MCL), follicular lymphoma (FL), and marginal zone lymphoma (MZL). Pomalidomide, an optimized IMiD drug, is more potent than lenalidomide and has demonstrated efficacy in relapsed MM patients, including patient's refractory to both lenalidomide and bortezomib. This intentional dual activity of degrading Ikaros and Aiolos, along with the distinct oncogenic target BTK, would provide compounds useful for the treatment or prevention of diseases and disorders amenable to BTK modulation and IMiD modulation.

### SUMMARY

Provided herein are methods of treating or preventing a disease, disorder, or condition in a subject in need thereof by administering a compound capable of degrading Bruton's tyrosine kinase with enhanced IMiD activity. In the examples provided herein, compounds are shown to recruit CRBN and degrade BTK with enhanced IMiD activity. Specifically, in certain embodiments, exemplary compounds degrade BTK while promoting degradation of Aiolos or Ikaros. In certain embodiments, the compounds also trigger IL-2, another marker of IMiD activity. By degrading BTK with significant IMiD activity, the compounds are useful for the treatment or prevention of diseases and disorders amenable to BTK modulation and IMiD modulation.

In one aspect, provided herein are methods of treating or preventing a disease, disorder, or condition in a subject in need thereof. The methods comprise the step of administering to the subject an amount of a bifunctional compound capable of inducing proteolytic degradation of Bruton's tyrosine kinase with enhanced IMiD activity. In certain embodiments, the amount is effective to treat or prevent the disease, disorder, or condition. In certain embodiments, the methods are for treating or preventing cancer, for instance a B-cell malignancy.

In one aspect, provided herein are methods of treating or preventing a B-cell malignancy, disorder, or condition in a subject in need thereof. The methods comprise the step of administering to the subject an amount of a bifunctional compound capable of inducing proteolytic degradation of Bruton's tyrosine kinase with enhanced IMiD activity. In certain embodiments, the amount is effective to treat or prevent the B-cell malignancy.

In another aspect, provided herein are methods of degrading Bruton's tyrosine kinase in a subject in need thereof. The methods comprise the step of administering to the subject an amount of a bifunctional compound capable of inducing proteolytic degradation of Bruton's tyrosine kinase with enhanced IMiD activity. In certain embodiments, the amount is effective to degrade Bruton's tyrosine kinase in the subject.

In another aspect, provided herein are methods of preventing B cell activation in a subject in need thereof. The methods comprise the step of administering to the subject an amount of a bifunctional compound capable of inducing proteolytic degradation of Bruton's tyrosine kinase with enhanced IMiD activity. In certain embodiments, the amount is effective to prevent B cell activation.

In another aspect, provided herein are methods of degrading a mutant Bruton's tyrosine kinase. The methods comprise the step of contacting a cell expressing the mutant Bruton's tyrosine kinase with an amount of a bifunctional compound capable of inducing proteolytic degradation of Bruton's tyrosine kinase with enhanced IMiD activity. In certain embodiments, the amount is effective to degrade the mutant Bruton's tyrosine kinase. In certain embodiments, the mutant Bruton's tyrosine kinase is a C481 mutant. In certain embodiments, the mutant Bruton's tyrosine kinase is a C481S mutant.

In the methods, the bifunctional compounds comprise a moiety capable of specifically binding BTK with enhanced IMiD activity. Particular compounds are described herein. The compounds can be administered in any form, including pharmaceutically acceptable salts and pharmaceutical compositions. In particular embodiments, the compounds are administered orally.

The methods provided herein are useful for treating or preventing diseases, conditions, and disorders mediated by Bruton's tyrosine kinase, including, for instance, cancer, including B-cell malignancies.

### BRIEF DESCRIPTION OF THE FIGURES

FIG. 1 provides the effects of compound 5 on a REC-1 human mantel cell line compared to a comparator compound and to ibrutinib, acalabrutinib, pomalidomide, and lenalidomide.

FIG. 2 shows Compound 5 degrades both BTK and immunomodulatory cereblon neosubstrate Aiolos.

FIG. 3 shows Compound 5 is active against Ibrutinib-resistant tumor cell lines

FIG. 4 shows BTK degradation of 80% drives potent anti-tumor activity in Preclinical Models. Ikaros and Aiolos degradation also achieve target range at therapeutic doses.

FIG. 5 shows robust BTK degradation observed with Compound 5 across all dose levels and malignancies.

FIG. 6 shows Compound 5 rapid and sustained degradation of BTK in patients with CLL

FIG. 7 shows Compound 5 demonstrates greater Ikaros degradation, consistent with cereblon immunomodulatory activity.

### DETAILED DESCRIPTION

Provided herein are methods of using bifunctional compounds that induce the proteolytic degradation of Bruton's tyrosine kinase (BTK) via a ubiquitin proteolysis pathway.

As used herein, the following definitions shall apply unless otherwise indicated.

### Definitions

For purposes herein, the chemical elements are identified in accordance with the Periodic Table of the Elements, CAS version, Handbook of Chemistry and Physics, 75th Ed. Additionally, general principles of organic chemistry are described in “Organic Chemistry,” Thomas Sorrell, University Science Books, Sausalito: 1999, and “March's Advanced Organic Chemistry,” 5th Ed., Ed.: Smith, M. B. and March, J., John Wiley & Sons, New York: 2001, the entire contents of which are hereby incorporated by reference.

As described herein, “IMiD” activity indicates Immunomodulatory imide Drug activity. In certain embodiments, IMiD activity is relative to an IMiD compound. In certain embodiments, the IMiD compound is selected from the group consisting of thalidomide, lenalidomide, pomalidomide, iberdomide, and apremilast. In certain embodiments, IMiD activity is measured with downregulation of an IMiD target. In certain embodiments, the target is Aiolos. In certain embodiments, the target is Ikaros. In certain embodiments, “enhanced IMiD activity” indicates a maximum degradation of Aiolos of greater than 50%, 60%, 70%, 75%, 80%, 85%, or 90% under physiological conditions. In certain embodiments, “Low IMiD activity” indicates a maximum degradation of Ikaros of greater than 50%, 60%, 70%, 75%, 80%, 85%, or 90% under physiological conditions. Exemplary assays for Aiolos degradation are provided in the Examples herein.

As described herein, “protecting group” refers to a moiety or functionality that is introduced into a molecule by chemical modification of a functional group in order to obtain chemoselectivity in a subsequent chemical reaction. Standard protecting groups are provided in Wuts and Greene: “Greene's Protective Groups in Organic Synthesis,” 4th Ed, Wuts, P. G. M. and Greene, T. W., Wiley-Interscience, New York: 2006.

As described herein, compounds herein optionally may be substituted with one or more substituents, such as those illustrated generally herein, or as exemplified by particular classes, subclasses, and species of the description.

As used herein, the term “hydroxyl” or “hydroxy” refers to an —OH moiety.

As used herein, the term “aliphatic” encompasses the terms alkyl, alkenyl, and alkynyl, each of which are optionally substituted as set forth below.

As used herein, an “alkyl” group refers to a saturated aliphatic hydrocarbon group containing 1-12 (e.g., 1-8, 1-6, or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, phospho, cycloaliphatic (e.g., cycloalkyl or cycloalkenyl), heterocycloaliphatic (e.g., heterocycloalkyl or heterocycloalkenyl), aryl, heteroaryl, alkoxy, aryl, heteroaryl, acyl (e.g., (aliphatic)carbonyl, (cycloaliphatic)carbonyl, or (heterocycloaliphatic)carbonyl), nitro, cyano, amido (e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, alkylaminocarbonyl, cycloalkylaminocarbonyl, heterocycloalkylaminocarbonyl, arylaminocarbonyl, or heteroarylaminocarbonyl), amino (e.g., aliphaticamino, cycloaliphaticamino, or heterocycloaliphaticamino), sulfonyl (e.g., aliphatic-SO₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl.

As used herein, an “alkenyl” group refers to an aliphatic carbon group that contains 2-8 (e.g., 2-12, 2-6, or 2-4) carbon atoms and at least one double bond. Like an alkyl group, an alkenyl group can be straight or branched. Examples of an alkenyl group include, but are not limited to, allyl, 1- or 2-isopropenyl, 2-butenyl, and 2-hexenyl. An alkenyl group can be optionally substituted with one or more substituents such as halo, phospho, cycloaliphatic (e.g., cycloalkyl or cycloalkenyl), heterocycloaliphatic (e.g., heterocycloalkyl or heterocycloalkenyl), aryl, heteroaryl, alkoxy, aroyl, heteroaroyl, acyl (e.g., (aliphatic)carbonyl, (cycloaliphatic)carbonyl, or (heterocycloaliphatic)carbonyl), nitro, cyano, amido (e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, alkylaminocarbonyl, cycloalkylaminocarbonyl, heterocycloalkylaminocarbonyl, arylaminocarbonyl, or heteroarylaminocarbonyl), amino (e.g., aliphaticamino, cycloaliphaticamino, heterocycloaliphaticamino, or aliphaticsulfonylamino), sulfonyl (e.g., alkyl-SO₂—, cycloaliphatic-SO₂—, or aryl-SO₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, or haloalkenyl.

As used herein, an “alkynyl” group refers to an aliphatic carbon group that contains 2-8 (e.g., 2-12, 2-6, or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl (e.g., aliphaticsulfanyl or cycloaliphaticsulfanyl), sulfinyl (e.g., aliphaticsulfinyl or cycloaliphaticsulfinyl), sulfonyl (e.g., aliphatic-SO₂—, aliphaticamino-SO₂—, or cycloaliphatic-SO₂—), amido (e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino, or heteroarylaminocarbonyl), urea, thiourea, sulfamoyl, sulfamide, alkoxycarbonyl, alkylcarbonyloxy, cycloaliphatic, heterocycloaliphatic, aryl, heteroaryl, acyl (e.g., (cycloaliphatic)carbonyl or (heterocycloaliphatic)carbonyl), amino (e.g., aliphaticamino), sulfoxy, oxo, carboxy, carbamoyl, (cycloaliphatic)oxy, (heterocycloaliphatic)oxy, or (heteroaryl)alkoxy.

As used herein, an “amido” encompasses both “aminocarbonyl” and “carbonylamino.” These terms when used alone or in connection with another group refer to an amido group such as —N(R^{X})—C(O)—R^{Y} or —C(O)—N(R^{X})₂, when used terminally, and —C(O)—N(R^{X})— or —N(R^{X})—C(O)— when used internally, wherein R^{X} and R^{Y} can be aliphatic, cycloaliphatic, aryl, araliphatic, heterocycloaliphatic, heteroaryl, or heteroaraliphatic. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NR^{X}R^{Y} wherein each of R^{X} and R^{Y} is independently hydrogen (H or —H), aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, or arylamino. When the term “amino” is not the terminal group (e.g., alkylcarbonylamino), it is represented by —NR^{X}—, where R^{X} has the same meaning as defined above.

As used herein, an “aryl” group used alone or as part of a larger moiety as in “aralkyl,” “aralkoxy,” or “aryloxyalkyl” refers to monocyclic (e.g., phenyl); bicyclic (e.g., indenyl, naphthalenyl, tetrahydronaphthyl, or tetrahydroindenyl); and tricyclic (e.g., fluorenyl tetrahydrofluorenyl, tetrahydroanthracenyl, or anthracenyl) ring systems in which the monocyclic ring system is aromatic or at least one of the rings in a bicyclic or tricyclic ring system is aromatic. The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl (e.g., (aliphatic)carbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl); sulfonyl (e.g., aliphatic-SO₂— or amino-SO₂—); sulfinyl (e.g., aliphatic-S(O)— or cycloaliphatic-S(O)—); sulfanyl (e.g., aliphatic-S—); cyano; halo; hydroxy; mercapto; sulfoxy; urea; thiourea; sulfamoyl; sulfamide; or carbamoyl. Alternatively, an aryl can be unsubstituted.

Non-limiting examples of substituted aryls include haloaryl (e.g., mono-, di- (such as p,m-dihaloaryl), and (trihalo)aryl); (carboxy)aryl (e.g., (alkoxycarbonyl)aryl, ((aralkyl)carbonyloxy)aryl, and (alkoxycarbonyl)aryl); (amido)aryl (e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl); aminoaryl (e.g., ((alkylsulfonyl)amino)aryl or ((dialkyl)amino)aryl); (cyanoalkyl)aryl; (alkoxy)aryl; (sulfamoyl)aryl (e.g., (aminosulfonyl)aryl); (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are defined herein. An example of an araliphatic such as an aralkyl group is benzyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic (e.g., alkyl, alkenyl, or alkynyl, including carboxyalkyl, hydroxyalkyl, or haloalkyl such as trifluoromethyl), cycloaliphatic (e.g., cycloalkyl or cycloalkenyl), (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido (e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino), cyano, halo, hydroxy, acyl, mercapto, alkylsulfanyl, sulfoxy, urea, thiourea, sulfamoyl, sulfamide, oxo, or carbamoyl.

As used herein, a “bicyclic ring system” includes 6-12 (e.g., 8-12 or 9-, 10-, or 11-) membered structures that form two rings, wherein the two rings have at least one atom in common (e.g., two atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which are optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, or ((aminocarbonyl)cycloalkyl)cycloalkyl.

A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, bicyclo[2.2.2]octenyl, or bicyclo[3.3.1]nonenyl.

A cycloalkyl or cycloalkenyl group can be optionally substituted with one or more substituents such as phospho, aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino), nitro, carboxy (e.g., HOOC—, alkoxycarbonyl, or alkylcarbonyloxy), acyl (e.g., (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl], cyano, halo, hydroxy, mercapto, sulfonyl (e.g., alkyl-SO₂— and aryl-SO₂—), sulfinyl (e.g., alkyl-S(O)—), sulfanyl (e.g., alkyl-S—), sulfoxy, urea, thiourea, sulfamoyl, sulfamide, oxo, or carbamoyl.

As used herein, the term “heterocycloaliphatic” encompasses heterocycloalkyl groups and heterocycloalkenyl groups, each of which being optionally substituted as set forth below.

As used herein, a “heterocycloalkyl” group refers to a 3-10 membered mono- or bicylic (fused, bridged, or spiro) (e.g., 5- to 10-membered mono- or bicyclic) saturated ring structure, in which one or more of the ring atoms is a heteroatom (e.g., nitrogen (N), oxygen (O), sulfur (S), or combinations thereof). Non-limiting examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholinyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, decahydro-2,7-naphthyridine, 2,8-diazaspiro[4.5]decane, 2,7-diazaspiro[3.5]nonane, octahydropyrrolo[3,4-c]pyrrole, octahydro-1H-pyrrolo[3,4-b]pyridine, and 2,6-dioxa-tricyclo[3.3.1.0^{3,7}]nonyl. A monocyclic heterocycloalkyl group can be fused with a phenyl moiety to form structures, such as tetrahydroisoquinoline, that would be categorized as heteroaryls.

A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicyclic heterocycloaliphatics are numbered according to standard chemical nomenclature.

A heterocycloalkyl or heterocycloalkenyl group can be optionally substituted with one or more substituents such as phospho, aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino], nitro, carboxy (e.g., HOOC—, alkoxycarbonyl, or alkylcarbonyloxy), acyl (e.g., (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl), nitro, cyano, halo, hydroxy, mercapto, sulfonyl (e.g., alkylsulfonyl or arylsulfonyl), sulfinyl (e.g., alkylsulfinyl), sulfanyl (e.g., alkylsulfanyl), sulfoxy, urea, thiourea, sulfamoyl, sulfamide, oxo, or carbamoyl.

A “heteroaryl” group, as used herein, refers to a monocyclic, bicyclic, or tricyclic ring system having four to fifteen ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having two to three rings. For example, a benzofused group includes benzo fused with one or two 4- to 8-membered heterocycloaliphatic moieties (e.g., indolizyl, indolyl, isoindolyl, 3H-indolyl, indolinyl, benzo[b]furyl, benzo[b]thiophene-yl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, phthalazyl, quinazolyl, quinoxalyl, isoquinolyl, 4H-quinolizyl, benzo-1,2,5-thiadiazolyl, or 1,8-naphthyridyl. Other examples of heteroaryls include 1,2,3,4-tetrahydroisoquinoline and 4,5,6,7-tetrahydropyrazolo[1,5-a]pyrazine.

Without limitation, monocyclic heteroaryls include furyl, thiophene-yl, 2H-pyrrolyl, oxazolyl, thiazolyl, imidazolyl, pyrazolyl, isoxazolyl, isothiazolyl, 1,3,4-thiadiazolyl, 2H-pyranyl, 4H-pranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl (e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl); sulfonyl (e.g., aliphaticsulfonyl or aminosulfonyl); sulfinyl (e.g., aliphaticsulfinyl); sulfanyl (e.g., aliphaticsulfanyl); nitro; cyano; halo; hydroxy; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl (e.g., mono- and di-(halo)heteroaryl); (carboxy)heteroaryl (e.g., (alkoxycarbonyl)heteroaryl); cyanoheteroaryl; aminoheteroaryl (e.g., ((alkylsulfonyl)amino)heteroaryl and ((dialkyl)amino)heteroaryl); (amido)heteroaryl (e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl); (cyanoalkyl)heteroaryl; (alkoxy)heteroaryl; (sulfamoyl)heteroaryl (e.g., (aminosulfonyl)heteroaryl); (sulfonyl)heteroaryl (e.g., (alkylsulfonyl)heteroaryl); (hydroxyalkyl)heteroaryl; (alkoxyalkyl)heteroaryl; (hydroxy)heteroaryl; ((carboxy)alkyl)heteroaryl; (((dialkyl)amino)alkyl)heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl (e.g., (alkylcarbonyl)heteroaryl); (alkyl)heteroaryl; or (haloalkyl)heteroaryl (e.g., trihaloalkylheteroaryl).

As used herein, a “heteroaraliphatic” (such as a heteroaralkyl group) refers to an aliphatic group (e.g., a C₁₋₄ alkyl group) that is substituted with a heteroaryl group. “Aliphatic,” “alkyl,” and “heteroaryl” have been defined above.

As used herein, a “heteroaralkyl” group refers to an alkyl group (e.g., a C₁₋₄ alkyl group) that is substituted with a heteroaryl group. Both “alkyl” and “heteroaryl” have been defined above. A heteroaralkyl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

As used herein, a “bridged bicyclic ring system” refers to a bicyclic heterocyclicalipahtic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.3.2]decyl, 2-oxabicyclo[2.2.2]octyl, 1-azabicyclo[2.2.2]octyl, 3-azabicyclo[3.2.1]octyl, and 2,6-dioxa-tricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, an “acyl” group refers to a formyl group or R^{X}—C(O)— (such as alkyl-C(O)—, also referred to as “alkylcarbonyl”) where R^{X} and “alkyl” have been defined previously. Acetyl and pivaloyl are examples of acyl groups.

As used herein, an “aroyl” or “heteroaroyl” refers to an aryl-C(O)— or a heteroaryl-C(O)—. The aryl and heteroaryl portion of the aroyl or heteroaroyl is optionally substituted as previously defined herein.

As used herein, an “alkoxy” group refers to an alkyl-O— group where “alkyl” has been defined previously herein.

As used herein, a “carbamoyl” group refers to a group having the structure —O—CO—NR^{X}R^{Y} or —NR^{X}—CO—O—R^{Z}, wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, when used as a terminal group; or —OC(O)— or —C(O)O— when used as an internal group.

As used herein, an ester refers to —COOR^{X} when used as a terminal group; or —COOR^{X}— when used as an internal group, wherein R^{X} has been defined above.

As used herein, a formate refers to —OC(O)H.

As used herein, an acetate refers to —OC(O)R^{X}, wherein R^{X} has been defined above.

As used herein, a “haloaliphatic” group refers to an aliphatic group substituted with one to three halogen. For instance, the term haloalkyl includes the group —CF₃.

As used herein, a “mercapto” or “sulfhydryl” group refers to —SH.

As used herein, a “sulfo” group refers to —SO₃H or —SO₃R^{X} when used terminally or —S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfamoyl” group refers to the structure —O—S(O)₂—NR^{Y}R^{Z} wherein R^{Y} and R^{Z} have been defined above.

As used herein, a “sulfonamide” group refers to the structure —S(O)₂—NR^{X}R^{Y} or —NR^{X}—S(O)₂—R^{Z} when used terminally; or —S(O)₂—NR^{X}— or —NR^{X}—S(O)₂— when used internally, wherein R^{X}, R^{Y}, and R^{Z} are defined above.

As used herein a “sulfanyl” group refers to —S—R^{X} when used terminally and —S— when used internally, wherein R^{X} has been defined above. Examples of sulfanyls include aliphatic-S—, cycloaliphatic-S—, aryl-S—, or the like.

As used herein a “sulfinyl” group refers to —S(O)—R^{X} when used terminally and —S(O)— when used internally, wherein R^{X} has been defined above. Examples of sulfinyl groups include aliphatic-S(O)—, aryl-S(O)—, (cycloaliphatic(aliphatic))—S(O)—, cycloalkyl-S(O)—, heterocycloaliphatic-S(O)—, heteroaryl-S(O)—, and/or the like.

As used herein, a “sulfonyl” group refers to —S(O)₂—R^{X} when used terminally and —S(O)₂— when used internally, wherein R^{X} has been defined above. Examples of sulfonyl groups include aliphatic-S(O)₂—, aryl-S(O)₂—, (cycloaliphatic(aliphatic))—S(O)₂—, cycloaliphatic-S(O)₂—, heterocycloaliphatic-S(O)₂—, heteroaryl-S(O)₂—, (cycloaliphatic(amido(aliphatic)))—S(O)₂—, and/or the like.

As used herein, a “sulfoxy” group refers to —O—S(O)—R^{X} or —S(O)—O—R^{X}, when used terminally and —O—S(O)— or —S(O)—O— when used internally, where R^{X} has been defined above.

As used herein, a “halogen” or “halo” group refers to fluorine (F), chlorine (Cl), bromine (Br), or iodine (I).

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refers to —C(O)—.

As used herein, an “oxo” refers to ═O.

As used herein, the term “phospho” refers to phosphinates and phosphonates. Examples of phosphinates and phosphonates include —P(O)(R^{P})₂, wherein R^{P} is aliphatic, alkoxy, aryloxy, heteroaryloxy, (cycloaliphatic)oxy, (heterocycloaliphatic)oxy, aryl, heteroaryl, cycloaliphatic or amino.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} each when used terminally and —NR^{X}—CO—NR^{Y}— or —NR^{X}—CS—NR^{Y}— each when used internally, wherein R^{X}, R^{Y}, and R^{Z} have been defined above.

As used herein, a “guanidine” group refers to the structure —N═C(N(R^{X}R^{Y}))N(R^{X}R^{Y}) or —NR^{X}—C(═NR^{X})NR^{X}R^{Y} wherein R^{X} and R^{Y} have been defined above.

As used herein, the term “amidino” group refers to the structure —C═(NR^{X})N(R^{X}R^{Y}) wherein R^{X} and R^{Y} have been defined above.

As used herein, the term “vicinal” generally refers to the placement of substituents on a group that includes two or more carbon atoms, wherein the substituents are attached to adjacent carbon atoms.

As used herein, the term “geminal” generally refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl (i.e., R^{X}O(O)C-alkyl) is an example of a carboxy group used terminally. A group is internal when the group is present in the middle of or within the termini of a substituent of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, an “aliphatic chain” refers to a branched or straight aliphatic group (e.g., alkyl groups, alkenyl groups, or alkynyl groups). A straight aliphatic chain has the structure —[CH₂]ᵥ—, where v is 1-12. A branched aliphatic chain is a straight aliphatic chain that is substituted with one or more aliphatic groups. A branched aliphatic chain has the structure —[CQQ]ᵥ— where each Q is independently a hydrogen (H or —H) or an aliphatic group; however, Q shall be an aliphatic group in at least one instance. The term aliphatic chain includes alkyl chains, alkenyl chains, and alkynyl chains, where alkyl, alkenyl, and alkynyl are defined above.

The phrase “optionally substituted” is used herein interchangeably with the phrase “substituted or unsubstituted.” As described herein, compounds herein can optionally be substituted with one or more substituents, such as are illustrated generally above, or as exemplified by particular classes, subclasses, and species of the description. As described herein, the variables R, R¹, R², L, Y, and Z, and other variables contained in Formula A-X or I-IV described herein encompass specific groups, such as alkyl and aryl. Unless otherwise noted, each of the specific groups for the variables R, R¹⁰, R^{A}, R¹, R², L, L¹, D, W, E, V, G, Y, and Z, and other variables contained therein can be optionally substituted with one or more substituents described herein. Each substituent of a specific group is further optionally substituted with one to three of halo, cyano, oxo, alkoxy, hydroxy, amino, nitro, aryl, cycloaliphatic, heterocycloaliphatic, heteroaryl, haloalkyl, and alkyl. For instance, an alkyl group can be substituted with alkylsulfanyl and the alkylsulfanyl can be optionally substituted with one to three of halo, cyano, oxo, alkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkoxy groups can form a ring together with the atom(s) to which they are bound.

As used herein, the term “substituted,” whether preceded by the term “optionally” or not, refers generally to the replacement of hydrogen atoms in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, for example, both rings share one common atom. Non-limiting examples of spiro heterocycloalkyls include

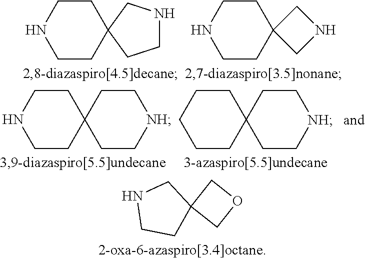

As one of ordinary skill in the art will recognize, combinations of substituents envisioned by this description are those combinations that result in the formation of stable or chemically feasible compounds.

As used herein, the phrase “stable or chemically feasible” refers to compounds that are not substantially altered when subjected to conditions to allow for their production, detection, and their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, an “effective amount” is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, New York, 537 (1970). As used herein, “patient” refers to a mammal, including a human.

As used herein, the term “about” means within +10% of a value. For example, a dose that is about 100 mg/kg provides that the does can 90 mg/kg to 110 mg/kg. By way of further example, an amount of an additional therapeutic agent ranging from about 50% to about 100% provides that the amount of additional therapeutic agent ranges from 45-55% to 90-110%. A person of skill in the art will appreciate the scope and application of the term “about” when used to describe other values disclosed herein.

Unless otherwise stated, structures depicted herein also are meant to include all isomeric (e.g., enantiomeric, diastereomeric, and geometric (or conformational)) forms of the structure; for example, the (R)- and (S)-configurations for each asymmetric center, (Z)- and (E)-double bond isomers, and (Z)- and (E)-conformational isomers. Therefore, single stereochemical isomers as well as enantiomeric, diastereomeric, and geometric (or conformational) mixtures of the present compounds are within the scope of the description. Alternatively, as used herein, “enantiomeric excess (ee)” refers to a dimensionless mol ratio describing the purity of chiral substances that contain, for example, a single stereogenic center. For instance, an enantiomeric excess of zero would indicate a racemic (e.g., 50:50 mixture of enantiomers, or no excess of one enantiomer over the other). By way of further example, an enantiomeric excess of ninety-nine would indicate a nearly stereopure enantiomeric compound (i.e., large excess of one enantiomer over the other). The percentage enantiomeric excess, % ee=([(R)-compound]−[(S)-compound])/([(R)-compound]+[(S)-compound])×100, where the (R)-compound>(S)-compound; or % ee=([(S)-compound]−[(R)-compound])/([(S)-compound]+[(R)-compound])×100, where the (S)-compound>(R)-compound. Moreover, as used herein, “diastereomeric excess (de)” refers to a dimensionless mol ratio describing the purity of chiral substances that contain more than one stereogenic center. For example, a diastereomeric excess of zero would indicate an equimolar mixture of diastereoisomers. By way of further example, diastereomeric excess of ninety-nine would indicate a nearly stereopure diastereomeric compound (i.e., large excess of one diastereomer over the other). Diastereomeric excess may be calculated via a similar method to ee. As would be appreciated by a person of skill, de is usually reported as percent de (% de). % de may be calculated in a similar manner to % ee.

In certain embodiments, the compounds or inhibitors described herein have an ee, de, % ee, or % de greater than zero. For example, in certain embodiments, the compounds or inhibitors described herein have an ee, de, % ee, or % de of ten. In certain embodiments, the compounds or inhibitors described herein have an ee, de, % ee, or % de of twenty-five. In certain embodiments, the compounds or inhibitors described herein have an ee, de, % ee, or % de of fifty. In certain embodiments, the compounds or inhibitors described herein have an ee, de, % ee, or % de of seventy-five.

In certain embodiments, the compounds or inhibitors described herein have an ee, de, % ee, or % de range from ninety to one hundred. In certain embodiments, the compounds or inhibitors described herein have an ee, de, % ee, or % de range from ninety-five to one hundred. In certain embodiments, the compounds or inhibitors described herein have an ee, de, % ee, or % de range from ninety-seven to one hundred. In certain embodiments, the compounds or inhibitors described herein have an ee, de, % ee, or % de range from ninety-eight to one hundred. In certain embodiments, the compounds or inhibitors described herein have an ee, de, % ee, or % de range from ninety-nine to one hundred.

In one embodiment of a compound or inhibitor described herein, the ee, de, % ee, or % de is one. In one embodiment of a compound or inhibitor described herein, the ee, de, % ee, or % de is two. In one embodiment of a compound or inhibitor described herein, the ee, de, % ee, or % de is three. In one embodiment of a compound or inhibitor described herein, the ee, de, % ee, or % de is four. In one embodiment of a compound or inhibitor described herein, the ee, de, % ee, or % de is five. In one embodiment of a compound or inhibitor described herein, the ee, de, % ee, or % de is six. In one embodiment of a compound or inhibitor described herein, the ee, de, % ee, or % de is seven. In one embodiment of a compound or inhibitor described herein, the ee, de, % ee, or % de is eight. In one embodiment of a compound or inhibitor described herein, the ee, de, % ee, or % de is nine. In one embodiment of a compound or inhibitor described herein, the ee, de, % ee, or % de is ten. In one embodiment of a compound or inhibitor described herein, the ee, de, % ee, or % de is eleven. In one embodiment of a compound or inhibitor described herein, the ee, de, % ee, or % de is twelve. In one embodiment of a compound or inhibitor described herein, the ee, de, % ee, or % de is thirteen. In one embodiment of a compound or inhibitor described herein, the ee, de, % ee, or % de is fourteen. In one embodiment of a compound or inhibitor described herein, the ee, de, % ee, or % de is fifteen. In one embodiment of a compound or inhibitor described herein, the ee, de, % ee, or % de is sixteen. In one embodiment of a compound or inhibitor described herein, the ee, de, % ee, or % de is seventeen. In one embodiment of a compound or inhibitor described herein, the ee, de, % ee, or % de is eighteen. In one embodiment of a compound or inhibitor described herein, the ee, de, % ee, or % de is nineteen. In one embodiment of a compound or inhibitor described herein, the ee, de, % ee, or % de is twenty. In one embodiment of a compound or inhibitor described herein, the ee, de, % ee, or % de is twenty-one. In one embodiment of a compound or inhibitor described herein, the ee, de, % ee, or % de is twenty-two. In one embodiment of a compound or inhibitor described herein, the ee, de, % ee, or % de is twenty-three. In one embodiment of a compound or inhibitor described herein, the ee, de, % ee, or % de is twenty-four. In one embodiment of a compound or inhibitor described herein, the ee, de, % ee, or % de is twenty-five. In one embodiment of a compound or inhibitor described herein, the ee, de, % ee, or % de is twenty-six. In one embodiment of a compound or inhibitor described herein, the ee, de, % ee, or % de is twenty-seven. In one embodiment of a compound or inhibitor described herein, the ee, de, % ee, or % de is twenty-eight. In one embodiment of a compound or inhibitor described herein, the ee, de, % ee, or % de is twenty-nine. In one embodiment of a compound or inhibitor described herein, the ee, de, % ee, or % de is thirty. In one embodiment of a compound or inhibitor described herein, the ee, de, % ee, or % de is thirty-one. In one embodiment of a compound or inhibitor described herein, the ee, de, % ee, or % de is thirty-two. In one embodiment of a compound or inhibitor described herein, the ee, de, % ee, or % de is thirty-three. In one embodiment of a compound or inhibitor described herein, the ee, de, % ee, or % de is thirty-four. In one embodiment of a compound or inhibitor described herein, the ee, de, % ee, or % de is thirty-five. In one embodiment of a compound or inhibitor described herein, the ee, de, % ee, or % de is thirty-six. In one embodiment of a compound or inhibitor described herein, the ee, de, % ee, or % de is thirty-seven. In one embodiment of a compound or inhibitor described herein, the ee, de, % ee, or % de is thirty-eight. In one embodiment of a compound or inhibitor described herein, the ee, de, % ee, or % de is thirty-nine. In one embodiment of a compound or inhibitor described herein, the ee, de, % ee, or % de is forty. In one embodiment of a compound or inhibitor described herein, the ee, de, % ee, or % de is forty-one. In one embodiment of a compound or inhibitor described herein, the ee, de, % ee, or % de is forty-two. In one embodiment of a compound or inhibitor described herein, the ee, de, % ee, or % de is forty-three. In one embodiment of a compound or inhibitor described herein, the ee, de, % ee, or % de is forty-four. In one embodiment of a compound or inhibitor described herein, the ee, de, % ee, or % de is forty-five. In one embodiment of a compound or inhibitor described herein, the ee, de, % ee, or % de is forty-six. In one embodiment of a compound or inhibitor described herein, the ee, de, % ee, or % de is forty-seven. In one embodiment of a compound or inhibitor described herein, the ee, de, % ee, or % de is forty-eight. In one embodiment of a compound or inhibitor described herein, the ee, de, % ee, or % de is forty-nine. In one embodiment of a compound or inhibitor described herein, the ee, de, % ee, or % de is fifty. In one embodiment of a compound or inhibitor described herein, the ee, de, % ee, or % de is fifty-one. In one embodiment of a compound or inhibitor described herein, the ee, de, % ee, or % de is fifty-two. In one embodiment of a compound or inhibitor described herein, the ee, de, % ee, or % de is fifty-three. In one embodiment of a compound or inhibitor described herein, the ee, de, % ee, or % de is fifty-four. In one embodiment of a compound or inhibitor described herein, the ee, de, % ee, or % de is fifty-five. In one embodiment of a compound or inhibitor described herein, the ee, de, % ee, or % de is fifty-six. In one embodiment of a compound or inhibitor described herein, the ee, de, % ee, or % de is fifty-seven. In one embodiment of a compound or inhibitor described herein, the ee, de, % ee, or % de is fifty-eight. In one embodiment of a compound or inhibitor described herein, the ee, de, % ee, or % de is fifty-nine. In one embodiment of a compound or inhibitor described herein, the ee, de, % ee, or % de is sixty. In one embodiment of a compound or inhibitor described herein, the ee, de, % ee, or % de is sixty-one. In one embodiment of a compound or inhibitor described herein, the ee, de, % ee, or % de is sixty-two. In one embodiment of a compound or inhibitor described herein, the ee, de, % ee, or % de is sixty-three. In one embodiment of a compound or inhibitor described herein, the ee, de, % ee, or % de is sixty-four. In one embodiment of a compound or inhibitor described herein, the ee, de, % ee, or % de is sixty-five. In one embodiment of a compound or inhibitor described herein, the ee, de, % ee, or % de is sixty-six. In one embodiment of a compound or inhibitor described herein, the ee, de, % ee, or % de is sixty-seven. In one embodiment of a compound or inhibitor described herein, the ee, de, % ee, or % de is sixty-eight. In one embodiment of a compound or inhibitor described herein, the ee, de, % ee, or % de is sixty-nine. In one embodiment of a compound or inhibitor described herein, the ee, de, % ee, or % de is seventy. In one embodiment of a compound or inhibitor described herein, the ee, de, % ee, or % de is seventy-one. In one embodiment of a compound or inhibitor described herein, the ee, de, % ee, or % de is seventy-two. In one embodiment of a compound or inhibitor described herein, the ee, de, % ee, or % de is seventy-three. In one embodiment of a compound or inhibitor described herein, the ee, de, % ee, or % de is seventy-four. In one embodiment of a compound or inhibitor described herein, the ee, de, % ee, or % de is seventy-five. In one embodiment of a compound or inhibitor described herein, the ee, de, % ee, or % de is seventy-six. In one embodiment of a compound or inhibitor described herein, the ee, de, % ee, or % de is seventy-seven. In one embodiment of a compound or inhibitor described herein, the ee, de, % ee, or % de is seventy-eight. In one embodiment of a compound or inhibitor described herein, the ee, de, % ee, or % de is seventy-nine. In one embodiment of a compound or inhibitor described herein, the ee, de, % ee, or % de is eighty. In one embodiment of a compound or inhibitor described herein, the ee, de, % ee, or % de is eighty-one. In one embodiment of a compound or inhibitor described herein, the ee, de, % ee, or % de is eighty-two. In one embodiment of a compound or inhibitor described herein, the ee, de, % ee, or % de is eighty-three. In one embodiment of a compound or inhibitor described herein, the ee, de, % ee, or % de is eighty-four. In one embodiment of a compound or inhibitor described herein, the ee, de, % ee, or % de is eighty-five. In one embodiment of a compound or inhibitor described herein, the ee, de, % ee, or % de is eighty-six. In one embodiment of a compound or inhibitor described herein, the ee, de, % ee, or % de is eighty-seven. In one embodiment of a compound or inhibitor described herein, the ee, de, % ee, or % de is eighty-eight. In one embodiment of a compound or inhibitor described herein, the ee, de, % ee, or % de is eighty-nine. In one embodiment of a compound or inhibitor described herein, the ee, de, % ee, or % de is ninety. In one embodiment of a compound or inhibitor described herein, the ee, de, % ee, or % de is ninety-one. In one embodiment of a compound or inhibitor described herein, the ee, de, % ee, or % de is ninety-two. In one embodiment of a compound or inhibitor described herein, the ee, de, % ee, or % de is ninety-three. In one embodiment of a compound or inhibitor described herein, the ee, de, % ee, or % de is ninety-four. In one embodiment of a compound or inhibitor described herein, the ee, de, % ee, or % de is ninety-five. In one embodiment of a compound or inhibitor described herein, the ee, de, % ee, or % de is ninety-six. In one embodiment of a compound or inhibitor described herein, the ee, de, % ee, or % de is ninety-seven. In one embodiment of a compound or inhibitor described herein, the ee, de, % ee, or % de is ninety-eight. In one embodiment of a compound or inhibitor described herein, the ee, de, % ee, or % de is ninety-nine In one embodiment of a compound or inhibitor described herein, the ee, de, % ee, or % de is one hundred. In certain embodiments, compounds or inhibitors described within Table 1 herein have an ee, de, % ee, or % de as described within this paragraph. In certain embodiments, any of compounds 1-22, as described in the Examples and/or Biological Examples have an ee, de, % ee, or % de as described within this paragraph. Unless otherwise stated, all tautomeric forms of the compounds of the description are within the scope of the description. Additionally, unless otherwise stated, structures depicted herein also are meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this description. Such compounds are useful, for example, as analytical tools or probes in biological assays, or as therapeutic agents.

As used herein, the term “& 1” means that a compound including the “& 1” notation at a particular chemical element or atom (e.g., carbon) within the compound was prepared as a mixture of two stereoisomers at the noted chemical element or atom (e.g., a diastereomeric mixture having a de or % de as described above).

Chemical structures and nomenclature are derived from ChemDraw, version 11.0.1, Cambridge, MA.

It is noted that the use of the descriptors “first,” “second,” “third,” or the like is used to differentiate separate elements (e.g., solvents, reaction steps, processes, reagents, or the like) and may or may not refer to the relative order or relative chronology of the elements described.

### Uses of the Compounds and Compositions

Bifunctional compounds that degrade BTK have been previously described, for example in PCT/US2019/56112, filed Oct. 14, 2019, published as WO 2020/081450, Apr. 23, 2020, and PCT/US2020/063176, filed Dec. 3, 2020, published as WO 2021/113557, Jun. 10, 2021, each of which is incorporated by reference in its entirety. Because many of these BTK degraders were discovered to have little or mixed IMiD activity. In contrast, in some embodiments, the bifunctional compounds described herein are useful for degrading BTK in biological samples or in patients with enhanced IMiD activity. Thus, an embodiment of this disclosure provides a method of treating a BTK-mediated disease or disorder. As used herein, the term “BTK-mediated disease or disorder” means any disease, disorder, or other deleterious condition in which a BTK is known to play a role. In some instances, a BTK-mediated disease or disorder is a proliferative disorder. Examples of proliferative disorders include cancer, for instance a B-cell malignancy.

The IMiD activity of the compound can be measured by any technique deemed suitable by the person of skill. In certain embodiments, IMiD activity is measured as Aiolos degradation. In certain embodiments, IMiD activity is measured as Ikaros degradation. In certain embodiments, IMiD activity is measured as IL2 activation. In certain embodiments, IMiD activity is measured as any combination of these. In certain embodiments, IMiD activity is measured in vivo. In certain embodiments, IMiD activity is measured in vitro, for instance in cell based assays.

In certain embodiments, IMiD activity of the compound at least 50% of the IMiD activity of a comparator compound. In certain embodiments, IMiD activity of the compound is at least 60% of the IMiD activity of a comparator compound. In certain embodiments, IMiD activity of the compound is at least 70% of the IMiD activity of a comparator compound. In certain embodiments, IMiD activity of the compound is at least 75% of the IMiD activity of a comparator compound. In certain embodiments, IMiD activity of the compound is at least 80% of the IMiD activity of a comparator compound. In certain embodiments, IMiD activity of the compound is at least 90% of the IMiD activity of a comparator compound. In certain embodiments, IMiD activity of the compound is at least 100% of the IMiD activity of a comparator compound. In certain embodiments, the comparator compound is thalidomide, lenalidomide, or pomalidomide. In certain embodiments, activity is measured as IC₅₀ or EC₅₀ or DC₅₀. In certain embodiments, activity is measured as Dₘₐₓ. In certain embodiments, activity is measured by Western blot.

In certain embodiments, the maximum degradation of Aiolos of greater than 50%, 60%, 70%, 75%, 80%, 85%, or 90% under physiological conditions. In certain embodiments, the maximum degradation of Ikaros of greater than 50%, 60%, 70%, 75%, 80%, 85%, or 90% under physiological conditions.

In the methods, the compounds comprise a moiety capable of specifically binding BTK and further comprise a moiety capable of recruiting an ubiquitin ligase to degrade the BTK. Particular compounds are described herein. The compounds can be administered in any form, including pharmaceutically acceptable salts and pharmaceutical compositions.

Due to the enhanced IMiD activity, the compounds described herein can yield increased immunomodulating activity compared to other BTK degrading compounds. The activity can provide for enhanced treatment or prevention of certain cancers, for instance B-cell malignancies.

In certain embodiments, the compound is administered for up to 14 days. In certain embodiments, the compound is administered for at least 15 days, at least 20 days, at least two weeks, at least three weeks, at least four weeks, at least one month, at least two months, at least three months, at least six months, at least one year, or longer.

In the dosing schedule, the doses can be administered on consecutive days or cyclically, according to the judgment of the practitioner of skill. In certain embodiments, the doses are administered on consecutive days. In certain embodiments, the doses are administered with an interval between doses. In certain embodiments, the interval is one day. In certain embodiments, the interval is two days. In certain embodiments, the interval is three days. In certain embodiments, the interval is four days. In certain embodiments, the interval is five days. In certain embodiments, the interval is six days.

In certain embodiments, the frequency of chronically administrating is daily. In certain embodiments, the frequency of chronically administering is twice a day. In certain embodiments, the frequency of chronically administering is thrice a day. In certain embodiments, the frequency of chronically administering is frice a day. In certain embodiments, the frequency of chronically administering is once a week. In certain embodiments, the frequency of chronically administering is twice a week.

In certain embodiments, the dose(s) are administered for a period of time with a first interval between dose(s), and then the dose(s) are re-administered for a period of time following the first interval between dose(s), wherein this dosing regimen can be repeated (i.e., cyclically or cyclically, for example, after a second, third, etc. interval between subsequent administrations of dose(s)) according to the judgment of the practitioner of skill. For example, in one embodiment, a first dose is administered for one week, followed by a first interval of one week without the first dose administration; then, a second dose is re-administered for another week, followed by a second interval of one week without the first or second dose administration, and so on cyclically. Other perturbations for first, second, third, etc. dose(s) followed by perturbations for first, second, third, etc. interval(s), and combinations thereof, are contemplated herein as would be appreciated by the practitioner of skill and the need of the patient. For example, in one embodiment, a first dose is administered daily for one week, followed by a first interval of three weeks without the first daily dose administration; then, a second dose is re-administered biweekly for another week, followed by a second interval of four weeks without the first daily or second biweekly dose administration, and so on cyclically.

The compound can be administered in any dose deemed suitable by the practitioner of skill. In certain embodiments, the dose is 0.1-1000 mg/kg. In certain embodiments, the dose is 0.1-900 mg/kg. In certain embodiments, the dose is 0.1-800 mg/kg. In certain embodiments, the dose is 0.1-700 mg/kg. In certain embodiments, the dose is 0.1-600 mg/kg. In certain embodiments, the dose is 0.1-500 mg/kg. In certain embodiments, the dose is 0.1-400 mg/kg. In certain embodiments, the dose is 0.1-300 mg/kg. In certain embodiments, the dose is 0.1-200 mg/kg. In certain embodiments, the dose is 0.1-100 mg/kg.

In certain embodiments, the dose is 100-600 mg/kg. In certain embodiments, the dose is 200-600 mg/kg. In certain embodiments, the dose is 250-600 mg/kg. In certain embodiments, the dose is 300-600 mg/kg. In certain embodiments, the dose is selected from the group consisting of 50 mg/kg. 100 mg/kg, 200 mg/kg, 300 mg/kg, 450 mg/kg, 600 mg/kg, 800 mg/kg, and 1000 mg/kg. In certain embodiments, the dose is about 50 mg/kg. In certain embodiments, the dose is about 75 mg/kg. In certain embodiments, the dose is about 100 mg/kg. In certain embodiments, the dose is about 150 mg/kg. In certain embodiments, the dose is about 200 mg/kg. In certain embodiments, the dose is about 250 mg/kg. In certain embodiments, the dose is about 300 mg/kg. In certain embodiments, the dose is about 400 mg/kg. In certain embodiments, the dose is about 450 mg/kg. In certain embodiments, the dose is about 500 mg/kg. In certain embodiments, the dose is about 600 mg/kg. In certain embodiments, the dose is about 700 mg/kg. In certain embodiments, the dose is about 750 mg/kg. In certain embodiments, the dose is about 800 mg/kg. In certain embodiments, the dose is about 900 mg/kg. In certain embodiments, the dose is about 1000 mg/kg.

In certain embodiments, the dose is selected from 100 mg, 200 mg, and 300 mg. In certain embodiments, the dose is 100 mg. In certain embodiments, the dose is 200 mg. In certain embodiments, the dose is 300 mg.

The compound can be administered by any route of administration deemed suitable by the practitioner of skill. In certain embodiments, the dose is administered orally. Formulations and techniques for administration are described in detail below.

In one aspect, provided herein are methods of treating or preventing cancer in a subject in need thereof. In certain embodiments, the methods comprise the step of orally administering to the subject an amount of a bifunctional compound capable of inducing proteolytic degradation of Bruton's tyrosine kinase. In certain embodiments, the amount is effective to treat or prevent the cancer.

In certain embodiments, the cancer is any cancer described below. In particular embodiments, the cancer comprises a solid tumor. In certain embodiments, the cancer is a B cell malignancy. In certain embodiments, the cancer is selected from the group consisting of chronic lymphocytic leukemia (CLL), small lymphocytic lymphoma (SLL), transformed CLL or Richter's transformation, small cell lymphoma, follicular lymphoma (FL), diffuse large B-cell lymphoma (DLBCL), non-Hodgkin lymphoma, mantle cell lymphoma (MCL), marginal zone lymphoma (MZL), Waldenstrom macroglobulinemia (WM), and central nervous system (CNS) lymphoma. In certain embodiments, the cancer is chronic lymphocytic leukemia. In certain embodiments, the cancer is small cell lymphoma. In certain embodiments, the cancer is follicular lymphoma. In certain embodiments, the cancer is diffuse large B-cell lymphoma. In certain embodiments, the cancer is non-Hodgkin lymphoma. In certain embodiments, the cancer is mantle cell lymphoma. In certain embodiments, the cancer is marginal zone lymphoma. In certain embodiments, the cancer is Waldenstrom macroglobulinemia. In certain embodiments, the cancer is small lymphocytic lymphoma (SLL). In certain embodiments, the cancer is CNS lymphoma. In certain embodiments, the cancer is transformed CLL or Richter's transformation.

In certain embodiments, the subject has a mutant Bruton's tyrosine kinase. In certain embodiments, the subject has a C481 mutant Bruton's tyrosine kinase. In certain embodiments, the subject has a C481S mutant Bruton's tyrosine kinase. In certain embodiments, the cancer is resistant to ibrutinib. Those of skill will recognize that certain ibrutinib-resistant cancers express a C481 mutant Bruton's tyrosine kinase, for instance C481S Bruton's tyrosine kinase. For example, in certain embodiments, the subject has a C481 mutant Bruton's tyrosine kinase and the cancer is chronic lymphocytic leukemia (CLL).

In certain embodiments, compounds described herein are capable of treating patients with ibrutinib-resistant cancer. In certain embodiments, the subject has a C481S, L528W, M437R, or V416L mutant Bruton's tyrosine kinase. In certain embodiments, the subject has a C481S mutant Bruton's tyrosine kinase. In certain embodiments, the subject has a L528W mutant Bruton's tyrosine kinase. In certain embodiments, the subject has a M437R mutant Bruton's tyrosine kinase. In certain embodiments, the subject has a V416L mutant Bruton's tyrosine kinase.

In certain embodiments, compounds described herein are capable of treating patients with a disease selected from the group consisting of Waldenstrom's macroglobulinemia, marginal zone lymphoma (MZL), mantle cell lymphoma (MCL), DLBCL, follicular lymphoma, and chronic lymphocytic leukemia. In certain embodiments, the disease is Waldenstrom's macroglobulinemia. In certain embodiments, the disease is marginal zone lymphoma (MZL). In certain embodiments, the disease is mantle cell lymphoma (MCL). In certain embodiments, the disease is DLBCL. In certain embodiments, the disease is follicular lymphoma. In certain embodiments, the disease is chronic lymphocytic leukemia.

In certain embodiments, compounds described herein are capable of treating patients with a disease or disorder selected from the group consisting of chronic lymphocytic leukemia (CLL) with BTK C481 mutation; CLL without BTK C481 mutation; mantle cell lymphoma (MCL), marginal zone lymphoma (MZL), Waldenstrom's macroglobulinemia (WM); follicular lymphoma (FL); and diffuse large B cell lymphoma (DLBCL). In certain embodiments, the disease or disorder is chronic lymphocytic leukemia (CLL) with BTK C481 mutation. In certain embodiments, the disease or disorder is CLL without BTK C481 mutation. In certain embodiments, the disease or disorder is mantle cell lymphoma (MCL). In certain embodiments, the disease or disorder is marginal zone lymphoma (MZL). In certain embodiments, the disease or disorder is Waldenstrom's macroglobulinemia (WM). In certain embodiments, the disease or disorder is follicular lymphoma (FL). In certain embodiments, the disease or disorder is diffuse large B cell lymphoma (DLBCL).

In another aspect, provided herein are methods of degrading Bruton's tyrosine kinase in a subject in need thereof. The methods comprise the step of orally administering to the subject an amount of a bifunctional compound capable of inducing proteolytic degradation of Bruton's tyrosine kinase. In certain embodiments, the amount is effective to degrade Bruton's tyrosine kinase in the subject. The Bruton's tyrosine kinase can be expressed in any cells or tissues of the subject. In certain embodiments, the Bruton's tyrosine kinase is expressed in splenocytes. In certain embodiments, the Bruton's tyrosine kinase is expressed in peripheral blood mononuclear cells.

In certain embodiments, the Bruton's tyrosine kinase is a mutant form. In certain embodiments, Bruton's tyrosine kinase comprises a C481 mutation. In certain embodiments, the Bruton's tyrosine kinase comprises a C481S mutation. In certain embodiments, the Bruton's tyrosine kinase is resistant to ibrutinib.

In another aspect, provided herein are methods of preventing B cell activation in a subject in need thereof. The methods comprise the step of orally administering to the subject an amount of a bifunctional compound capable of inducing proteolytic degradation of Bruton's tyrosine kinase. In certain embodiments, the amount is effective to prevent B cell activation. In certain embodiments, the B cell expresses CD69. In certain embodiments, the B cell expresses CD86. In certain embodiments, the B cell expresses CD69 and CD86.

In another aspect, provided herein are methods of degrading a mutant Bruton's tyrosine kinase. The methods comprise the step of contacting a cell expressing the mutant Bruton's tyrosine kinase with an amount of a bifunctional compound capable of inducing proteolytic degradation of Bruton's tyrosine kinase. In certain embodiments, the amount is effective to degrade the mutant Bruton's tyrosine kinase. In certain embodiments, the mutant Bruton's tyrosine kinase is a C481 mutant. In certain embodiments, the mutant Bruton's tyrosine kinase is a C481S mutant.

In certain embodiments, term “cancer” includes, but is not limited to, the following cancers: epidermoid Oral: buccal cavity, lip, tongue, mouth, pharynx, squamous cell carcinoma of the head and neck (HNSCC); Cardiac: sarcoma (angiosarcoma, fibrosarcoma, rhabdomyosarcoma, liposarcoma), myxoma, rhabdomyoma, fibroma, lipoma, and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma, non-small cell lung cancer (NSCLC); Gastrointestinal: gastric cancer, esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, microsatellite stable colorectal cancer (MSS CRC), rectum; Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma), metastatic castrate-resistant prostate cancer (mCRPC), muscle-invasive urothelial cancer; Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma (MM), malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical cancer, cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast, triple-negative breast cancer (TNBC), platinum-resistant epithelial ovarian cancer (EOC); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma) hairy cell; lymphoid disorders (e.g., mantle cell lymphoma, Waldenstrom's macroglobulinemia, Marginal zone lymphoma, and Follicular lymphoma); Skin: malilymphgnant melanoma, basal cell carcinoma, squamous cell carcinoma, Karposi's sarcoma, keratoacanthoma, moles dysplastic nevi, lipoma, angioma, dermatofibroma, keloids, psoriasis; Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; Adrenal glands: neuroblastoma; and metastatic melanoma.

In certain embodiments, the cancer is B-cell malignancy. In certain embodiments, the B-cell malignancy is diffuse large B-cell lymphoma (DLBCL). In certain embodiments, the B-cell malignancy is mediastinal B-cell lymphoma. In certain embodiments, the B-cell malignancy is follicular lymphoma. In certain embodiments, the B-cell malignancy is chronic lymphocytic leukemia (CLL)/small lymphocytic lymphoma (SLL). In certain embodiments, the B-cell malignancy is mantle cell lymphoma (MCL). In certain embodiments, the B-cell malignancy is marginal zone lymphomas. In certain embodiments, the B-cell malignancy is extranodal marginal zone B-cell lymphoma. In certain embodiments, the B-cell malignancy is nodal marginal zone B-cell lymphoma. In certain embodiments, the B-cell malignancy is Burkitt lymphoma. In certain embodiments, the B-cell malignancy is lymphoplasmacytic lymphoma (Waldenstrom macroglobulinemia). In certain embodiments, the B-cell malignancy is hairy cell leukemia. In certain embodiments, the B-cell malignancy is primary central nervous system (CNS) lymphoma. In certain embodiments, the B-cell malignancy is primary intraocular lymphoma.

In certain embodiments, the cancer is multiple myeloma. In certain embodiments, the cancer is myelodysplastic syndrome. In certain embodiments, the cancer is karposi sarcoma.

In certain embodiments, the disease or disorder is graft-versus-host disease (GVHID).

In certain embodiments, provided herein are methods of degrading a mutant Bruton's tyrosine kinase. The methods comprise the step of contacting a cell expressing the mutant Bruton's tyrosine kinase with an amount of a bifunctional compound capable of inducing proteolytic degradation of Bruton's tyrosine kinase. In certain embodiments, the amount of a bifunctional compound capable of inducing proteolytic degradation of Bruton's tyrosine kinase is the amount effective to degrade the mutant Bruton's tyrosine kinase. In certain embodiments, the mutant Bruton's tyrosine kinase is a C481 mutant. In certain embodiments, the mutant Bruton's tyrosine kinase is a C481S mutant. The contacting can be in vitro or in vivo. In certain embodiments, the contacting is in vitro. In certain embodiments, the contacting is in vivo. In certain embodiments, the contacting is in a subject in need thereof.

### Compounds

The methods provided herein comprise administration of a compound. The compound can be any compound described herein. In certain embodiments, the compound comprises at least two moieties. One moiety is capable of specifically binding Bruton's tyrosine kinase (BTK). The other moiety is capable of recruiting an ubiquitin ligase to degrade the BTK. In certain embodiments, the ubiquitin ligase is an E3 ligase. In certain embodiments, the ubiquitin ligase is cereblon (CRBN) or comprises cereblon as a component.

In the methods, the compound can be a compound of Formula (A1)

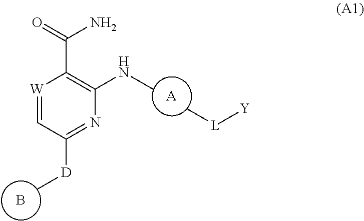

or a pharmaceutically acceptable salt thereof, wherein W is CH or N; D is a bond or a linker; Ring A is aryl or heteroaryl; Ring B is aryl or heteroaryl; L is a bond or a linker; and Y is a moiety capable of binding an ubiquitin ligase.

In the methods, the compound can be a compound of Formula (A)

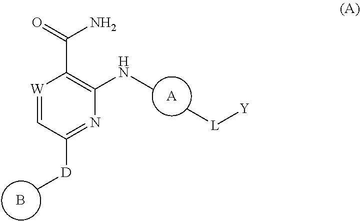

or a pharmaceutically acceptable salt thereof, wherein W is CH or N; D is a bond or —NH—; Ring A is phenyl, a 9-10 membered bicyclic aryl, a 5-6 membered partially or fully unsaturated monocyclic heterocycle, or a 9-10 membered bicyclic heteroaryl, wherein the monocyclic heterocycle and bicyclic heteroaryl of Ring A each possess one to three heteroatoms independently selected from N, O, or S, wherein Ring A is optionally and independently substituted with up to three substituents selected from halo, —CN, —COOH, NH₂, and optionally substituted C₁₋₆ alkyl; Ring B is a phenyl, a 5-6 membered heteroaryl, a 4-6 membered heterocycloalkyl, or a 8-10 membered (e.g., 8-9 membered or 9-10 membered) spiro bicyclic heterocycle, wherein Ring B is optionally substituted, and wherein the heteroaryl and heterocycloalkyl of Ring B has one to three heteroatoms independently selected from N, O, or S; L is —X¹—X²—X³—X⁴—X⁵—; X¹ is a bond, —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro or fused bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S; X³ is a bond, —C₁₋₈ alkyl-, —C≡C—, 4-6 membered cycloalkyl, —N(R)—, —N(R)—C(O)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, a 5-6 membered saturated, partially unsaturated, or fully unsaturated carbocycle, or a 5-6 membered saturated, partially unsaturated, or fully unsaturated heterocycle having one to three heteroatoms independently selected from N, O, or S; X⁵ is a bond, —C₁₋₄ alkyl-, —N(R)—, —O—, —C(O)—, or —C(O)—N(R)—; each R is independently —H or —C₁₋₃ alkyl (e.g., methyl, ethyl, propyl, or iso-propyl); and each of m, n, and p is independently an integer from one to three (e.g., one, two, or three); and Y is

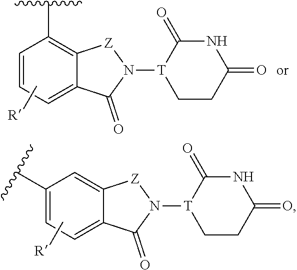

wherein each T is independently CH or N; and each Z is independently —CH₂— or —C(O)—; and each R′ is hydrogen, methyl, or NH₂.

With the exception of the moieties of group R, all moieties of the linking group L as defined in the compound of Formula (A) are bivalent moieties unless otherwise specified. For example, any alkyl (e.g., n-propyl, n-butyl, n-hexyl, and the like), aryl (e.g., phenyl), cycloalkyl (e.g., cyclopropyl, cyclohexyl, and the like), heteroaryl, heterocycloalkyl (e.g., piperidine, piperazine, and the like) that is present in L is bivalent unless otherwise specified.

In some embodiments, Ring B is an optionally substituted 5-6 membered heterocycloalkyl having one to two nitrogen atoms. For example, Ring B is piperidine-yl, piperizine-yl, or pyrrolidine-yl, any of which is optionally substituted.

In some embodiments, Ring B is an optionally substituted 5-6 membered heteroaryl having one to two heteroatoms independently selected from N and S. For example, Ring B is pyridine-yl, pyrazine-yl, or pyrimidine, any of which is optionally substituted.

In some embodiments, Ring B is

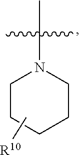

wherein R¹⁰ is halo, —H, —C₁₋₅ alkyl (e.g., —C₁₋₃ alkyl), 3-6 membered cycloalkyl, 5-6 membered heterocycloalkyl, —CN, —OH, —CF₃, —CH₂OH, —CH₂CH₂OH, —C(O)OH,

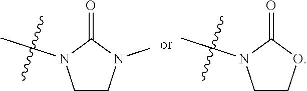

In some embodiments, Ring B is

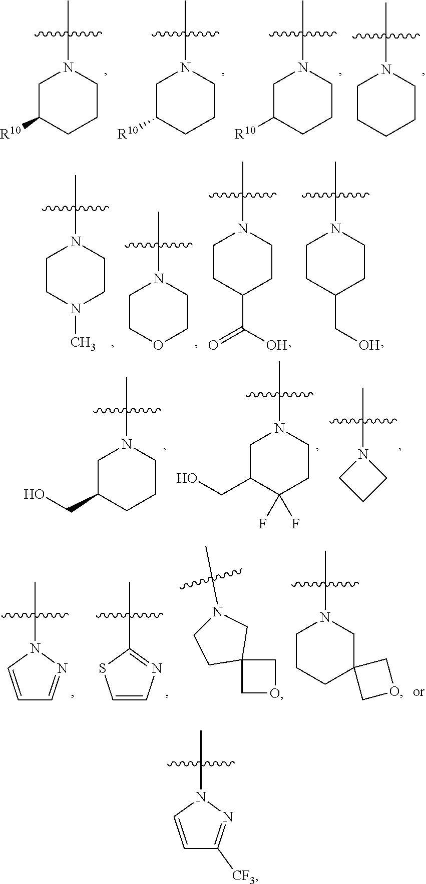

wherein R¹⁰ is

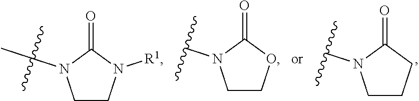

and wherein R¹ is a C₁₋₄ alkyl group. For example, Ring B is

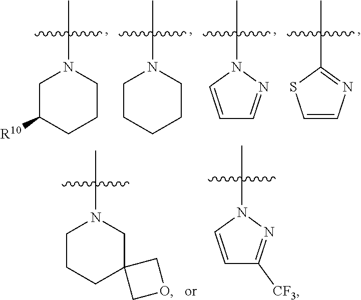

wherein R¹⁰ is

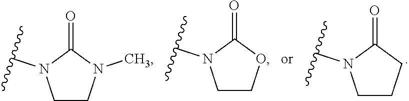

And, in some instances, Ring B is

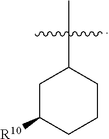

In other instances, R¹⁰ is

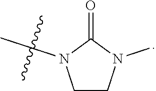 [SMILES: *N1CCN(C)C1=O]

In some embodiments, Ring A is

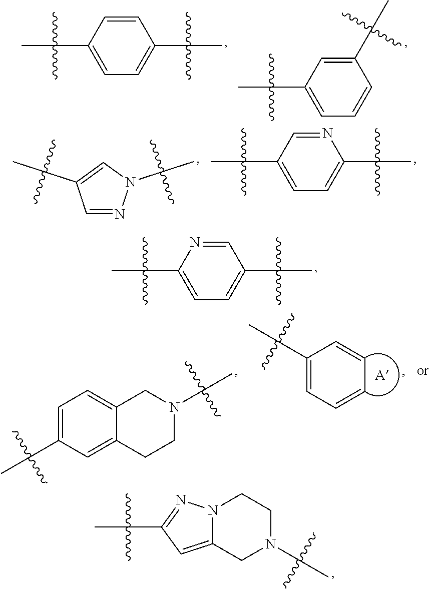

wherein Ring A′ together with the phenyl ring to which Ring A′ is fused form a 9-10 membered bicyclic aryl or a 9-10 membered bicyclic heteroaryl wherein the bicyclic heteroaryl (i.e., the bicyclic heteroaryl including Ring A′) has one to three heteroatoms independently selected from N, O, or S. For example, Ring A is

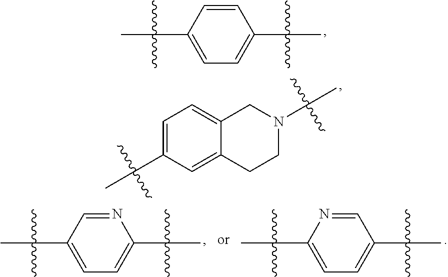

In some embodiments, at least one of X¹, X², and X⁵ is —N(R)—, —C(O)—N(R)—, or —CH₂—.

In some embodiments, X¹ is —C(O)—N(R)—.

In some embodiments, X² is —(O—CH₂—CH₂)ₙ—, —(CH₂—CH₂—O)ₙ—, or —C₁₋₅ alkyl-.

In some embodiments, X³ is a bond, —C≡C—, —C₁₋₄ alkyl-, or —(R)—.

In some embodiments, X⁴ is a bond, —CH₂—, or —N(R)—.

In some embodiments, X⁵ is a bond.

In some embodiments, X¹ is —(O—CH₂—CH₂—CH₂)ₘ—, m is one, and X² is —C(O)—N(R)—.

In some embodiments X¹ is —CH₂—, —C(O)—,

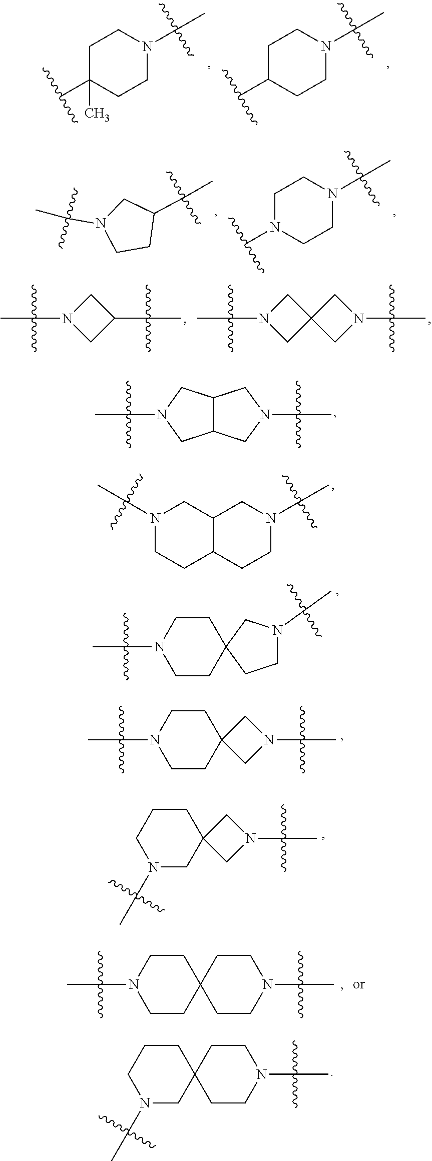

In some embodiments, X² is a bond, —C(O)—, —C₁₋₅ alkyl-,

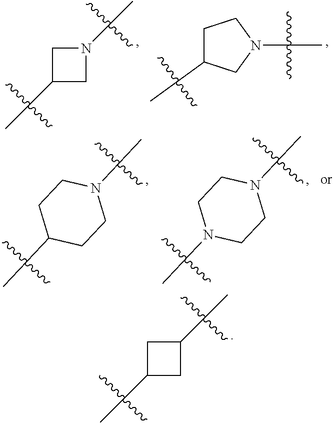 [SMILES: *C1CN(*)C1 ; *C1CCN(*)C1 ; *C1CCN(*)CC1 ; *N1CCN(*)CC1 ; *C1CC(*)C1]

In some embodiments, X³ is bond, —C₁₋₄ alkyl-, 4-6 membered cycloalkyl, or —N(R)—.

In some embodiments, X³ is a bond, —C₁₋₄ alkyl-, —NH—,

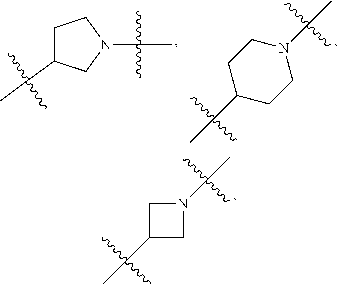 [SMILES: *C1CCN(*)C1 ; *C1CCN(*)CC1 ; *C1CN(*)C1]

### or —C≡C—.

In some embodiments, X⁴ is a bond,

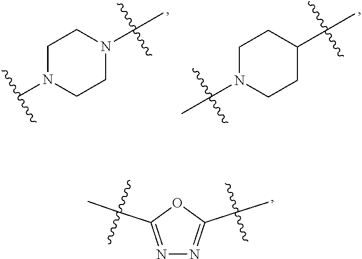 [SMILES: *N1CCN(*)CC1 ; *C1CCN(*)CC1 ; *c1nnc(*)o1]

—C₁₋₄ alkyl-, —CH₂—CH₂—N(R)—, or —N(R)—.

In some embodiments, X⁵ is a bond, —C₁₋₄ alkyl-, —N(R)—, or —C(O)—N(R)—.

In some embodiments, L is

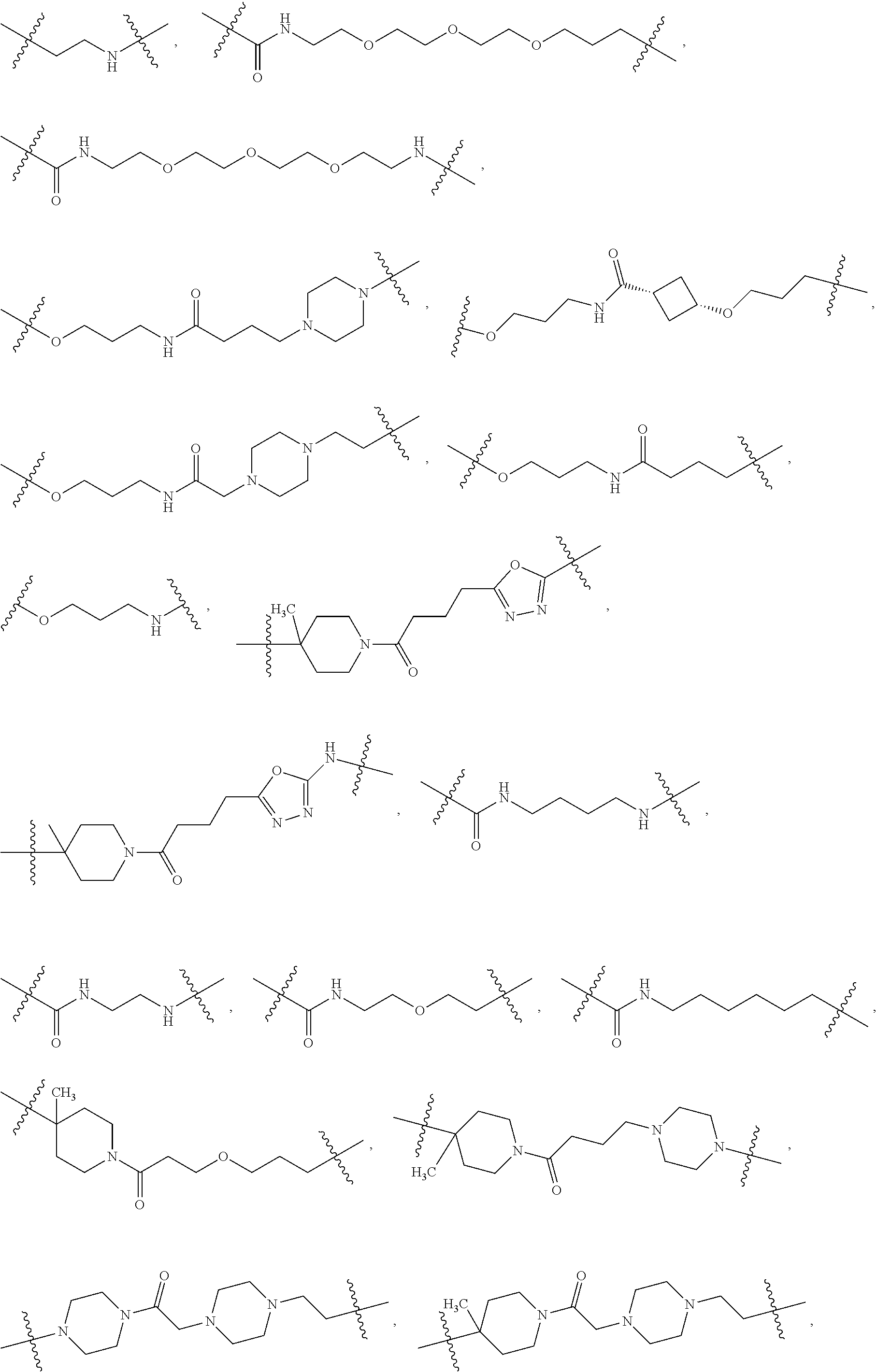

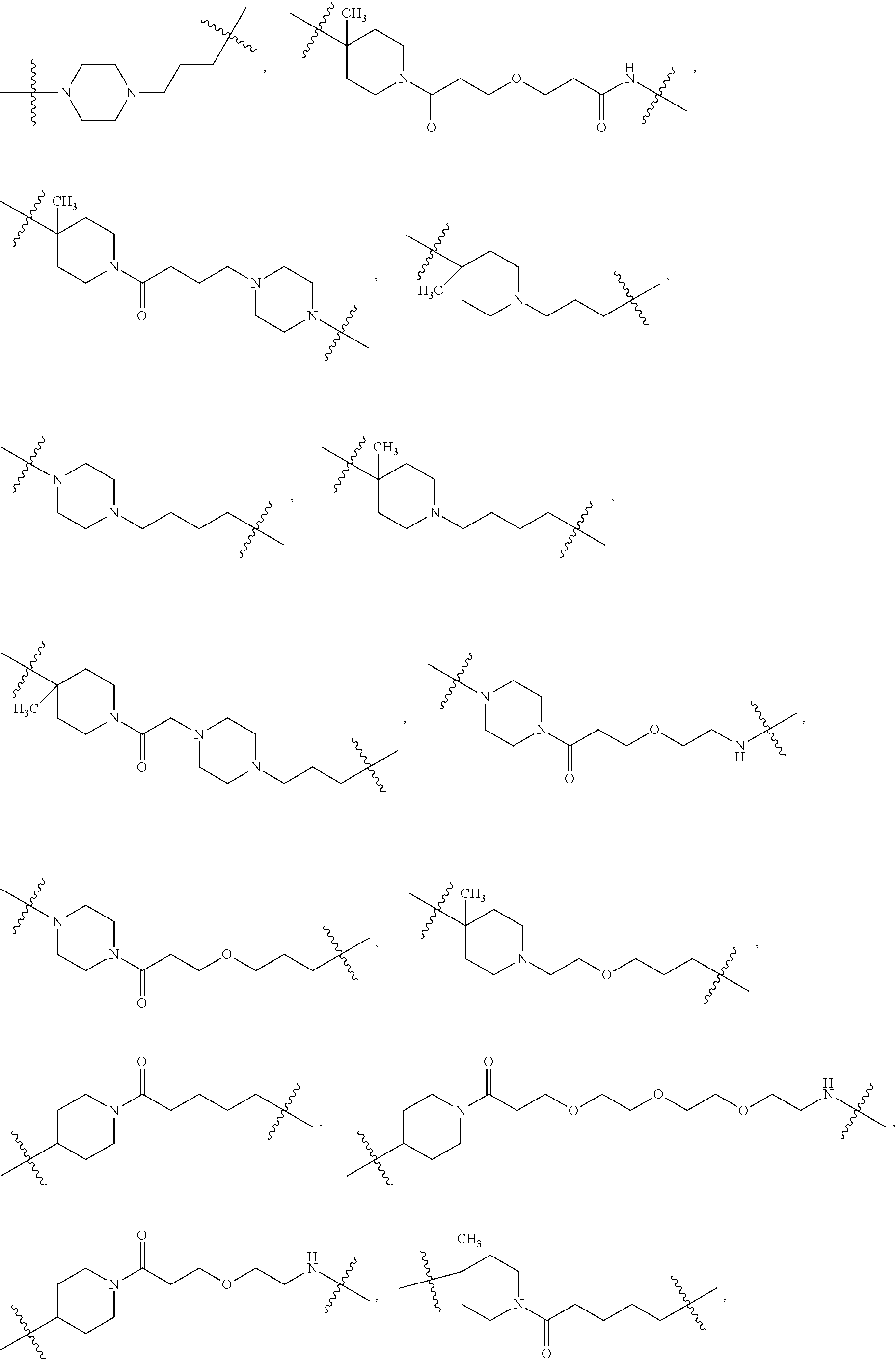

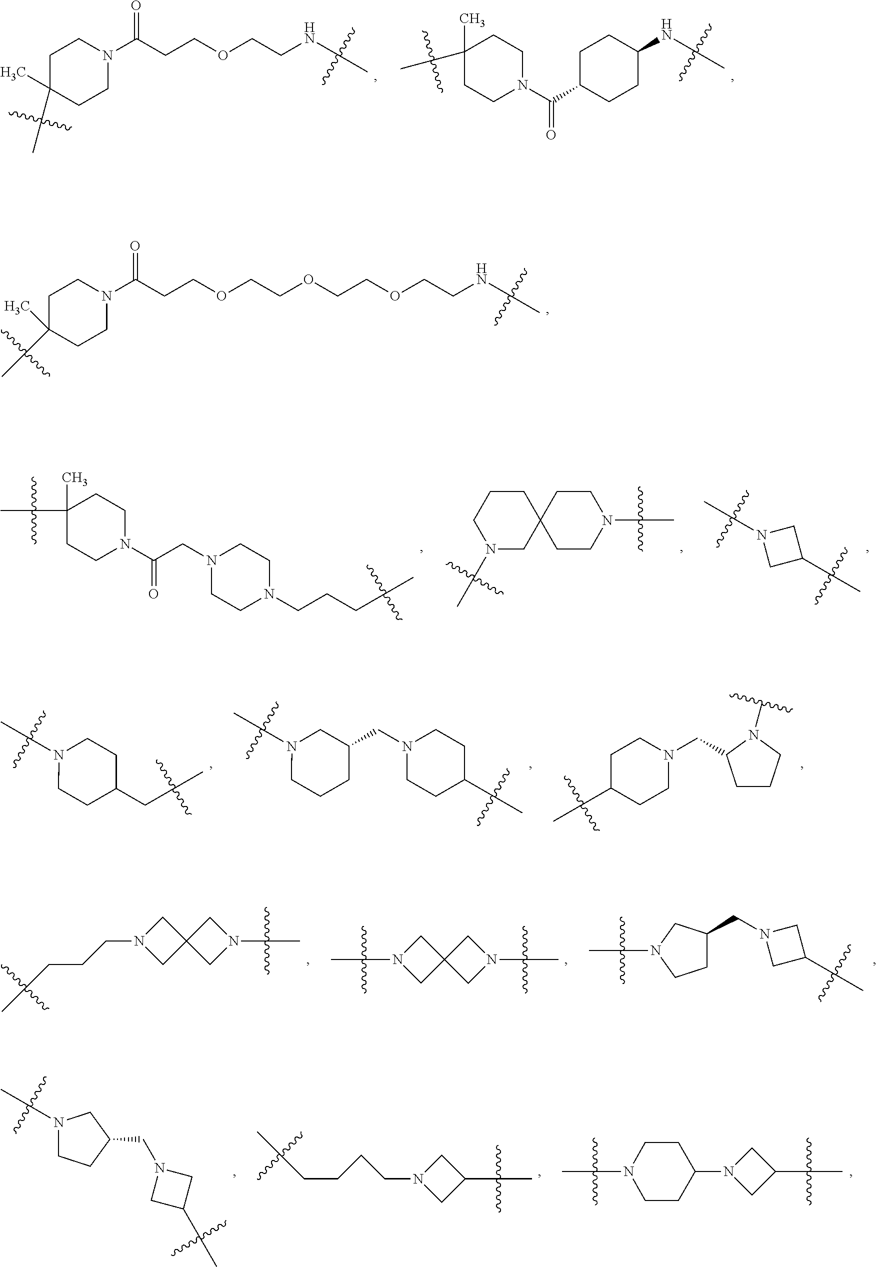

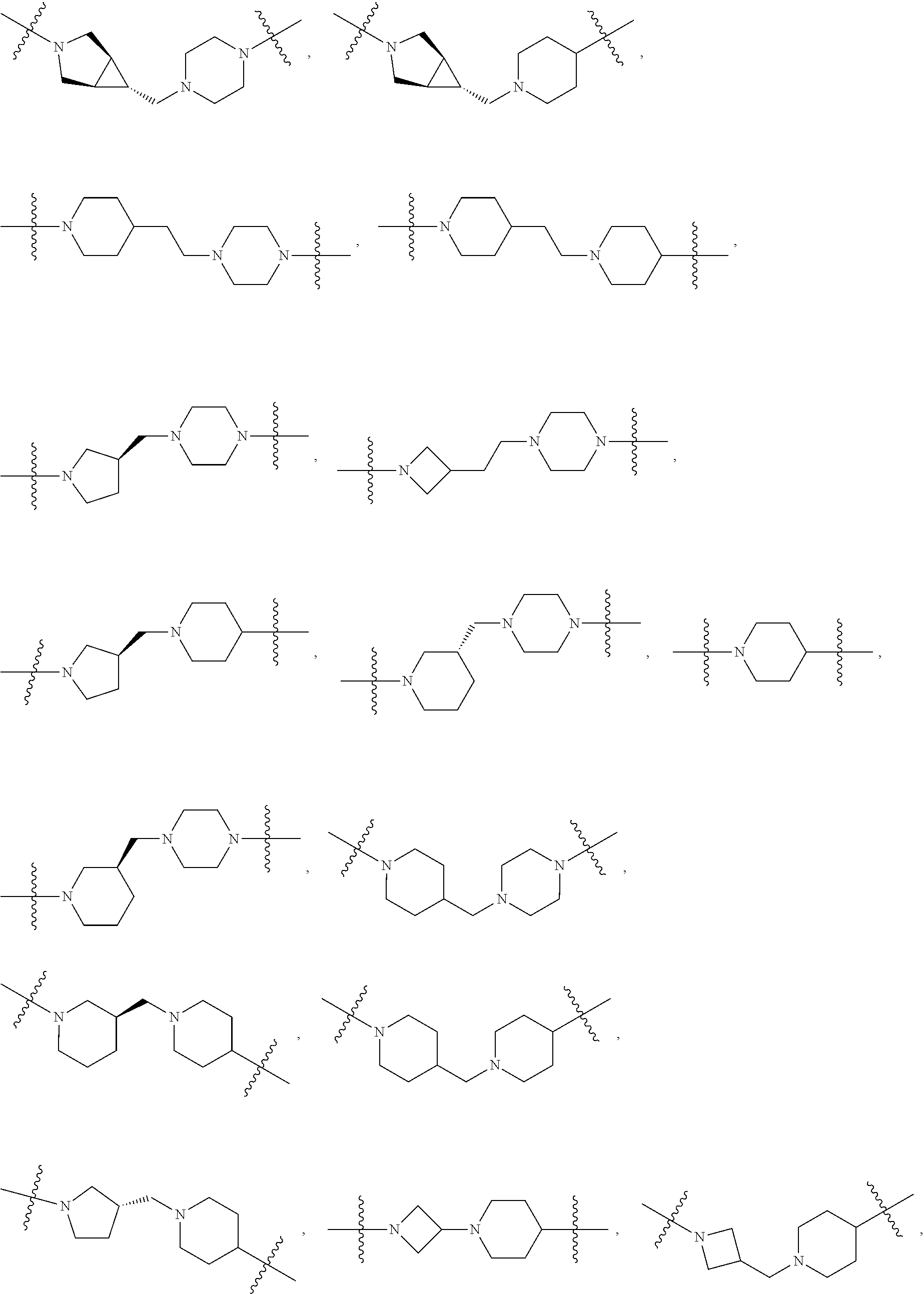

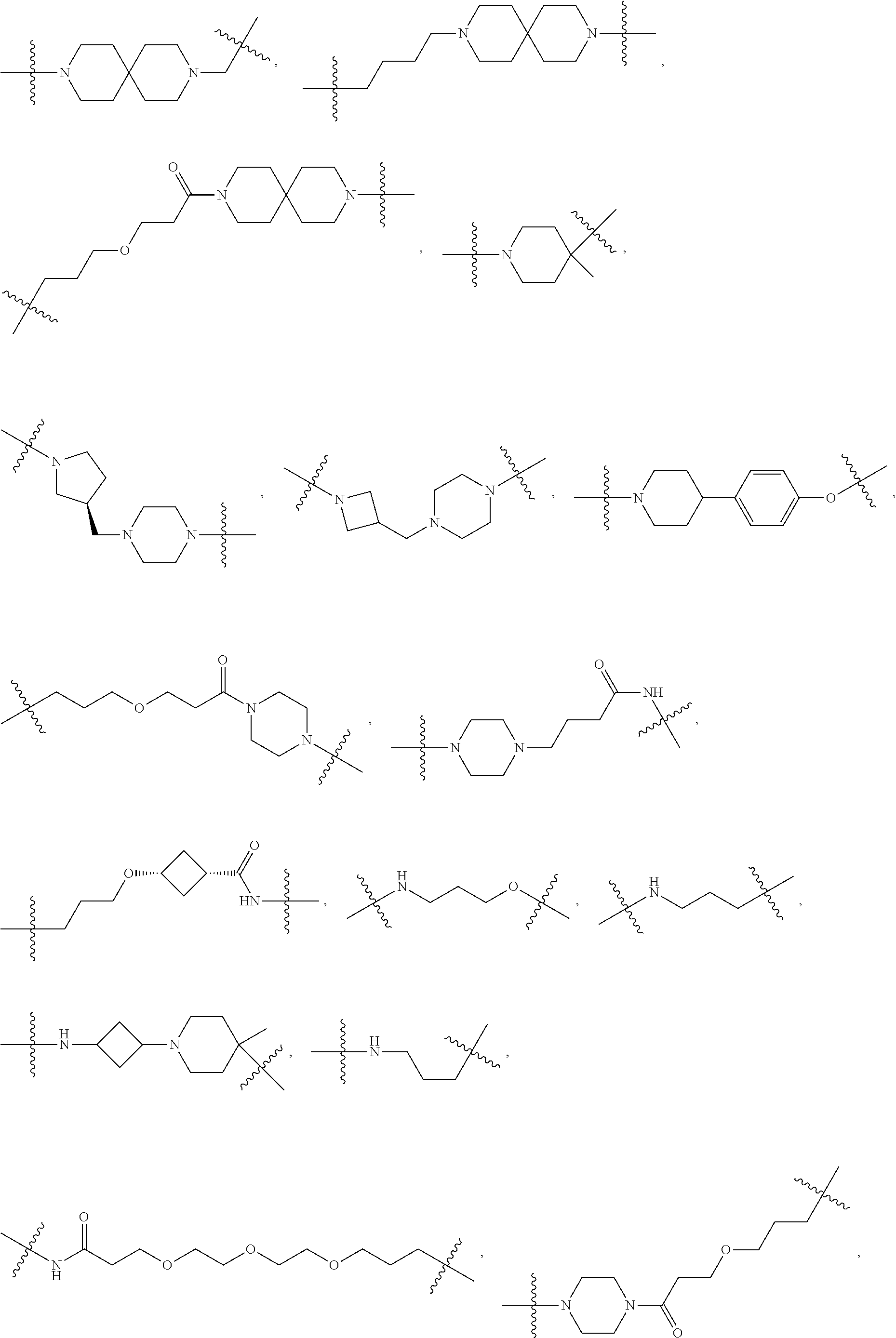

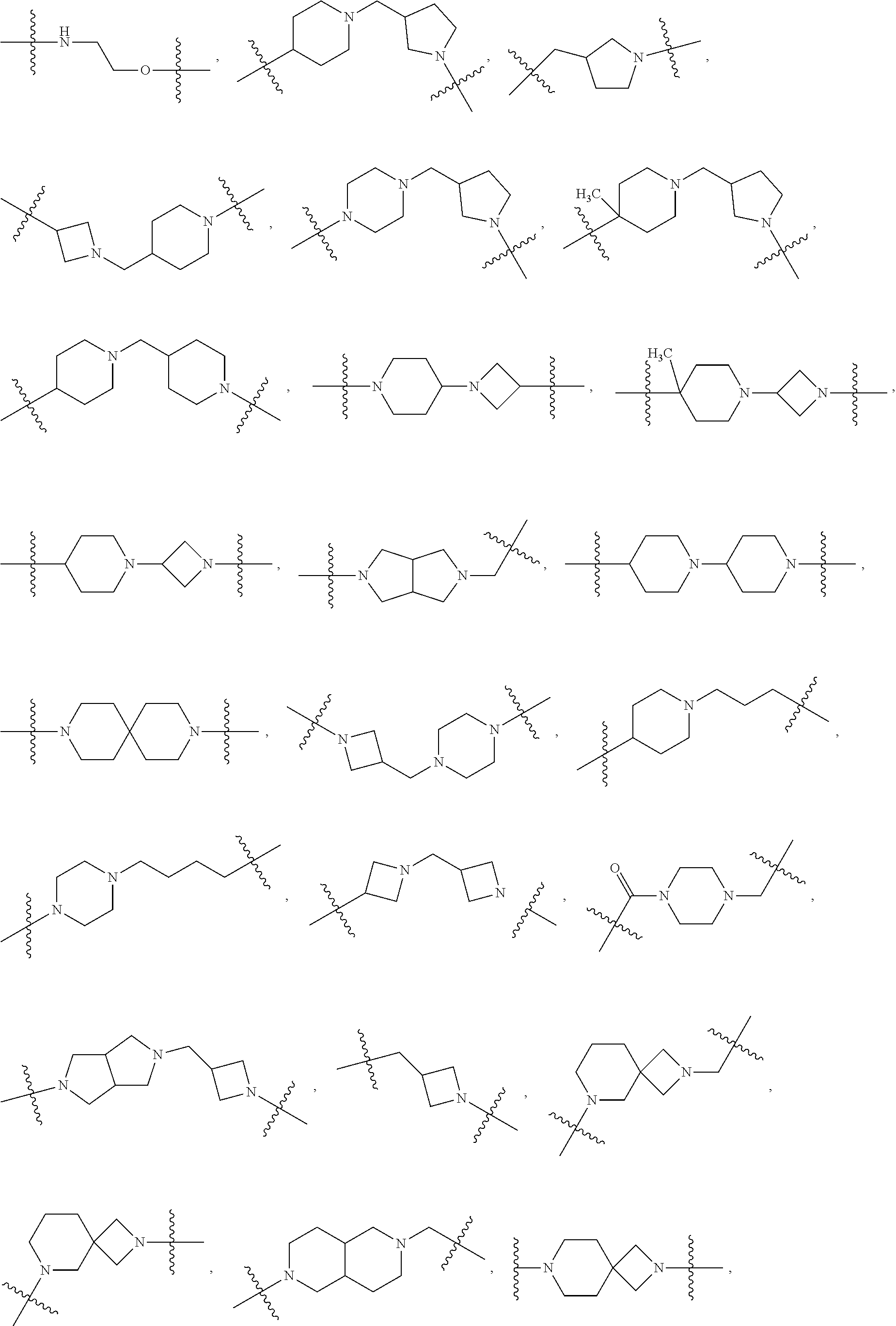

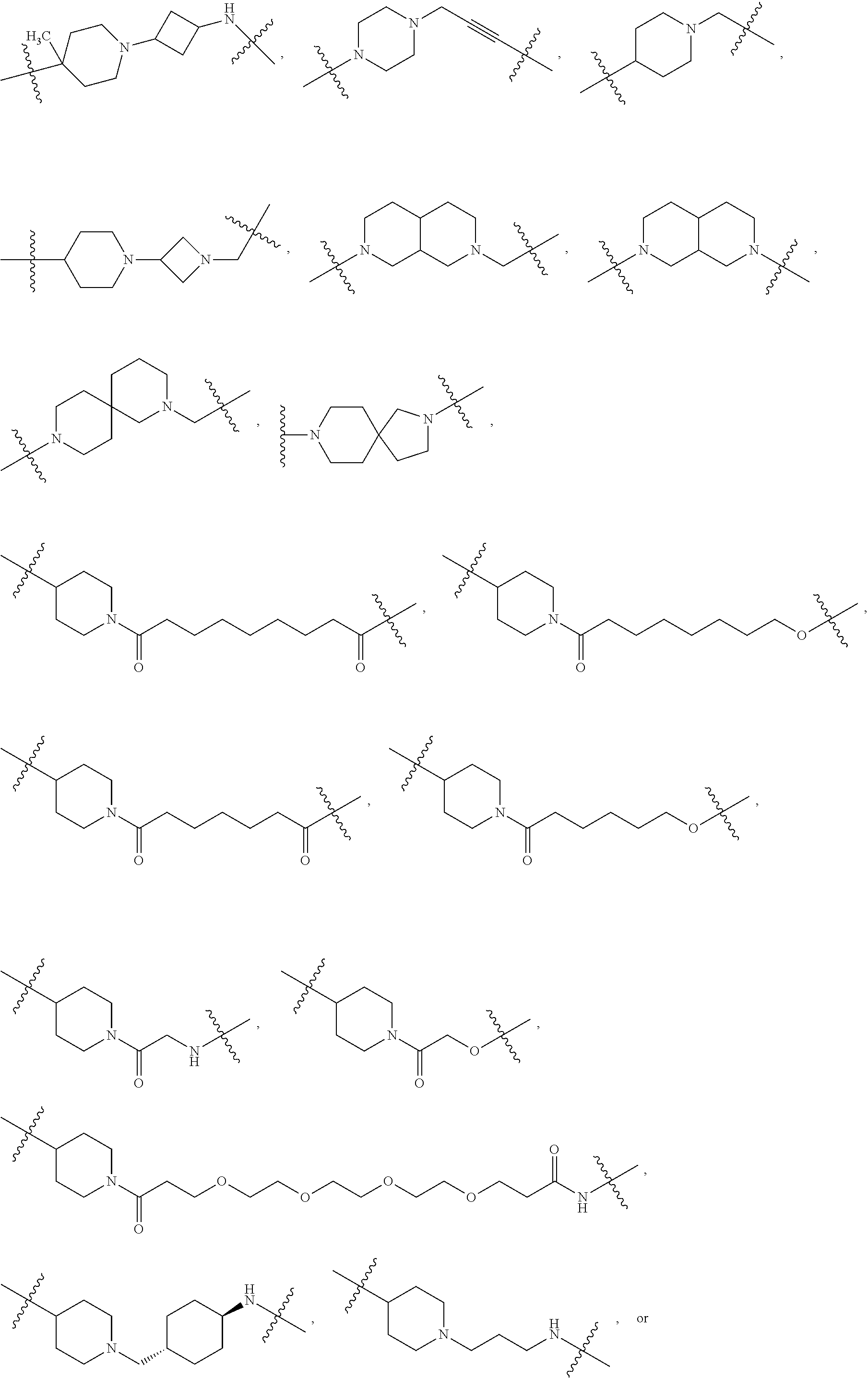

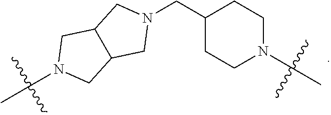 [SMILES: *N1CCC(CN2CC3CN(*)CC3C2)CC1]

In some embodiments, Y is

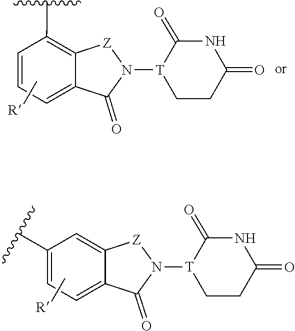

wherein each T is independently CH or N; and each Z is independently —CH₂— or —C(O)—; and each R′ is hydrogen, methyl, or NH₂.

In some embodiments, Y is

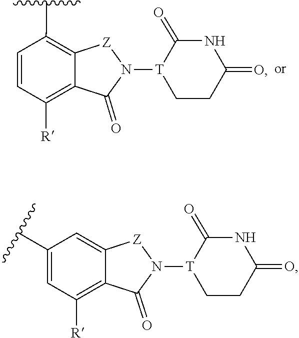

wherein each T is independently CH or N; and each Z is independently —CH₂— or —C(O)—; and each R′ is hydrogen, methyl, or NH₂.

This disclosure also provides a compound of Formula (B)

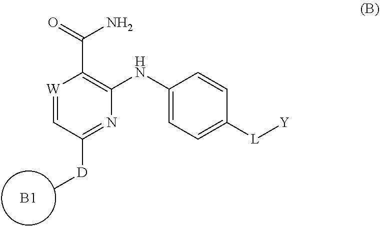

or a pharmaceutically acceptable salt thereof, wherein W is CH or N; D is a bond or —NH—; Ring B1 is a 4-6 membered, fully saturated, partially unsaturated, or fully unsaturated monocyclic heterocycle or a 8-10 membered, fully saturated, spiro bicyclic heterocycle, wherein Ring B1 has one to three heteroatoms independently selected from N, O, or S, and is optionally substituted with one to three groups selected from halo, —CH₃, —CF₃, —C(O)OH, —CH₂OH, or a 5-membered heterocycloalkyl optionally substituted with oxo and having one to two heteroatoms independently selected from N or O; L is —X¹—X²—X³—; X¹ is —C(O)—N(R), —N(R)—C(O), (O—CH₂—CH₂)ₘ—, —O(C₆H₄)—, —(O—CH₂—CH₂—CH₂)ₘ—, —C₁₋₅ alkyl-, 7-12 membered spiro or fused bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃; each R is independently —H or —C₁₋₃ alkyl; each of m, n, and p is independently an integer from one to three; and Y is as described above.

In some embodiments, Ring B1 is

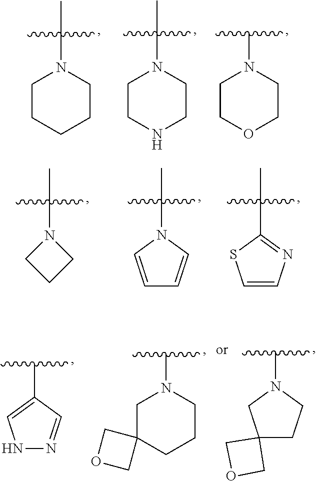

and Ring B1 is optionally substituted one to three groups selected from —CH₃, —CH₂OH, —CH₂CH₂OH, —C(O)OH, —CF₃, —F,

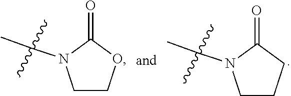 [SMILES: *N1CCOC1=O ; *N1CCCC1=O]

For example, Ring B1 is

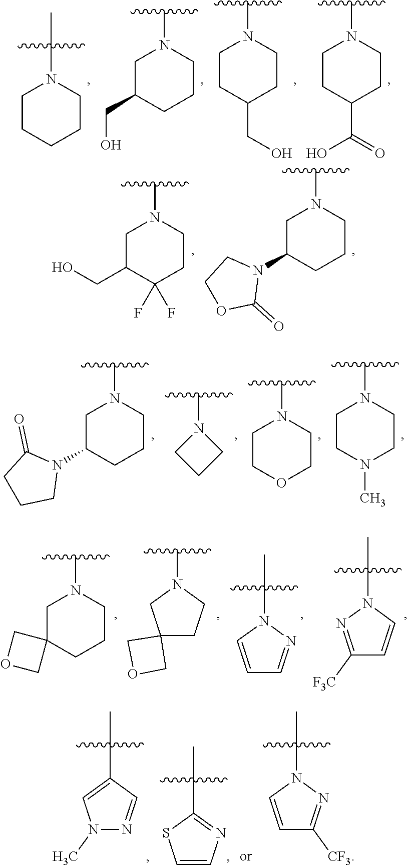

In other examples, Ring B1 is

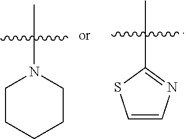 [SMILES: *N1CCCCC1 ; *c1nccs1]

In some embodiments, X¹ is

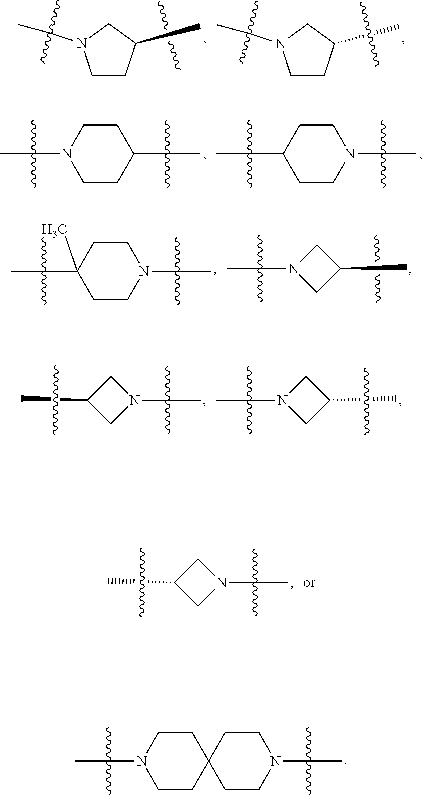

In some embodiments, X² is a bond, —C₁₋₅ alkyl-, 4-6 membered monocyclic cycloalkyl, or 4-6 membered monocyclic heterocycloalkyl having one to two heteroatoms independently selected from N, O, or S. For example, X² is a bond, —C₁₋₃ alkyl-, —C(O)—,

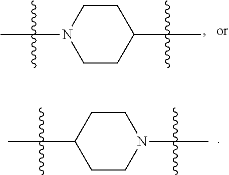 [SMILES: *C1CCN(*)CC1 ; *C1CCN(*)CC1]

In some embodiments, X³ is a bond, —C₁₋₄ alkyl-, —N(R)—, —(O—CH₂—CH₂)ₚ—, —(CH₂—CH₂—O)ₚ—, or a 4-6 membered heterocycloalkyl having one to two heteroatoms independently selected from N, O, or S, wherein the heterocycloalkyl is optionally substituted with —CH₃. For example, X³ is a bond,

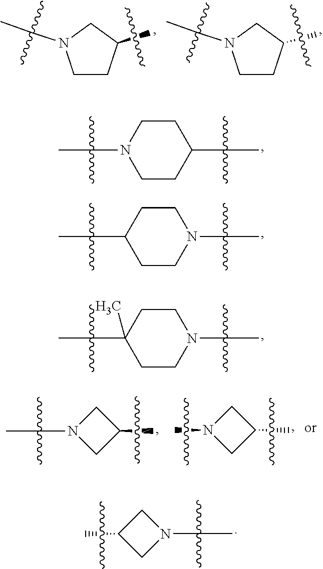

In some embodiments L is

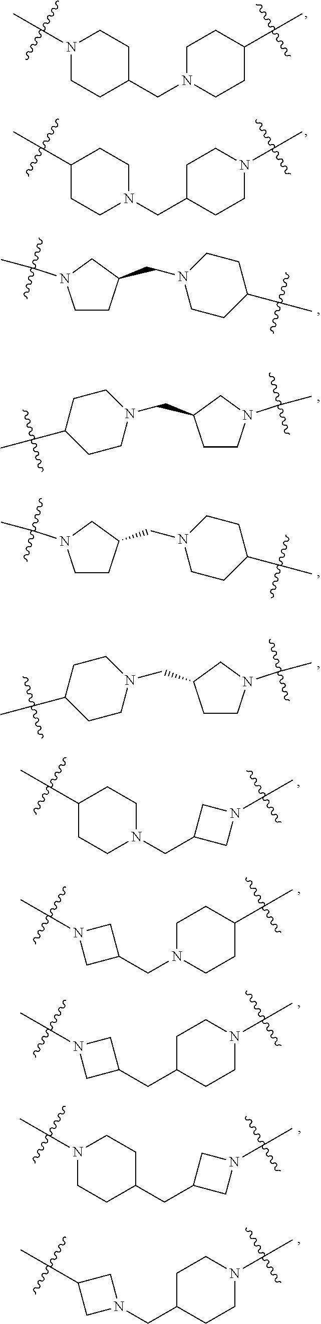

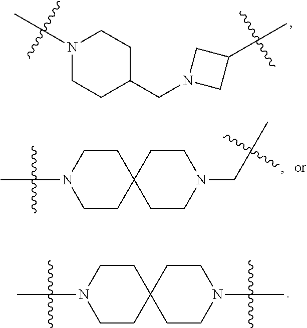 [SMILES: *C1CN(CC2CCN(*)CC2)C1 ; *CN1CCC2(CCN(*)CC2)CC1 ; *N1CCC2(CC1)CCN(*)CC2]

In some embodiments, W is N and D is a bond.

This disclosure also provides a compound of Formula (C)

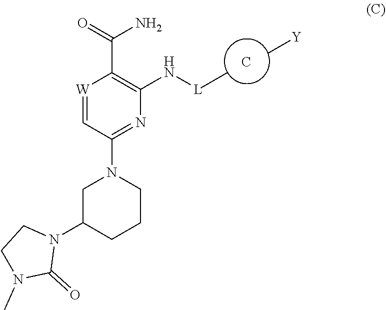

or a pharmaceutically acceptable salt thereof, wherein W is CH or N; Ring C is phenyl or a saturated, partially unsaturated, or fully unsaturated 5-6 membered monocyclic heterocycle having one to two heteroatoms independently selected from N, O, or S, wherein each of the phenyl and heterocycle of Ring C is optionally substituted; L is —X¹—X²—X³—; X¹ is —C(O)—N(R), —N(R)—C(O), (O—CH₂—CH₂)ₘ—, O—(C₆H₄), (O—CH₂—CH₂—CH₂)ₘ—, —C₁₋₅ alkyl-, 7-12 membered spiro bicyclic heterocycloalkyl having one to three heteroatoms independently selected from N, O, or S, or 4-6 membered monocyclic heterocycloalkyl having one to two heteroatoms independently selected from N, O, or S, wherein each of the bicyclic heterocycloalkyl and the monocyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃; each R is independently —H or —C₁₋₃ alkyl; and each of m, n, and p is independently an integer from one to three.

In some embodiments, W is N.

In some embodiments, Ring C is

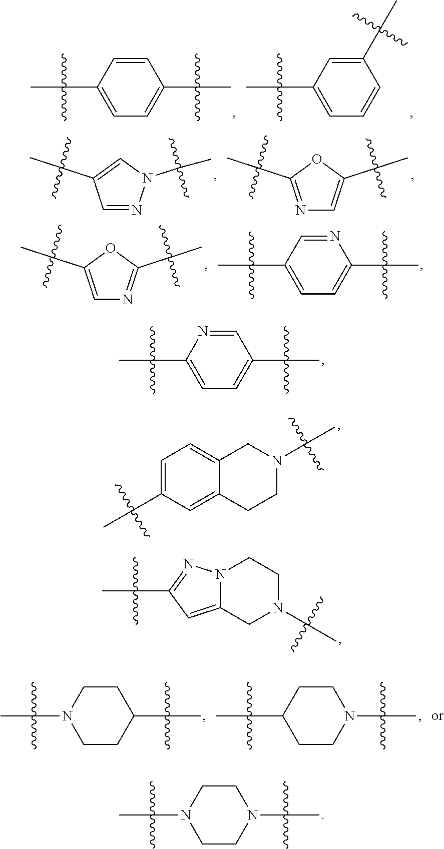

For example, Ring C is

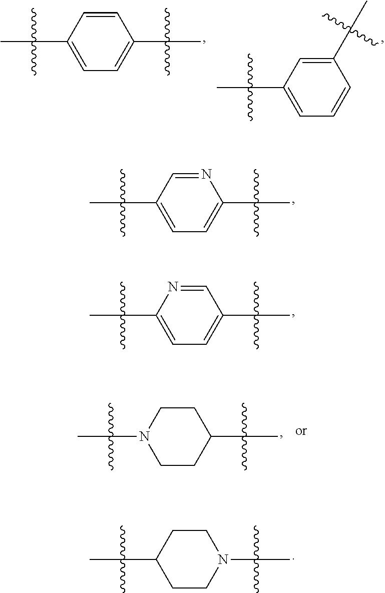 [SMILES: *c1ccc(*)cc1 ; *c1cccc(*)c1 ; *c1ccc(*)nc1 ; *c1ccc(*)nc1 ; *C1CCN(*)CC1 ; *C1CCN(*)CC1]

In other examples, Ring C is

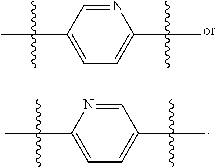

In some embodiments, X¹ is a 4-6 membered monocyclic heterocycloalkyl having one to two heteroatoms independently selected from N, O, or S. For example, X¹ is

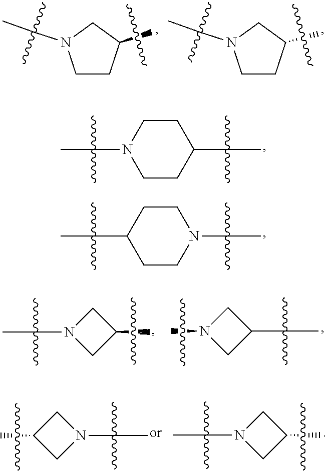

In some examples, X¹ is

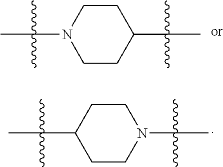

In some embodiments, X² is a bond, —C₁₋₅ alkyl-, 4-6 membered monocyclic cycloalkyl, or 4-6 membered monocyclic heterocycloalkyl having one to two heteroatoms independently selected from N, O, or S. For example, X² is a bond or —C₁₋₃ alkyl- (e.g., —CH₂—).

In some embodiments, X³ is a 4-6 membered cycloalkyl, —N(R)—, or a 4-6 membered heterocycloalkyl having one to two heteroatoms independently selected from N, O, or S, wherein the heterocycloalkyl is optionally substituted with —CH₃. For example, X³ is

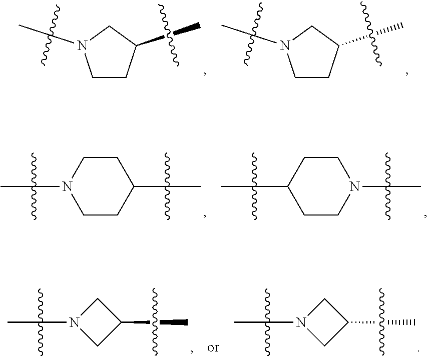

In other embodiments, X³ is

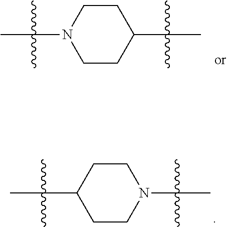 [SMILES: *C1CCN(*)CC1 ; *C1CCN(*)CC1]

In some embodiments, L is

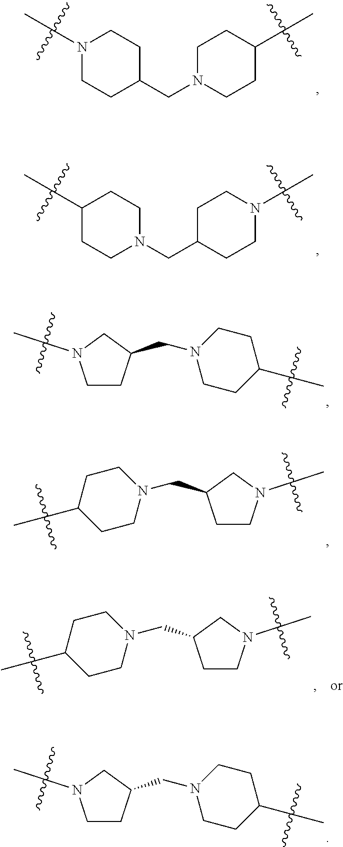 [SMILES: *C1CCN(CC2CCN(*)CC2)CC1 ; *C1CCN(CC2CCN(*)CC2)CC1 ; *C1CCN(C[C@H]2CCN(*)C2)CC1 ; *C1CCN(C[C@@H]2CCN(*)C2)CC1 ; *C1CCN(C[C@H]2CCN(*)C2)CC1 ; *C1CCN(C[C@@H]2CCN(*)C2)CC1]

For example, L is

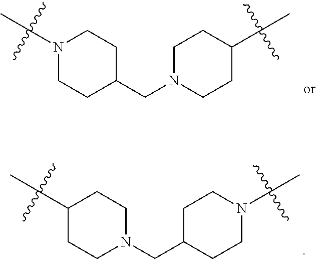 [SMILES: *C1CCN(CC2CCN(*)CC2)CC1 ; *C1CCN(CC2CCN(*)CC2)CC1]

This disclosure also provides a compound of Formula (D)

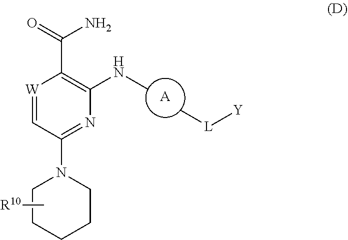

or a pharmaceutically acceptable salt thereof, wherein W is CH or N; Ring A is

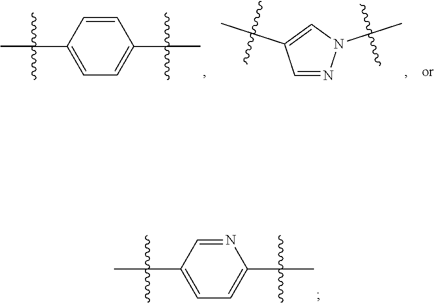 [SMILES: *c1ccc(*)cc1 ; *c1cnn(*)c1 ; *c1ccc(*)nc1]

L is —X¹—X²—X³—; X¹ is —C₁₋₅ alkyl- or 4-6 membered monocyclic heterocycloalkyl having one to two heteroatoms independently selected from N, O, or S, wherein the monocyclic heterocycloalkyl of X¹ is optionally substituted with —CH₃; X² is a bond, —C₁₋₅ alkyl-, or 4-6 membered monocyclic heterocycloalkyl having one to two heteroatoms independently selected from N, O, or S, wherein the monocyclic heterocycloalkyl of X is optionally substituted with —CH₃; X³ is a bond, —C₁₋₄ alkyl-, 4-6 membered monocyclic cycloalkyl, or 4-6 membered heterocycloalkyl having one to two heteroatoms independently selected from N, O, or S, wherein the heterocycloalkyl is optionally substituted with —CH₃; Y is as described herein; and R¹⁰ is halo, —H, —C₁₋₅ alkyl, 3-6 membered cycloalkyl, 5-6 membered heterocycloalkyl, —CN, —OH, —CF₃, —CH₂OH, —CH₂CH₂OH, —C(O)OH,

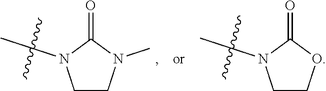

In some embodiments, Ring A is

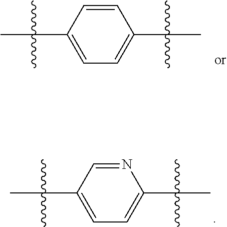 [SMILES: *c1ccc(*)cc1 ; *c1ccc(*)nc1]

In some embodiments, X¹ is a 4-6 membered monocyclic heterocycloalkyl having one to two heteroatoms independently selected from N, O, or S, wherein the monocyclic heterocycloalkyl of X¹ is optionally substituted with —CH₃. For example, X¹ is

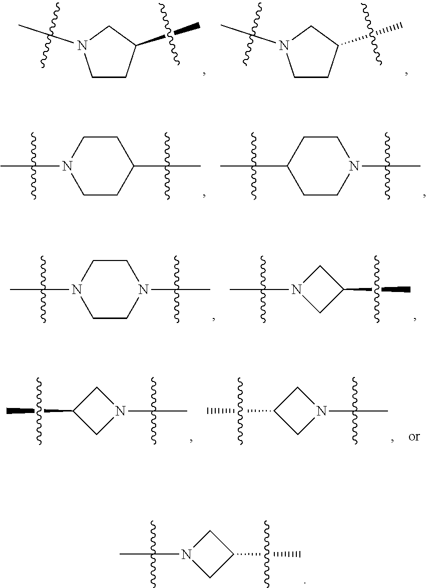

In some embodiments, X² is a bond, —C₁₋₅ alkyl-, 4-6 membered monocyclic cycloalkyl, or 4-6 membered monocyclic heterocycloalkyl having one to two heteroatoms independently selected from N, O, or S. For example, X² is a bond or —C₁₋₄ alkyl-.

In some embodiments, X³ is a bond, a 4-6 membered monocyclic cycloalkyl, or 4-6 membered monocyclic heterocycloalkyl having one to two heteroatoms independently selected from N, O, or S. For example, X³ is

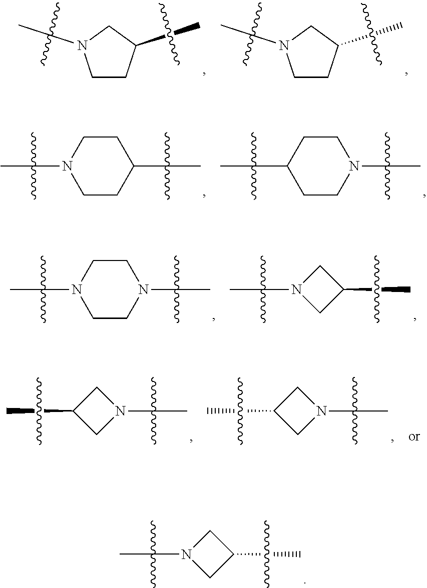

In some embodiments, L is

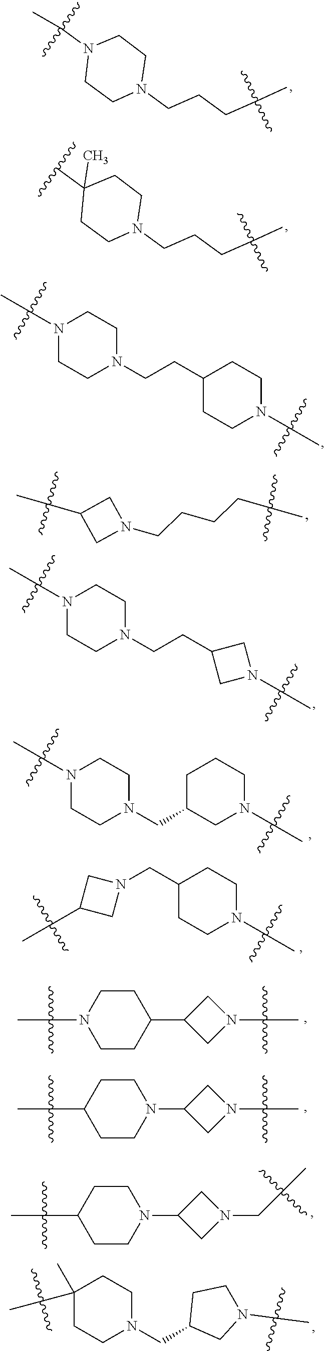

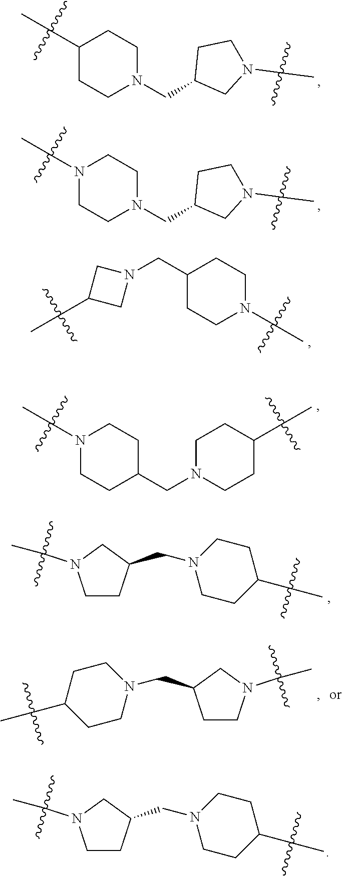

In some embodiments, R¹⁰ is halo, —H, —C₁₋₅ alkyl (e.g., —C₁₋₃ alkyl), 3-6 membered cycloalkyl, 5-6 membered heterocycloalkyl, —CN, —OH, —CF₃, —CH₂OH, —C(O)OH, or —CH₂CH₂OH. For instance, R¹⁰ is halo, —H, C₁₋₃ alkyl, CF₃, —CH₂OH, —C(O)OH, or —CH₂CH₂OH. In other instances, R¹⁰ is

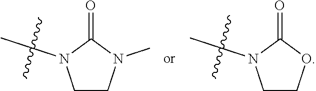

In some embodiments, R¹⁰ is

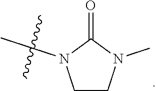 [SMILES: *N1CCN(C)C1=O]

In some embodiments, R¹⁰ is

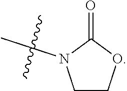 [SMILES: *N1CCOC1=O]

In some embodiments, the compound of Formula (D) is a compound of (D-1)

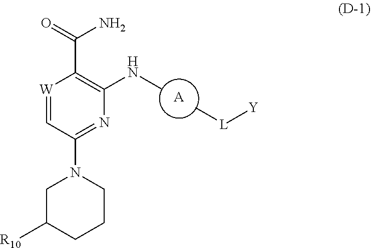

or a pharmaceutically acceptable salt thereof, wherein W is CH or N; Ring A is

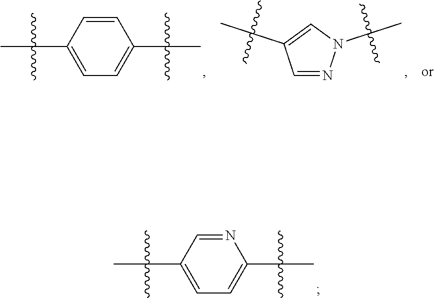 [SMILES: *c1ccc(*)cc1 ; *c1cnn(*)c1 ; *c1ccc(*)nc1]

L is —X¹—X²—X³—; X is —C₁₋₅ alkyl- or 4-6 membered monocyclic heterocycloalkyl having one to two heteroatoms independently selected from N, O, or S, wherein the monocyclic heterocycloalkyl of X is optionally substituted with —CH₃; X² is a bond, —C₁₋₅ alkyl-, or 4-6 membered monocyclic heterocycloalkyl having one to two heteroatoms independently selected from N, O, or S, wherein the monocyclic heterocycloalkyl of X is optionally substituted with —CH₃; X³ is a bond, —C₁₋₄ alkyl-, 4-6 membered monocyclic cycloalkyl, or 4-6 membered heterocycloalkyl having one to two heteroatoms independently selected from N, O, or S, wherein the heterocycloalkyl is optionally substituted with —CH₃; Y is as described herein; and R¹⁰ is

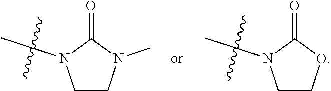

In some embodiments, Ring A is

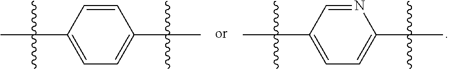 [SMILES: *c1ccc(*)cc1 ; *c1ccc(*)nc1]

In some embodiments, X¹ is a 4-6 membered monocyclic heterocycloalkyl having one to two heteroatoms independently selected from N, O, or S, wherein the monocyclic heterocycloalkyl of X¹ is optionally substituted with —CH₃. For example, X¹ is

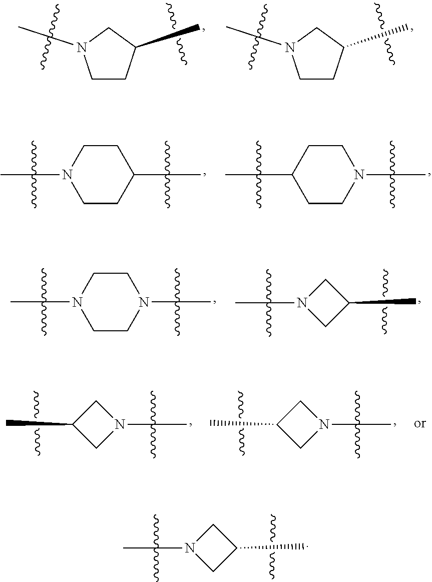

In some embodiments, X² is a bond, —C₁₋₅ alkyl-, 4-6 membered monocyclic cycloalkyl, or 4-6 membered monocyclic heterocycloalkyl having one to two heteroatoms independently selected from N, O, or S. For example, X² is a bond or —C₁₋₄ alkyl-.

In some embodiments, X³ is a bond, a 4-6 membered monocyclic cycloalkyl, or 4-6 membered monocyclic heterocycloalkyl having one to two heteroatoms independently selected from N, O, or S. For example, X³ is

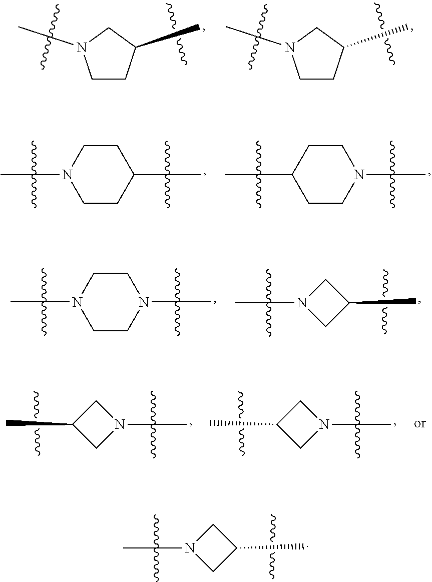

In some embodiments, L is

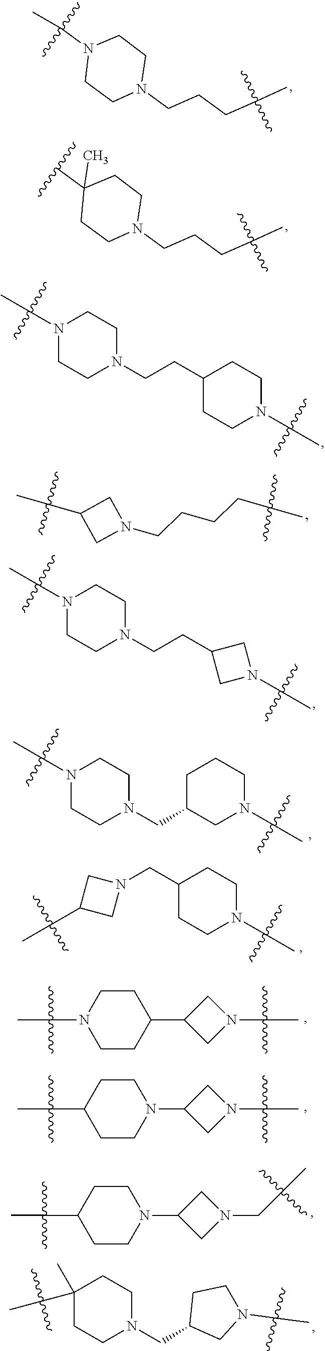

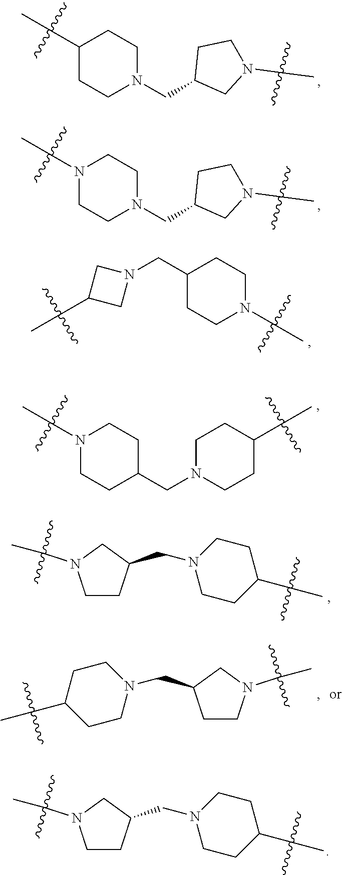

In some embodiments, R¹⁰ is

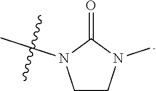 [SMILES: *N1CCN(C)C1=O]

In some embodiments, R¹⁰ is

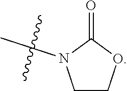 [SMILES: *N1CCOC1=O]

In some embodiments, the compound of Formula (D) or the compound of Formula (D-1) is a compound of Formula (D-2)

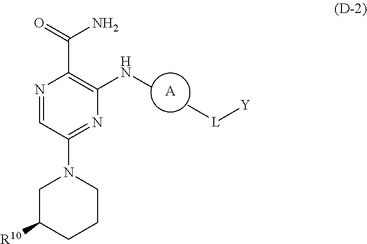

or a pharmaceutically acceptable salt thereof, wherein the terms Ring A, L, Y, and R¹⁰ are as defined in the compound of Formula (A), the compound of Formula (D), and the compound of Formula (D-1).

In some embodiments, Ring A is

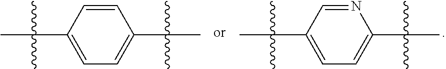 [SMILES: *c1ccc(*)cc1 ; *c1ccc(*)nc1]

In some embodiments, X¹ is a 4-6 membered monocyclic heterocycloalkyl having one to two heteroatoms independently selected from N, O, or S, wherein the monocyclic heterocycloalkyl of X¹ is optionally substituted with —CH₃. For example, X¹ is

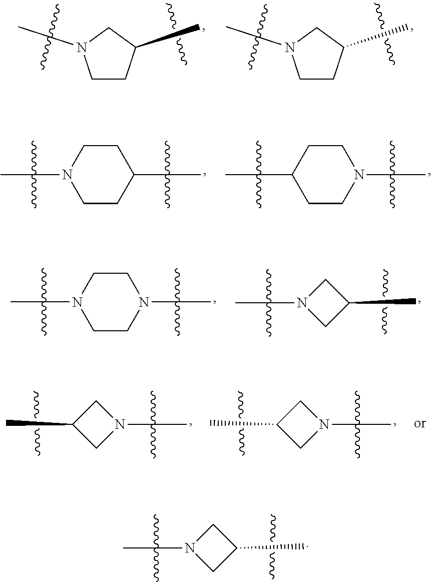

In some embodiments, X² is a bond, —C₁₋₅ alkyl-, 4-6 membered monocyclic cycloalkyl, or 4-6 membered monocyclic heterocycloalkyl having one to two heteroatoms independently selected from N, O, or S. For example, X² is a bond or —C₁₋₄ alkyl-.

In some embodiments, X³ is a bond, a 4-6 membered monocyclic cycloalkyl, or 4-6 membered monocyclic heterocycloalkyl having one to two heteroatoms independently selected from N, O or S. For example, X³ is

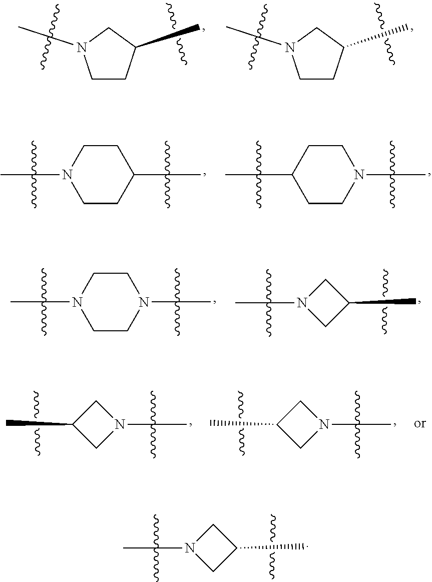

In some embodiments, L is

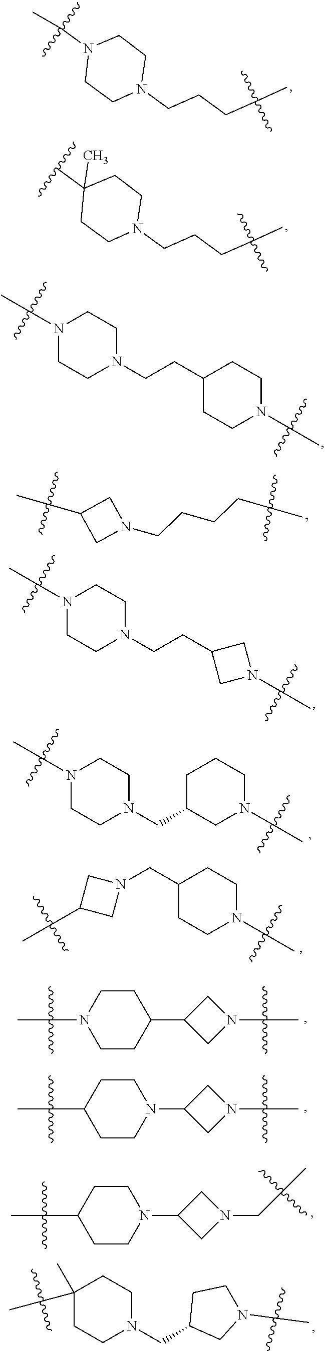

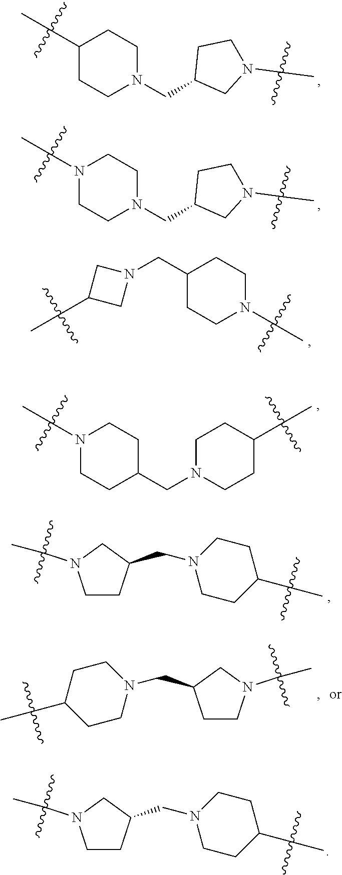

In some embodiments, R¹⁰ is

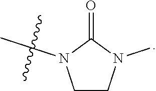 [SMILES: *N1CCN(C)C1=O]

In some embodiments, R¹⁰ is

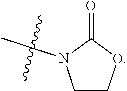 [SMILES: *N1CCOC1=O]

This disclosure also provides a compound of Formula (E)

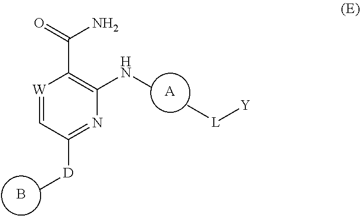

or a pharmaceutically acceptable salt thereof, wherein D is a bond or —NH—; W is N or CH; Ring A is phenyl, a 9-10 membered bicyclic aryl, a 5-6 membered partially or fully unsaturated monocyclic heterocycle, or a 9-10 membered bicyclic heteroaryl, wherein the monocyclic heterocycle and bicyclic heteroaryl of Ring A each possess one to three heteroatoms independently selected from N, O, or S; Ring B is an optionally substituted 5-6 membered saturated, partially unsaturated, or fully unsaturated monocyclic heterocycle, or an optionally substituted 8-10 membered (e.g., 8-9 membered or 9-10 membered) spiro bicyclic heterocycle, wherein Ring B has one to three heteroatoms independently selected from N, O, or S; L is —X¹—X²—X³—X⁴—X⁵—; X¹ is a bond, —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, a 5-6 membered saturated, partially unsaturated, or fully unsaturated carbocycle, or a 5-6 membered saturated, partially unsaturated, or fully unsaturated heterocycle having one to three heteroatoms independently selected from N, O, or S; X⁵ is a bond, —N(R)—, or —C(O)—N(R)—; each R is independently —H or —C₁₋₃ alkyl; each of m, n, and p is independently an integer from one to three; and Y is as described herein, wherein at least one of X¹, X², X³, X⁴, and X⁵ has a nitrogen atom, and Y is directly bonded to L at a nitrogen atom of X¹, X², X³, X⁴, or X⁵.

In some embodiments, Ring B is

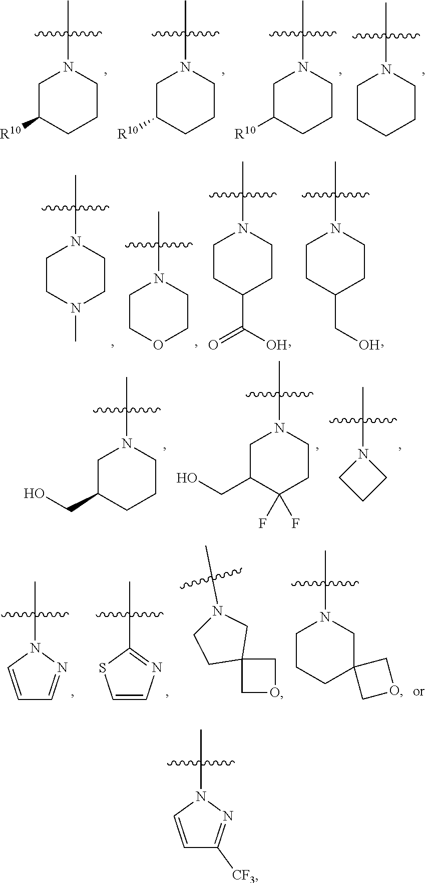

wherein R¹⁰ is

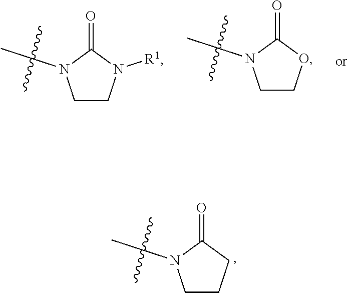

and wherein R¹ is a C₁₋₄ alkyl group. For example, Ring B is

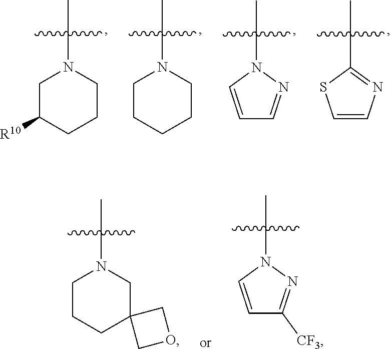

wherein R¹⁰ is

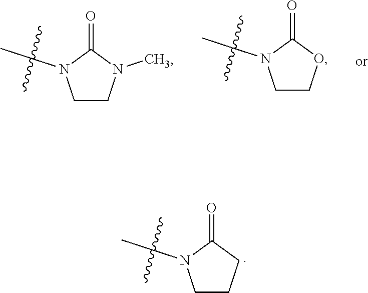 [SMILES: *N1CCN(C)C1=O ; *N1CCOC1=O ; *N1CCCC1=O]

In other examples, Ring B is

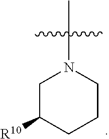

In some embodiments, R¹⁰ is

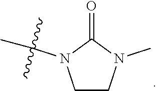 [SMILES: *N1CCN(C)C1=O]

In some embodiments, Ring A is

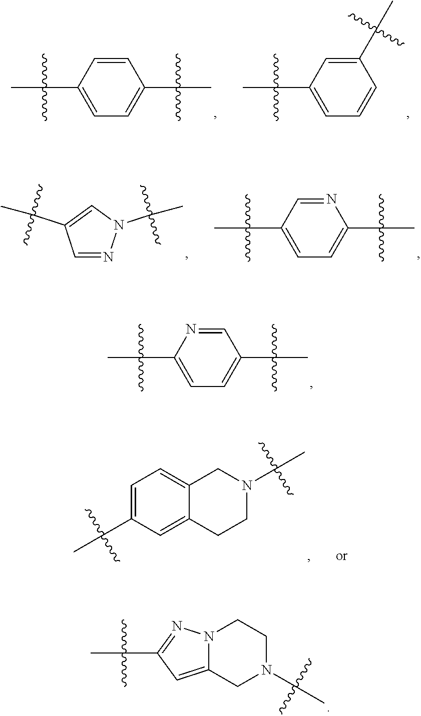

In some embodiments, X⁵ is —N(R)—.

In some embodiments, X⁵ is —C(O)—N(R)—.

In some embodiments, X⁵ is a bond.

In some embodiments, L is

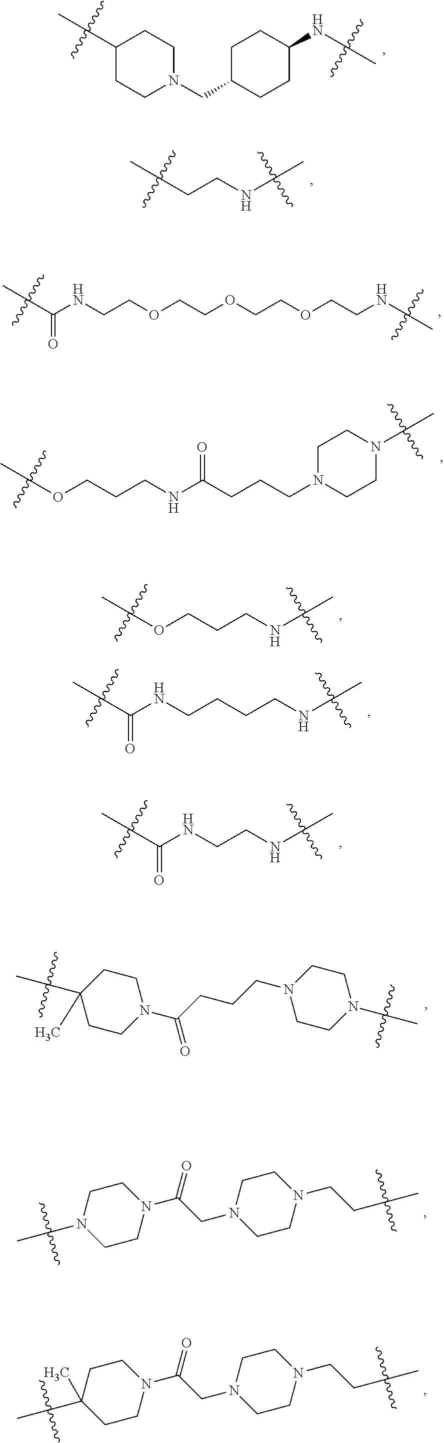

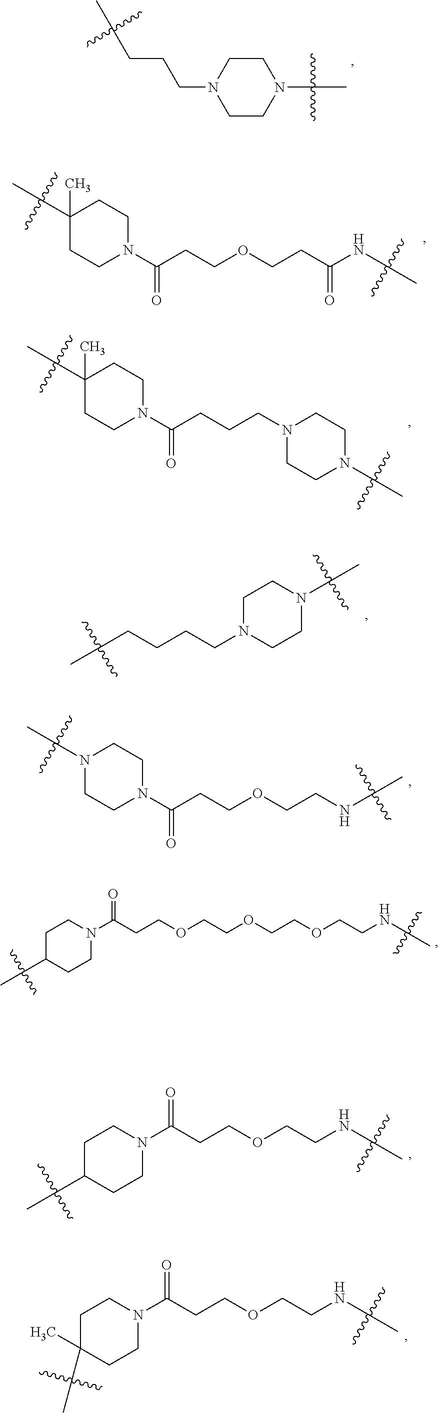

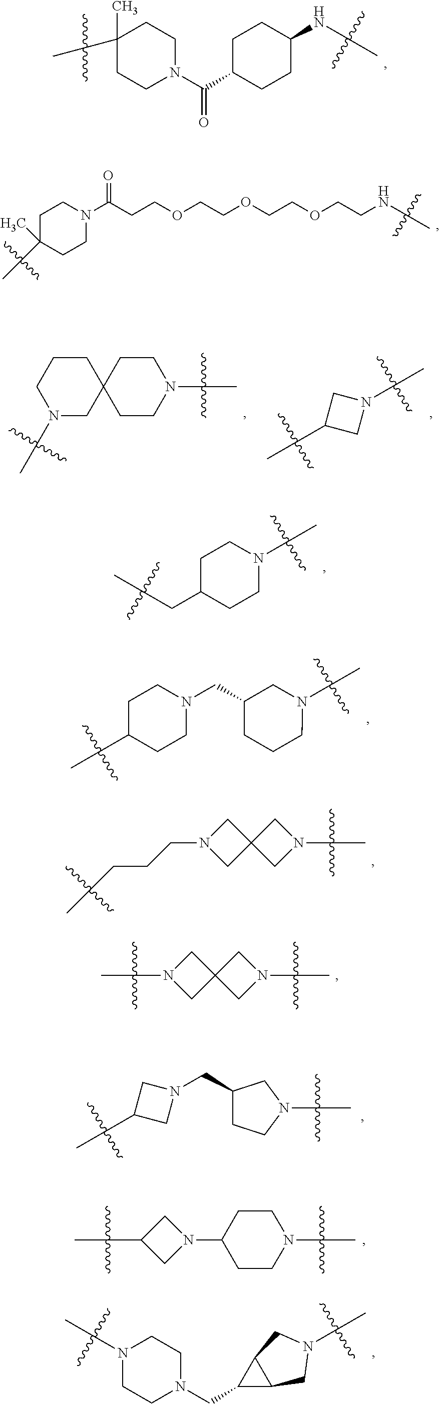

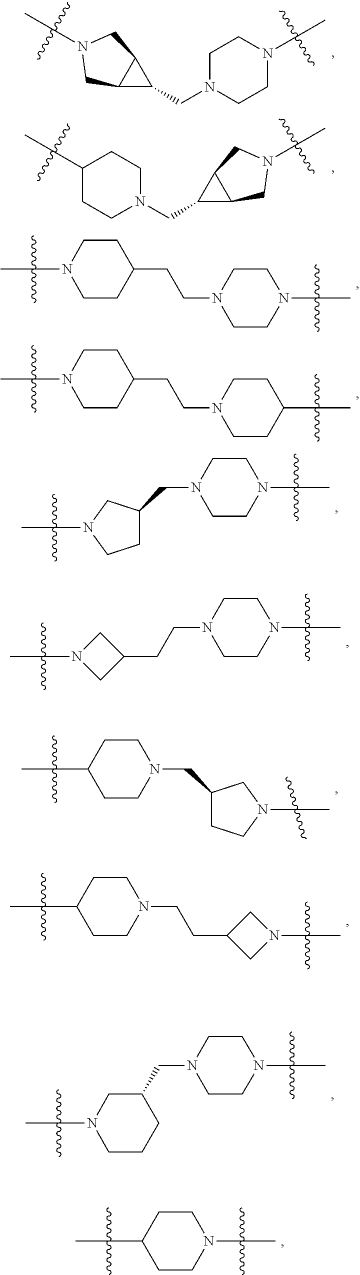

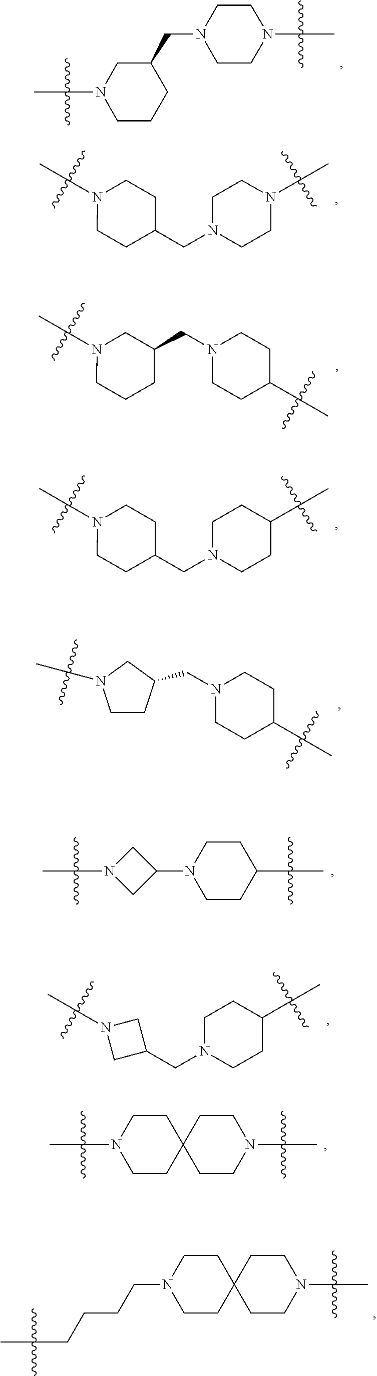

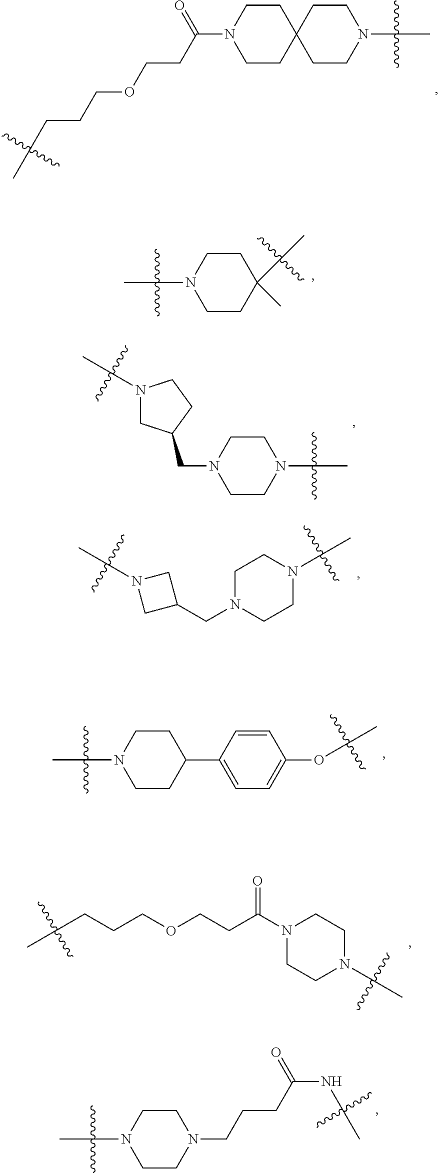

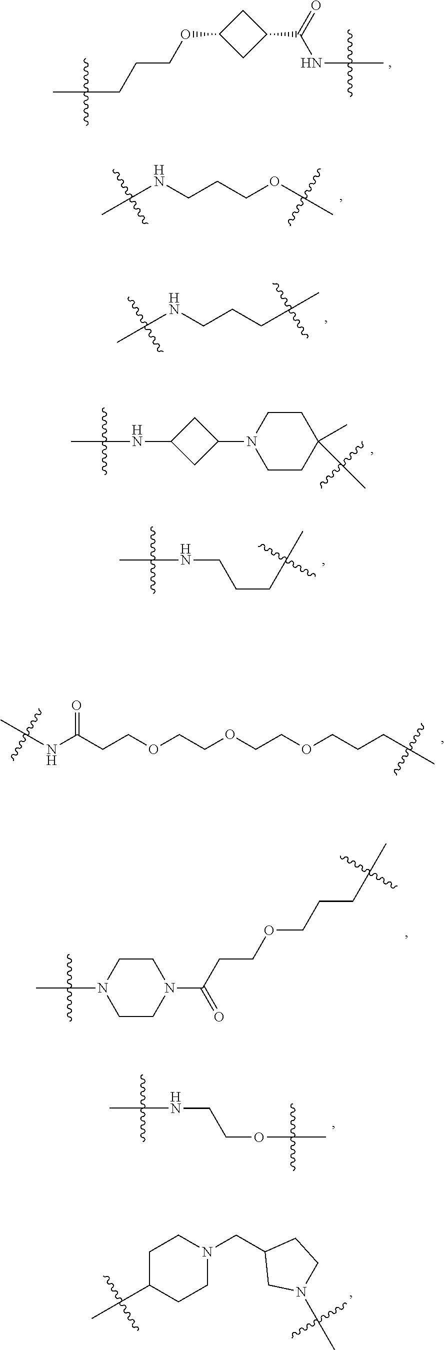

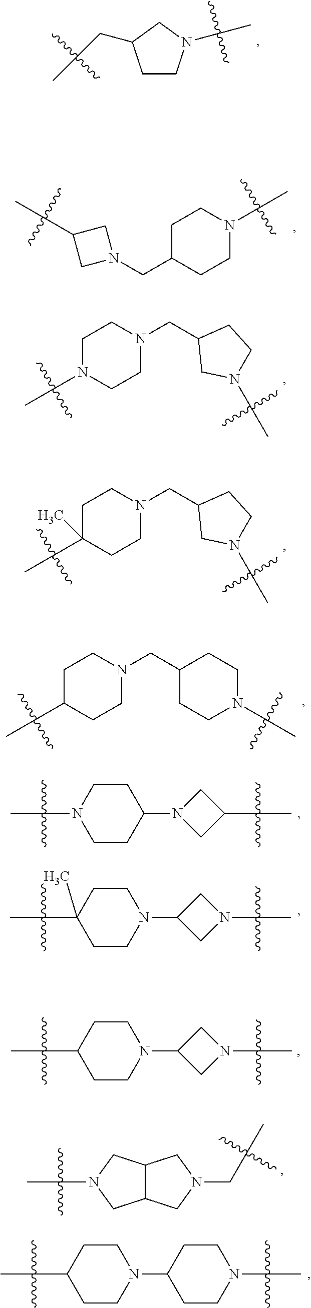

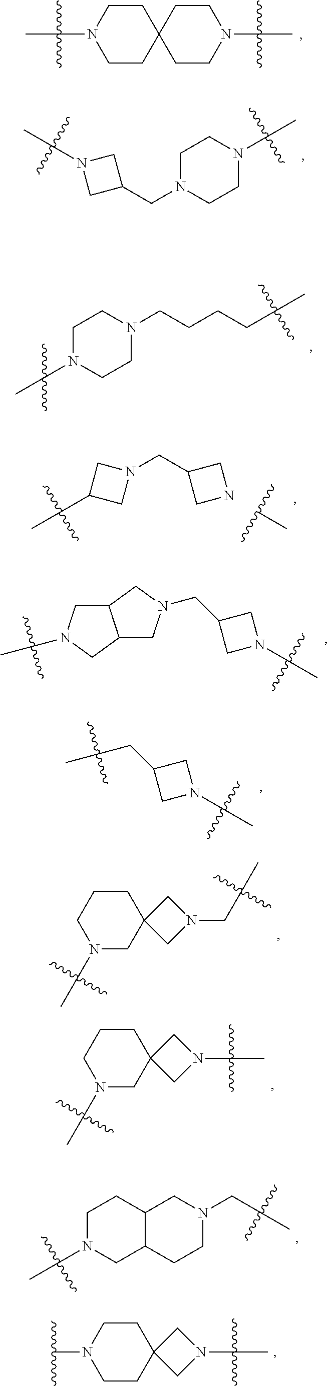

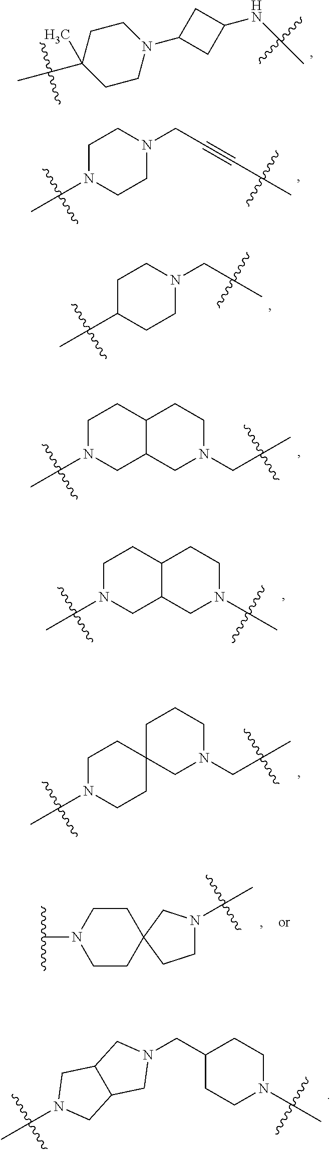

This disclosure also provides a compound of Formula (F)

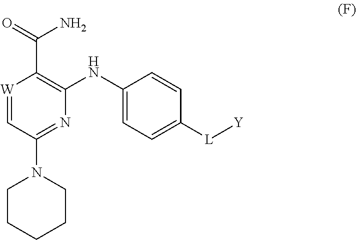

or a pharmaceutically acceptable salt thereof, wherein W is CH or N; L is —X¹—X²—X³—; X¹ is —C(O)—N(R), —N(R)—C(O), (O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is optionally substituted with —CH₃; X² is a bond, —C₁₋₅ alkyl-, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃; each R is independently —H or —C₁₋₃ alkyl; each of m, n, and p is independently an integer from one to three; and Y is as described herein.

In some embodiments, W is N.

In some embodiments, X¹ is a 4-6 membered monocyclic heterocycloalkyl having one to two heteroatoms independently selected from N, O, or S, wherein each of the monocyclic heterocycloalkyl of X¹ is optionally substituted with —CH₃. For example X¹ is

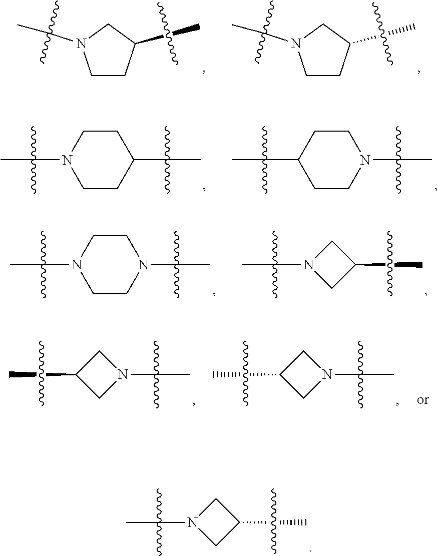

In some instances, X¹ is

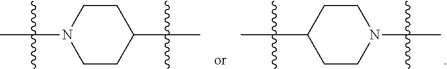 [SMILES: *C1CCN(*)CC1 ; *C1CCN(*)CC1]

In some embodiments, X² is a bond or —C₁₋₅ alkyl-.

In some embodiments, X³ is a 4-6 membered monocyclic heterocycloalkyl having one to two heteroatoms independently selected from N, O, or S. For example, X³ is

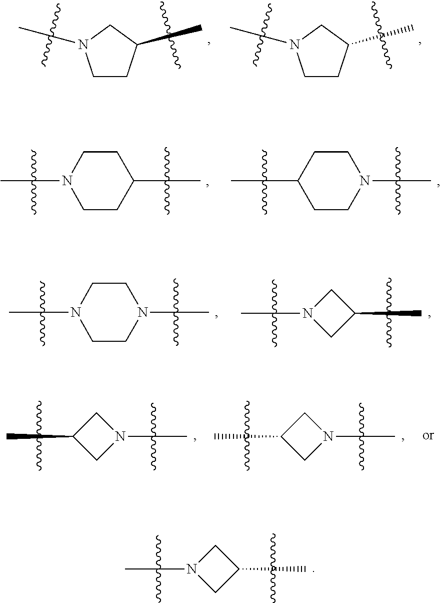

In some instances X³ is

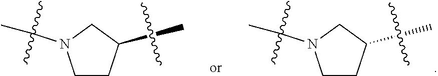

In some embodiments, L is

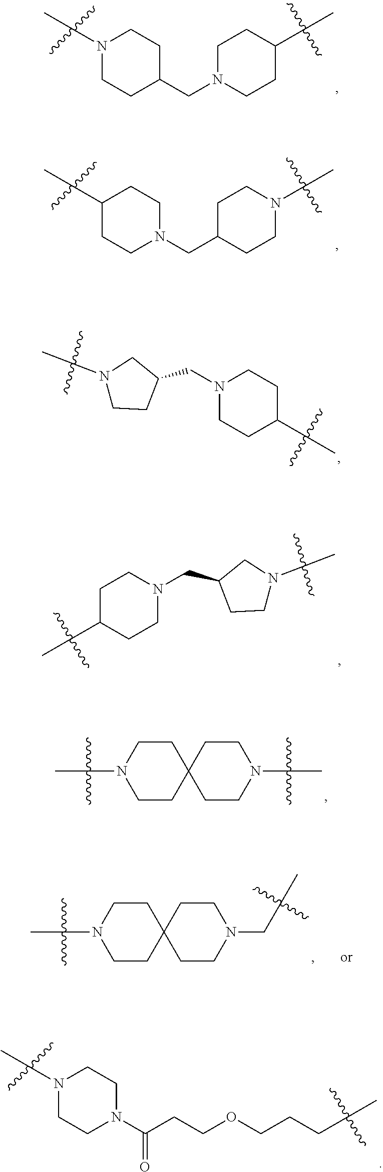

In some embodiments, L is

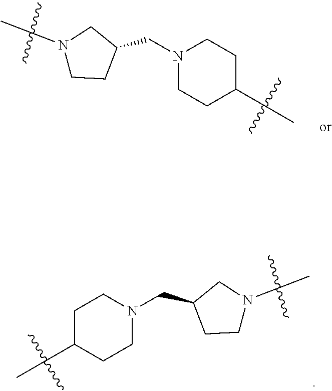 [SMILES: *C1CCN(C[C@@H]2CCN(*)C2)CC1 ; *C1CCN(C[C@@H]2CCN(*)C2)CC1]

In some embodiments, W is N; and L is

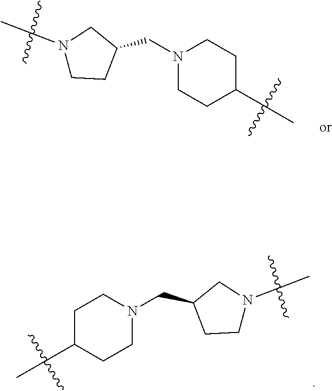 [SMILES: *C1CCN(C[C@@H]2CCN(*)C2)CC1 ; *C1CCN(C[C@@H]2CCN(*)C2)CC1]

This disclosure also provides a compound of Formula (G)

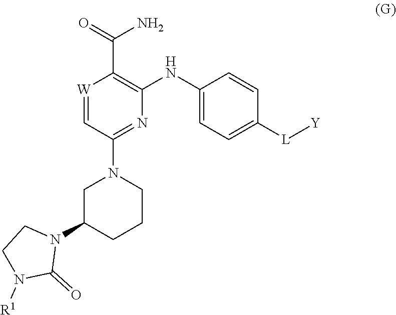

or a pharmaceutically acceptable salt thereof, wherein R¹, L, and Y are as defined for compounds of Formula (A).

In some embodiments, R¹ is methyl.

In some embodiments, W is N.

This disclosure also provides a compound of Formula (M)

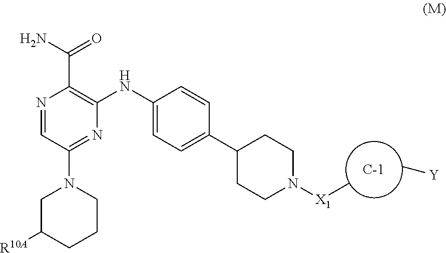

or a pharmaceutically acceptable salt thereof, wherein R^{10A} is —H,

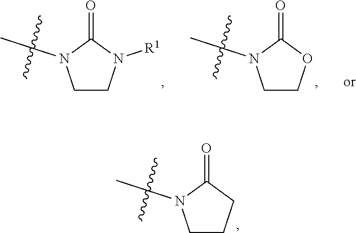

wherein R¹ is C₁₋₄ alkyl; X is —C₁₋₅ alkyl-; Ring C-1 is a 5-6 membered heterocycloalkyl having one nitrogen atom; and Y is as described herein.

In some embodiments, R^{10A} is —H or

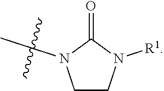

In some embodiments, R^{10A} is

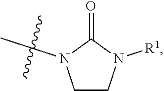

and R¹ is methyl, ethyl, propyl, iso-propyl, butyl, sec-butyl, or iso-butyl. For example, R¹ is methyl.

In some embodiments, X is methylene (—CH₂—), ethylene (—CH₂CH₂—), or propylene (—CH₂CH₂CH₂—). For instance, X¹ is methylene (—CH₂—).

In some embodiments, Ring C-1 is

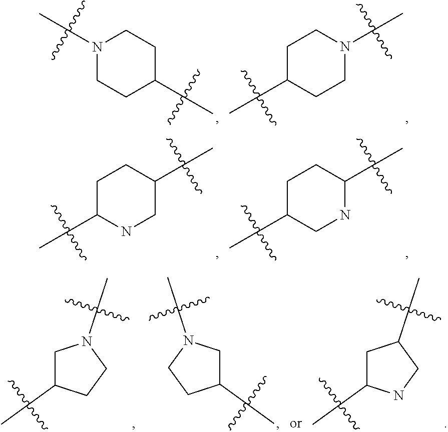

For instance, Ring C-1 is

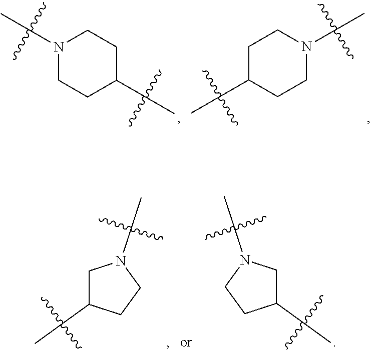 [SMILES: *C1CCN(*)CC1 ; *C1CCN(*)CC1 ; *C1CCN(*)C1 ; *C1CCN(*)C1]

This disclosure provides a compound of Formula (X)

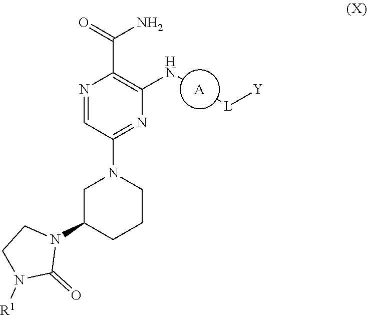

or a pharmaceutically acceptable salt thereof, wherein R¹ is C₁₋₃ alkyl; Ring A is phenyl, 5-6 membered partially or fully unsaturated monocyclic heterocycle, 9-10 membered bicyclic aryl, or 9-10 membered bicyclic heteroaryl, wherein the heterocycle and the bicyclic heteroaryl of Ring A each independently have one to three heteroatoms independently selected from N, O, or S; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, wherein the bicyclic heterocycloalkyl of X¹ is optionally substituted with —CH₃, or 4-6 membered monocyclic heterocycloalkyl having one to two heteroatoms independently selected from N, O, or S, wherein the monocyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated carbocycle having zero to three heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from one to three;
Y is as described herein, wherein each R² is independently halo or C₁₋₄ alkyl; each Z is —
C(R^{A})₂— or —C(O)—; each R^{A} is independently —H or C₁₋₄ alkyl; and q is zero, one, or two.

In some instances, the compound of Formula (X) is a compound of Formula (I)

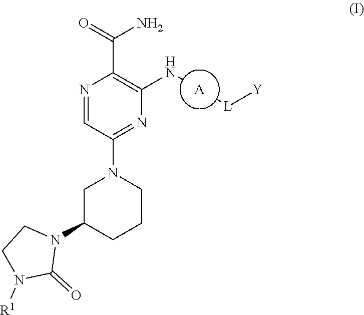

or a pharmaceutically acceptable salt thereof, wherein R¹ is C₁₋₃ alkyl; Ring A is phenyl, 9-10 membered bicyclic aryl, or 9-10 membered bicyclic heteroaryl having one to three heteroatoms independently selected from N, O, or S; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, wherein the heterocycloalkyl is optionally substituted with —CH₃, or 4-6 membered heterocycloalkyl having one to two heteroatoms independently selected from N, O, or S, wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having one to two heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having one to three heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from one to three (e.g., one, two, or three); Y is as described herein, wherein each R² is independently halo or —C₁₋₄ alkyl; each Z is —C(R^{A})₂— or —C(O)—; each R^{A} is independently —H or —C₁₋₄ alkyl; and q is zero, one, or two.

In some embodiments, q is zero. In other embodiments, q is one and R² is —F.

In some embodiments, Z is —CH₂— or —C(O)—.

In some embodiments, R¹ is —C₁₋₃ alkyl. For example, R¹ is methyl, ethyl, propyl, or iso-propyl. In other embodiments, R¹ is methyl.

In some embodiments, each R is independently —H or —CH₃. For instance, each R is —H.

In some embodiments, X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, or 4-6 membered heterocycloalkyl having one to two heteroatoms independently selected from N, O, or S, wherein the heterocycloalkyl is optionally substituted with —CH₃. In some embodiments, X¹ is —C(O)—N(R)—. For example, X¹ is —C(O)—N(H)—, —C(O)—N(CH₃)—, or —C(O)—N(CH₂CH₃)—. In other embodiments, X¹ is a 5-6 membered heterocycloalkyl having one to two heteroatoms independently selected from N, O, or S, wherein the heterocycloalkyl is optionally substituted with —CH₃. For example, X¹ is,

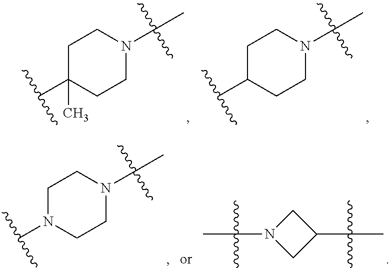

In other examples, X¹ is a 7-10 membered spiro bicyclic heterocycloalkyl ring having one to three heteroatoms independently selected from N, O, or S (e.g., N). For example, X¹ is

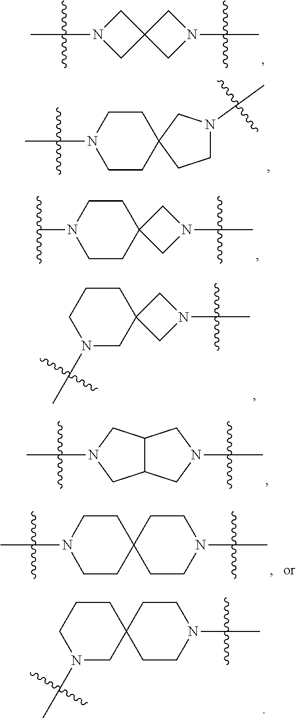

In other embodiments, X¹ is —(O—CH₂—CH₂)ₘ— or —(O—CH₂—CH₂—CH₂)ₘ—, wherein m is one, two, three. For example, X¹ is —(O—CH₂—CH₂)ₘ— or —(O—CH₂—CH₂—CH₂)ₘ—, and m is one. In another example, X¹ is —(O—CH₂—CH₂)ₘ— or —(O—CH₂—CH₂—CH₂)ₘ—, and m is two. In some embodiments, X¹ is —C₁₋₅ alkyl-. For example, X¹ is methylene (—CH₂—), ethylene (—CH₂CH₂—), propylene (—CH₂CH₂CH₂—), butylene (—CH₂CH₂CH₂CH₂—), or the like. In some embodiments, X¹ is —CH₂—, —C(O)—,

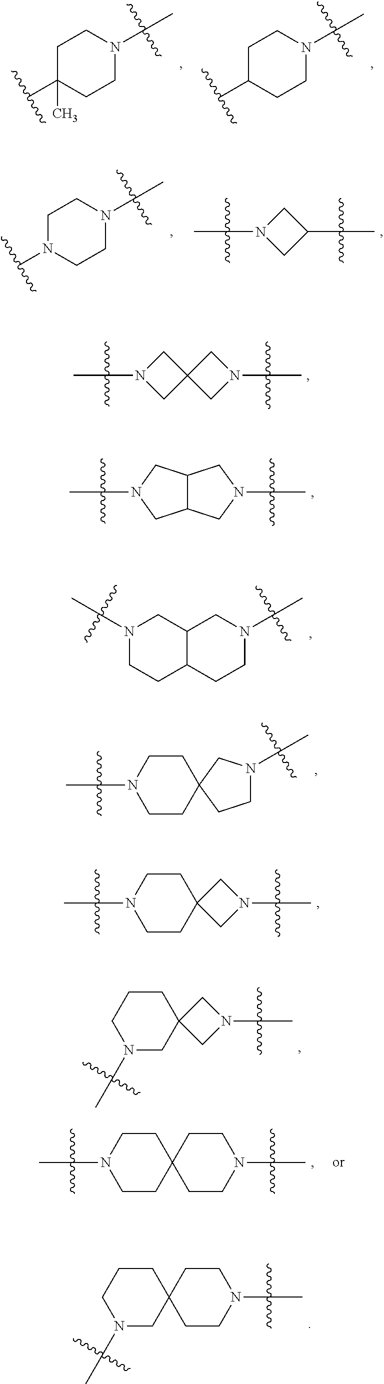

In some embodiments, X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having one to two heteroatoms independently selected from N, O, or S. In some embodiments, X² is a bond. In some embodiments, X² is —(O—CH₂—CH₂)ₙ—, —(CH₂—CH₂—O)ₙ—, or —C₁₋₅ alkyl-, wherein n is one, two, or three. For example, X¹ is —C(O)—N(R)—, and X² is —(O—CH₂—CH₂)ₙ—, —(CH₂—CH₂—O)ₙ—, or —C₁₋₅ alkyl-. In some examples, X² is —(O—CH₂—CH₂)ₙ— or —(CH₂—CH₂—O)ₙ—, where n is one or two. In other examples, X² is —C₁₋₅ alkyl-. For instance, X² is methylene (—CH₂—), ethylene (—CH₂CH₂—), propylene (—CH₂CH₂CH₂—), butylene (—CH₂CH₂CH₂CH₂—), or the like. In other examples, X² is a bond, —CH₂—, —CH₂CH₂—, or —CH₂CH₂CH₂—. In some examples, X² is 4-6 membered cycloalkyl. For instance, X² is

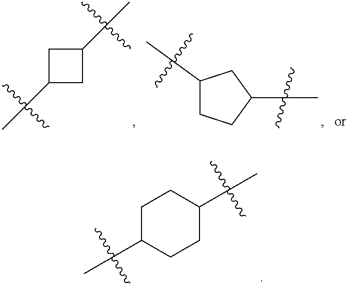 [SMILES: *C1CC(*)C1 ; *C1CCC(*)C1 ; *C1CCC(*)CC1]

In other examples X² is 4-6 membered heterocycloalkyl having one to two heteroatoms independently selected from N, O, or S. For instance, X² is

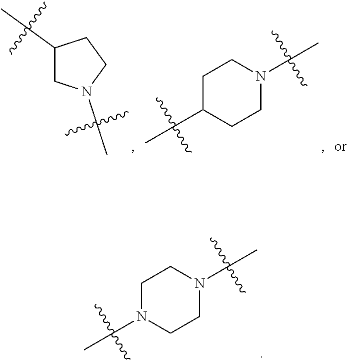 [SMILES: *C1CCN(*)C1 ; *C1CCN(*)CC1 ; *N1CCN(*)CC1]

In some embodiments, X³ is a bond, —C₁₋₄ alkyl-, 4-6 membered cycloalkyl, —N(R)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is optionally substituted with —CH₃. In some embodiments, X³ is a bond. In some embodiments, X³ is methyl, ethyl, propyl, iso-propyl, butyl, or the like. In some embodiments, X³ is cyclopentyl or cyclohexyl. In some embodiments, X³—N(H)—. And, in other embodiments, X³ is —(O—CH₂—CH₂)ₚ— or —(CH₂—CH₂—O)ₚ—, wherein p is one or two.

In some embodiments, X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having one to three heteroatoms independently selected from N, O, or S. In some embodiments, X⁴ is a bond,

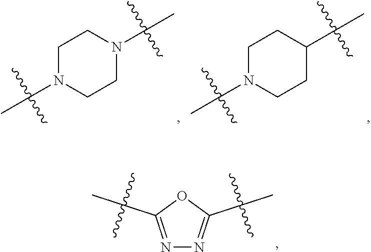 [SMILES: *N1CCN(*)CC1 ; *C1CCN(*)CC1 ; *c1nnc(*)o1]

—C₁₋₄ alkyl-, —CH₂—CH₂—N(R)—, or —N(R)—. For example, X⁴ is —CH₂—CH₂—N(H)—, or —N(H)—. In other examples, X⁴ is methyl, ethyl, propyl, iso-propyl, butyl, sec-butyl, or the like.

In some embodiments, X⁵ is a bond, —C₁₋₄ alkyl-, —N(R)—, or —C(O)—N(R)—. In some embodiments, X⁵ is a bond. In some embodiments, X⁵ is methyl, ethyl, propyl, iso-propyl, butyl, or the like. In some embodiments, X⁵ is —N(H)— or —C(O)—N(H)—.

In some embodiments, L is selected from

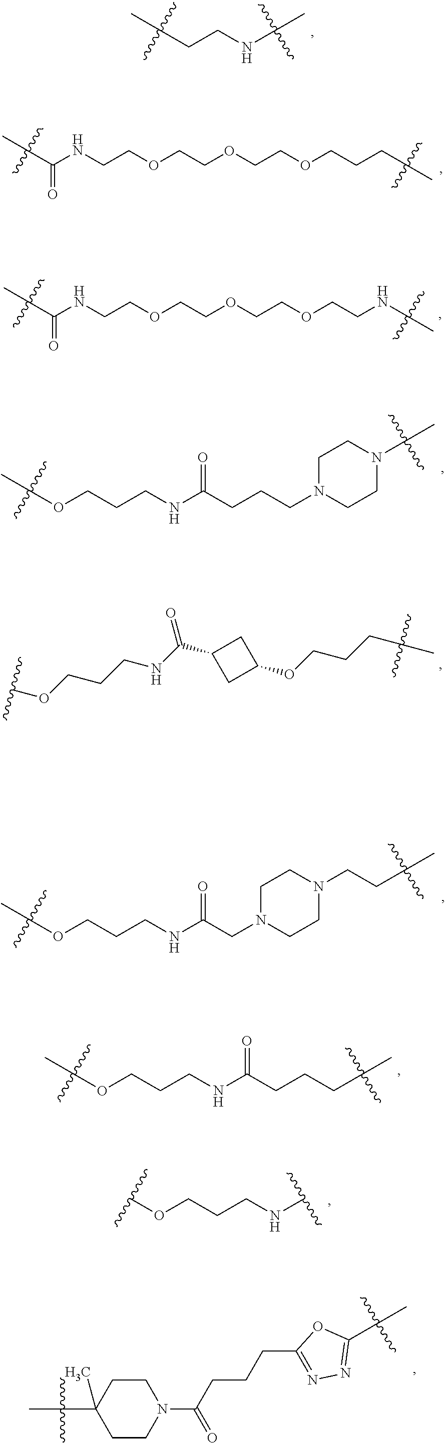

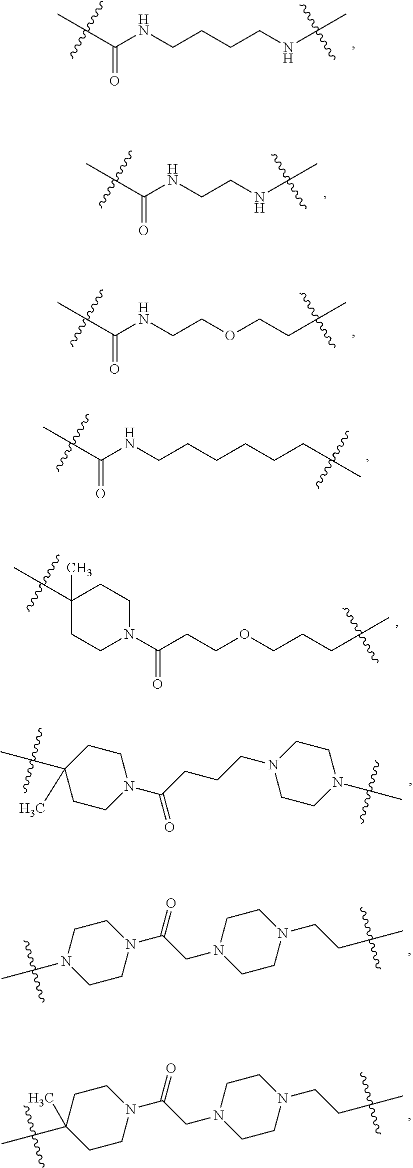

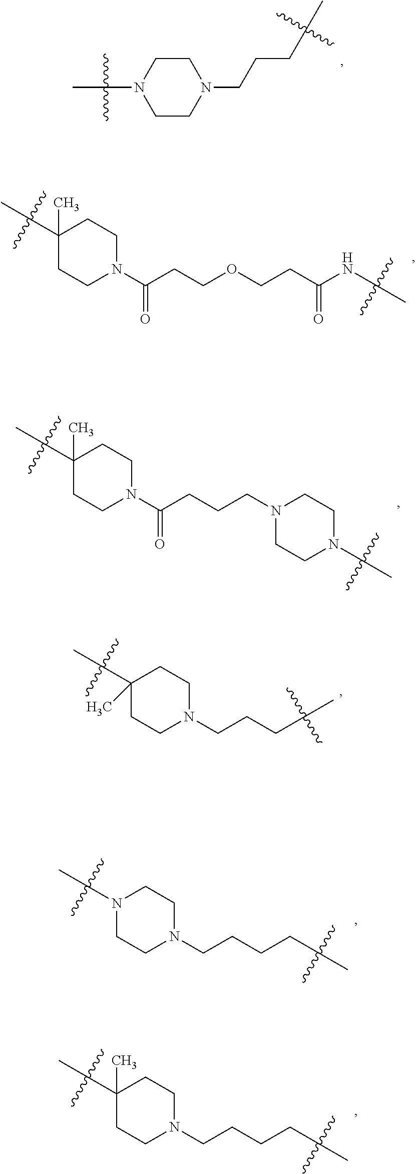 [SMILES: *CCCN1CCN(*)CC1 ; *NC(=O)CCOCCC(=O)N1CCC(*)(C)CC1 ; *N1CCN(CCCC(=O)N2CCC(*)(C)CC2)CC1 ; *CCCN1CCC(*)(C)CC1 ; *CCCCN1CCN(*)CC1 ; *CCCCN1CCC(*)(C)CC1]

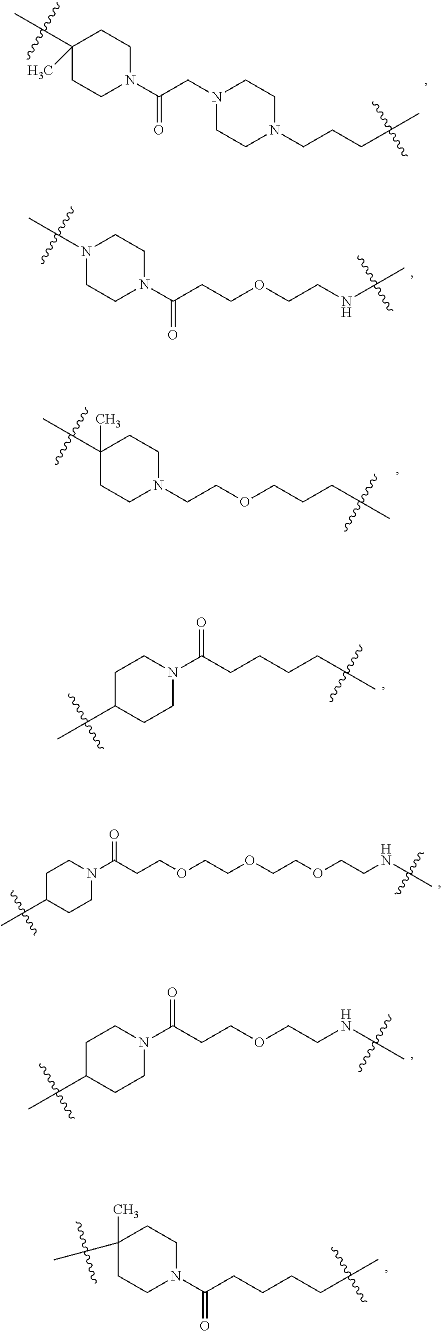

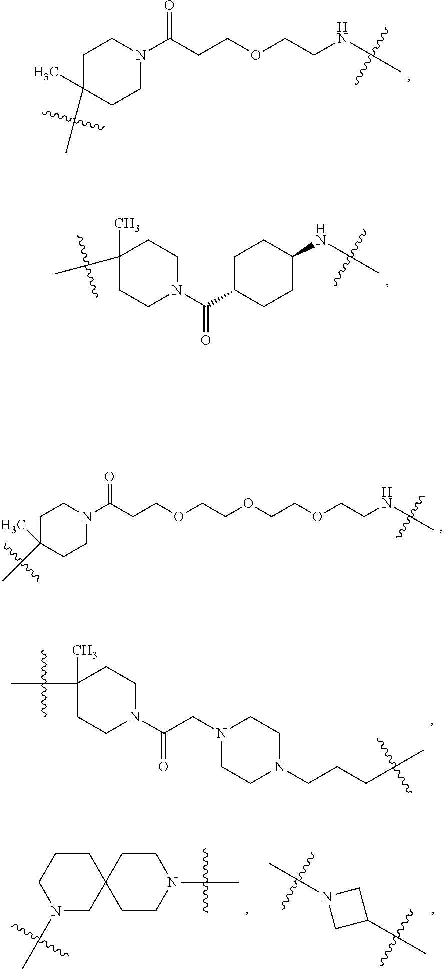 [SMILES: *NCCOCCC(=O)N1CCC(*)(C)CC1 ; *N[C@H]1CC[C@H](C(=O)N2CCC(*)(C)CC2)CC1 ; *NCCOCCOCCOCCC(=O)N1CCC(*)(C)CC1 ; *CCCN1CCN(CC(=O)N2CCC(*)(C)CC2)CC1 ; *N1CCC2(CCCN(*)C2)CC1 ; *C1CN(*)C1]

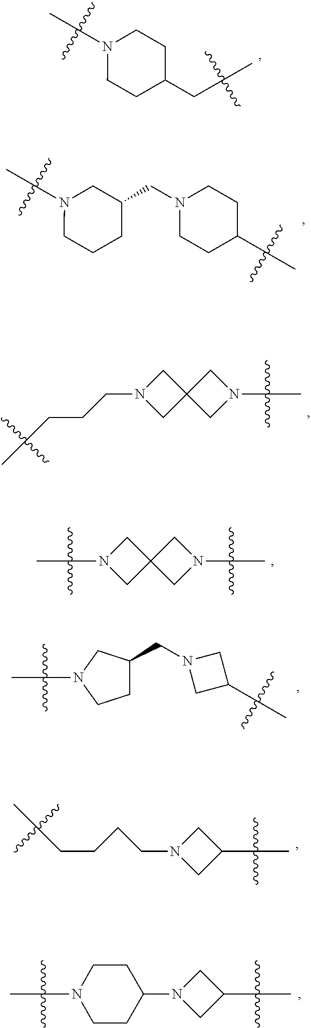

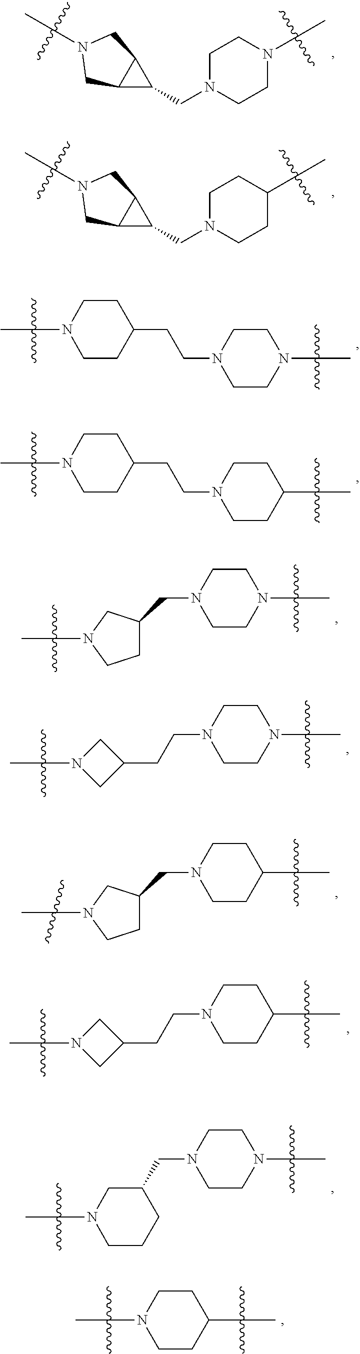

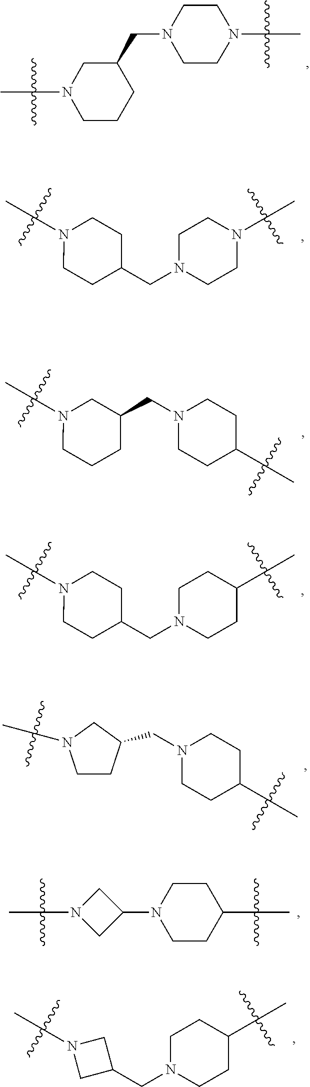

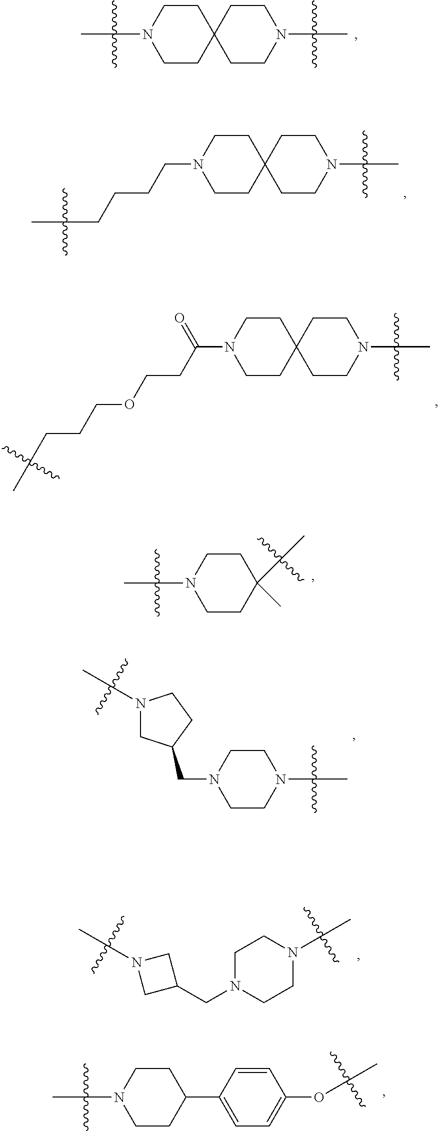

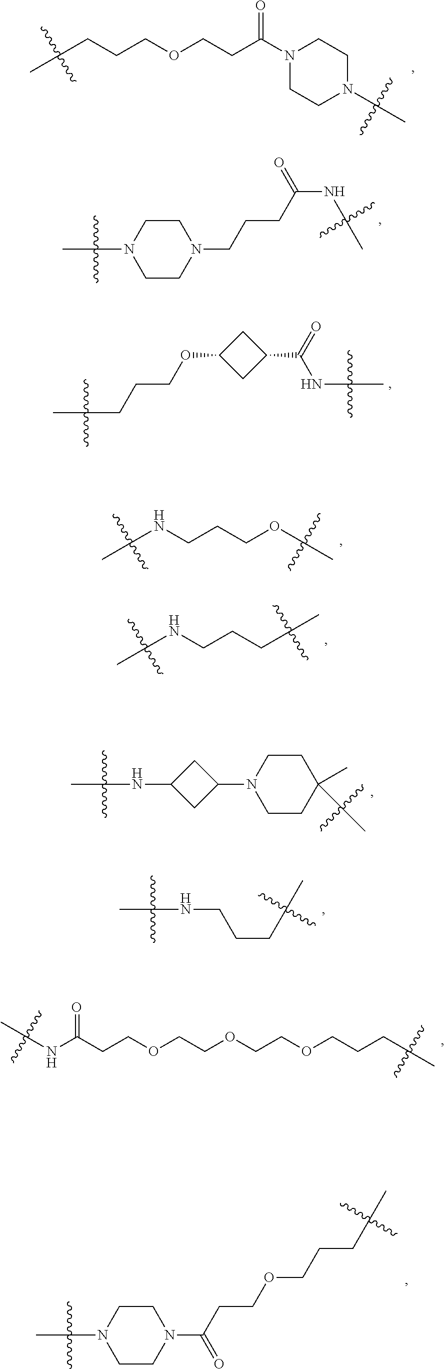

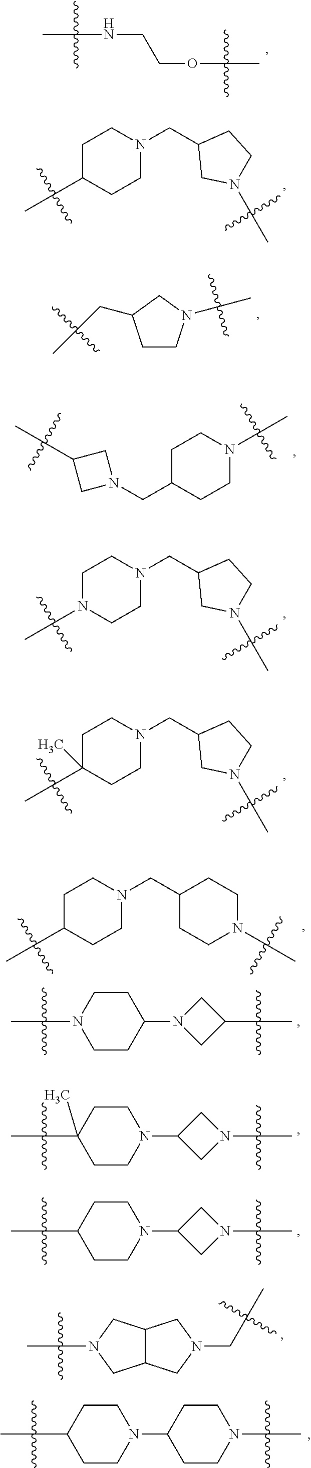

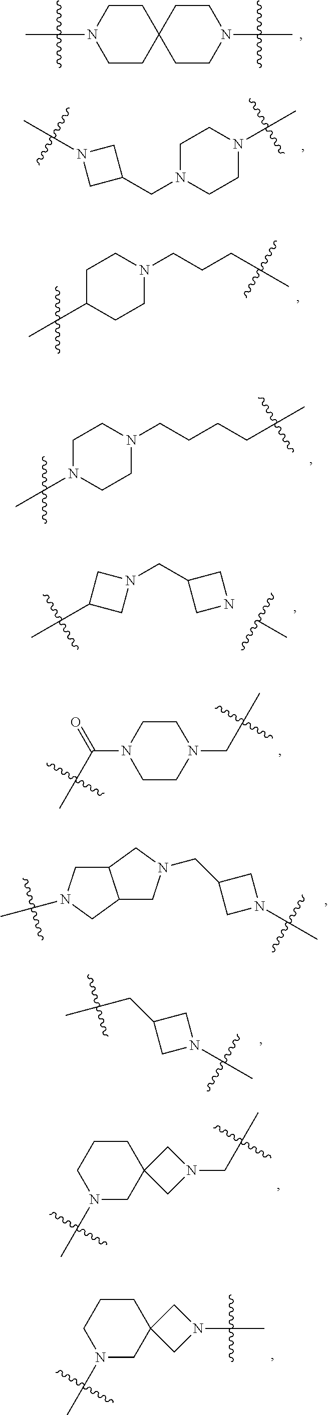

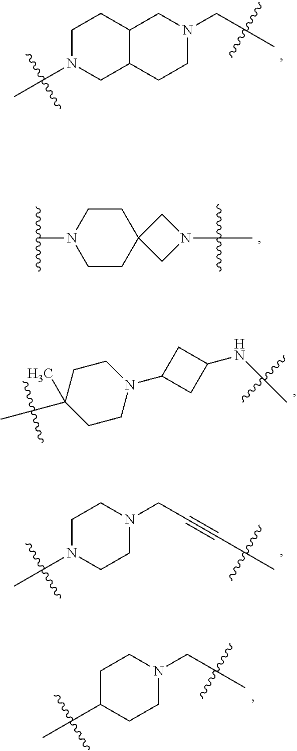 [SMILES: *CN1CCC2CN(*)CCC2C1 ; *N1CCC2(CC1)CN(*)C2 ; *NC1CC(N2CCC(*)(C)CC2)C1 ; *C#CCN1CCN(*)CC1 ; *CN1CCC(*)CC1]

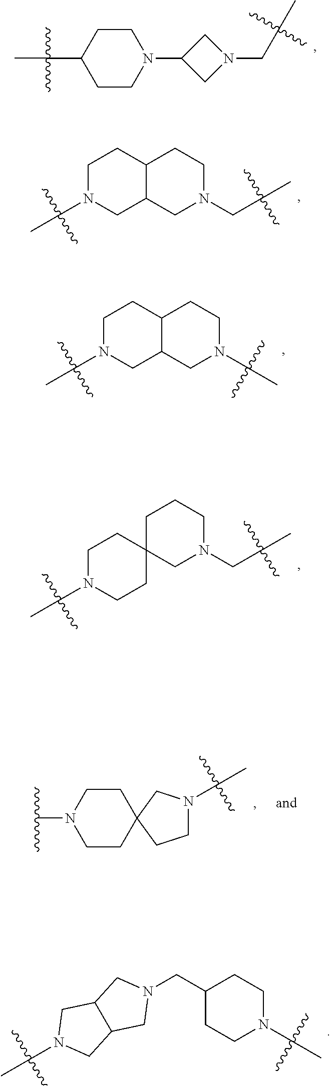 [SMILES: *CN1CC(N2CCC(*)CC2)C1 ; *CN1CCC2CCN(*)CC2C1 ; *N1CCC2CCN(*)CC2C1 ; *CN1CCCC2(CCN(*)CC2)C1 ; *N1CCC2(CC1)CCN(*)C2 ; *N1CCC(CN2CC3CN(*)CC3C2)CC1]

This disclosure also provides a compound of Formula (I-A):

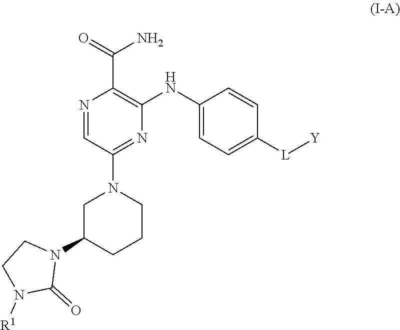

or a pharmaceutically acceptable salt thereof, wherein R¹ is C₁₋₃ alkyl; L is —X¹—X²—X³—X⁴—X⁵—; X¹ is —C(O)—N(R)—, —N(R)—C(O), —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, wherein the heterocycloalkyl is optionally substituted with —CH₃, or 4-6 membered heterocycloalkyl having one to two heteroatoms independently selected from N, O, or S, wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having one to two heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, or 4-6 membered heterocycloalkyl having one to two heteroatoms independently selected from N, O, or S, wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having one to three heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from one to three; Y is as described herein, wherein each R² is independently halo or —C₁₋₄ alkyl; each Z is —C(R^{A})₂— or —C(O)—; each R^{A} is independently —H or —C₁₋₄ alkyl; and q is zero, one, or two.

In other embodiments, each of the variables in Formula (I-A) is as defined herein for the compound of Formula (X) or (I).

This disclosure also provides a compound of Formula (I-B)

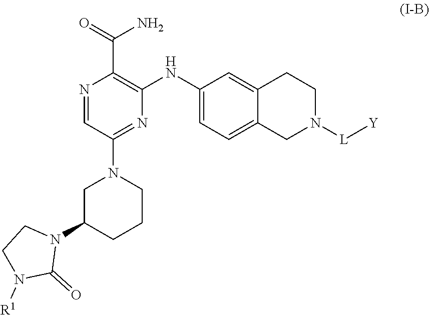

or a pharmaceutically acceptable salt thereof, wherein R¹ is C₁₋₃ alkyl; L is —X¹—X²—X³—X⁴—X⁵—; X¹ is —C(O)—N(R)—, —N(R)—C(O), (O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl ring having one to three heteroatoms independently selected from N, O, or S, wherein the heterocycloalkyl is optionally substituted with —CH₃, or 4-6 membered heterocycloalkyl having one to two heteroatoms independently selected from N, O, or S, wherein the heterocycloalkyl is optionally substituted with —CH₃; X² is a bond, —(O—CH₂—CH₂)ₚ—, —(CH₂—CH₂—O)ₚ—, —N(R)—C(O)—, —N(R)—, —C(O)—, —C₁₋₅ alkyl-, 4-6 membered cycloalkyl, or 4-6 membered heterocycloalkyl having one to two heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, or 4-6 membered heterocycloalkyl having one to two heteroatoms independently selected from N, O, or S, wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having one to three heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from one to three; Y is as described herein, wherein each R² is independently halo or C₁₋₄ alkyl; each Z is —C(R^{A})₂— or —C(O)—; each R^{A} is independently —H or C₁₋₄ alkyl; and q is zero, one, or two.

In other embodiments, each of the variables in Formula (I-B) is as defined herein for the compound of Formula (X) or (I).

This disclosure also provides a compound of Formula (III)

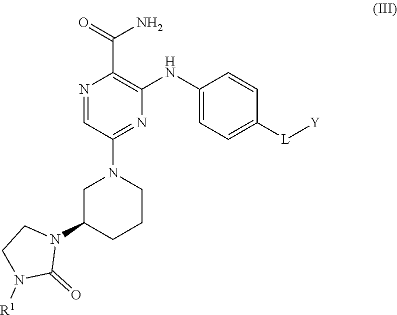

or a pharmaceutically acceptable salt thereof, wherein R¹ is C₁₋₃ alkyl; L is —X¹—X²—X³—; X¹ is 7-12 membered spiro bicyclic heterocycloalkyl having one to three heteroatoms independently selected from N, O, or S, wherein the heterocycloalkyl is optionally substituted with —CH₃, or 4-6 membered heterocycloalkyl having one to two heteroatoms independently selected from N, O, or S, wherein the heterocycloalkyl is optionally substituted with —CH₃; X² is a bond or —C₁₋₅ alkyl-; X³ is a bond, —C₁₋₄ alkyl-, 4-6 membered heterocycloalkyl having one to two heteroatoms independently selected from N, O, or S, wherein the heterocycloalkyl is optionally substituted with —CH₃; Y is as described herein, wherein each R² is independently halo or —C₁₋₄ alkyl; each Z is —C(R^{A})₂— or —C(O)—; each R^{A} is independently —H; and q is zero, one, or two.

This disclosure also provides a compound of Formula (IV)

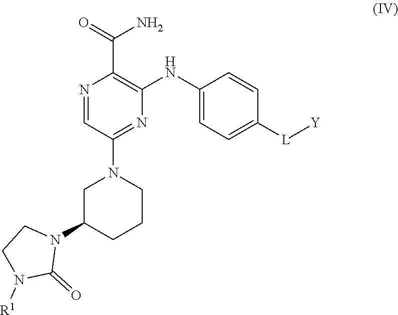

or a pharmaceutically acceptable salt thereof, wherein R¹ is C₁₋₃ alkyl; L is —X¹—X²—X³—X⁴—X⁵—; X¹ is —C(O)—N(R)—, —N(R)—C(O), (O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, wherein the heterocycloalkyl is optionally substituted with —CH₃, or 4-6 membered heterocycloalkyl having one to two heteroatoms independently selected from N, O, or S, wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having one to two heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, or 4-6 membered heterocycloalkyl having one to two heteroatoms independently selected from N, O, or S, wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having one to three heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from one to three; Y is as described herein, wherein each R² is independently halo or —C₁₋₄ alkyl; each Z is —C(R^{A})₂— or —C(O)—; each R^{A} is independently —H or —C₁₋₄ alkyl; and q is zero, one, or two.

In certain embodiments of any of Formulas A-X or I-IV, Y is

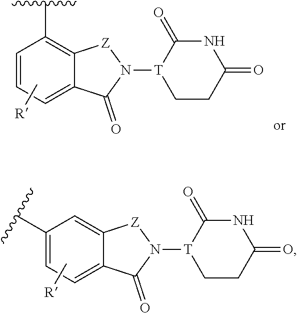

wherein each T is independently CH or N; and each Z is independently —CH₂— or —C(O)—; and each R′ is hydrogen, methyl, or NH₂.

In certain embodiments of any of Formulas A-X or I-IV, Y is In some embodiments, Y is

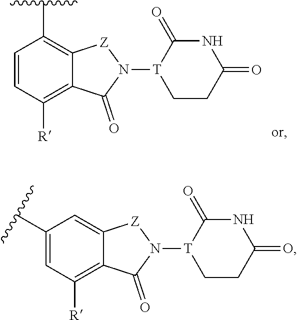

wherein each T is independently CH or N; and each Z is independently —CH₂— or —C(O)—; and each R′ is hydrogen, methyl, or NH₂.

In certain embodiments of any of Formulas A-X or I-IV, Y is any of the following:

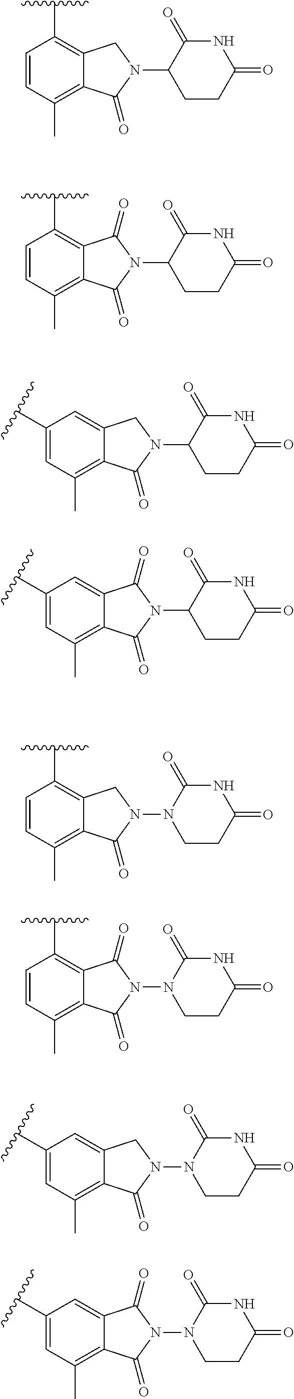

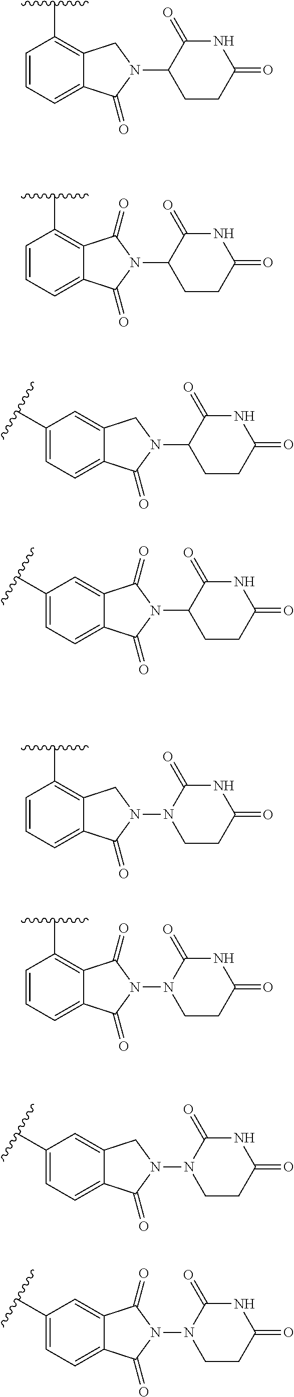

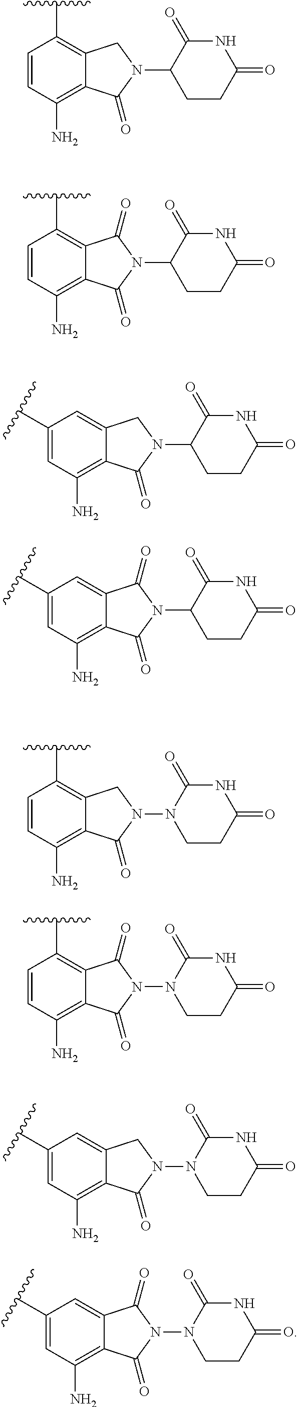

### General Synthetic Schemes

Compounds can be prepared or synthesized according to any technique deemed suitable by the person of skill in the art. In certain embodiments, compounds are prepared according to International Application No. PCT/US2019/56112, filed Oct. 14, 2019, incorporated by reference herein in its entirety. Exemplary synthetic schemes are described below.

### General Procedure 1: Amide Coupling

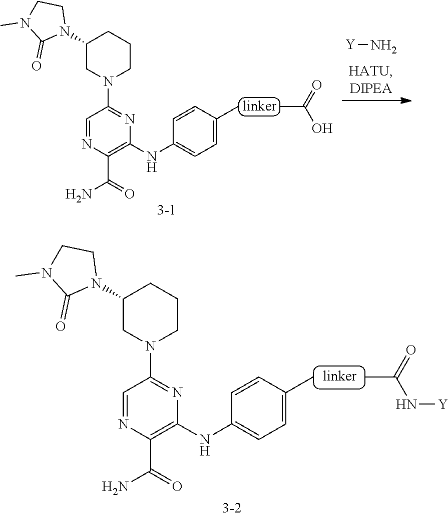

Intermediate (3-1), which can be generated by de-esterifying intermediate (1-6), is treated with amine, Y—NH₂, under coupling conditions to generate compounds of this disclosure (3-2), wherein the terminal linking group of L is an amide.

### General Procedure 2: Reductive Amination.

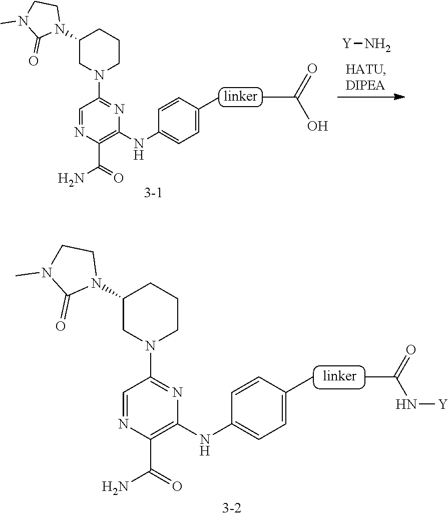

Intermediate (3-1), which can be generated by de-esterifying intermediate (1-6), is treated with amine, Y—NH₂, under coupling conditions to generate compounds of the present invention (3-2), wherein the terminal linking group of L is an amide.

### General Procedure 3: Aryl Fluoride Displacement

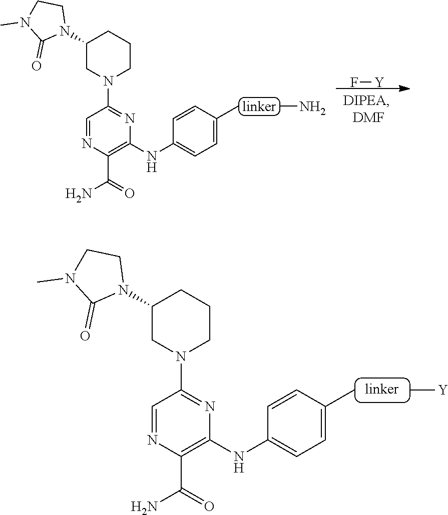

Intermediate (3-1), which can be generated by de-esterifying intermediate (1-6), is treated with any aryl fluoride, Y—F, under coupling conditions to generate compounds of the present invention (3-2), wherein the terminal linking group of L is an NH₂.

The abovementioned synthetic schemes can be used to synthesize the compounds in Table 1.

TABLE 1
Example compounds and/or pharmaceutically acceptable salts thereof for use
in the methods described herein.

| Compound | |
|---|---|
| Number | Structure |
| 1 | 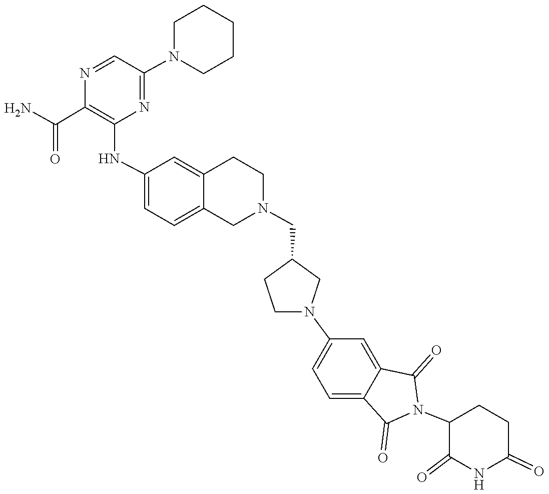 [SMILES: NC(=O)c1ncc(N2CCCCC2)nc1Nc1ccc2c(c1)CCN(C[C@H]1CCN(c3ccc4c(c3)C(=O)N(C3CCC(=O)NC3=O)C4=O)C1)C2] |
| 2 | 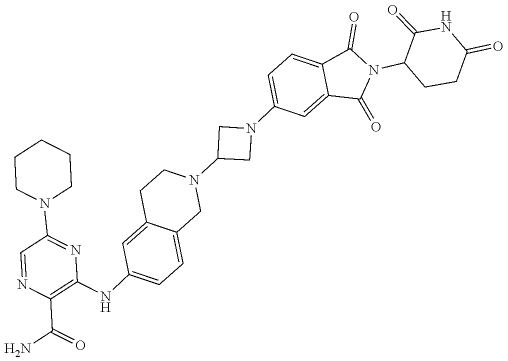 [SMILES: NC(=O)c1ncc(N2CCCCC2)nc1Nc1ccc2c(c1)CCN(C1CN(c3ccc4c(c3)C(=O)N(C3CCC(=O)NC3=O)C4=O)C1)C2] |
| 3 | 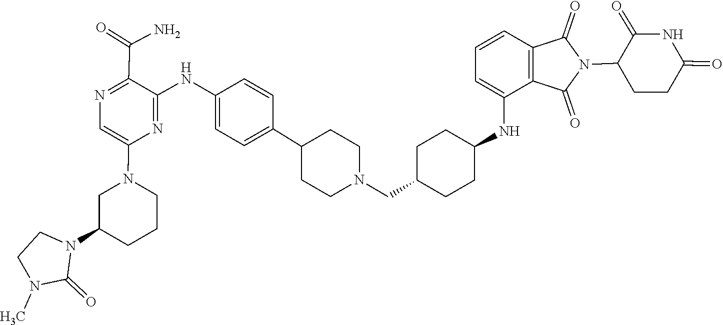 [SMILES: CN1CCN([C@@H]2CCCN(c3cnc(C(N)=O)c(Nc4ccc(C5CCN(C[C@H]6CC[C@H](Nc7cccc8c7C(=O)N(C7CCC(=O)NC7=O)C8=O)CC6)CC5)cc4)n3)C2)C1=O] |
| 4 | 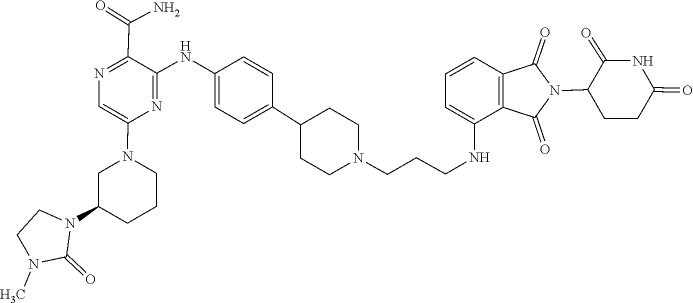 [SMILES: CN1CCN([C@@H]2CCCN(c3cnc(C(N)=O)c(Nc4ccc(C5CCN(CCCNc6cccc7c6C(=O)N(C6CCC(=O)NC6=O)C7=O)CC5)cc4)n3)C2)C1=O] |
| 5 | 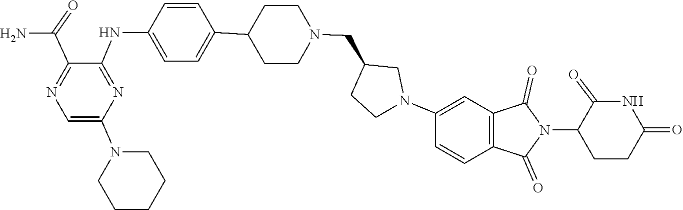 [SMILES: NC(=O)c1ncc(N2CCCCC2)nc1Nc1ccc(C2CCN(C[C@@H]3CCN(c4ccc5c(c4)C(=O)N(C4CCC(=O)NC4=O)C5=O)C3)CC2)cc1] |
| 6 | 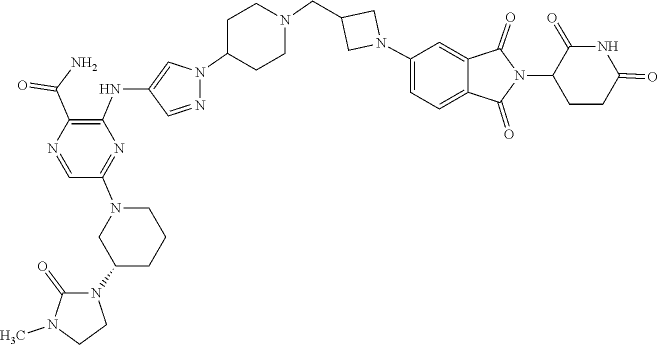 [SMILES: CN1CCN([C@H]2CCCN(c3cnc(C(N)=O)c(Nc4cnn(C5CCN(CC6CN(c7ccc8c(c7)C(=O)N(C7CCC(=O)NC7=O)C8=O)C6)CC5)c4)n3)C2)C1=O] |
| 7 | 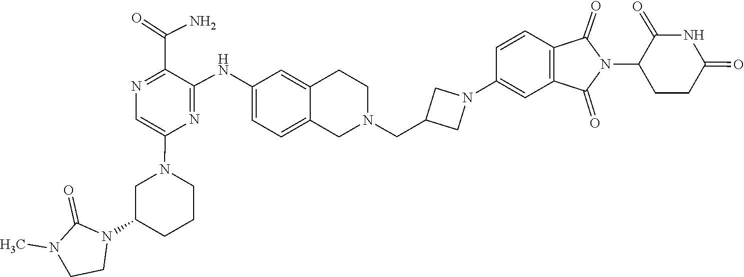 [SMILES: CN1CCN([C@H]2CCCN(c3cnc(C(N)=O)c(Nc4ccc5c(c4)CCN(CC4CN(c6ccc7c(c6)C(=O)N(C6CCC(=O)NC6=O)C7=O)C4)C5)n3)C2)C1=O] |
| 8 | 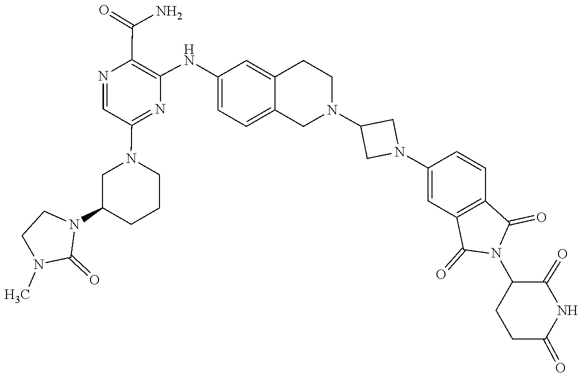 [SMILES: CN1CCN([C@@H]2CCCN(c3cnc(C(N)=O)c(Nc4ccc5c(c4)CCN(C4CN(c6ccc7c(c6)C(=O)N(C6CCC(=O)NC6=O)C7=O)C4)C5)n3)C2)C1=O] |
| 9 | 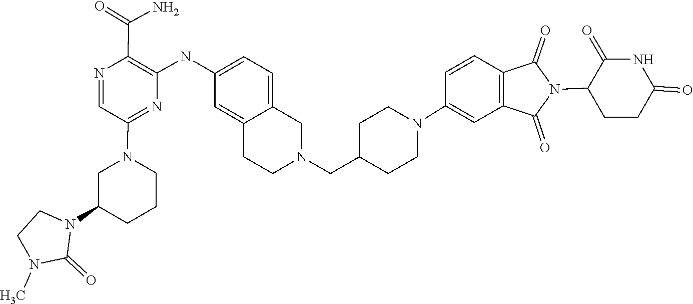 |
| 10 | 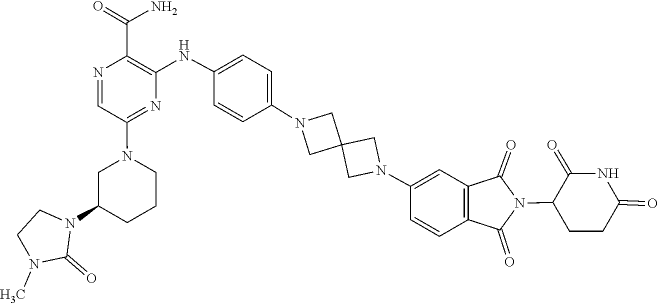 [SMILES: CN1CCN([C@@H]2CCCN(c3cnc(C(N)=O)c(Nc4ccc(N5CC6(C5)CN(c5ccc7c(c5)C(=O)N(C5CCC(=O)NC5=O)C7=O)C6)cc4)n3)C2)C1=O] |
| 11 | 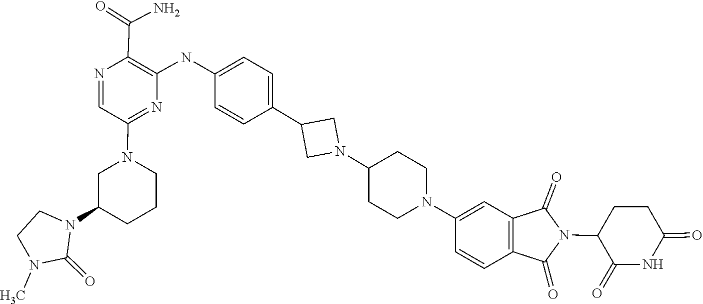 |
| 12 | 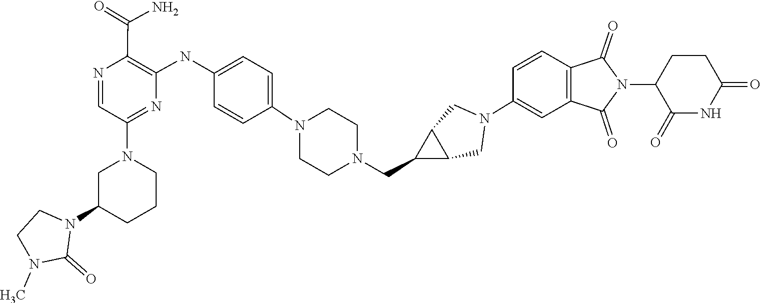 |
| 13 | 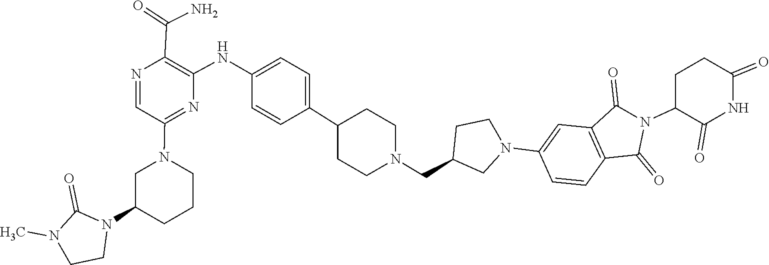 [SMILES: CN1CCN([C@@H]2CCCN(c3cnc(C(N)=O)c(Nc4ccc(C5CCN(C[C@H]6CCN(c7ccc8c(c7)C(=O)N(C7CCC(=O)NC7=O)C8=O)C6)CC5)cc4)n3)C2)C1=O] |
| 14 | 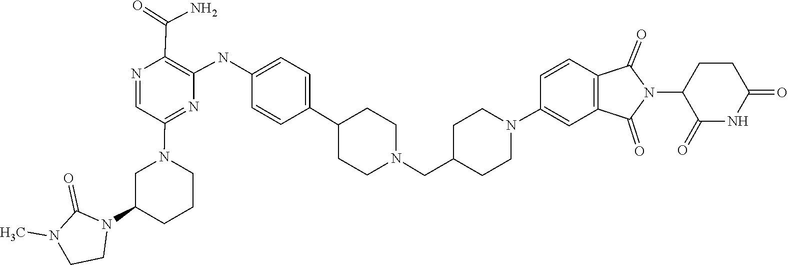 |
| 15 | 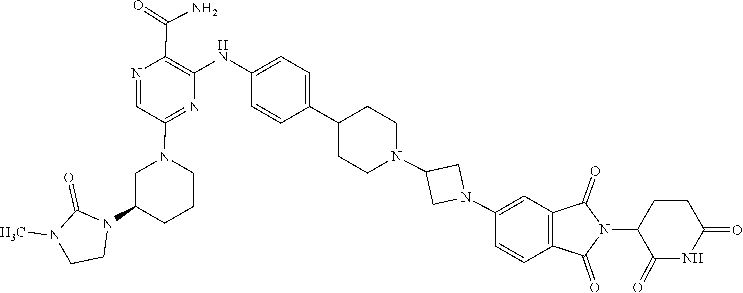 [SMILES: CN1CCN([C@@H]2CCCN(c3cnc(C(N)=O)c(Nc4ccc(C5CCN(C6CN(c7ccc8c(c7)C(=O)N(C7CCC(=O)NC7=O)C8=O)C6)CC5)cc4)n3)C2)C1=O] |
| 16 | 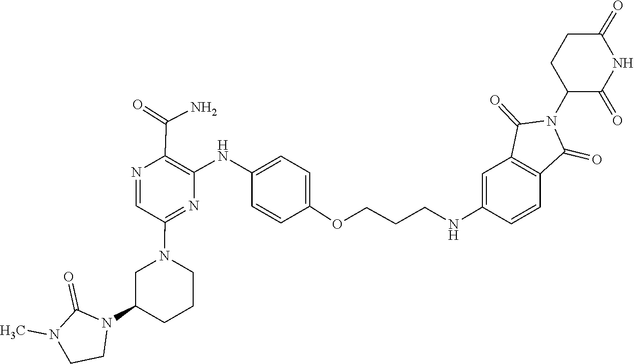 [SMILES: CN1CCN([C@@H]2CCCN(c3cnc(C(N)=O)c(Nc4ccc(OCCCNc5ccc6c(c5)C(=O)N(C5CCC(=O)NC5=O)C6=O)cc4)n3)C2)C1=O] |
| 17 | 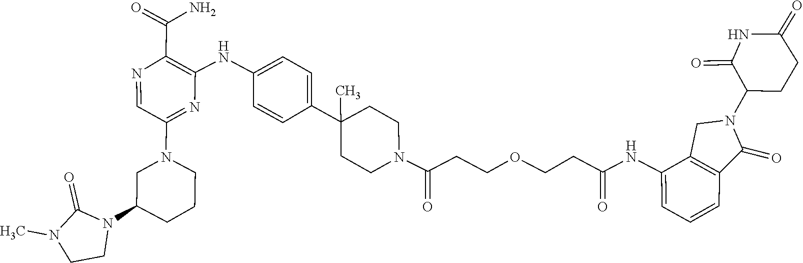 [SMILES: CN1CCN([C@@H]2CCCN(c3cnc(C(N)=O)c(Nc4ccc(C5(C)CCN(C(=O)CCOCCC(=O)Nc6cccc7c6CN(C6CCC(=O)NC6=O)C7=O)CC5)cc4)n3)C2)C1=O] |
| 18 | 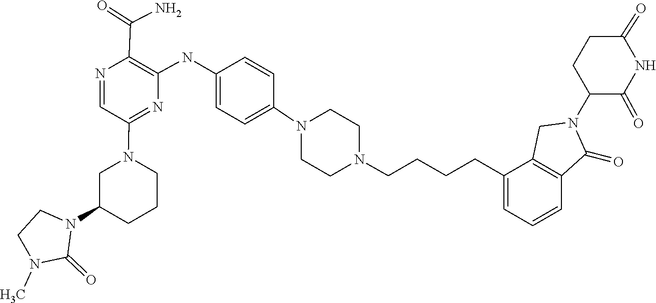 |
| 19 | 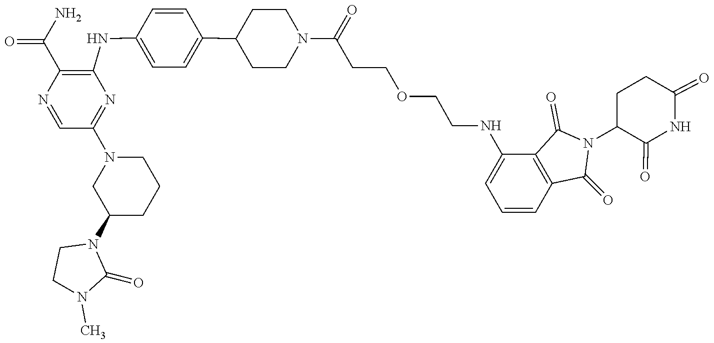 |

### Formulations and Administration

### Pharmaceutical Compositions

The compounds described herein can be formulated into pharmaceutical compositions that further comprise a pharmaceutically acceptable carrier, diluent, adjuvant, or vehicle. In one embodiment, this disclosure provides a pharmaceutical composition comprising a compound described above, and a pharmaceutically acceptable carrier, diluent, adjuvant, or vehicle. In one embodiment, this disclosure is a pharmaceutical composition comprising an effective amount of a compound of this disclosure or a pharmaceutically acceptable salt thereof and a pharmaceutically acceptable carrier, diluent, adjuvant, or vehicle. Pharmaceutically acceptable carriers include, for example, pharmaceutical diluents, excipients, or carriers suitably selected with respect to the intended form of administration, and consistent with conventional pharmaceutical practices.

According to another embodiment, the description provides a composition comprising a compound herein or a pharmaceutically acceptable salt thereof and a pharmaceutically acceptable carrier, adjuvant, or vehicle. Pharmaceutical compositions of this description comprise a therapeutically effective amount of a compound of Formula A-X or I-IV wherein a “therapeutically effective amount” is an amount that is (a) effective to measurably degrade BTK (or reduce the amount of BTK) in a biological sample or in a patient; or (b) effective in treating and/or ameliorating a disease or disorder that is mediated by BTK.

The term “patient,” as used herein, means an animal, alternatively a mammal, and alternatively a human.

It also will be appreciated that certain compounds of this disclosure can exist in free form for treatment, or where appropriate, as a pharmaceutically acceptable derivative (e.g., a salt) thereof. According to this disclosure, a pharmaceutically acceptable derivative includes, but is not limited to, pharmaceutically acceptable prodrugs, salts, esters, salts of such esters, or any other adduct/educt or derivative that upon administration to a patient in need is capable of providing, directly or indirectly, a compound as otherwise described herein, or a metabolite or residue thereof.

As used herein, the term “pharmaceutically acceptable salt” refers to those salts that are, within the scope of sound medical judgement, suitable for use in contact with the tissues of humans and lower animals without undue toxicity, irritation, allergic response, and the like.

Pharmaceutically acceptable salts are well known in the art. For example, S. M. Berge et al., describe pharmaceutically acceptable salts in detail in J. Pharmaceutical Sciences 1977, 66, 1-19, incorporated herein by reference. Pharmaceutically acceptable salts of the compounds of this description include those derived from suitable inorganic and organic acids and bases. Examples of pharmaceutically acceptable, nontoxic acid addition salts include salts of an amino group formed with inorganic acids such as hydrochloric acid, hydrobromic acid, phosphoric acid, sulfuric acid, and perchloric acid; or with organic acids such as acetic acid, oxalic acid, maleic acid, tartaric acid, citric acid, succinic acid, or malonic acid; or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. This description also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A pharmaceutically acceptable carrier may contain inert ingredients that do not unduly inhibit the biological activity of the compounds. The pharmaceutically acceptable carriers should be biocompatible, for example, non-toxic, non-inflammatory, non-immunogenic, or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants, and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, the use of such conventional carrier medium is contemplated to be within the scope of this description. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful, uncomfortable, or risky. Side effects include, but are not limited to, fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain, and headache), hair loss, asthenia, dizziness, extra-pyramidal symptoms, akathisia, cardiovascular disturbances, and sexual dysfunction.

Some examples of materials that can serve as pharmaceutically acceptable carriers include, but are not limited to, ion exchangers, alumina, aluminum stearate, lecithin, serum proteins (such as human serum albumin), buffer substances (such as tween 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring, and perfuming agents. Preservatives and antioxidants can also be present in the composition, according to the judgment of the formulator.

As used herein, the term “measurably degrade,” means a measurable reduction in (a) BTK activity, between a sample comprising a compound of this description and a BTK and an equivalent sample comprising a BTK in the absence of said compound; or (b) the concentration of the BTK in a sample over time.

### Administration

The compositions of this disclosure are administered orally. The pharmaceutically acceptable compositions of this description may be orally administered in any orally acceptable dosage form including, but not limited to, capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring, or coloring agents also may be added.

Liquid dosage forms for oral administration include, but are not limited to, pharmaceutically acceptable emulsions, microemulsions, solutions, suspensions, syrups, and elixirs. In addition to the active compounds herein, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions also can include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound herein is mixed with at least one inert, pharmaceutically acceptable excipient or carrier such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets, and pills, the dosage form also may comprise buffering agents.

Solid compositions of a similar type also may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. Solid dosage forms optionally may contain opacifying agents. These solid dosage forms also can be of a composition such that they release the active ingredient(s) only, for example, in a certain part of the intestinal tract, optionally, in a delayed manner. Examples of embedding compositions that can be used include polymeric substances and waxes. Solid compositions of a similar type also may be employed as fillers in soft and hard-filled gelatin capsules using such excipients as lactose or milk sugar as well as high molecular weight polyethylene glycols and the like.

The active compounds herein also can be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose, or starch. Such dosage forms also may comprise, as is normal practice, additional substances other than inert diluents, for example, tableting lubricants and other tableting aids such a magnesium stearate and microcrystalline cellulose. In the case of capsules, tablets and pills, the dosage forms also may comprise buffering agents. They may optionally contain opacifying agents and also can be of a composition such that they release the active ingredient(s) only, for example, in a certain part of the intestinal tract, optionally, in a delayed manner. Examples of embedding compositions that can be used include polymeric substances and waxes.

The compounds of the description are formulated in dosage unit form for ease of administration and uniformity of dosage. As used herein, the phrase “dosage unit form” refers to a physically discrete unit of agent appropriate for the patient to be treated. It will be understood, however, that the total daily usage of the compounds and compositions of this disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex, and diet of the patient; the time of administration, route of administration, and rate of excretion of the specific compound employed; the duration of the treatment; drugs used in combination or coincidental with the specific compound employed, and like factors well known in the medical arts.

The amount of the compounds of this disclosure that may be combined with the carrier materials to produce a composition in a single dosage form will vary depending upon the host treated, the particular mode of administration, and other factors. The compositions should be formulated so that a dosage of between 0.01-100 mg/kg body weight/day of the compound or inhibitor can be administered to a patient receiving these compositions.

Depending upon the particular condition, or disease, to be treated or prevented, additional therapeutic agents, which are normally administered to treat or prevent that condition, also may be present in the compositions of this disclosure. As used herein, additional therapeutic agents that are normally administered to treat or prevent a particular disease, or condition, are known as “appropriate for the disease, or condition, being treated.”

For example, chemotherapeutic agents or other anti-proliferative agents may be combined with the compounds of this disclosure to treat proliferative diseases and cancer. Examples of known chemotherapeutic agents include, but are not limited to, PI3K inhibitors (e.g., idelalisib and copanlisib), BCL-2 inhibitors (e.g., venetoclax), BTK inhibitors (e.g., ibrutinib and acalabrutinib), etoposide, CD20 antibodies (e.g., rituximab, ocrelizumab, obinutuzumab, ofatumumab, ibritumomab tiuxetan, tositumomab, and ublituximab), aletuzumab, bendamustine, cladribine, doxorubicin, chlorambucil, prednisone, midostaurin, lenalidomide, pomalidomide, checkpoint inhibitors (e.g., ipilimumab, nivolumab, pembolizumab, atezolizumab, avelumab, durvalumab), engineered cell therapy (e.g., CAR-T therapy—Kymriah®, Yescarta®), Gleevec™, adriamycin, dexamethasone, vincristine, cyclophosphamide, fluorouracil, topotecan, taxol, interferons, and platinum derivatives.

And, in some instances, radiation therapy is administered during the treatment course wherein a compound of this disclosure (or a pharmaceutically acceptable salt thereof) is administered to a patient in need thereof.

Other examples of agents with which the compounds or inhibitors of this disclosure also may be combined include, without limitation, treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

The amount of additional therapeutic agent present in the compositions of this disclosure will be no more than the amount that would normally be administered in a composition comprising that therapeutic agent as the only active agent. The amount of additional therapeutic agent in the presently disclosed compositions will range from about 50% to 100% of the amount normally present in a composition comprising that agent as the only therapeutically active agent.

### EXAMPLES

Additional embodiments are disclosed in further detail in the following examples, which are not in any way intended to limit the scope of the claims.

### Example 1

### General Procedure 1: Amide Coupling

A mixture of amine (0.03 mmol), acid (0.03 mmol), HATU (0.04 mmol), DIPEA (0.15 mmol), and DMF was allowed to stir at room temperature for 30 minutes. The mixture was purified by HPLC (H₂O/MeCN with 0.1% TFA) to afford the amide product. Compound 21 was prepared according to procedure 1.

### General Procedure 2: Reductive Amination

A mixture of amine TFA salt (0.07 mmol), aldehyde (0.1 mmol), triethylamine (0.28 mmol), and DCE were allowed to stir at room temperature for 10 minutes. NaBH(OAc)₃ (0.14 mmol) was added and the mixture was allowed to stir at room temperature for 2 h. The mixture was filtered through celite, washed with CH₂Cl₂, concentrated, and purified by HPLC (H₂O/MeCN with 0.1% TFA) to afford the amine product. Compounds 6, 7, and 199 were prepared according to procedure 2.

### General Procedure 3: Aryl Fluoride Displacement

A mixture of amine (0.22 mmol), aryl fluoride (0.22 mmol), DIPEA (0.88 mmol), and DMF (1 mL) was allowed to stir at 90° C. for 16 h. The mixture was purified by HPLC (H₂O/MeCN with 0.1% TFA) to afford the desired product. Compound 20 was prepared according to procedure 3.

Compounds 3-19 are prepared according to PCT/US2019/56112, filed Oct. 14, 2019, which is hereby incorporated by reference in its entirety.

### Example 1—Compound 1

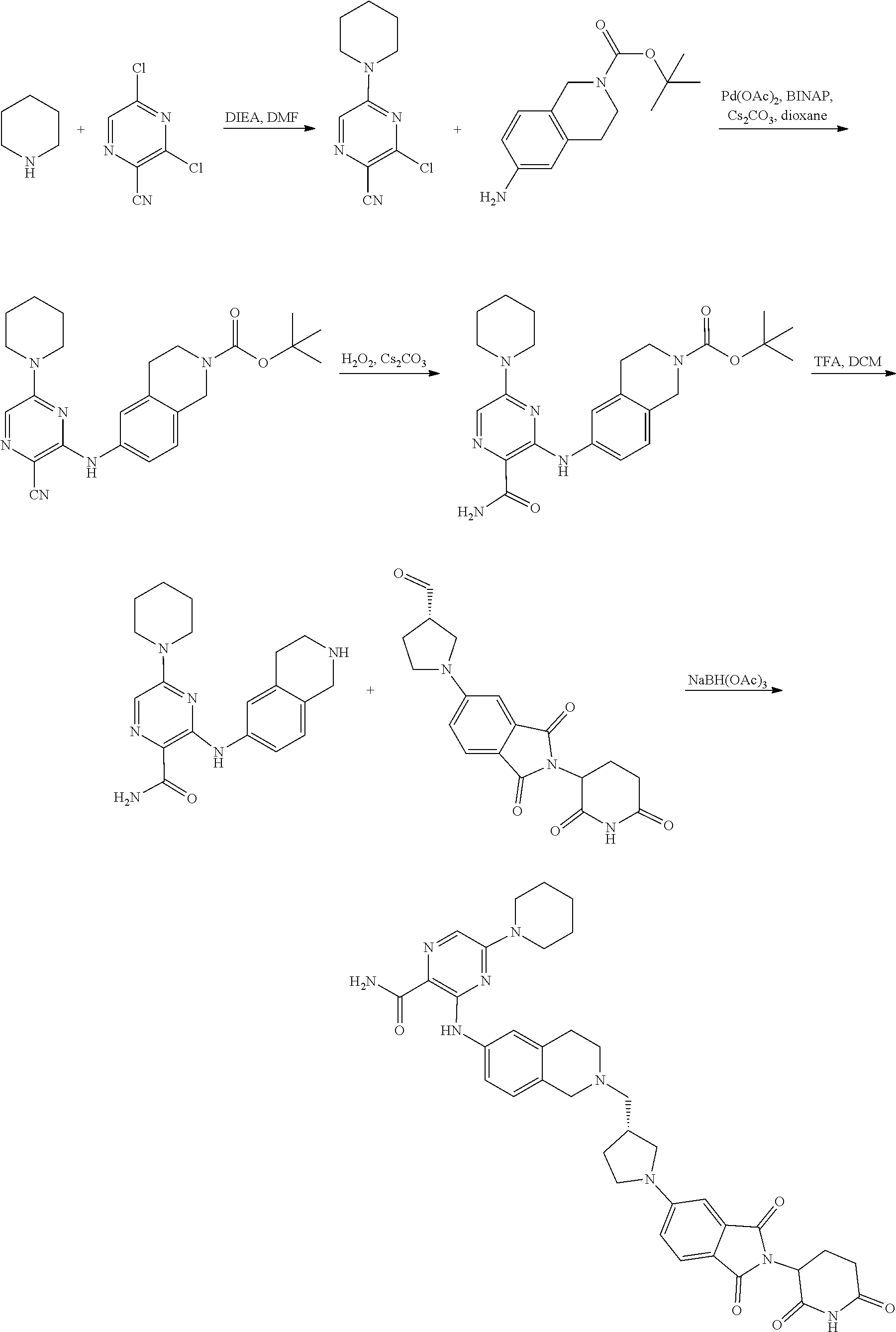

A mixture of 3,5-dichloropyrazine-2-carbonitrile (850 mg, 4.89 mmol), piperidine (0.48 mL, 4.89 mmol), ethylbis(propan-2-yl)amine (1.70 mL, 9.77 mmol) and DMF (20 mL) was allowed to stir at r.t. for 2 h. EtOAc and H2O were added. The organic layer was dried with MgSO₄, filtered, concentrated and purified by MPLC (0-100% EtOAc in hexanes) to afford 3-chloro-5-(piperidin-1-yl)pyrazine-2-carbonitrile (1079.6 mg, 99.2%). LCMS: C₁₀H₁₁ClN₄ requires: 222, found: m/z=223 [M+H]⁺.

A mixture of tert-butyl 6-amino-3,4-dihydro-1H-isoquinoline-2-carboxylate (1398 mg, 5.63 mmol), 3-chloro-5-(piperidin-1-yl)pyrazine-2-carbonitrile (1254 mg, 5.63 mmol), palladium acetate (253 mg, 1.13 mmol), [2′-(diphenylphosphanyl)-[1,1′-binaphthalen]-2-yl]diphenylphosphane (701 mg, 1.13 mmol) and cesium carbonate (5504 mg, 16.89 mmol) was degassed and backfilled with N₂ 5 times. The mixture was allowed to stir at 100° C. for 90 min. The mixture was filtered through celite washing with MeOH/EtOAc, concentrated and purified by MPLC (0-100% EtOAc in CH₂Cl₂) to afford tert-butyl 6-((3-cyano-6-(piperidin-1-yl)pyrazin-2-yl)amino)-3,4-dihydroisoquinoline-2(1H)-carboxylate (1.830 g, 74.8%). LCMS: C₂₄H₃₀N₆O₂ requires: 434, found: m/z=435 [M+H]⁺.

H₂O₂ 30% aqueous solution (7.11 mL) was added to a mixture of cesium carbonate (1372 mg, 4.21 mmol), DMSO (2.5 mL), MeOH (50 mL) and tert-butyl 6-((3-cyano-6-(piperidin-1-yl)pyrazin-2-yl)amino)-3,4-dihydroisoquinoline-2(1H)-carboxylate (1830 mg, 4.21 mmol). The mixture was allowed to stir at rt for 30 min. The mixture was concentrated. EtOAc was added and the organic phase was washed with H₂O and brine. The organic layer was dried with MgSO₄, filtered, concentrated and purified by MPLC (0-10% MeOH in CH2Cl2) to afford tert-butyl 6-((3-carbamoyl-6-(piperidin-1-yl)pyrazin-2-yl)amino)-3,4-dihydroisoquinoline-2(1H)-carboxylate (1.6470 g, 86.4%). LCMS: C₂₄H₃₂N₆O₃ requires: 452, found: m/z=453 [M+H]⁺.

A mixture of tert-butyl 6-((3-carbamoyl-6-(piperidin-1-yl)pyrazin-2-yl)amino)-3,4-dihydroisoquinoline-2(1H)-carboxylate (1647 mg, 3.64 mmol), CH₂Cl₂ (30 mL) and TFA (6 mL) was allowed to stir at rt for 2 h. The volatiles were removed. The mixture was filtered through a NaHCO₃ cartridge, concentrated and purified by reverse phase MPLC (5-90% MeCN in H₂O) to afford 5-(piperidin-1-yl)-3-((1,2,3,4-tetrahydroisoquinolin-6-yl)amino)pyrazine-2-carboxamide (1.2800 g, 99.8%). LCMS: C₁₉H₂₄N₆O requires: 352, found: m/z=353 [M+H]⁺.

### Example 2—Compound 2

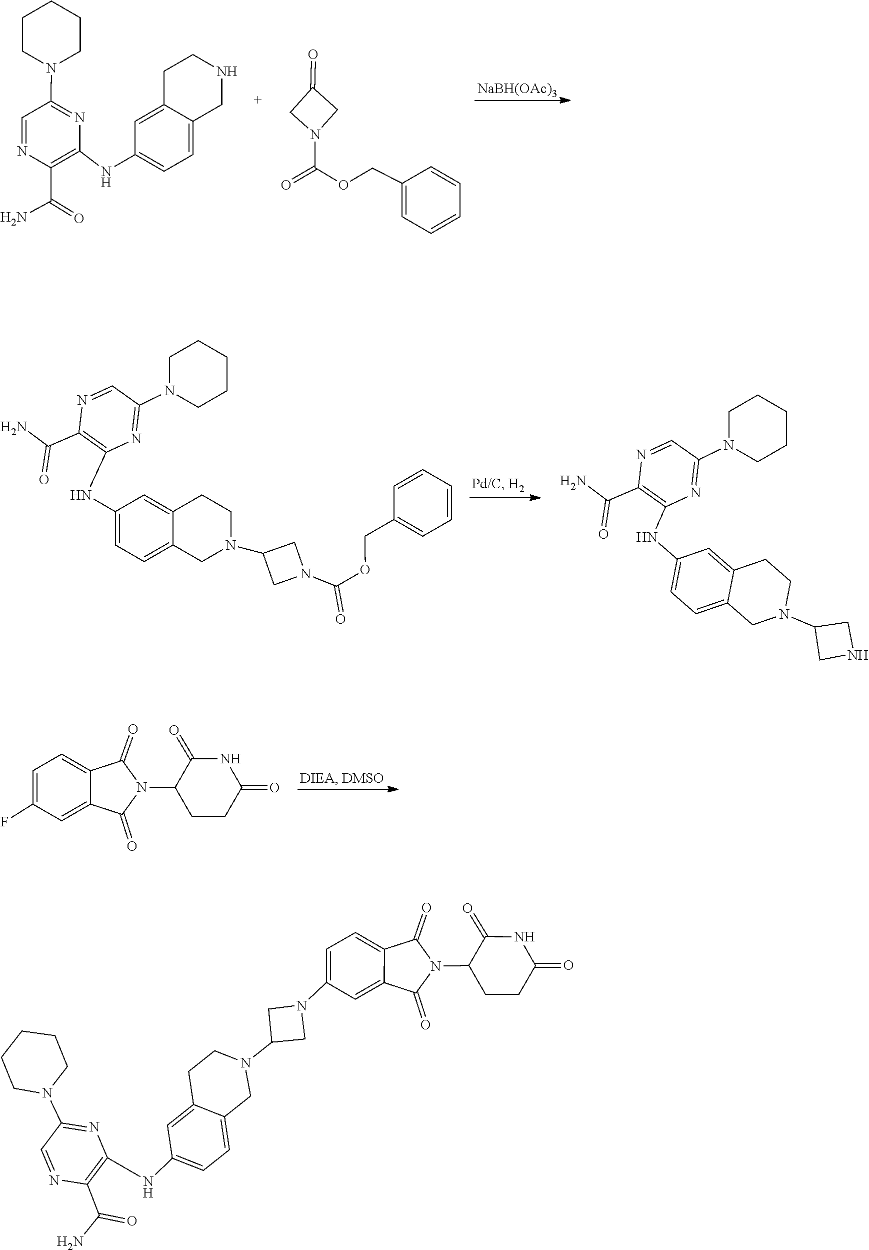

Sodium triacetoxyborohydride (372 mg, 1.75 mmol) was added to a mixture of AcOH (1 drop), benzyl 3-oxoazetidine-1-carboxylate (120 mg, 0.58 mmol), 5-(piperidin-1-yl)-3-(1,2,3,4-tetrahydroisoquinolin-6-ylamino)pyrazine-2-carboxamide (206 mg, 0.58 mmol) and DCE (5 mL). The mixture was allowed to stir at rt for 1 h. The mixture was diluted with EtOAc, aq NaHCO₃ was added, the organic layer was dried with MgSO₄, filtered, concentrated and carried to the next step without further purification. LCMS: C₃₀H₃₅N₇O₃ requires: 541, found: m/z=542 [M+H]⁺

A mixture of benzyl 3-(6-{[3-carbamoyl-6-(piperidin-1-yl)pyrazin-2-yl]amino}-3,4-dihydro-1H-isoquinolin-2-yl)azetidine-1-carboxylate (315 mg, 0.58 mmol), Pd/C (62 mg, 0.58 mmol) and EtOH (10 mL) was evacuated and backfilled with H₂ 5 times. The mixture was allowed to stir at rt overnight. The mixture was filtered washing with MeOH/EtOAc, concentrated and carried to the next step. LCMS: C₂₂H₂₉N₇O requires: 407, found: m/z=408 [M+H]⁺.

A mixture of rac-2-[(3R)-2,6-dioxopiperidin-3-yl]-5-fluoroisoindole-1,3-dione (24.40 mg, 0.09 mmol), 3-{[2-(azetidin-3-yl)-3,4-dihydro-1H-isoquinolin-6-yl]amino}-5-(piperidin-1-yl)pyrazine-2-carboxamide (30 mg, 0.07 mmol), N,N-diisopropylethylamine (0.04 mL, 0.22 mmol) and DMSO (1 mL) was allowed to stir at 90° C. for 4 h. The mixture was purified by HPLC (5-95% MeCN in H₂O with 0.1% TFA) to afford rac-3-{[2-(1-{2-[(3R)-2,6-dioxopiperidin-3-yl]-1,3-dioxoisoindol-5-yl}azetidin-3-yl)-3,4-dihydro-1H-isoquinolin-6-yl]amino}-5-(piperidin-1-yl)pyrazine-2-carboxamide (0.0096 g, 19.6%). ¹H NMR (500 MHz, Acetonitrile-d3) δ 11.22 (s, 1H), 8.89 (s, 1H), 7.72-7.65 (m, 2H), 7.59 (s, 1H), 7.49 (d, J=8.6 Hz, 1H), 7.41 (s, 1H), 7.13 (d, J=8.5 Hz, 1H), 6.89 (d, J=2.1 Hz, 1H), 6.78-6.70 (m, 1H), 5.81 (s, 1H), 4.97 (dd, J=12.3, 5.5 Hz, 1H), 4.51-4.35 (m, 3H), 4.20 (s, 2H), 3.73 (t, J=5.4 Hz, 4H), 3.15 (s, 2H), 2.74 (td, J=20.0, 19.1, 11.0 Hz, 3H), 1.71 (d, J=35.4 Hz, 8H). LCMS: C₃₅H₃₇N₉O₅ requires: 663, found: m/z=664 [M+H]⁺.

| Com- | | |
|---|---|---|
| pound | | Mass Spec |
| No. | ¹H NMR | (LCMS) |
| 1 | ¹H NMR (500 MHz, Acetonitrile-d3) δ 11.40 (s, | LCMS: |
| | 1H), 11.24 (s, 1H), 8.89 (s, 1H), 7.70 (d, J = 2.1 | C₃₇H₄₁N₉O₅ |
| | Hz, 1H), 7.66 (d, J = 8.4 Hz, 1H), 7.60 (s, 1H), | requires: |
| | 7.50 (dd, J = 8.5, 2.1 Hz, 1H), 7.42 (d, J = 3.6 | 691, found: |
| | Hz, 1H), 7.15 (d, J = 8.4 Hz, 1H), 6.96 (s, 1H), | m/z = 692 |
| | 6.87-6.74 (m, 1H), 5.83 (s, 1H), 4.96 (dd, J = | [M + H]⁺. |
| | 12.3, 5.4 Hz, 1H), 4.61 (s, 1H), 4.20 (s, 1H), | |
| | 3.85-3.66 (m, 6H), 3.59 (td, J = 9.3, 8.4, 3.1 Hz, | |
| | 1H), 3.48 (q, J = 9.1, 8.6 Hz, 1H), 3.33 (d, J = | |
| | 33.0 Hz, 5H), 3.09 (s, 0H), 2.99 (q, J = 7.7 Hz, | |
| | 1H), 2.75 (tdd, J = 17.7, 10.4, 6.4 Hz, 3H), | |
| | 1.71 (dt, J = 31.2, 5.5 Hz, 7H). | |
| 2 | ¹H NMR (500 MHz, Acetonitrile-d3) δ 11.22 | LCMS: |
| | (s, 1H), 8.89 (s, 1H), 7.72-7.65 (m, 2H), 7.59 | C₃₅H₃₇N₉O₅ |
| | (s, 1H), 7.49 (d, J = 8.6 Hz, 1H), 7.41 (s, 1H), | requires: |
| | 7.13 (d, J = 8.5 Hz, 1H), 6.89 (d, J = 2.1 Hz, | 663, found: |
| | 1H), 6.78-6.70 (m, 1H), 5.81 (s, 1H), 4.97 (dd, | m/z = 664 |
| | J = 12.3, 5.5 Hz, 1H), 4.51-4.35 (m, 3H), 4.20 | [M + H]⁺. |
| | (s, 2H), 3.73 (t, J = 5.4 Hz, 4H), 3.15 (s, 2H), | |
| | 2.74 (td, J = 20.0, 19.1, 11.0 Hz, 3H), 1.71 (d, | |
| | J = 35.4 Hz, 8H). | |
| 3 | ¹H NMR (500 MHz, Acetonitrile-d₃) δ 11.12 (s, | LCMS: |
| | 1H), 9.79 (s, 1H), 8.92 (s, 1H), 7.68-7.50 (m, | C₄₅H₅₅N₁₁O₆ |
| | 4H), 7.43 (s, 1H), 7.21 (d, J = 8.2 Hz, 2H), | requires: |
| | 7.08 (dd, J = 21.8, 7.8 Hz, 2H), 6.17 (s, 1H), | 845, found: |
| | 5.84 (s, 1H), 4.96 (dd, J = 12.4, 5.3 Hz, 1H), | m/z = 846 |
| | 4.36 (dd, J = 48.4, 13.3 Hz, 2H), 3.68 (t, J = | [M + H]⁺. |
| | 14.8 Hz, 3H), 3.52 (s, 1H), 3.46-3.22 (m, 3H), | |
| | 3.16-2.89 (m, 5H), 2.88-2.62 (m, 6H), 2.19- | |
| | 2.01 (m, 5H), 1.68 (t, J = 12.6 Hz, 1H), 1.46- | |
| | 1.12 (m, 4H). | |
| 4 | ¹H NMR (500 MHz, Acetonitrile-d₃) δ 11.11 | LCMS: |
| | (s, 1H), 10.44 (s, 1H), 8.93 (s, 1H), 7.66-7.52 | C₄₁H₄₉N₁₁O₆ |
| | (m, 4H), 7.43 (s, 1H), 7.20 (d, J = 8.2 Hz, 2H), | requires: |
| | 7.10 (d, J = 7.7 Hz, 2H), 6.47 (s, 1H), 5.83 (s, | 791, found: |
| | 1H), 4.97 (dd, J = 12.5, 5.4 Hz, 1H), 4.42 (d, | m/z = 792 |
| | J = 12.6 Hz, 1H), 4.31 (d, J = 13.6 Hz, 1H), | [M + H]⁺. |
| | 3.66 (dd, J = 31.4, 11.6 Hz, 2H), 3.52-3.22 (m, | |
| | 8H), 3.22-2.89 (m, 5H), 2.89-2.64 (m, 9H), | |
| | 2.19-2.04 (m, 6H), 1.93-1.56 (m, 4H). | |
| 5 | ¹H NMR (500 MHz, DMSO-d₆) δ 11.28 (s, | LCMS: |
| | 1H), 11.06 (s, 1H), 7.73 (d, J = 2.8 Hz, 1H), | C₃₉H₄₅N₉O₅ |
| | 7.66 (s, 1H), 7.64 (d, J = 8.4 Hz, 1H), 7.55- | requires: |
| | 7.48 (m, 2H), 7.31 (d, J = 2.9 Hz, 1H), 7.20 | 719, found: |
| | (s, 1H), 7.18 (s, 1H), 6.90 (d, J = 2.2 Hz, 1H), | m/z = 720 |
| | 6.82 (dd, J = 8.6, 2.2 Hz, 1H), 5.05 (dd, J = | [M + H]⁺ |
| | 12.9, 5.4 Hz, 1H), 3.67 (t, J = 5.5 Hz, 4H), | |
| | 3.56 (dd, J = 10.3, 7.2 Hz, 1H), 3.54-3.46 | |
| | (m, 1H), 3.40 (dt, J = 10.3, 7.5 Hz, 1H), 3.15 | |
| | (dd, J = 10.4, 6.8 Hz, 1H), 3.04 (d, J = 10.9 | |
| | Hz, 1H), 2.97 (d, J = 11.0 Hz, 1H), 2.88 (ddd, | |
| | J = 17.4, 14.1, 5.5 Hz, 1H), 2.67-2.57 (m, 2H), | |
| | 2.59-2.51 (m, 1H), 2.50-2.40 (m, 1H), 2.37 (d, | |
| | J = 7.6 Hz, 2H), 2.18-2.08 (m, 1H), 2.09-1.96 | |
| | (m, 3H), 1.82-1.71 (m, 3H), 1.71-1.61 (m, 4H), | |
| | 1.64-1.54 (m, 4H). | |
| 6 | ¹H NMR (500 MHz, DMSO-d₆) δ 11.08 (s, 1H), | LCMS: |
| | 10.86 (s, 1H), 7.98 (s, 1H), 7.74-7.68 (m, 1H), | C₃₉H₄₇N₁₃O₆ |
| | 7.65 (d, J = 8.3 Hz, 1H), 7.61 (s, 1H), 7.56 (s, | requires: |
| | 1H), 7.29 (d, J = 2.9 Hz, 1H), 6.80 (d, J = 2.1 | 793, found: |
| | Hz, 1H), 6.67 (dd, J = 8.4, 2.1 Hz, 1H), 5.06 | m/z = 794 |
| | (dd, J = 12.7, 5.4 Hz, 1H), 4.34 (dd, J = 46.2, | [M + H]⁺. |
| | 13.0 Hz, 2H), 4.16 (s, 3H), 3.79-3.54 (m, 3H), | |
| | 3.14-2.81 (m, 4H), 2.76-2.56 (m, 5H), 2.24-1.69 | |
| | (m, 10H), 1.57 (d, J = 12.8 Hz, 1H). | |
| 7 | ¹H NMR (500 MHz, DMSO-d₆) δ 11.24 (s, 1H), | LCMS: |
| | 11.08 (s, 1H), 7.78 (s, 1H), 7.67 (q, J = 10.7, | C₄₀H₄₅N₁₁O₆ |
| | 8.1 Hz, 2H), 7.57 (s, 1H), 7.36 (s, 1H), 7.16 (d, | requires: |
| | J = 8.2 Hz, 1H), 6.98 (d, J = 8.4 Hz, 1H), 6.80 (s, | 775.9, found: |
| | 1H), 6.66 (d, J = 8.4 Hz, 1H), 5.07 (dd, J = 12.9, | m/z = 776.7 |
| | 5.5 Hz, 1H), 4.34 (dd, J = 43.8, 11.9 Hz, 2H), | [M + H]⁺ |
| | 4.17 (t, J = 8.4 Hz, 2H), 3.75 (t, J = 6.8 Hz, 2H), | |
| | 3.64 (d, J = 11.4 Hz, 1H), 3.55 (s, 2H), 3.08 | |
| | (dd, J = 24.9, 13.1 Hz, 2H), 3.03-2.85 (m, 3H), | |
| | 2.77 (t, J = 6.3 Hz, 3H), 2.70 (s, 5H), 2.11-1.96 | |
| | (m, 1H), 1.81 (q, J = 21.3, 17.4 Hz, 3H), 1.59 | |
| | (d, J = 14.4 Hz, 1H). | |
| 8 | ¹H NMR (500 MHz, DMSO-d₆) δ 11.44 (s, 1H), | LCMS: |
| | 11.09 (d, J = 10.0 Hz, 1H), 10.81 (s, 1H), 7.83 (s, | C₃₉H₄₃N₁₁O₆ |
| | 1H), 7.79-7.63 (m, 3H), 7.41 (s, 2H), 7.16 (d, J = | expected: |
| | 8.2 Hz, 1H), 6.93 (s, 1H), 6.84-6.73 (m, 1H), 5.08 | 761.8. Found |
| | (ddd, J = 15.5, 10.1, 5.4 Hz, 1H), 4.65-4.02 (m, | [M + H}: |
| | 10H), 3.28 (dd, J = 9.9, 7.2 Hz, 3H), 3.10 (d, J = | 762.7 |
| | 15.2 Hz, 5H), 3.04-2.74 (m, 4H), 2.71 (s, 4H), | |
| | 2.60 (d, J = 19.6 Hz, 2H), 2.04 (dq, J = 11.9, 6.0, | |
| | 4.8 Hz, 1H), 1.81 (td, J = 18.8, 16.1, 10.1 Hz, | |
| | 3H), 1.59 (d, J = 11.5 Hz, 1H). | |
| 9 | ¹H NMR (500 MHz, DMSO-d₆) δ 11.44 (d, | LCMS |
| | J = 9.7 Hz, 1H), 11.09 (s, 1H), 9.58 (s, 1H), | C₄₂H₄₉N₁₁O₆ |
| | 7.83 (s, 1H), 7.80-7.62 (m, 3H), 7.51-7.25 (m, | requires: |
| | 4H), 7.16 (d, J = 8.5 Hz, 1H), 5.08 (dd, J = | 804, found: |
| | 12.8, 5.4 Hz, 1H), 4.58 (d, J = 15.3 Hz, 1H), | m/z = 805 |
| | 4.43-4.22 (m, 3H), 4.13 (d, J = 13.2 Hz, 2H), | [M + H]⁺. |
| | 3.10-2.85 (m, 8H), 2.70 (d, J = 2.7 Hz, 4H), | |
| | 2.15-1.68 (m, 7H), 1.59 (d, J = 12.4 Hz, | |
| | 1H), 1.31 (qd, J = 17.9, 16.1, 8.7 Hz, 2H). | |
| 10 | ¹H NMR (500 MHz, DMSO-d₆) δ 11.08 (s, | LCMS: |
| | 1H), 10.94 (s, 1H), 7.73-7.65 (m, 2H), 7.60 | C₃₈H₄₁N₁₁O₆ |
| | (s, 1H), 7.41 (d, J = 8.7 Hz, 2H), 7.27 (d, | requires: |
| | J = 3.0 Hz, 1H), 6.84 (d, J = 2.0 Hz, 1H), | 747, found: |
| | 6.70 (dd, J = 8.4, 2.1 Hz, 1H), 6.44 (d, J = | m/z = 748 |
| | 8.6 Hz, 2H), 5.07 (dd, J = 12.8, 5.4 Hz, 1H), | [M + H]⁺. |
| | 4.37 (d, J = 12.4 Hz, 1H), 4.26 (s, 4H), 3.99 | |
| | (s, 4H), 3.05-2.83 (m, 3H), 2.73 (s, 3H), | |
| | 2.67-2.55 (m, 2H), 2.08-1.97 (m, 1H), 1.91- | |
| | 1.69 (m, 3H), 1.56 (d, J = 12.9 Hz, 1H). | |
| 11 | ¹H NMR (500 MHz, DMSO-d₆) δ 11.26 (s, | LCMS: |
| | 1H), 11.08 (s, 1H), 7.77 (d, J = 2.9 Hz, 1H), | C₄₁H₄₇N₁₁O₆ |
| | 7.70-7.64 (m, 2H), 7.55 (d, J = 8.3 Hz, 2H), | requires |
| | 7.37-7.29 (m, 2H), 7.30-7.23 (m, 3H), 5.08 | 789, found: |
| | (dd, J = 12.8, 5.4 Hz, 1H), 4.40 (d, J = 12.6 | m/z = 790 |
| | Hz, 1H), 4.30 (d, J = 13.6 Hz, 1H), 3.88 (dt, | [M + H]⁺. |
| | J = 13.5, 4.5 Hz, 2H), 3.67-3.49 (m, 4H), | |
| | 3.41-3.22 (m, 2H), 3.16 (ddd, J = 13.0, 9.8, | |
| | 3.1 Hz, 2H), 3.10-2.83 (m, 5H), 2.72 (s, 3H), | |
| | 2.65-2.53 (m, 2H), 2.42-2.33 (m, 1H), | |
| | 2.08-1.96 (m, 1H), 1.86-1.71 (m, 6H), 1.60- | |
| | 1.50 (m, 1H), 1.33-1.22 (m, 3H). | |
| 12 | ¹H NMR (500 MHz, DMSO-d₆) δ 11.06 (s, | LCMS: |
| | 1H), 10.99 (s, 1H), 7.71 (d, J = 3.0 Hz, 1H), | C₄₃H₅₀N₁₂O₆ |
| | 7.65-7.58 (m, 2H), 7.42 (d, J = 8.8 Hz, 2H), | requires: |
| | 7.27 (d, J = 2.9 Hz, 1H), 6.94-6.86 (m, 2H), | 830, found: |
| | 6.89-6.79 (m, 2H), 5.05 (dd, J = 12.9, 5.4 Hz, | m/z = 831 |
| | 1H), 4.35 (d, J = 12.1 Hz, 1H), 4.27 (d, J = | [M + H]⁺. |
| | 13.4 Hz, 1H), 3.68 (d, J = 10.3 Hz, 2H), 3.60 | |
| | (ddt, J = 10.9, 7.8, 4.3 Hz, 1H), 3.46 (d, J = | |
| | 9.4 Hz, 2H), 3.38-3.33 (m, 2H), 3.30-3.17 (m, | |
| | 3H), 3.08 (t, J = 4.7 Hz, 4H), 3.05-2.82 (m, | |
| | 3H), 2.70 (s, 3H), 2.65-2.53 (m, 5H), 2.48- | |
| | 2.44 (m, 1H), 2.35 (d, J = 6.8 Hz, 2H), 2.01 | |
| | (ddt, J = 12.5, 7.3, 4.4 Hz, 1H), 1.80 (s, 2H), | |
| | 1.74 (td, J = 12.2, 4.1 Hz, 1H), 1.66 (s, 1H), | |
| | 1.56 (s, 1H), 0.76 (tt, J = 6.5, 3.3 Hz, 1H). | |
| 13 | ¹H NMR (500 MHz, DMSO-d₆) δ 11.19 (s, | LCMS: |
| | 1H), 11.07 (s, 1H), 7.76 (d, J = 2.8 Hz, 1H), | C₄₃H₅₁N₁₁O₆ |
| | 7.70-7.63 (m, 2H), 7.51 (d, J = 8.5 Hz, 2H), | requires: |
| | 7.34 (d, J = 2.7 Hz, 1H), 7.18 (d, J = 8.5 Hz, | 817, found: |
| | 2H), 6.91 (d, J = 2.2 Hz, 1H), 6.83 (dd, J = | m/z = 818 |
| | 8.6, 2.2 Hz, 1H), 5.07 (dd, J = 12.9, 5.4 Hz, | [M + H]⁺. |
| | 1H), 4.38 (d, J = 12.3 Hz, 1H), 4.30 (d, J = | |
| | 13.2 Hz, 1H), 3.69-3.47 (m, 2H), 3.33 (s, | |
| | 4H), 3.18 (dd, J = 10.3, 6.8 Hz, 1H), 3.10- | |
| | 2.82 (m, 3H), 2.75 (s, 3H), 2.51 (p, J = 1.8 | |
| | Hz, 9H), 2.19-1.97 (m, 4H), 1.90-1.47 (m, | |
| | 10H). | |
| 14 | ¹H NMR (500 MHz, DMSO-d₆) δ 11.20 (s, | LCMS |
| | 1H), 11.08 (s, 1H), 7.76 (s, 1H), 7.71-7.61 (m, | C₄₄H₅₃N₁₁O₆ |
| | 2H), 7.54-7.48 (m, 2H), 7.37-7.30 (m, 2H), | requires: |
| | 7.25 (dd, J = 8.9, 2.4 Hz, 1H), 7.18 (d, J = 8.5 | 832, found: |
| | Hz, 2H), 5.08 (dd, J = 12.8, 5.4 Hz, 1H), 4.34 | m/z = 833 |
| | (dd, J = 39.9, 12.8 Hz, 2H), 4.06 (d, J = 13.1 | [M + H]⁺. |
| | Hz, 2H), 3.62 (d, J = 11.3 Hz, 1H), 3.11-2.80 | |
| | (m, 8H), 2.73 (s, 4H), 2.69-2.55 (m, 3H), | |
| | 2.20 (s, 2H), 2.09-1.96 (m, 3H), 1.88-1.70 | |
| | (m, 9H), 1.70-1.48 (m, 4H), 1.17 (d, J = | |
| | 12.6 Hz, 2H). | |
| 15 | ¹H NMR (500 MHz, DMSO-d₆) δ 11.30 | LCMS |
| | (s, 1H), 11.10 (s, 1H), 10.08 (s, 1H), 7.86- | C₄₁H₄₇N₁₁O₆ |
| | 7.74 (m, 2H), 7.69 (s, 1H), 7.58 (d, J = 8.3 | requires: |
| | Hz, 2H), 7.36 (s, 1H), 7.19 (d, J = 8.2 Hz, | 790, found: |
| | 2H), 6.95 (s, 1H), 6.80 (d, J = 8.3 Hz, 1H), | m/z = 791 |
| | 5.09 (dd, J = 12.8, 5.3 Hz, 1H), 4.36 (d, | [M + H]⁺. |
| | J = 44.5 Hz, 7H), 2.73 (s, 3H), 2.08 (d, J = | |
| | 37.7 Hz, 3H), 1.90-1.72 (m, 6H), 1.58 (d, | |
| | J = 13.4 Hz, 1H). | |
| 16 | ¹H NMR (500 MHz, DMSO-d₆) δ 11.11- | LCMS |
| | 10.93 (m, 2H), 7.66 (s, 1H), 7.56 (s, 1H), | C₃₆H₄₀N₁₀O₇ |
| | 7.50 (d, J = 8.3 Hz, 1H), 7.41 (dd, J = 8.9, | requires: |
| | 1.8 Hz, 3H), 7.19 (d, J = 42.2 Hz, 2H), 6.92 | 725, found: |
| | (d, J = 2.1 Hz, 1H), 6.89-6.74 (m, 4H), 4.96 | m/z = 726 |
| | (dd, J = 12.7, 5.4 Hz, 1H), 4.23 (dd, J = 36.2, | [M + H]⁺. |
| | 13.0 Hz, 3H), 3.93 (dt, J = 45.1, 6.2 Hz, 3H), | |
| | 3.22-3.10 (m, 6H), 3.02-2.67 (m, 5H), 2.62 | |
| | (d, J = 10.8 Hz, 5H), 2.07-1.86 (m, 4H), | |
| | 1.83-1.62 (m, 5H). | |
| 17 | ¹H NMR (500 MHz, DMSO-d₆) δ 11.22 (d, | LCMS: |
| | J = 56.8 Hz, 1H), 10.94 (s, 1H), 9.73 (s, 1H), | C₄₅H₅₅N₁₁O₈ |
| | 7.77-7.66 (m, 2H), 7.60 (d, J = 5.3 Hz, 1H), | requires: |
| | 7.44 (ddd, J = 20.1, 15.6, 8.0 Hz, 4H), 7.25 | 877, found: |
| | (d, J = 13.9 Hz, 1H), 7.23-7.16 (m, 2H), | m/z = 878 |
| | 5.07 (dd, J = 13.3, 5.1 Hz, 1H), 4.38-4.15 (m, | [M + H]⁺. |
| | 5H), 3.63 (t, J = 6.3 Hz, 2H), 3.60-3.48 (m, | |
| | 2H), 3.39 (d, J = 8.8 Hz, 1H), 3.35-3.11 (m, | |
| | 5H), 3.01-2.77 (m, 3H), 2.63 (s, 3H), 2.60- | |
| | 2.47 (m, 4H), 2.34-2.20 (m, 1H), 2.01-1.85 | |
| | (m, 1H), 1.85-1.63 (m, 3H), 1.60-1.39 (m, | |
| | 0H), 1.10 (d, J = 3.3 Hz, 3H). | |
| 18 | ¹H NMR (500 MHz, DMSO-d₆) δ 10.91 (d, | LCMS: |
| | J = 6.2 Hz, 2H), 7.64 (d, J = 2.8 Hz, 1H), | C₄₁H₅₁N₁₁O₅ |
| | 7.53 (s, 1H), 7.50 (dd, J = 5.8, 2.8 Hz, 1H), | requires: |
| | 7.43-7.37 (m, 2H), 7.37-7.32 (m, 2H), 7.20 | 777, found: |
| | (d, J = 2.9 Hz, 1H), 6.82-6.76 (m, 2H), 5.06 | m/z = 778 |
| | (dd, J = 13.3, 5.1 Hz, 1H), 4.40 (d, J = 17.1 | [M + H]⁺. |
| | Hz, 1H), 4.33-4.14 (m, 3H), 3.58-3.49 (m, | |
| | 3H), 3.04-2.79 (m, 8H), 2.65-2.47 (m, 7H), | |
| | 2.33-2.24 (m, 2H), 2.15 (t, J = 7.4 Hz, 1H), | |
| | 1.94 (dd, J = 11.4, 6.0 Hz, 1H), 1.80-1.65 | |
| | (m, 6H), 1.63-1.36 (m, 5H), 1.17 (s, 3H). | |
| 19 | ¹H NMR (500 MHz, DMSO-d₆) δ 11.13 | LCMS: |
| | (d, J = 2.7 Hz, 1H), 11.02 (s, 1H), 7.68 (s, | C₄₃H₅₁N₁₁O₈ |
| | 1H), 7.59 (s, 1H), 7.51 (dd, J = 8.5, 7.1 Hz, | requires: |
| | 1H), 7.44-7.38 (m, 2H), 7.26 (s, 1H), 7.07 | 849, found: |
| | (dd, J = 8.6, 6.7 Hz, 3H), 6.97 (d, J = 7.0 Hz, | m/z = 850 |
| | 1H), 6.53 (s, 1H), 4.97 (dd, J = 12.9, 5.4 | [M + H]⁺. |
| | Hz, 1H), 4.45 (d, J = 12.9 Hz, 1H), 4.34- | |
| | 4.14 (m, 2H), 3.91 (d, J = 13.4 Hz, 1H), | |
| | 3.63 (t, J = 6.6 Hz, 2H), 3.58-3.44 (m, 8H), | |
| | 3.44-3.34 (m, 3H), 3.29-3.09 (m, 4H), 3.04- | |
| | 2.75 (m, 4H), 2.62 (s, 4H), 1.92 (qd, J = 6.9, | |
| | 4.2, 3.5 Hz, 1H), 1.78-1.20 (m, 7H). | |

### Biological Example 1

Compounds 1-19 were assayed for IMiD activity.

Frozen human peripheral blood mononuclear cells (PBMCs) were thawed and treated with DMSO or compound for 24 hours and then fixed and permeabilized using a Foxp3/Transcription Factor Fixation/Permeabilization Kit (eBioscience, 00-5523). Cells were stained with fluorophore-conjugated antibodies against CD20 (Biolegend 302330), CD3 (BD Pharmingen 552127), and Aiolos (Biolegend 371106). An additional set of DMSO-treated PBMCs was stained for CD20, CD3, and an AlexaFluor 647-conjugated mouse IgG1 isotype control antibody (Biolegend 400136). Stained cells were run on an Attune NxT Acoustic Focusing Flow Cytometer (Thermo-Fisher A29004), and data was analyzed using FlowJo (v10.5.3) and GraphPad Prism (v7.00) software. Single lymphocytes were gated for B cells (CD20+CD3−) and T cells (CD3+CD20−), and the geometric mean fluorescence intensity (MFI) of Aiolos was calculated for each population. The MFI of the isotype control was calculated for each population and used to quantify background staining. Percent Aiolos degradation was calculated for each compound-treated sample using the following equation:

$$\% Degradation = 100 * ( Sample MFI - Isotype MFI ) / ( DMSO MFI - Isotype MFI )$$

Each compound showed enhanced measurable IMiD activity, as shown in the Table 2 below. DC50 is the compound concentration degrading 5000 of Aiolos. Dmax is the maximum percent Aiolos degradation in the assay.

TABLE 2

| Compound | IMiD activity DC50/μM | Dmax/% |
|---|---|---|
| 1 | 0.027 | 77.4 |
| 2 | 0.0052 | 94.7 |
| 3 | 0.093 | 84.9 |
| 4 | 0.008 | 87.0 |
| 5 | 0.022 | 89.1 |
| 6 | 0.023 | 70.8 |
| 7 | 0.004 | 98.2 |
| 8 | 0.001 | 98.4 |
| 9 | 0.012 | 86.5 |
| 10 | 0.009 | 83.1 |
| 11 | 0.020 | 78.7 |
| 12 | 0.007 | 65.1 |
| 13 | 0.033 | 73.9 |
| 14 | 0.011 | 74.0 |
| 15 | 0.017 | 74.7 |
| 16 | 0.024 | 95.7 |
| 17 | 0.56 | 51.9 |
| 18 | 0.009 | 81.6 |
| 19 | 0.001 | 98.6 |

### Biological Example 2

Compound 5 provided loss of viability in a lymphoma cell line.

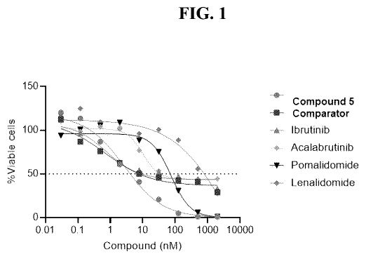

Data presented in FIG. 1 demonstrate that in a REC-1 human mantel cell line, which is only partially depended on BTK for its survival, compound 5 treatment leads to a strong loss of viability in a concentration-dependent manner.

The compound 5 effect is significantly more pronounced when compared to the one of comparator compound, a BTK degrader that does not have IMiD activity, and when compared to covalent BTK inhibitors ibrutinib and acalabrutinib. Comparator compound was prepared as described in PCT/US2020/063176, filed Dec. 3, 2020, published as WO 2021/113557, Jun. 10, 2021, which is incorporated by reference in its entirety. Compound 5 is also more pronounced when compared to IMiD molecules pomalidomide and lenalidomide, which efficiently degrade Aiolos and Ikaros but not BTK. These data suggest that the combination of BTK degradation and IMiD activity can be more efficacious in treating B-cell malignancies than either activity alone.

In FIG. 1, A) Daily oral treatment with Compound 5 at 30 mg/kg resulted in lower mean arthritis score than ibrutinib at 30 mg/kg. Compound 5 effect provided similar clinical benefit as dexamethasone with minimal body weight loss (B) as compared to dexamethasone and vehicle. Significance of clinical arthritis score (A) was determined from the area under the curve (AUC) of mean paw scores calculated for individual mice. (C) Serum levels of anti-type II collagen IgG. Statistical significance was determined between vehicle control and treated groups with one-way Kruskal-Wallis ANOVA and Dunn's multiple comparisons test.

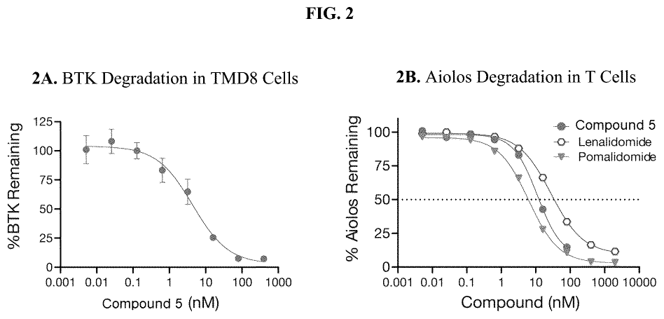

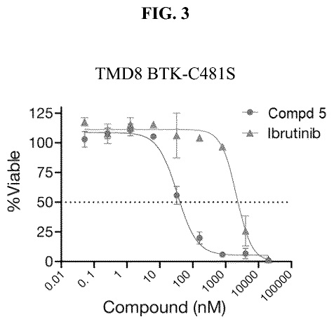

Data presented in FIG. 2, 2A shows that Compound 5 potently degrades BTK in TMD8 cells (human DLBCL cell line). FIG. 2B demonstrates that Compound 5 degradation of Aiolos in human T cells occurs at a similar potency to lenalidomide and pomalidomide. Compound 5 is active against Ibrutinib-resistant tumor cell lines (see, FIG. 3). BTK-C481 mutations are the most common resistance mutations to ibrutinib and other covalent BTK inhibitors. Thus, the activity of Compound 5 against BTK-C481 offers a therapeutic option for patients with resistance to BTK inhibitors.

### Preclinical Models

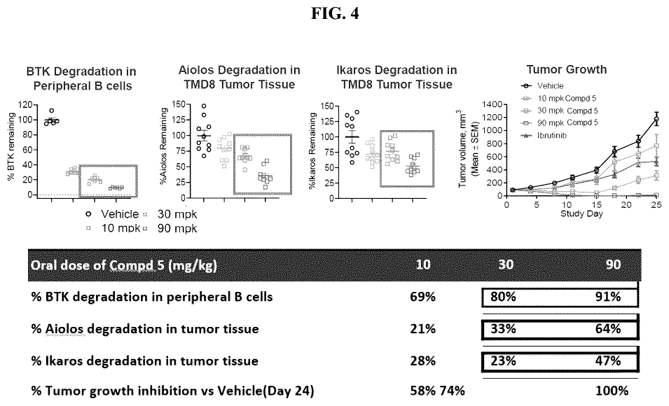

BTK degradation of 80%+ drives potent anti-tumor activity in preclinical models achieved with Compound 5 (see, FIG. 4). Ikaros and Aiolos degradation also achieve target ranges at therapeutic doses.

### Biological Example 3

### Trial Design of Oral Dosing of Compound 5

This trial was conducted at 12 centers, including Memorial Sloan Kettering Cancer Center, MD Anderson Cancer Center, City of Hope (Duarte, California), National Institutes of Health Clinical Center, Sarah Cannon Research Institute, Colorado Blood Cancer Institute, Florida Cancer Specialists, Tennessee Oncology, University of California (San Francisco), University of California (Irvine), OSU Wexner Medical Center, and Swedish Cancer Institute (Seattle).

Patients were treated at four different dosage levels of Compound 5: dose level 1 (100 mg), dose level 2 (200 mg), dose level 3 (300 mg), and dose level 4 (400 mg). The objective of the study was to assess safety and tolerability, identify maximum tolerated dose, and evaluate PK/PD.

Patients evaluated in this study were treated for diseases or disorders selected from the group consisting of: chronic lymphocytic leukemia (CLL) with BTK C481 mutation (n 20); CLL without BTK C481 mutation (n˜20); mantle cell lymphoma (MCL), marginal zone lymphoma (MZL), Waldenstrom's macroglobulinemia (WM) (n˜20); follicular lymphoma (FL) (n˜20); and diffuse large B cell lymphoma (DLBCL) (n˜20). Tables 3 and 4 below summarize patient characteristics. The patient population heavily pretreated, including double-refractory CLL patients.

TABLE 3

| | Overall Population | CLL | Non-CLL |
|---|---|---|---|
| Characteristics | (N = 21) ** | (N = 13) | (N = 7) |
| Median Age, years | 76.0 (61-92) | 76 (65-86) | 77 (67-92) |
| (range) | | | |
| Female, n (%) | 7 (33.3%) | 7 (53.8%) | 0 |
| Male, n (%) | 14 (66.7%) | 6 (46.2%) | 7 (100%) |
| Prior Therapy*, | 4.5 (1-8) | 6.0 (2-8) | 2.0 (1-5) |
| median (range) | | | |
| BTK inhibitor, n (%) | 16 (76.2%) | 12 (92.3% | 4 (57.1%) |
| BCL2 inhibitor, n (%) | 7 (33.3%) | 53.8%) | 0 |

TABLE 4

| Type of Disease | Cohort 1 | Cohort 2 | Cohort 3 | Total |
|---|---|---|---|---|
| Chronic lymphocytic | 8 (66.7%) | 3 (50%) | 2 (66.7%) | 13 (61.9%) |
| leukemia | | | | |
| mantle cell lymphoma | 1 (8.3%) | 1 (16.7%) | 1 (33.3%) | 3 (14.3%) |
| diffuse large B cell | 2 (16.7%) | 1 (16.7%) | 0 (0%) | 3 (14.3%) |
| lymphoma | | | | |
| Waldenstrom's | 0 (0%) | 1 (16.7%) | 0 (0%) | 1 (4.8%) |
| macrolglobulinemia | | | | |
| TBD*** | 1 (8.3%) | 0 (0%) | 0 (0%) | 1 (4.8%) |

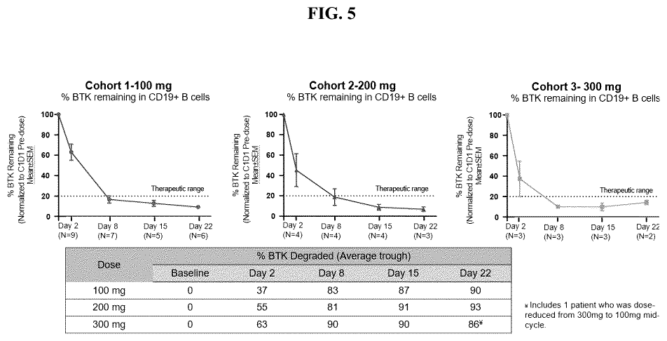

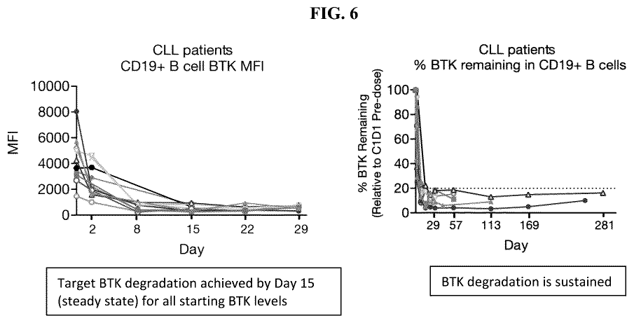

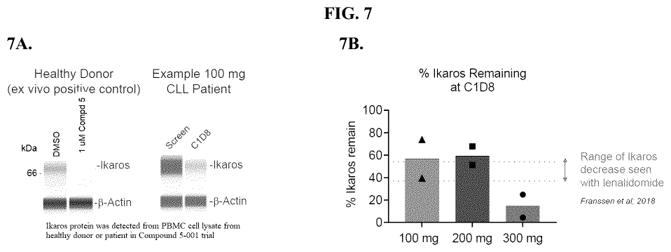

FIG. 5 demonstrates robust BTK degradation observed with Compound 5 across all dose levels and malignancies. Compound 5's rapid and sustained degradation of BTK in patients with CLL was achieved by day 15 as shown in FIG. 6. Treatment with Compound 5 at 100 mg resulted in greater Ikaros degradation in patients with (confirmed by Western Blot, see FIG. 7), consistent with published reports for cereblon immunomodulatory activity. * Prior therapies were not entered into the database for all enrolled patients at the time of Data Cut. Some data pending/ongoing.**One patient's disease type wasn't identified in the EDC at the time of extract, but disease type was coded based on source data***One subject was screened into the study, but the indication and cohort weren't entered in the EDC at the time of data extract

### Compound 5 Demonstrated Degradation of Cereblon Neo-Substrate Ikaros

Frozen patient PBMCs collected at baseline and at Cycle 1 Day 8 were processed into protein lysate and degradation of cereblon neo-substrate Ikaros was determined using Western blot via Jess™ Simple Western automated system. After one week of Compound 5 treatment, Ikaros degradation (FIG. 7) was detected in all patients. Similar degree of Ikaros degradation (range 26.1-60.5% degradation) was observed between patients receiving either 100 or 200 mg. Patients receiving 300 mg Compound 5, 75.2-95.7% Ikaros degradation was detected.

Frozen PBMCs from patients were prepared and Ikaros protein levels were assessed using Western blot via Jess™ Simple Western automated system.

FIG. 7A, western blot analysis showed reduction of Ikaros protein band in one of the CLL patient receiving 100 mg of Compound at Cycle 1 Day 8. A healthy donor PBMC treated ex vivo with 1 μM Compound 5 for 4 hours was used as a positive control.

FIG. 7A, densitometric analysis of Ikaros degradation in all patients were obtained after normalization to β-actin and % Ikaros degradation was calculated relative to Baseline values from each patient.

List of Materials used for flow cytometry are shown in Table 5 below.

TABLE 5

| | | | Vendor | | |
|---|---|---|---|---|---|
| Material | Vendor | Vendor Catalog # | Lot # | RM/PN# | Lot |
| 1X BD PharmLyse | N/A | N/A | 0119703 | PN2236 | 1-03278 |
| FcBlock | Invitrogen | 14-9161-73 | 2161225 | RM3158 | 20-1147 |
| Brilliant Stain Buffer | BD Biosciences | 566349 | 38709 | RM3478 | 20-0477 |
| Staining Buffer, 1% BSA | N/A | N/A | N/A | PN2214 | 1-03263 |
| BD FACSDiva CS&T Beads | BD Biosciences | 655051 | 9042577 | RM3246 | 19-0491 |
| Compensation Beads | Life Technologies | 01-2222-42 | 2268280 | RM3157 | 20-1556 |
| FoxP3 Stain kit | eBioscience | 00-5523-00 | 2178652 | RM3501 | 20-0729 |
| Water | Quality Biological | 351-029-131 | 723901 | RM0924 | 20-1588 |
| BD TruCount ™ Tubes | BD Biosciences | 340334 | 20-1749 | RM3217 | 20-1749 |
| CD19 | BioLegend | 363018 | B313190 | RM3158 | 20-1732 |
| CD56 | BD Biosciences | 563041 | 23877 | RM3158 | 20-1642 |
| CD14 | BioLegend | 301836 | B318037 | RM3158 | 20-1730 |
| CD16 | BioLegend | 360734 | B299811 | RM3158 | 20-1595 |
| BTK unconjugated | CST | 8547S | 13 | RM3158 | 20-1662 |
| Anti-rabbit Alexa488 | BioLegend | 406416 | B294808 | RM3158 | 20-1693 |
| CD8 | BD Biosciences | 560662 | 9331325 | RM3158 | 20-1640 |
| CD4 | BD Biosciences | 560644 | 86585 | RM3158 | 20-1641 |
| CD3 | BD Biosciences | 557943 | 9185576 | RM3158 | 20-1452 |
| CD45 | BD Biosciences | 563792 | 121733 | RM3158 | 20-1639 |

### Biological Example 4

### Western Blot Assay for Ikaros Degradation

For Ikaros degradation assessment, patient PBMCs were lysed in lysis buffer RIPA buffer (Fisher, PI89901), complete Mini EDTA-free protease inhibitor (Sigma 11836170001), Protease Inhibitor Cocktail (Sigma, P2714) and Phosphatase Inhibitor Cocktail 2 and 3 (Sigma, P5726 and P0044) and stored overnight at −80° C. Cells were then thawed and centrifuged for 5 min at 8000×g and lysate supernatants were transferred to a fresh tube. Protein levels were determined by BCA Assay performed according to manufacturer's protocol (EMD Millipore, cat. no. 71285-3). A total of 3 μg protein was loaded and 1:50 dilution of primary anti-Ikaros (Cell Signaling Technology, cat. 14859) and 1:50 anti-β-Actin (Cell Signaling Technology, cat. 8457) was used. Analysis of Ikaros was performed using the Jess™ Simple Western automated system (Protein Simple, San Jose, CA) according to the manufacturer's instructions.

### Other Embodiments

It is to be understood that the foregoing description is intended to illustrate and not limit the scope of this disclosure, which is defined by the scope of the appended claims. Other aspects, advantages, and modifications are within the scope of the following claims.

1. A method of treating or preventing a disease in a subject in need thereof, comprising a step of administering to the subject an amount of a bifunctional compound, wherein said bifunctional compound is capable of inducing proteolytic degradation of Bruton's tyrosine kinase, wherein said compound has enhanced IMiD activity, wherein said amount is effective to treat or prevent the disease, wherein the administering is for at least 15 days, and wherein the bifunctional compound is a compound of Formula (A) 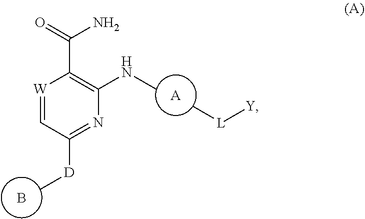
or a pharmaceutically acceptable salt thereof, wherein
W is CH or N;
D is a bond or —NH—;
Ring A is phenyl, a 9-10 membered bicyclic aryl, a 5-6 membered partially or fully unsaturated monocyclic heterocycle, or a 9-10 membered bicyclic heteroaryl, wherein the monocyclic heterocycle and bicyclic heteroaryl of Ring A each possess one to three heteroatoms independently selected from N, O, or S, wherein Ring A is unsubstituted or is independently substituted with up to three substituents selected from halo, —CN, —COOH, NH₂, and substituted or unsubstituted C₁₋₆ alkyl;
Ring B is a phenyl, a 5-6 membered heteroaryl, a 4-6 membered heterocycloalkyl, or a 8-10 membered spiro bicyclic heterocycle, wherein Ring B is substituted or is unsubstituted, and wherein the heteroaryl and heterocycloalkyl of Ring B has one to three heteroatoms independently selected from N, O, or S;
L is —X¹—X²—X³—X⁴—X⁵—;
X¹ is a bond, —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro or fused bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is substituted with —CH₃ or is unsubstituted;
X² is a bond, —(O—CH₂—CH₂)ₚ—, —(CH₂—CH₂—O)ₚ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S;
X³ is a bond, —C₁₋₈ alkyl-, —C≡C—, 4-6 membered cycloalkyl, —N(R)—, —N(R)—C(O)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is substituted with —CH₃ or is unsubstituted;
X⁴ is a bond, —CH₂—CH₂—N(R)—, —N(R)—, —C₁₋₄ alkyl-, —(O—CH₂—CH₂—CH₂)ₘ—, a 5-6 membered saturated, partially unsaturated, or fully unsaturated carbocycle, or a 5-6 membered saturated, partially unsaturated, or fully unsaturated heterocycle having one to three heteroatoms independently selected from N, O, or S;
X⁵ is a bond, —C₁₋₄ alkyl-, —N(R)—, —O—, —C(O)—, or —C(O)—N(R)—;
each R is independently hydrogen or —C₁₋₃ alkyl; and
each of m, n, and P is independently an integer from one to three; and
Y is 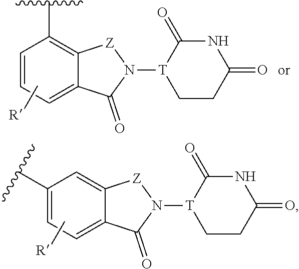
wherein each T is independently CH or N; and each Z is independently —CH₂— or —C(O)—; and each R′ is hydrogen, methyl, or NH₂.

2.-110. (canceled)

111. The method of claim 1, wherein the administering is chronic and the frequency of the chronic administering is (a), (b), (c) or a combination thereof:
(a) once a week, twice a week, once a day, twice a day, three times a day, or four times a day;
(b) one or more month or one or more years;
(c) 100-600 mg/kg body weight/day, 200-600 mg/kg body weight/day, or 300-600 mg/kg body weight/day.

112. The method of claim 1, wherein the disease or disorder is cancer.

113. The method of claim 1, wherein the disease or disorder is a B cell malignancy.

114. The method of claim 1, wherein:
the disease or disorder is cancer and the cancer is selected from the group consisting of chronic lymphocytic leukemia (CLL), small lymphocytic lymphoma (SLL), transformed CLL or Richter's transformation, small cell lymphoma, follicular lymphoma (FL), diffuse large B-cell lymphoma (DLBCL), non-Hodgkin lymphoma, mantle cell lymphoma (MCL), marginal zone lymphoma (MZL), Waldenstrom macroglobulinemia (WM), central nervous system (CNS) lymphoma, metastatic melanoma, squamous cell carcinoma of the head and neck (HNSCC), non-small cell lung cancer (NSCLC), platinum-resistant epithelial ovarian cancer (EOC), gastric cancer, metastatic castrate-resistant prostate cancer (mCRPC), triple-negative breast cancer (TNBC), muscle-invasive urothelial cancer, mesothelioma, cervical cancer, microsatellite stable colorectal cancer (MSS CRC), and multiple myeloma (MM); or
the disease or disorder is selected from the group consisting of multiple myeloma, myelodysplastic syndrome, karposi sarcoma, or post transplant lymphoproliferative disorder; or
the disease or disorder is graft versus host disease; or
the disease or disorder is selected from the group consisting of chronic lymphocytic leukemia (CLL) with BTK C481 mutation; CLL without BTK C481 mutation; mantle cell lymphoma (MCL), marginal zone lymphoma (MZL), Waldenstrom's macroglobulinemia (WM); follicular lymphoma (FL); and diffuse large B cell lymphoma (DLBCL); or
the disease or disorder is cancer and the cancer is ibrutinib-resistant.

115. The method of claim 1, wherein the subject has:
a C481 mutant Bruton's tyrosine kinase; or
a C481S, L528W, M437R, or V416L mutant Bruton's tyrosine kinase.

116. The method of claim 1, wherein:
Ring B is:
a substituted or unsubstituted 5-6 membered heterocycloalkyl having one to two nitrogen atoms; or
a substituted or unsubstituted 5-6 membered heteroaryl having one to two heteroatoms independently selected from N and S; or 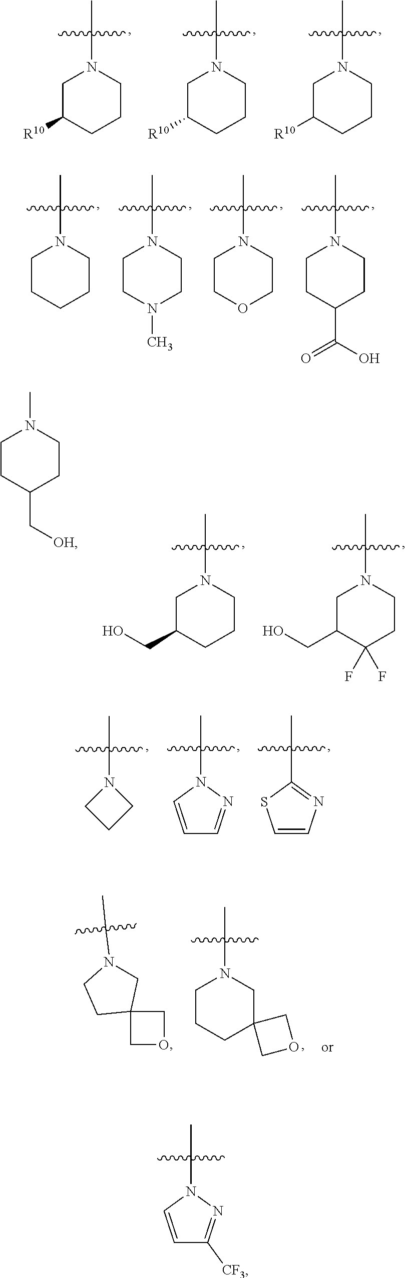
wherein R¹⁰ is 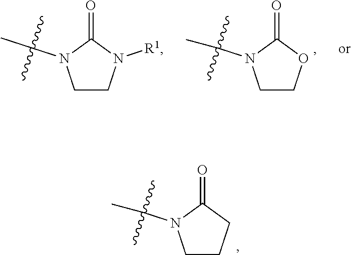
and wherein R¹ is a C₁₋₄ alkyl group; or
Ring A is: 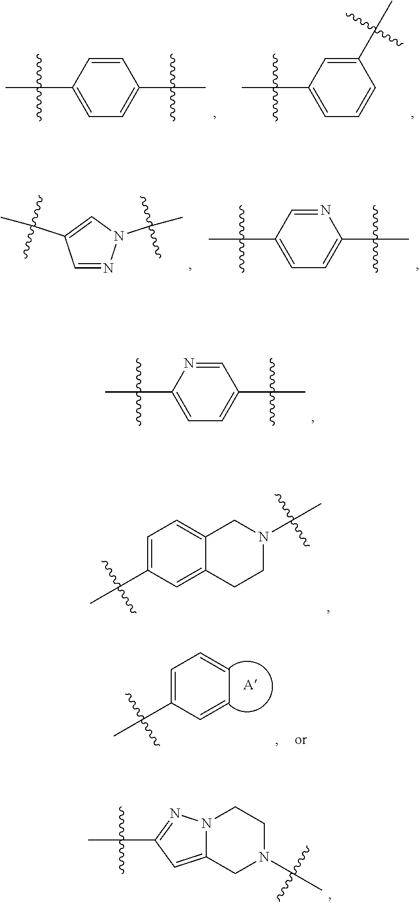
wherein Ring A′ together with the phenyl ring to which Ring A′ is fused forms a 9-10 membered bicyclic aryl or a 9-10 membered bicyclic heteroaryl wherein the bicyclic heteroaryl has one to three heteroatoms independently selected from N, O, or S; or
at least one of X¹, X², and X⁵ is —N(R)—, —C(O)—N(R)—, or —CH₂—; or
X is —(O—CH₂—CH₂—CH₂)ₘ—, m is one, and X² is —C(O)—N(R)—; or
X¹ is:
—CH₂—, —C(O)—, 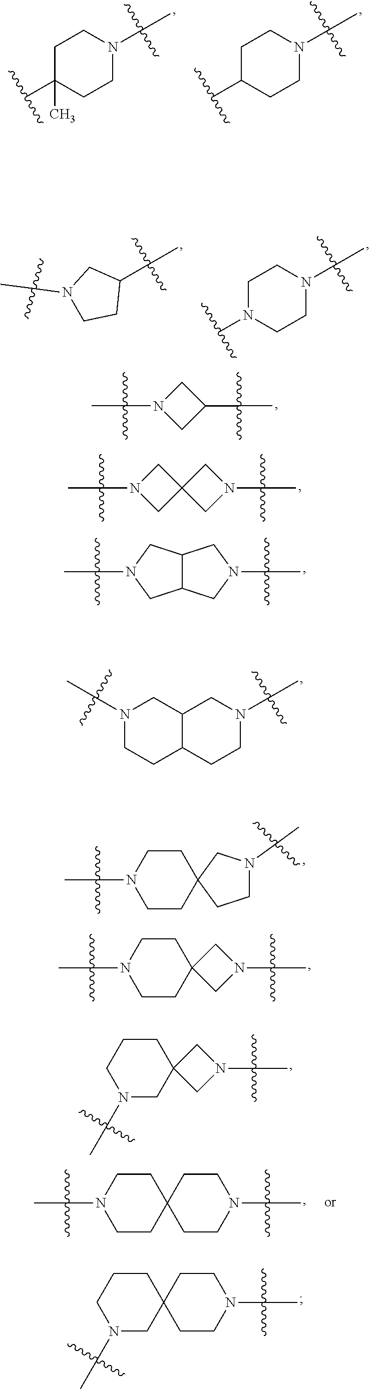
or
X² is:
(—CH₂—CH₂)ₙ—, —(CH₂—CH₂)ₙ—, or —C₁₋₅ alkyl-; or
a bond, —C(O)—, —C₁₋₅ alkyl-, 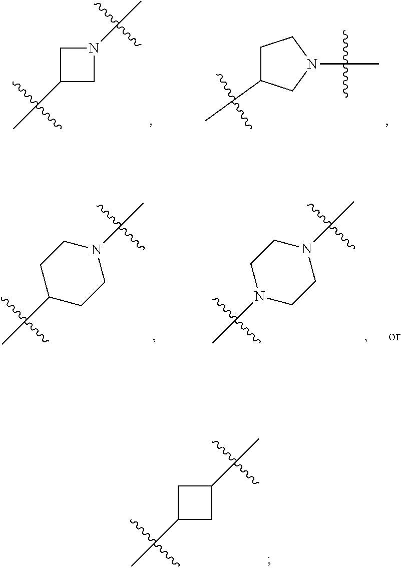 [SMILES: *C1CN(*)C1 ; *C1CCN(*)C1 ; *C1CCN(*)CC1 ; *N1CCN(*)CC1 ; *C1CC(*)C1]
or
X³ is:
a bond, —C≡C—, —C₁₋₄ alkyl-, or —N(R)—; or
a bond, —C₁₋₄ alkyl-, 4-6 membered cycloalkyl, or —N(R)—; or
a bond, —C₁₋₄ alkyl-, —NH—, 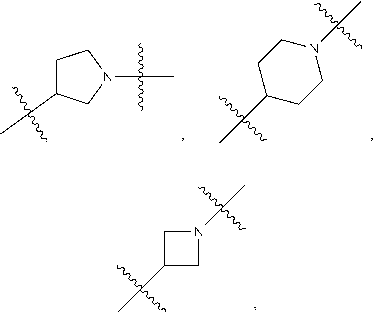 [SMILES: *C1CCN(*)C1 ; *C1CCN(*)CC1 ; *C1CN(*)C1]
or —C≡C—; or
X⁴ is:
a bond, —CH₂—, or —N(R)—; or
a bond, 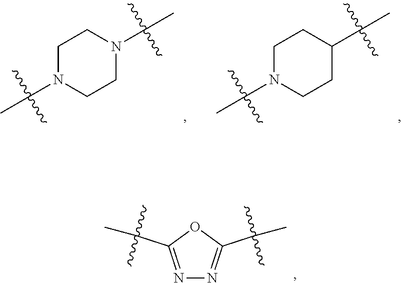 [SMILES: *N1CCN(*)CC1 ; *C1CCN(*)CC1 ; *c1nnc(*)o1]
—C₁₋₄ alkyl-, —CH₂—CH₂—N(R)—, or —N(R)—; or
X⁵ is a bond, —C₁₋₄ alkyl-, —N(R)—, or —C(O)—N(R)—; or
L is 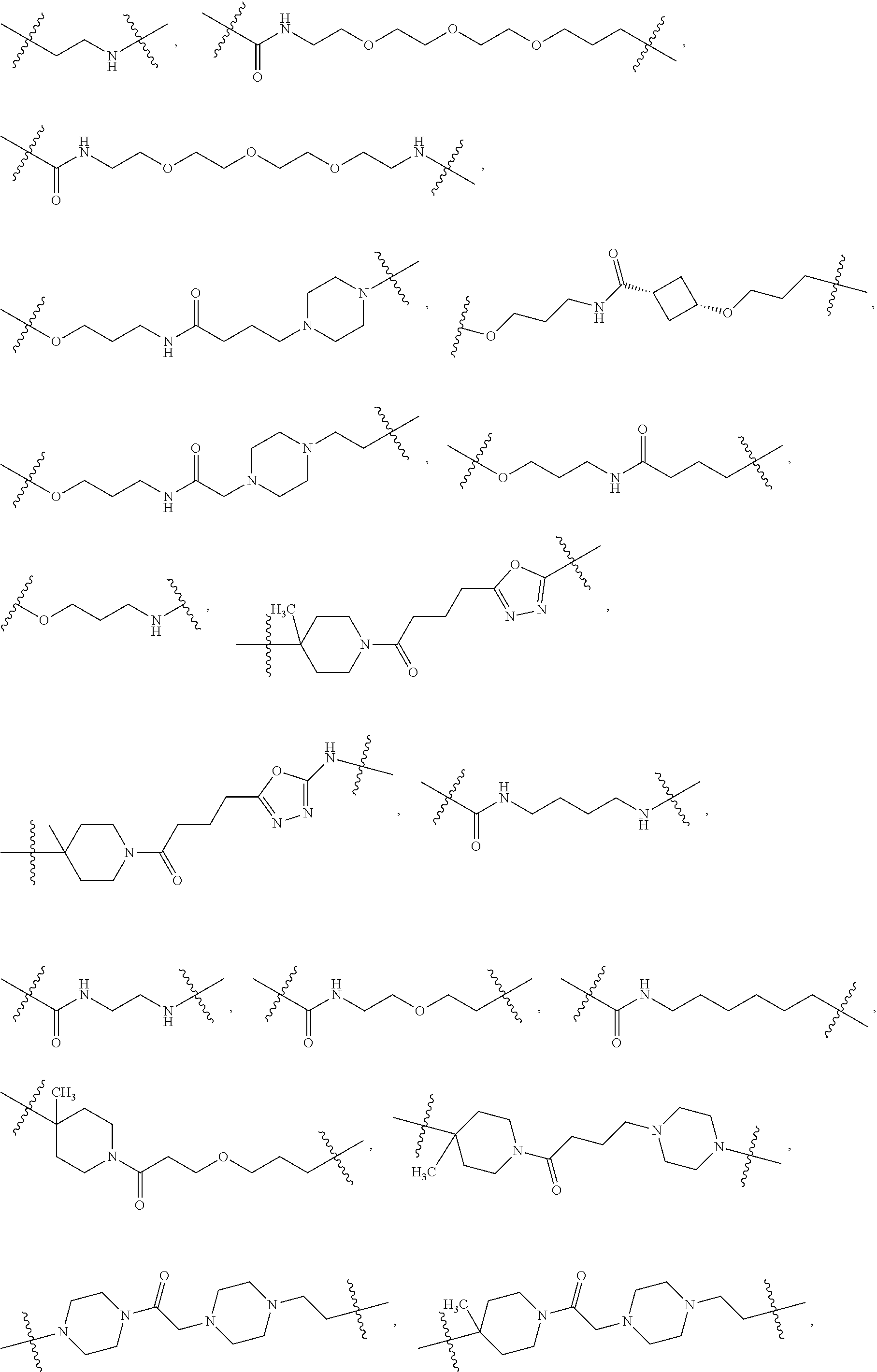 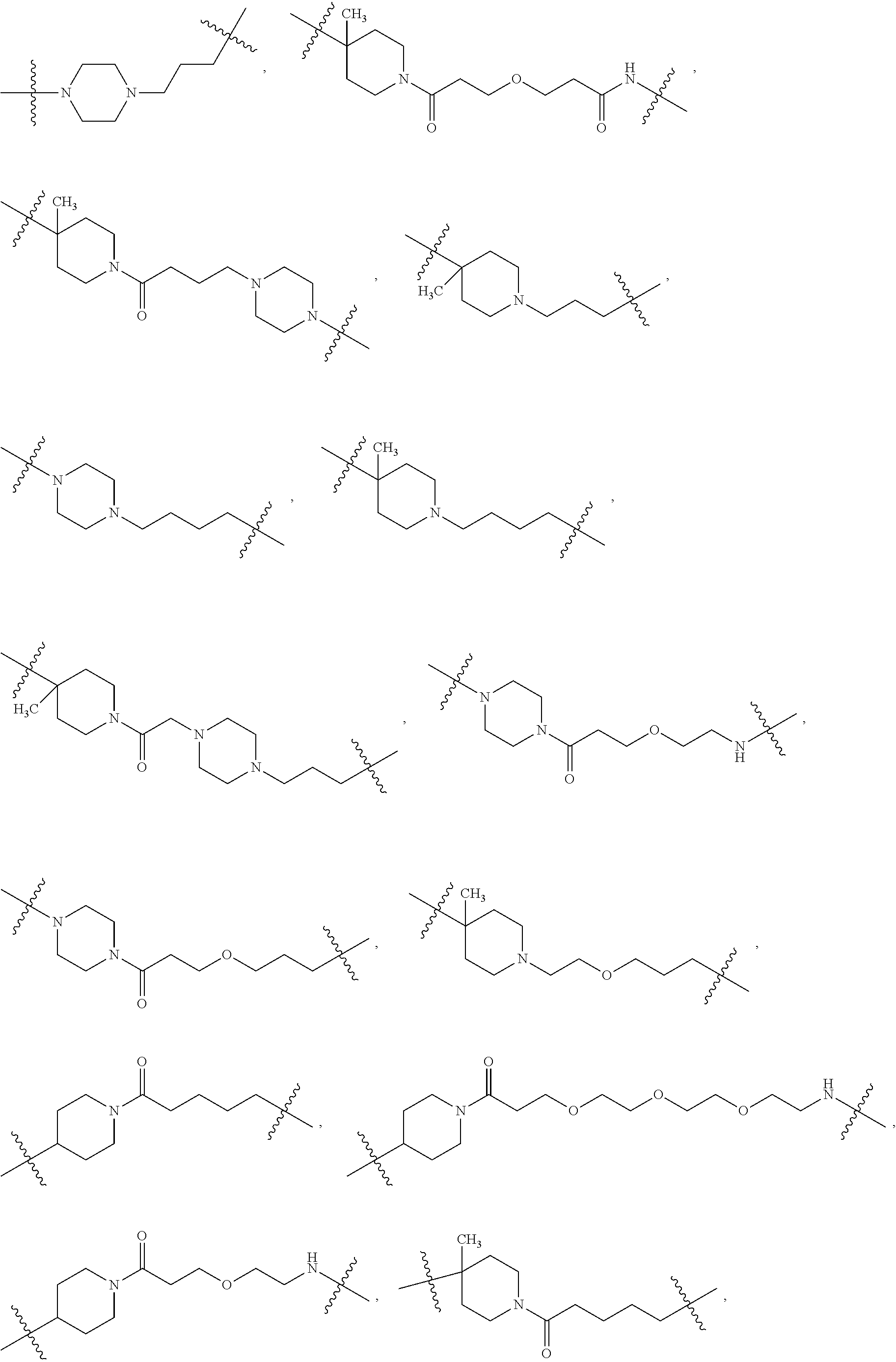 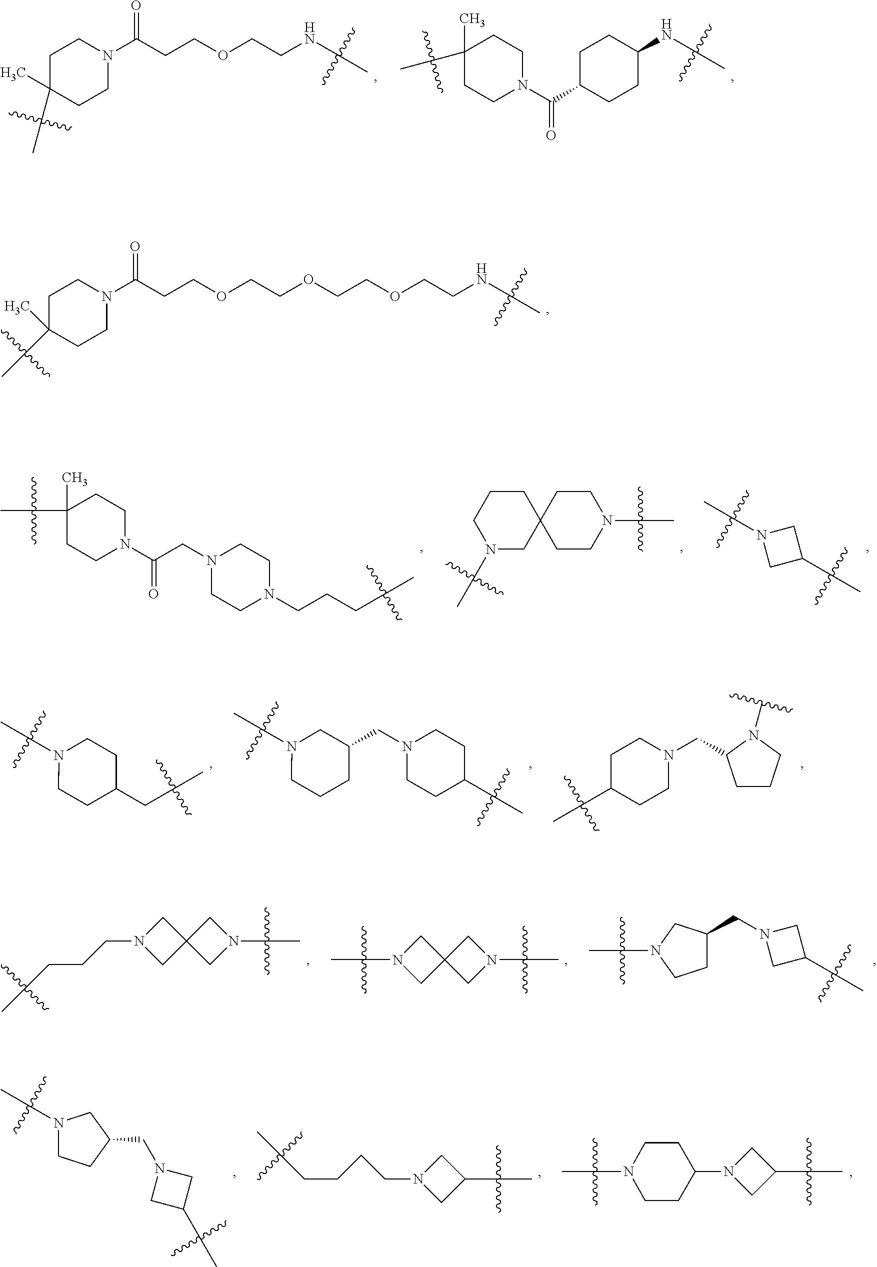 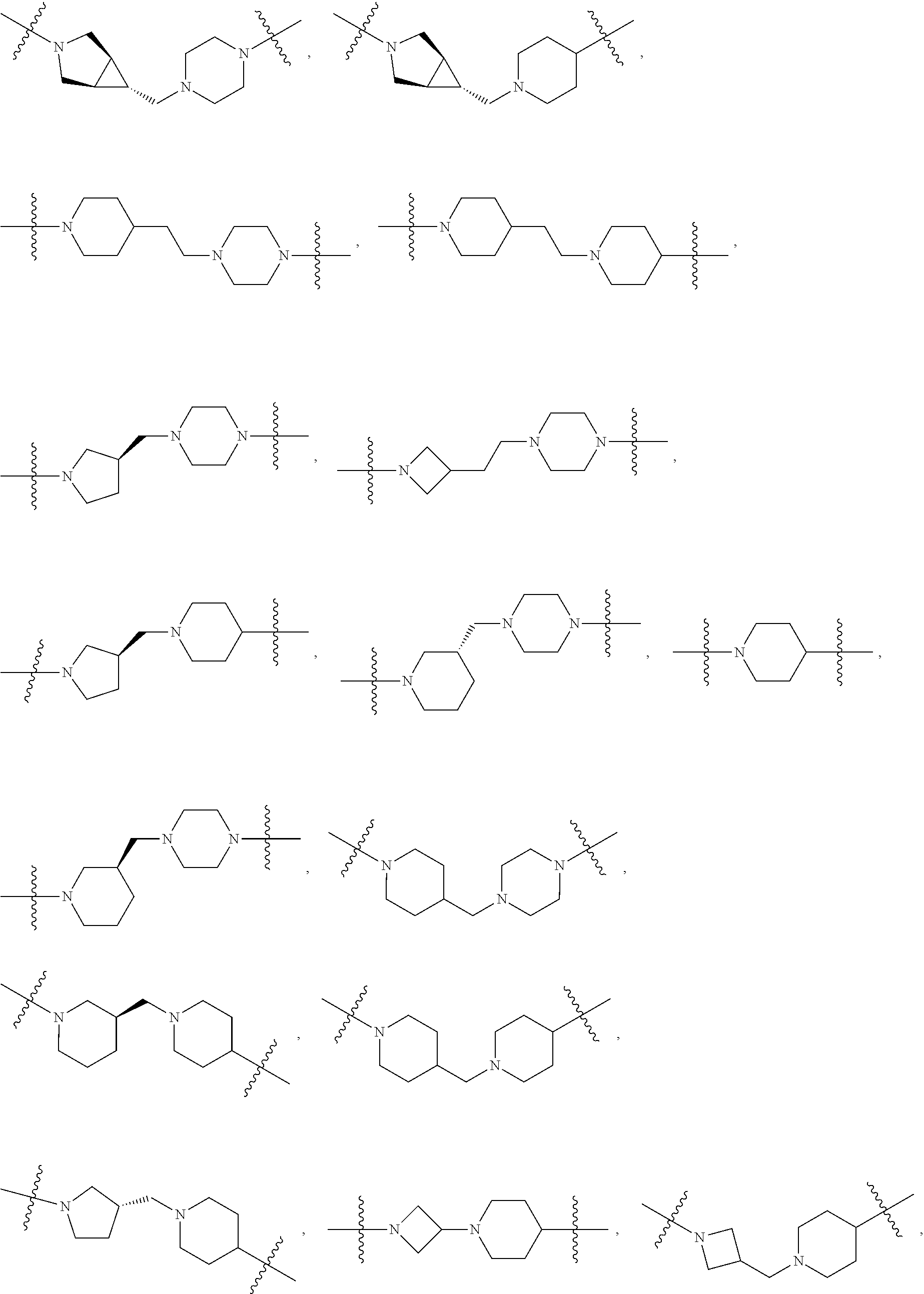 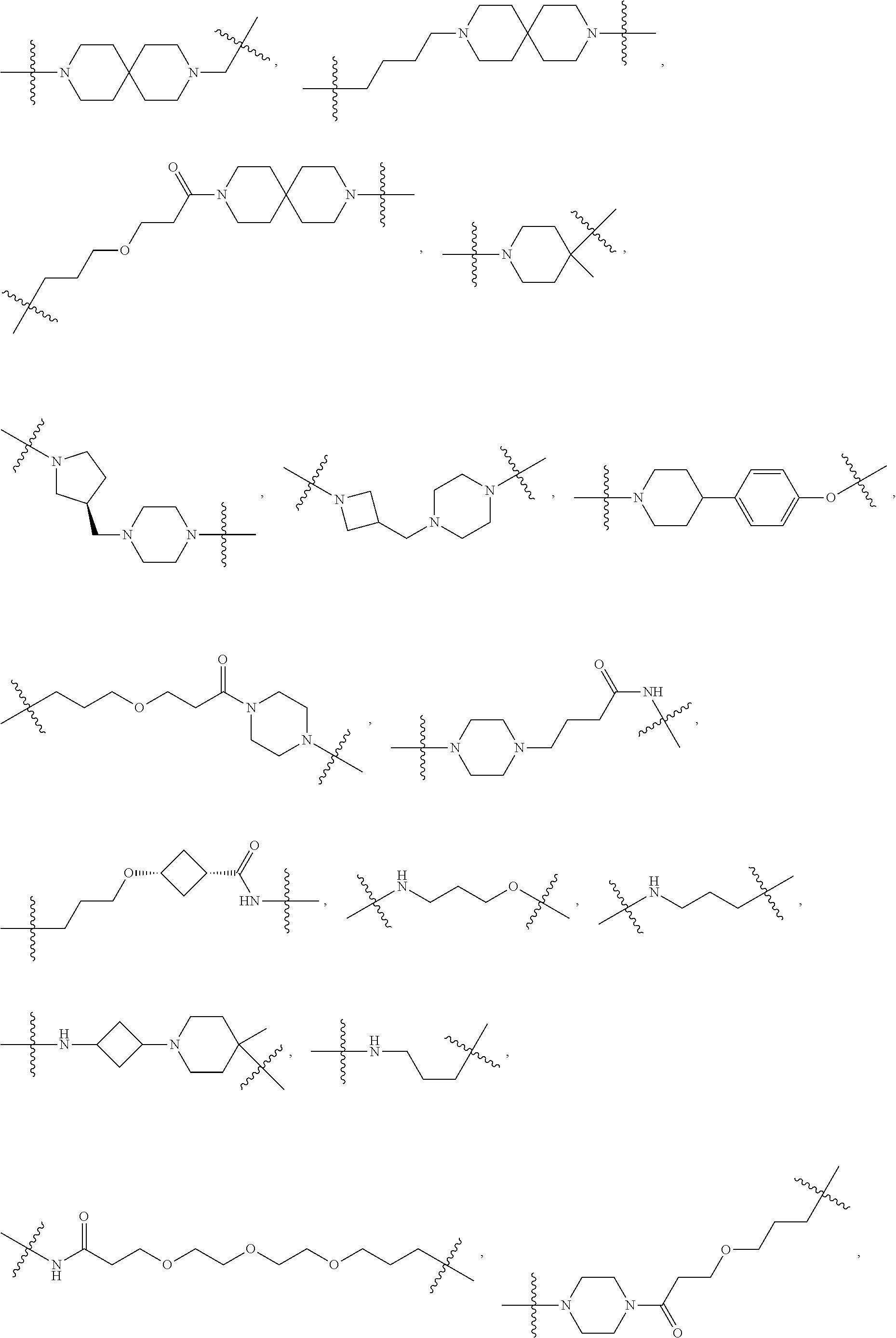 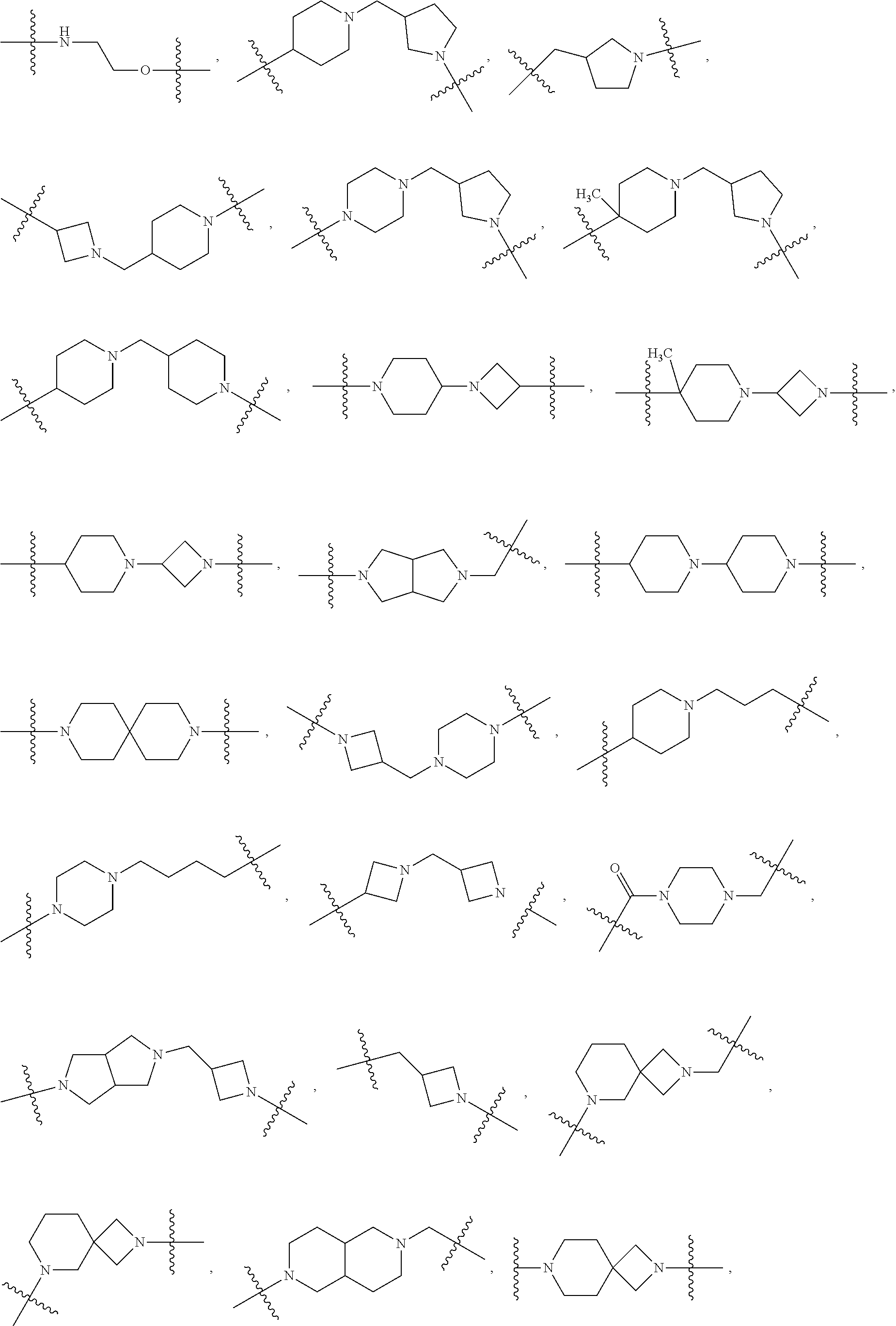 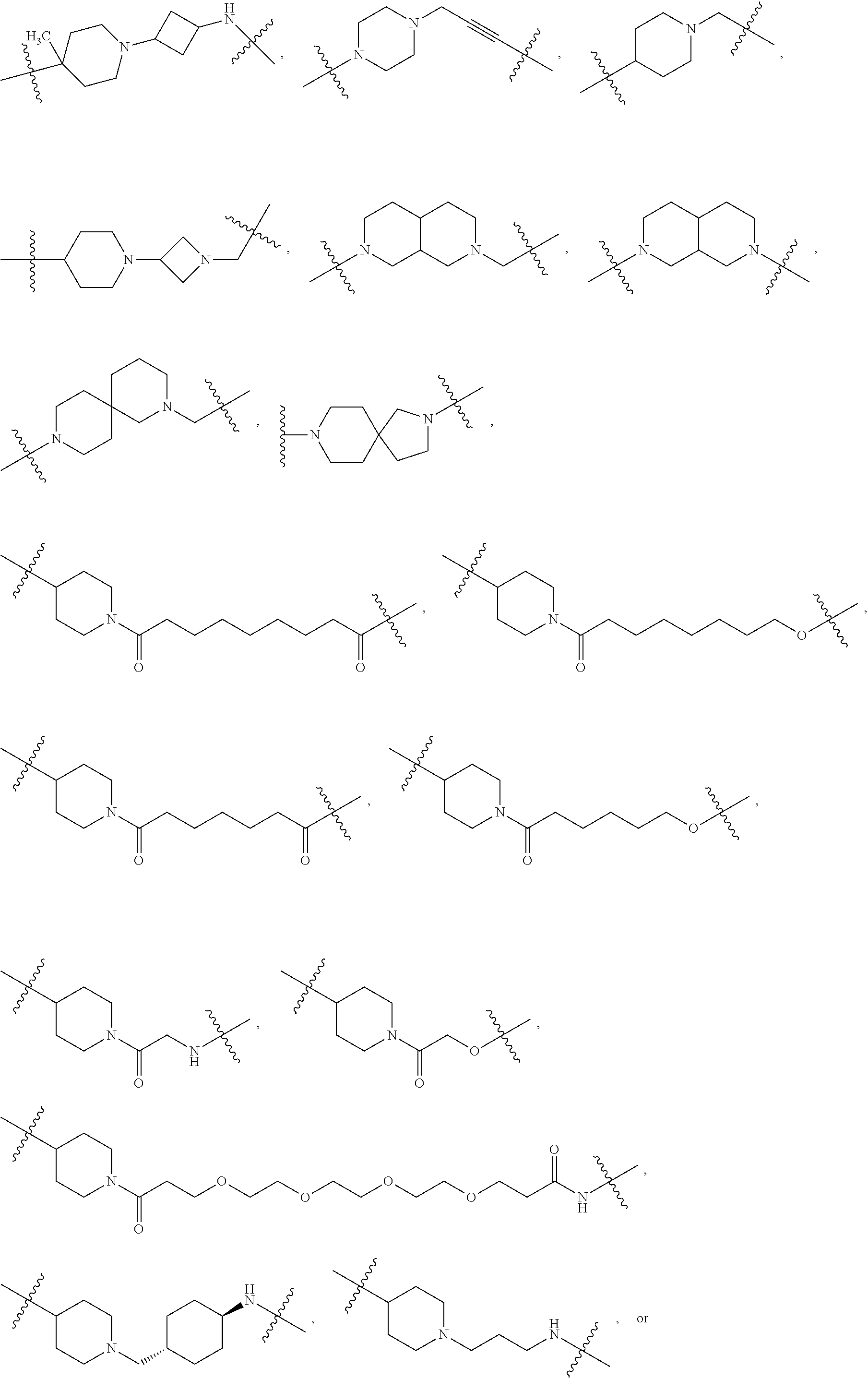 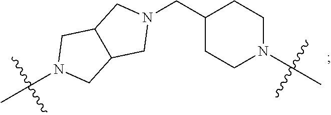 [SMILES: *N1CCC(CN2CC3CN(*)CC3C2)CC1]
or
Y is

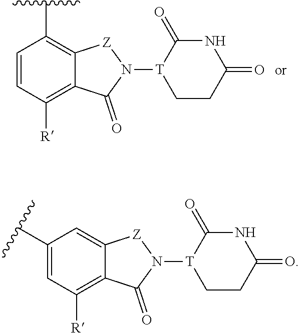

117. The method of claim 1, wherein the compound of Formula (A) is a compound of any one of Formulas (B), (C), (D), (E), (F), (G), or (M):
Formula (B) is: 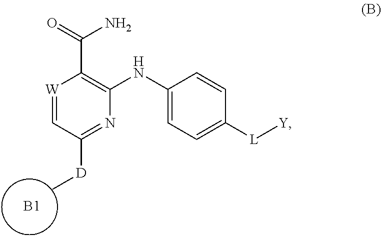
or a pharmaceutically acceptable salt thereof, wherein
W is CH or N;
D is a bond or —NH—;
Ring B1 is a 4-6 membered, fully saturated, partially unsaturated, or fully unsaturated monocyclic heterocycle or a 8-10 membered, fully saturated, spiro bicyclic heterocycle, wherein Ring B1 has one to three heteroatoms independently selected from N, O, or S, and Ring B is unsubstituted or is substituted with one to three groups selected from halo, —CH₃, —CF₃, —C(O)OH, —CH₂OH, or a five membered heterocycloalkyl that is unsubstituted or substituted with oxo and having one to two heteroatoms independently selected from N or O;
L is —X¹—X²—X³—;
X¹ is —C(O)—N(R), N(R)—C(O), (O—CH₂—CH₂)ₘ—, —O(C₆H₄)—, —(O—CH₂—CH₂—CH₂)ₘ—, —C₁₋₅ alkyl-, 7-12 membered spiro or fused bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is unsubstituted or is substituted with —CH₃;
X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S;
X³ is a bond, —C₁₋₄ alkyl-, —C≡C—, 4-6 membered cycloalkyl, —N(R)—, —(O—CH₂—CH₂)ₚ, —(CH₂—CH₂—O)ₚ—, 4-6 membered heterocycloalkyl having one to two heteroatoms independently selected from N, O, or S, wherein the heterocycloalkyl is unsubstituted or is substituted with —CH₃;
each R is independently hydrogen or —C₁₋₃ alkyl; and
each of m, n, and p is independently an integer from one to three;
Formula (C) is: 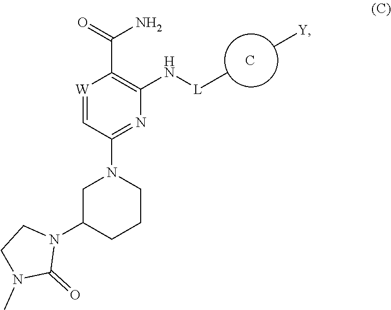
or a pharmaceutically acceptable salt thereof, wherein
W is CH or N;
Ring C is phenyl or a saturated, partially unsaturated, or fully unsaturated 5-6 membered monocyclic heterocycle having one to two heteroatoms independently selected from N, O, or S, wherein each of the phenyl and heterocycle of Ring C is unsubstituted or is substituted;
L is —X¹—X²—X³—;
X¹ is —C(O)—N(R), N(R)—C(O), (O—CH₂—CH₂)ₘ—, O—(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, or 4-6 membered monocyclic heterocycloalkyl having one to two heteroatoms independently selected from N, O, or S, wherein each of the bicyclic heterocycloalkyl and the monocyclic heterocycloalkyl of X¹ is unsubstituted or is substituted with —CH₃;
X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S;
X³ is a bond, —C₁₋₄ alkyl-, —C≡C—, 4-6 membered cycloalkyl, —N(R)—, —(O—CH₂—CH₂)ₚ, —(CH₂—CH₂—O)ₚ, 4-6 membered heterocycloalkyl having one to two heteroatoms independently selected from N, O, or S, wherein the heterocycloalkyl is unsubstituted or is substituted with —CH₃;
each R is independently hydrogen or —C₁₋₃ alkyl; and
each of m, n, and p is independently an integer from one to three; 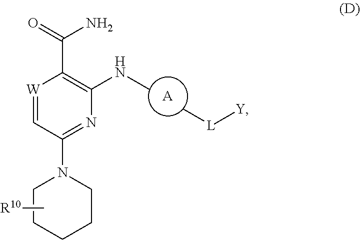
or a pharmaceutically acceptable salt thereof, wherein
W is CH or N;
Ring A is 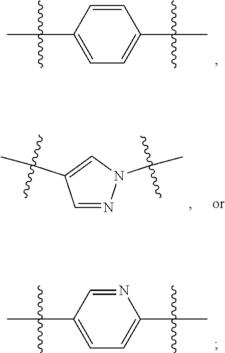 [SMILES: *c1ccc(*)cc1 ; *c1cnn(*)c1 ; *c1ccc(*)nc1]
L is —X¹—X²—X³—;
X¹ is —C₁₋₅ alkyl- or 4-6 membered monocyclic heterocycloalkyl having one to two heteroatoms independently selected from N, O, or S, wherein the monocyclic heterocycloalkyl of X¹ is unsubstituted or is substituted with —CH₃;
X² is a bond, —C₁₋₅ alkyl-, or 4-6 membered monocyclic heterocycloalkyl having one to two heteroatoms independently selected from N, O, or S, wherein the monocyclic heterocycloalkyl of X¹ is unsubstituted or is substituted with —CH₃;
X³ is a bond, —C₁₋₄ alkyl-, 4-6 membered monocyclic cycloalkyl, or 4-6 membered heterocycloalkyl having one to two heteroatoms independently selected from N, O, or S, wherein the heterocycloalkyl is unsubstituted or is substituted with —CH₃; and
R¹⁰ is halo, —C₁₋₅ alkyl, 3-6 membered cycloalkyl, 5-6 membered heterocycloalkyl, —CN, —OH, —CF₃, —C(O)OH, —CH₂OH, —CH₂CH₂OH, 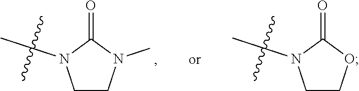
Formula (E) is: 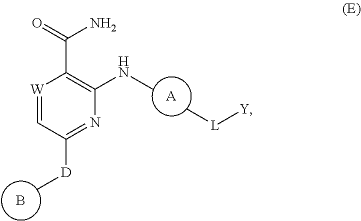
or a pharmaceutically acceptable salt thereof, wherein
D is a bond or —NH—;
W is N or CH;
Ring A is phenyl, a 9-10 membered bicyclic aryl, a 5-6 membered partially or fully unsaturated monocyclic heterocycle, or a 9-10 membered bicyclic heteroaryl, wherein the monocyclic heterocycle and bicyclic heteroaryl of Ring A each possess one to three heteroatoms independently selected from N, O, or S;
Ring B is substituted or unsubstituted 5-6 membered saturated, partially unsaturated, or fully unsaturated monocyclic heterocycle, or substituted 8-10 membered spiro bicyclic heterocycle, wherein Ring B has one to three heteroatoms independently selected from N, O, or S;
L is —X¹—X²—X³—X⁴—X⁵—;
X¹ is a bond, —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is substituted with —CH₃ or is unsubstituted;
X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S;
X³ is a bond, —C₁₋₄ alkyl-, —C≡C—, 4-6 membered cycloalkyl, —N(R)—, —(O—CH₂—CH₂)ₚ, —(CH₂—CH₂—O)ₚ—, 4-6 membered heterocycloalkyl having one to two heteroatoms independently selected from N, O, or S, wherein the heterocycloalkyl is substituted with —CH₃ or is unsubstituted;
X⁴ is a bond, —CH₂—CH₂—N(R)—, —N(R)—, —C₁₋₄ alkyl-, —(O—CH₂—CH₂—CH₂)ₘ—, a 5-6 membered saturated, partially unsaturated, or fully unsaturated carbocycle, or a 5-6 membered saturated, partially unsaturated, or fully unsaturated heterocycle having one to three heteroatoms independently selected from N, O, or S;
X⁵ is a bond, —N(R)—, or —C(O)—N(R)—;
each R is independently hydrogen or —C₁₋₃ alkyl;
each of m, n, and p is independently an integer from one to three; and
wherein at least one of X¹, X², X³, X⁴, and X⁵ has a nitrogen atom, and Y is directly bonded to L at a nitrogen atom of X¹, X², X³, X⁴, or X⁵;
Formula (F) is: 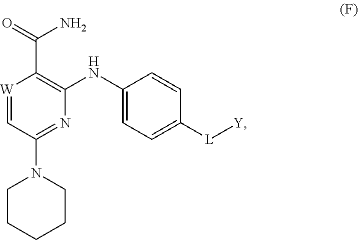
or a pharmaceutically acceptable salt thereof, wherein
W is CH or N;
L is —X¹—X²—X³—;
X¹ is —C(O)—N(R), N(R)—C(O), (O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is substituted with —CH₃ or is unsubstituted;
X² is a bond, —C₁₋₅ alkyl-, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S;
X³ is a bond, —C₁₋₄ alkyl-, —C≡C—, 4-6 membered cycloalkyl, —N(R)—, —(O—CH₂—CH₂)ₚ, —(CH₂—CH₂—O)ₚ—, 4-6 membered heterocycloalkyl having one to two heteroatoms independently selected from N, O, or S, wherein the heterocycloalkyl is substituted with —CH₃ or is unsubstituted;
each R is independently hydrogen or —C₁₋₃ alkyl; and
each of m, n, and p is independently an integer from one to three;
Formula (G) is: 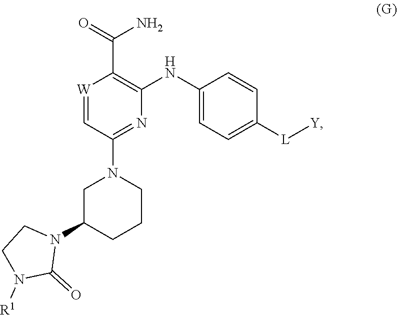
or a pharmaceutically acceptable salt thereof;
Formula (M) is: 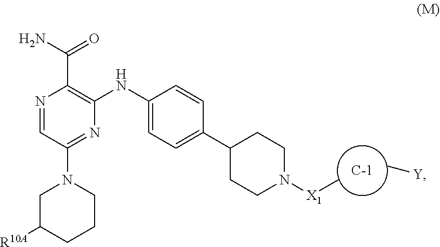
or a pharmaceutically acceptable salt thereof, wherein
R^{10A} is hydrogen, 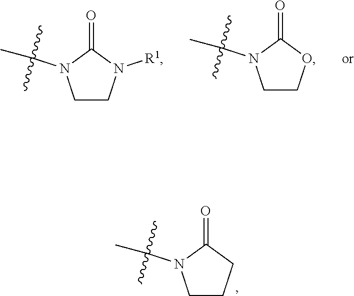
wherein R¹ is C₁₋₄ alkyl;
X¹ is —C₁₋₅ alkyl-;
Ring C-1 is a 5-6 membered heterocycloalkyl having one nitrogen atom; and
Y is 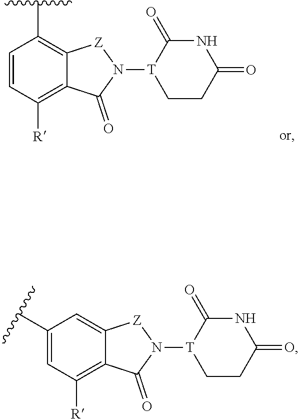
wherein each T is independently CH or N; and each Z is independently —CH₂— or —C(O)—; and each R′ is hydrogen, methyl, or NH₂.

118. The method of claim 117, wherein in the compound of Formula (B):
Ring B1 is: 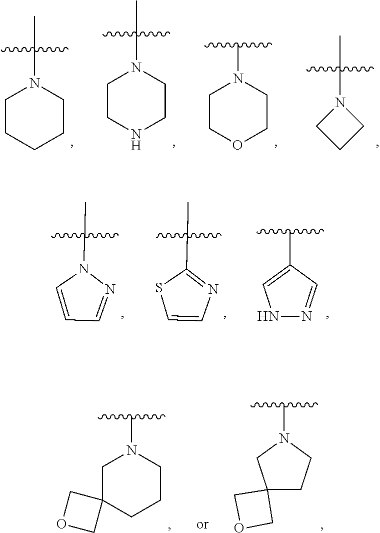
and Ring B1 is unsubstituted or Ring B1 is substituted one to three groups selected from —CH₃, —CH₂OH, —C(O)OH, —CF₃, fluorine, 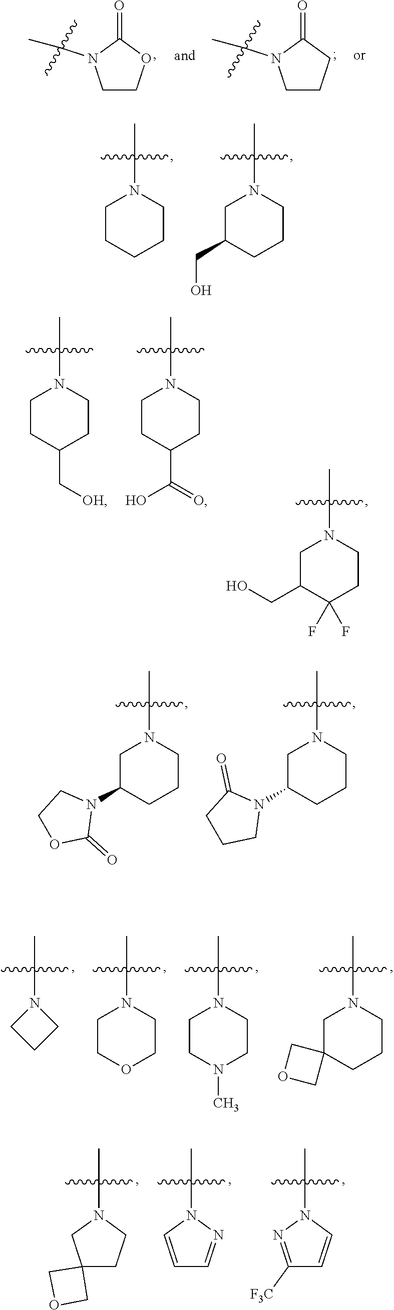 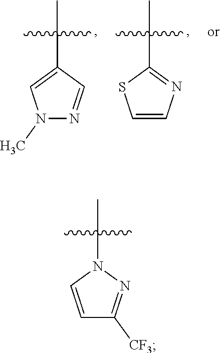
X¹ is 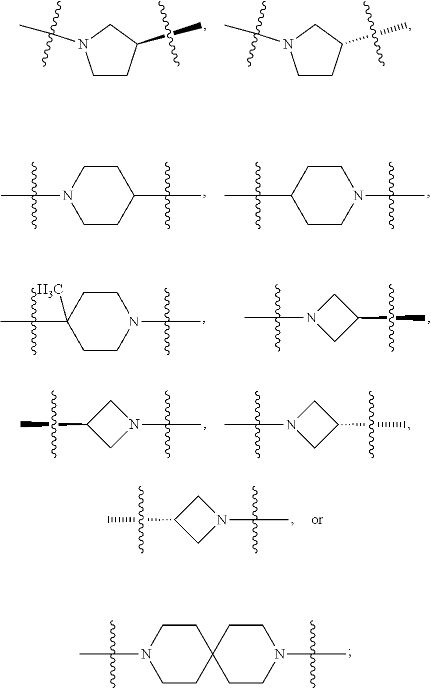
or
X² is:
a bond, —C₁₋₅ alkyl-, 4-6 membered monocyclic cycloalkyl, or 4-6 membered monocyclic heterocycloalkyl having one to two heteroatoms independently selected from N, O, or S; or
a bond, —C₁₋₃ alkyl-, —C(O)—, 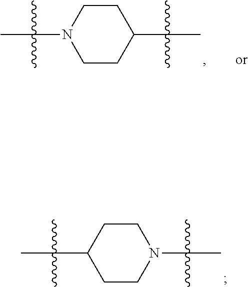 [SMILES: *C1CCN(*)CC1 ; *C1CCN(*)CC1]
or
X³ is:
a bond, —C₁₋₄ alkyl-, —N(R)—, —(O—CH₂—CH₂)ₚ—, —(CH₂—CH₂—O)ₚ—, or a 4-6 membered heterocycloalkyl having one to two heteroatoms independently selected from N, O, or S, wherein the heterocycloalkyl is substituted with —CH₃ or is unsubstituted; or 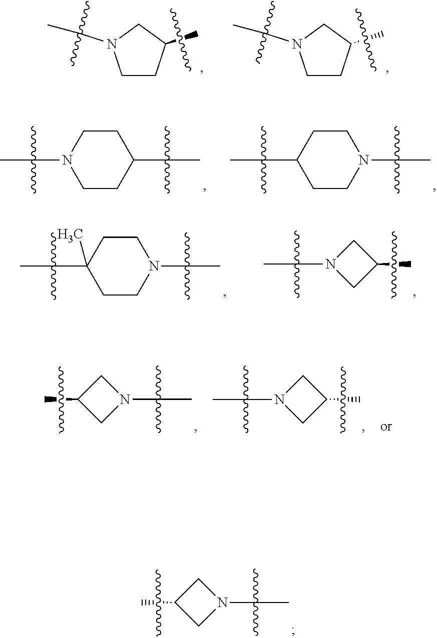
or
L is 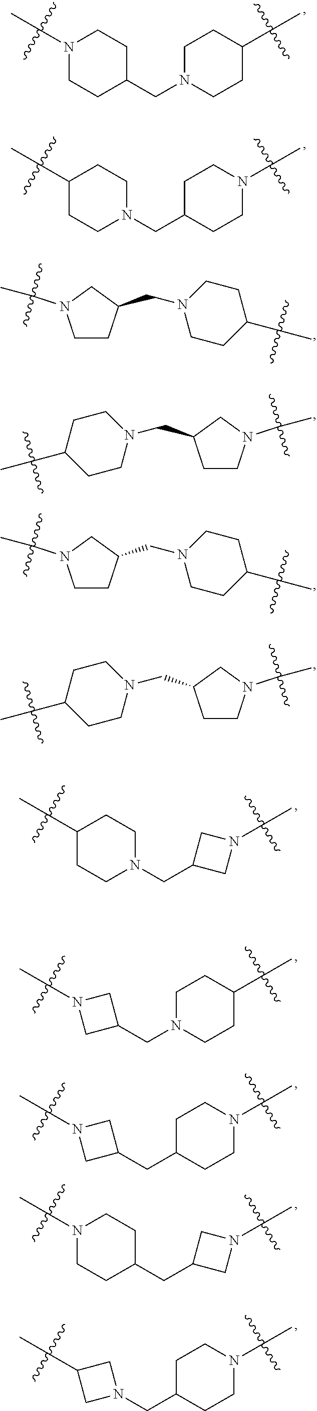 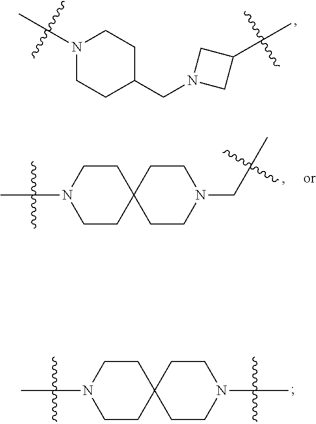 [SMILES: *C1CN(CC2CCN(*)CC2)C1 ; *CN1CCC2(CCN(*)CC2)CC1 ; *N1CCC2(CC1)CCN(*)CC2]
or
W is N and D is a bond.

119. The method of claim 117, wherein in the compound of Formula (C):
Ring C is 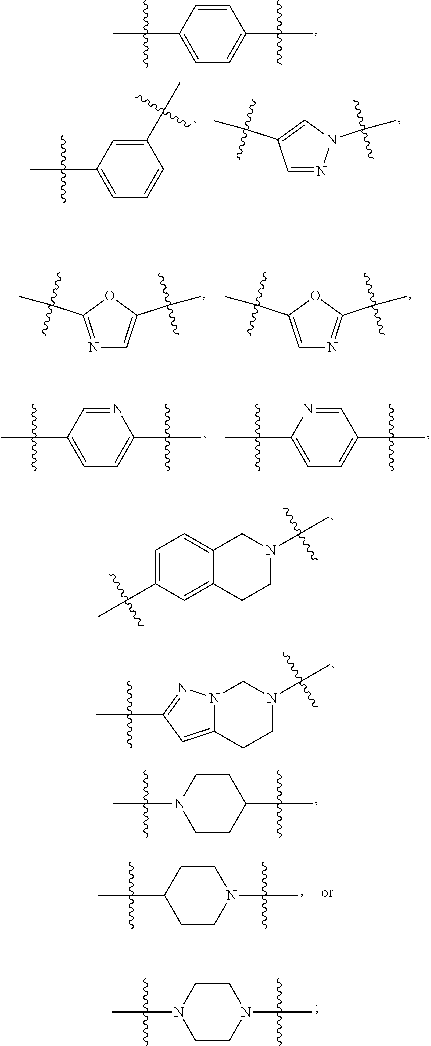
or
X¹ is:
a 4-6 membered monocyclic heterocycloalkyl having one to two heteroatoms independently selected from N, O, or S; or 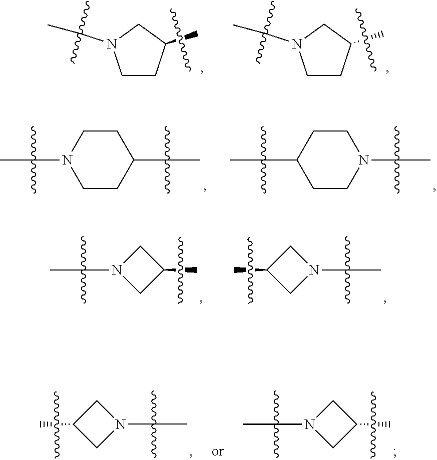
or
X² is a bond, —C₁₋₅ alkyl-, 4-6 membered monocyclic cycloalkyl, or 4-6 membered monocyclic heterocycloalkyl having one to two heteroatoms independently selected from N, O, or S; or
X³ is:
a 4-6 membered cycloalkyl, —N(R)—, or a 4-6 membered heterocycloalkyl having one to two heteroatoms independently selected from N, O, or S, wherein the heterocycloalkyl is substituted with —CH₃ or is unsubstituted; or 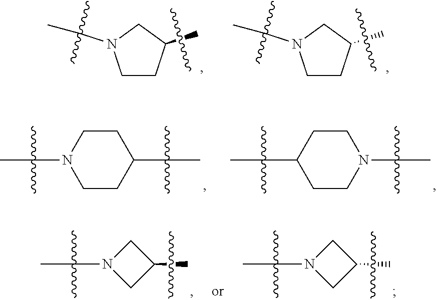
or
L is

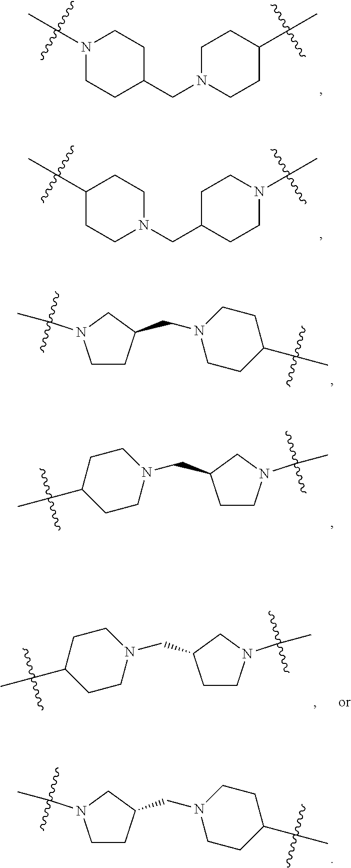 [SMILES: *C1CCN(CC2CCN(*)CC2)CC1 ; *C1CCN(CC2CCN(*)CC2)CC1 ; *C1CCN(C[C@H]2CCN(*)C2)CC1 ; *C1CCN(C[C@@H]2CCN(*)C2)CC1 ; *C1CCN(C[C@H]2CCN(*)C2)CC1 ; *C1CCN(C[C@@H]2CCN(*)C2)CC1]

120. The method of claim 117, wherein the compound of Formula (D) is a compound of Formula (D-1) or a compound of Formula (D-2):
Formula (D-1) 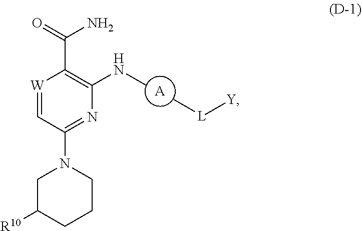
or a pharmaceutically acceptable salt thereof, wherein
W is CH or N;
Ring A is 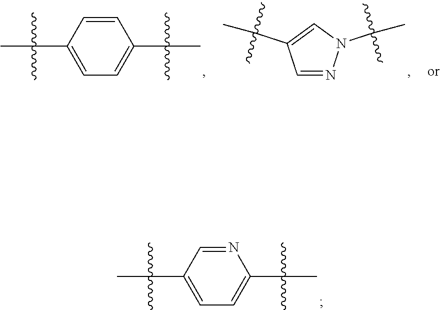 [SMILES: *c1ccc(*)cc1 ; *c1cnn(*)c1 ; *c1ccc(*)nc1]
L is —X¹—X²—X³—;
X¹ is —C₁₋₅ alkyl- or 4-6 membered monocyclic heterocycloalkyl having one to two heteroatoms independently selected from N, O, or S, wherein the monocyclic heterocycloalkyl of X¹ is substituted with —CH₃ or is unsubstituted; or
X² is a bond, —C₁₋₅ alkyl-, or 4-6 membered monocyclic heterocycloalkyl having one to two heteroatoms independently selected from N, O, or S, wherein the monocyclic heterocycloalkyl of X¹ is substituted with —CH₃ or is unsubstituted;
X³ is a bond, —C₁₋₄ alkyl-, 4-6 membered monocyclic cycloalkyl, or 4-6 membered heterocycloalkyl having one to two heteroatoms independently selected from N, O, or S, wherein the heterocycloalkyl is substituted with —CH₃ or is unsubstituted; and
R¹⁰ is 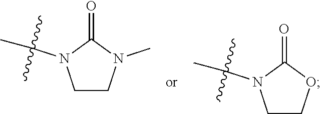
or
Formula (D-2) 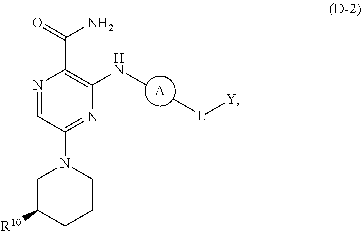
or a pharmaceutically acceptable salt thereof.

121. The method of claim 117, wherein in the compound of Formula (D):
Ring A is 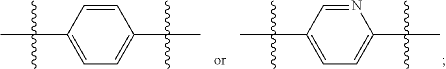 [SMILES: *c1ccc(*)cc1 ; *c1ccc(*)nc1]
or
X¹ is:
a 4-6 membered monocyclic heterocycloalkyl having one to two heteroatoms independently selected from N, O, or S, wherein the monocyclic heterocycloalkyl of X¹ is substituted with —CH₃ or is unsubstituted; or 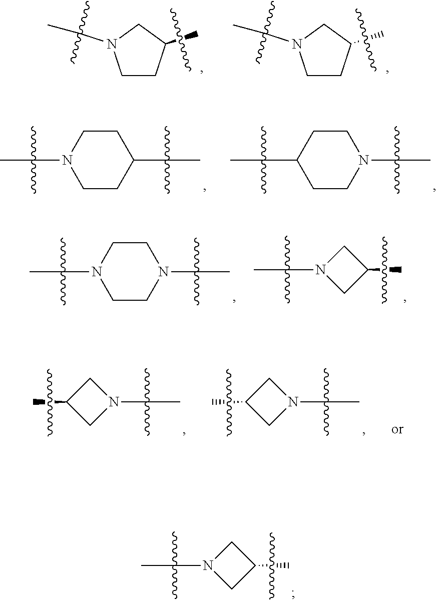
or
X² is a bond, —C₁₋₅ alkyl-, 4-6 membered monocyclic cycloalkyl, or 4-6 membered monocyclic heterocycloalkyl having one to two heteroatoms independently selected from N, O, or S; or
X³ is:
a bond, a 4-6 membered monocyclic cycloalkyl, or 4-6 membered monocyclic heterocycloalkyl having one to two heteroatoms independently selected from N, O, or S; or 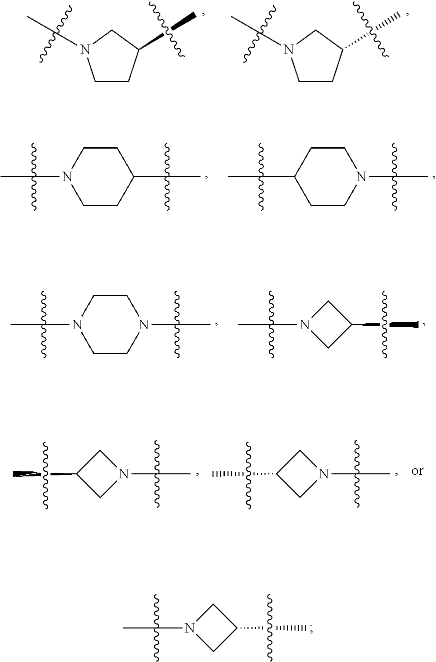
or
L is 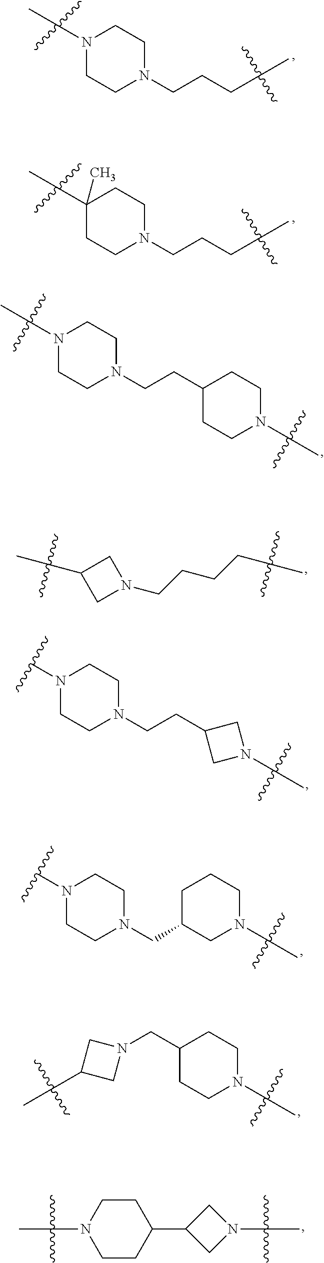 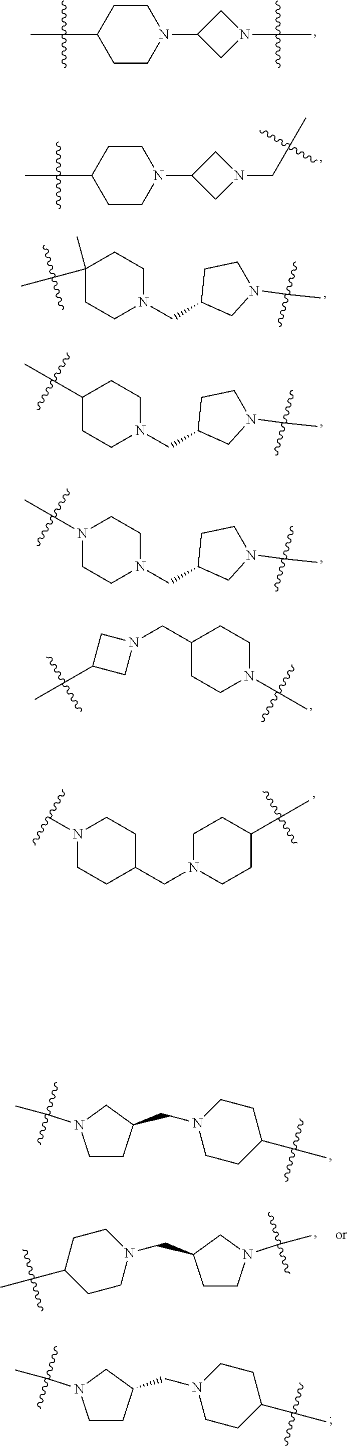
or
R¹⁰ is:

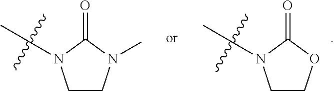

122. The method of claim 117, wherein in the compound of Formula (E):
Ring B is 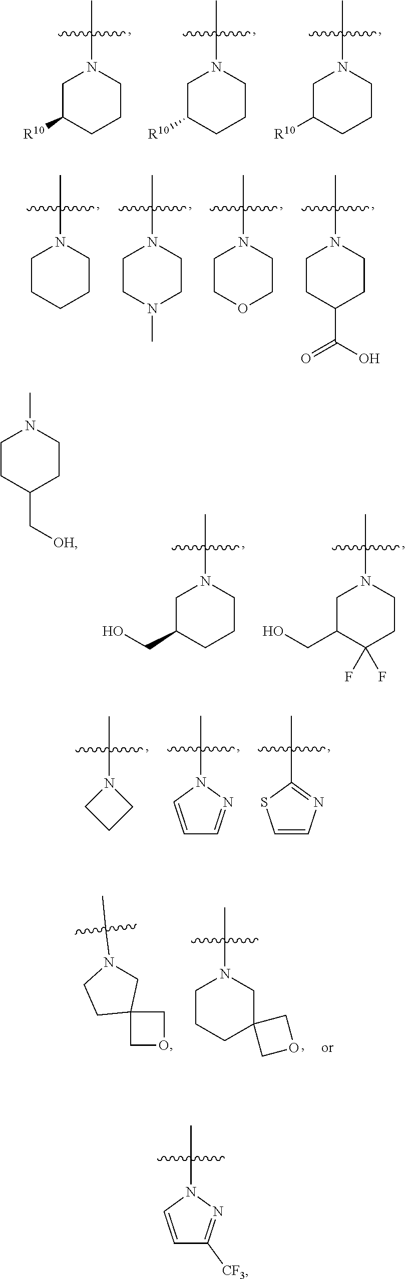
wherein R¹⁰ is 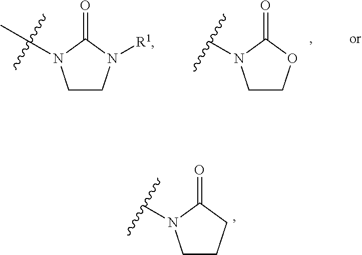
and wherein R¹ is a C₁₋₄ alkyl group; or
Ring A is
or 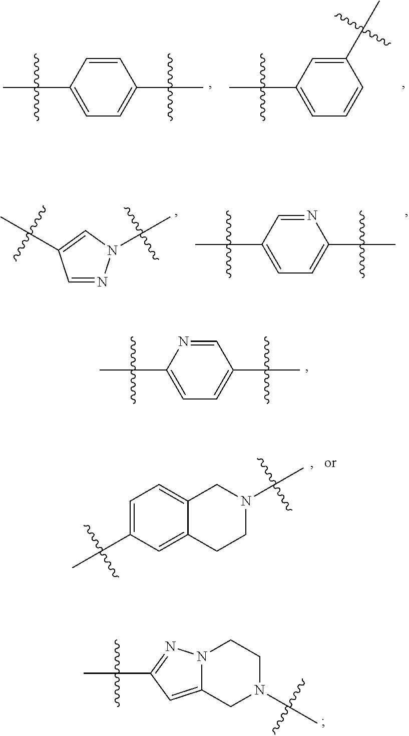
X⁵ is a bond, —N(R)—, or —C(O)—N(R)—; or
L is 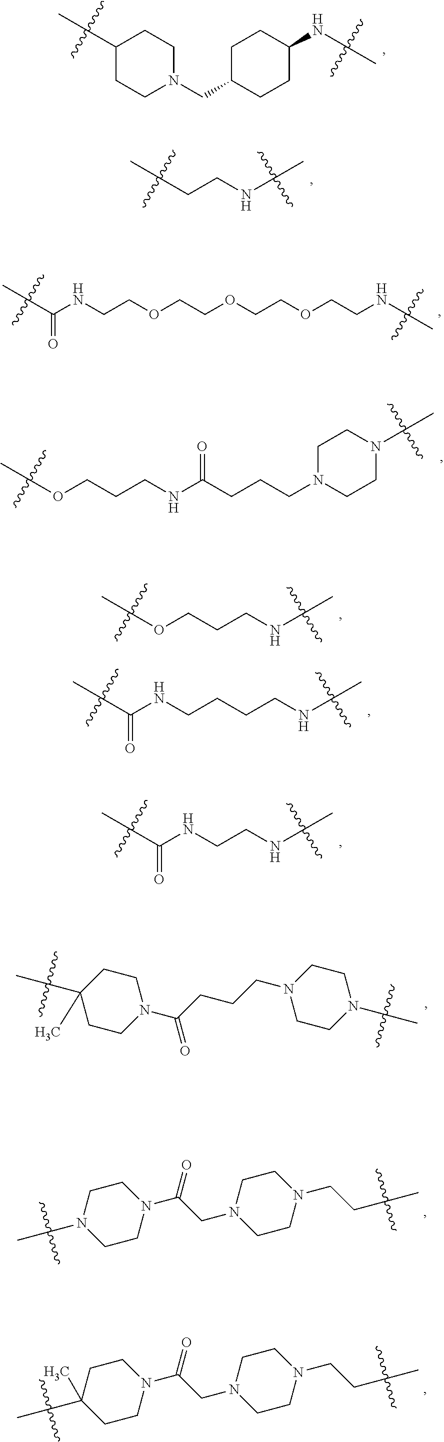 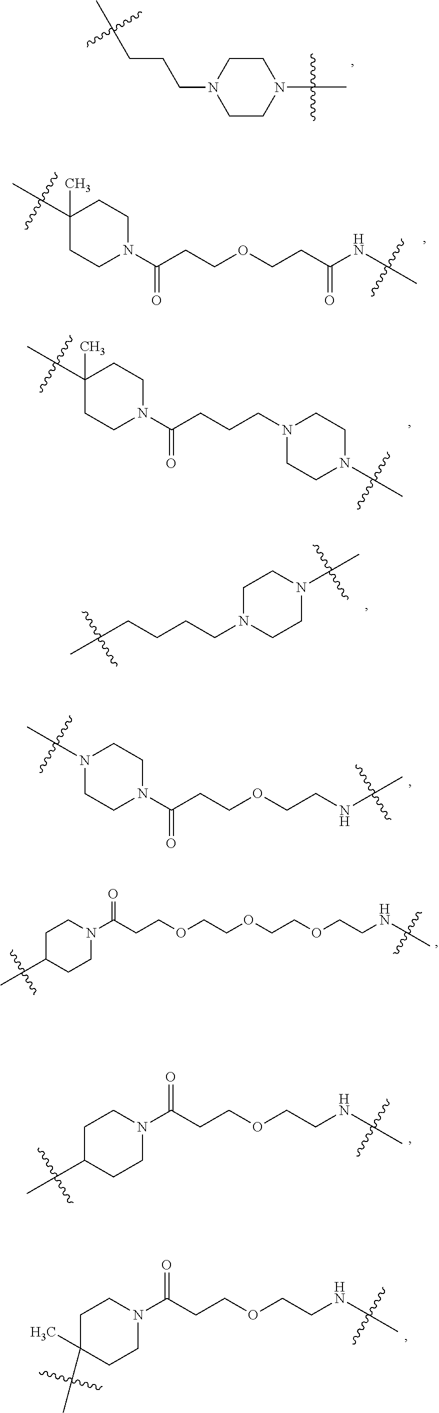 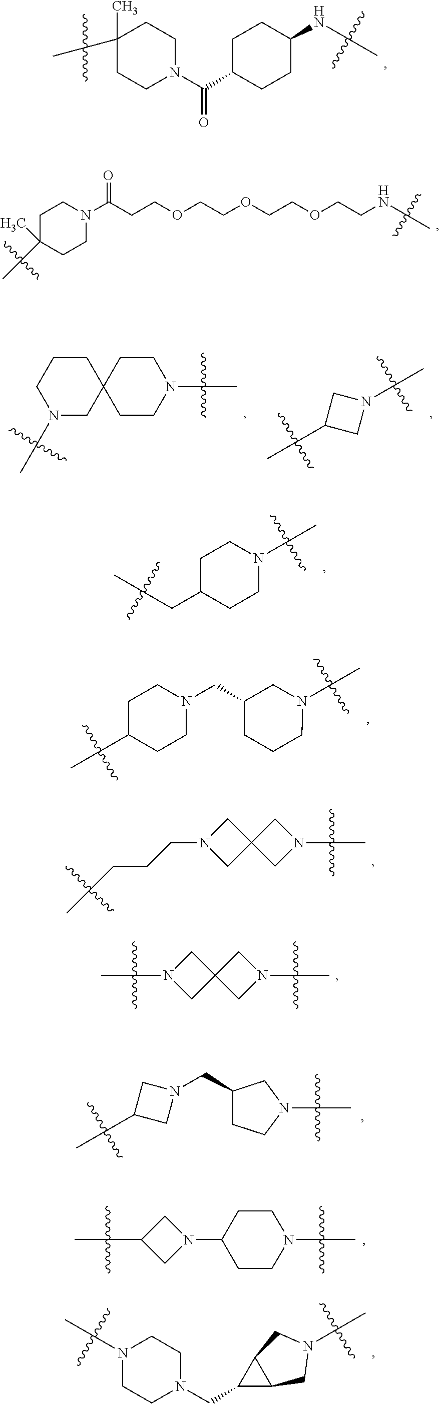 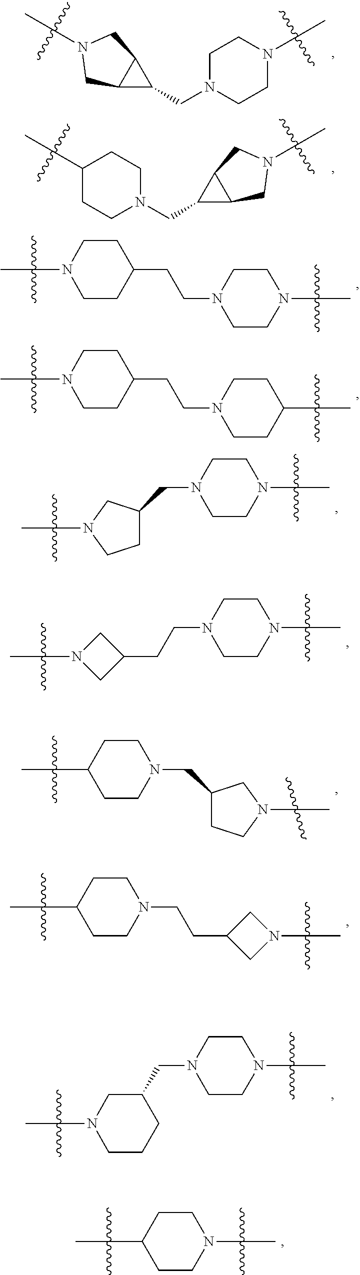 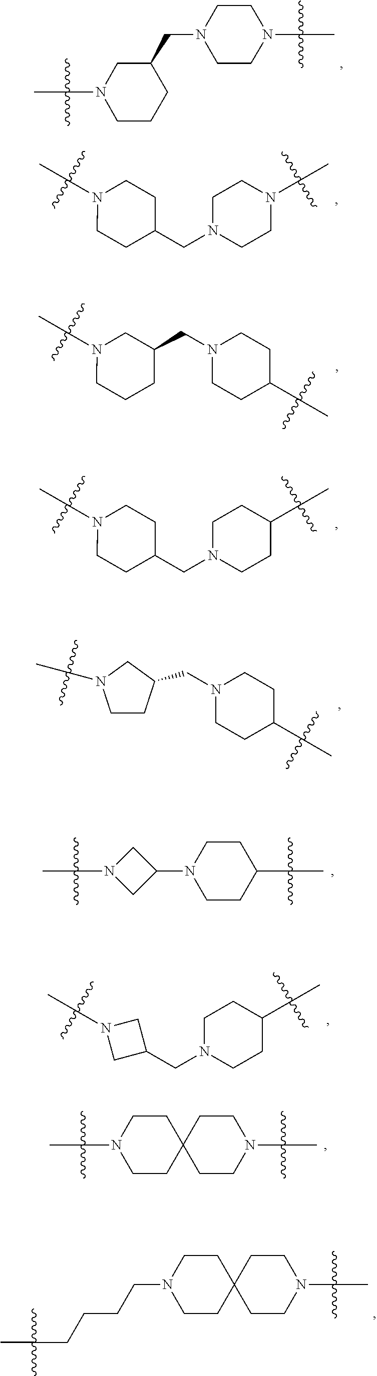 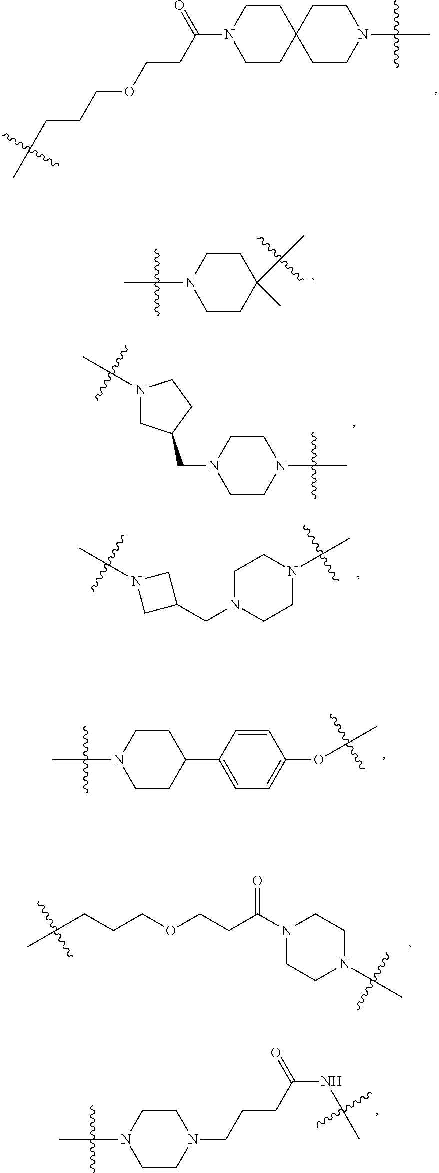 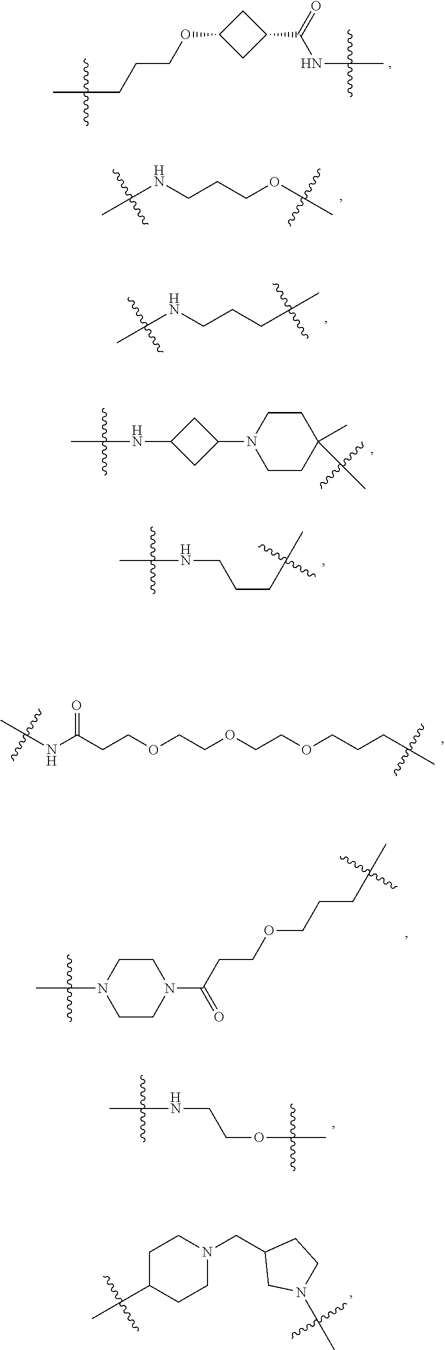 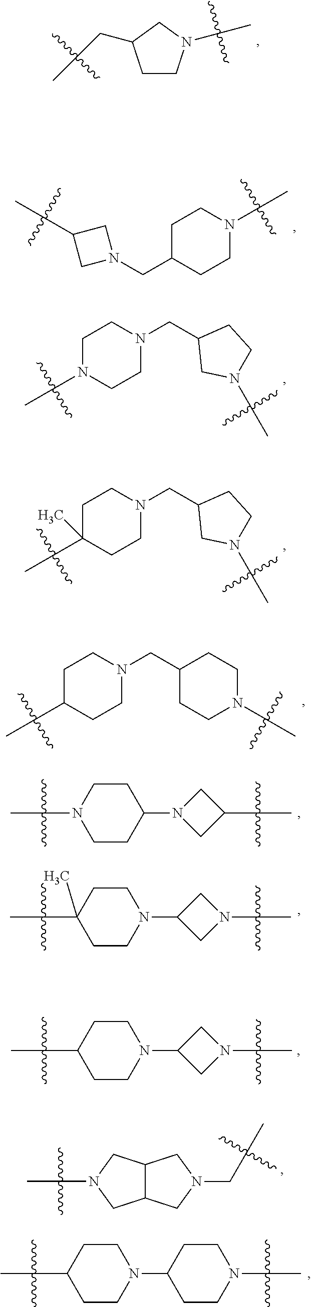 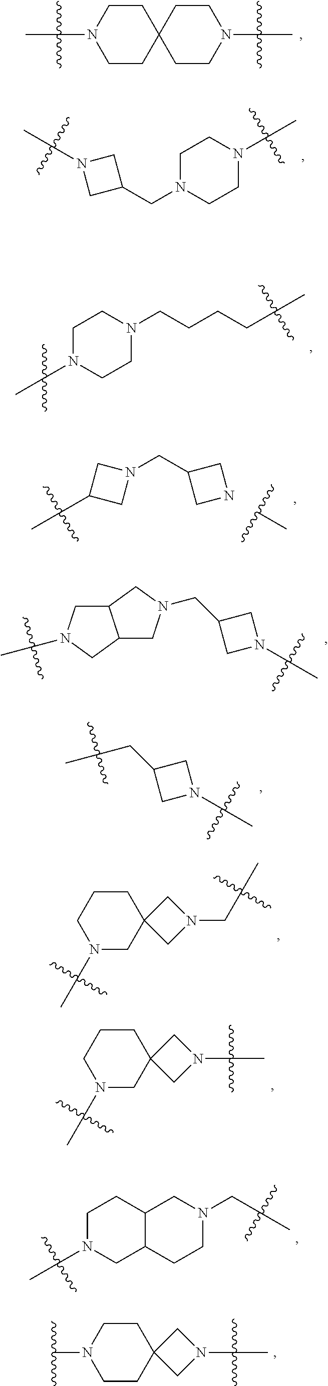 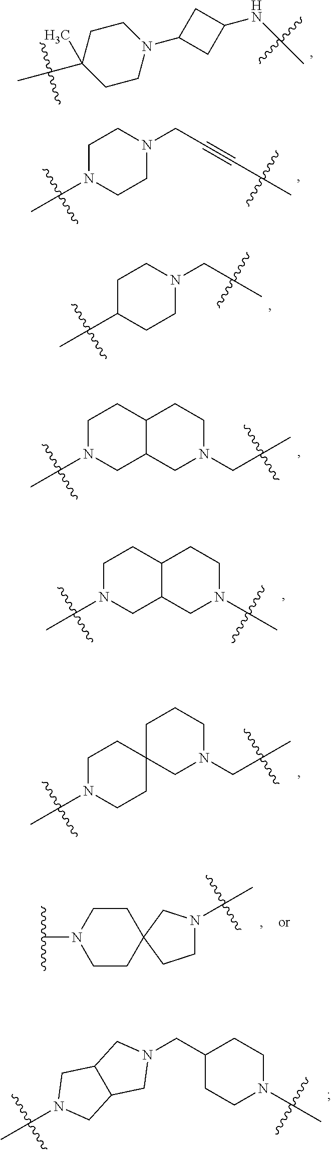
or
Y is

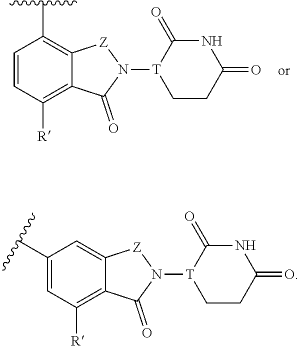

123. The method of claim 117, wherein in the compound of Formula (F):
W is N; or
Y is 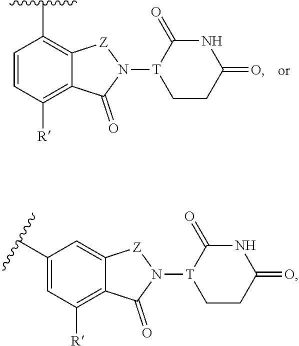
wherein each T is independently CH or N; and each Z is independently —CH₂— or —C(O)—; and each R′ is hydrogen, methyl, or NH₂; or
X¹ is:
a 4-6 membered monocyclic heterocycloalkyl having one to two heteroatoms independently selected from N, O, or S, wherein each of the monocyclic heterocycloalkyl of X¹ is substituted with —CH₃ or is unsubstituted; or 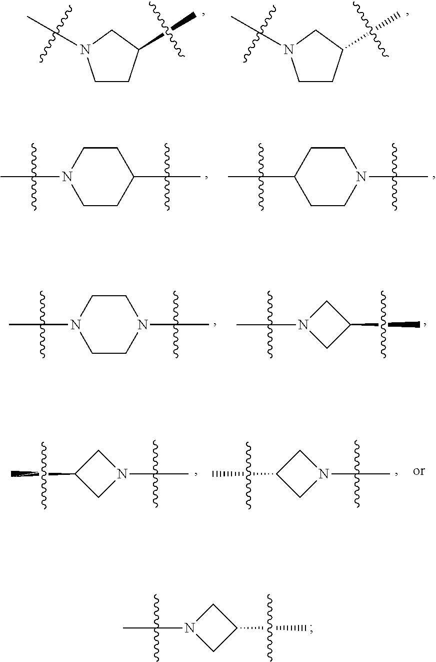
or
X² is a bond or —C₁₋₅ alkyl-; or
X³ is:
a 4-6 membered monocyclic heterocycloalkyl having one to two heteroatoms independently selected from N, O, or S; or 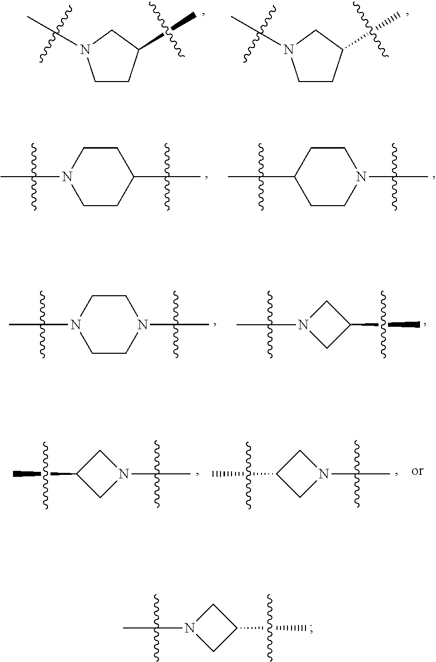
or
L is

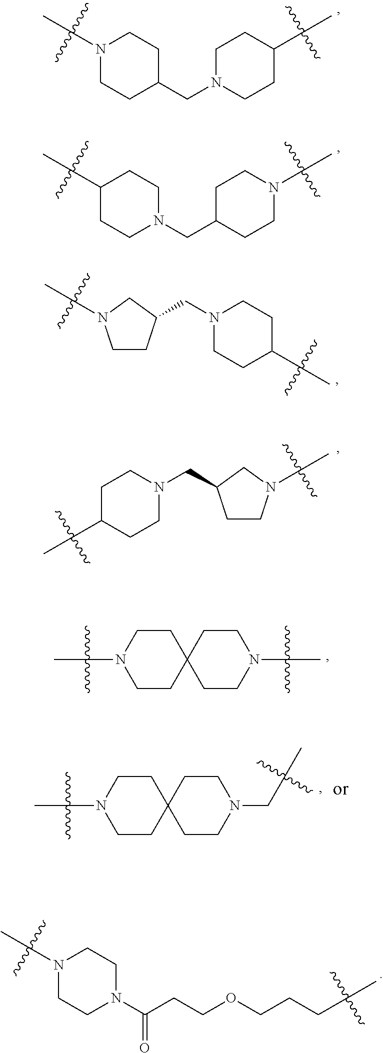

124. The method of claim 117, wherein:
in the compound of Formula (G):
R¹ is methyl; or
Y is 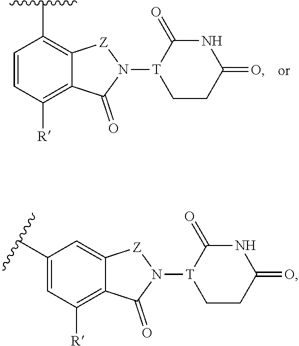
wherein each T is independently CH or N; and each Z is independently —CH₂— or —C(O)—; and each R′ is hydrogen, methyl, or NH₂; or
W is N; or
in the compound of Formula (M):
R^{10A} is hydrogen or 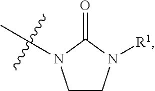
and R¹ is methyl, ethyl, propyl, iso-propyl, butyl, sec-butyl, or iso-butyl; or
X¹ is —CH₂—, —CH₂CH₂—, or —CH₂CH₂CH₂—; or
Ring C-1 is

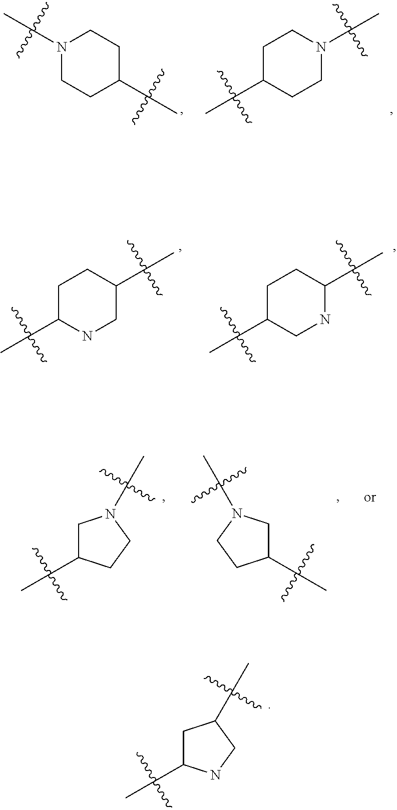

125. The method of claim 1, wherein the compound is selected from the following, or a pharmaceutically acceptable salt thereof:

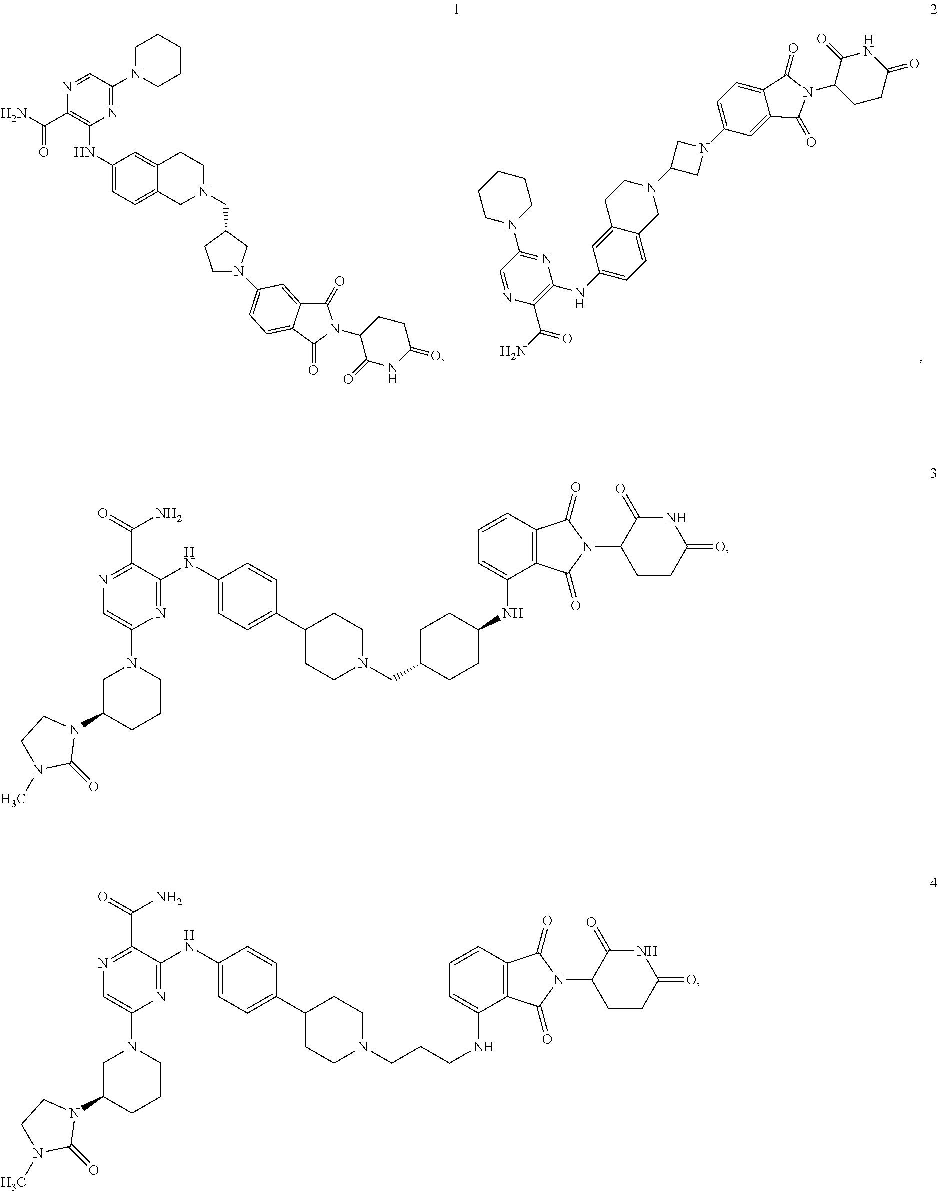 [SMILES: NC(=O)c1ncc(N2CCCCC2)nc1Nc1ccc2c(c1)CCN(C[C@H]1CCN(c3ccc4c(c3)C(=O)N(C3CCC(=O)NC3=O)C4=O)C1)C2 ; NC(=O)c1ncc(N2CCCCC2)nc1Nc1ccc2c(c1)CCN(C1CN(c3ccc4c(c3)C(=O)N(C3CCC(=O)NC3=O)C4=O)C1)C2 ; CN1CCN([C@@H]2CCCN(c3cnc(C(N)=O)c(Nc4ccc(C5CCN(C[C@H]6CC[C@H](Nc7cccc8c7C(=O)N(C7CCC(=O)NC7=O)C8=O)CC6)CC5)cc4)n3)C2)C1=O ; CN1CCN([C@@H]2CCCN(c3cnc(C(N)=O)c(Nc4ccc(C5CCN(CCCNc6cccc7c6C(=O)N(C6CCC(=O)NC6=O)C7=O)CC5)cc4)n3)C2)C1=O]

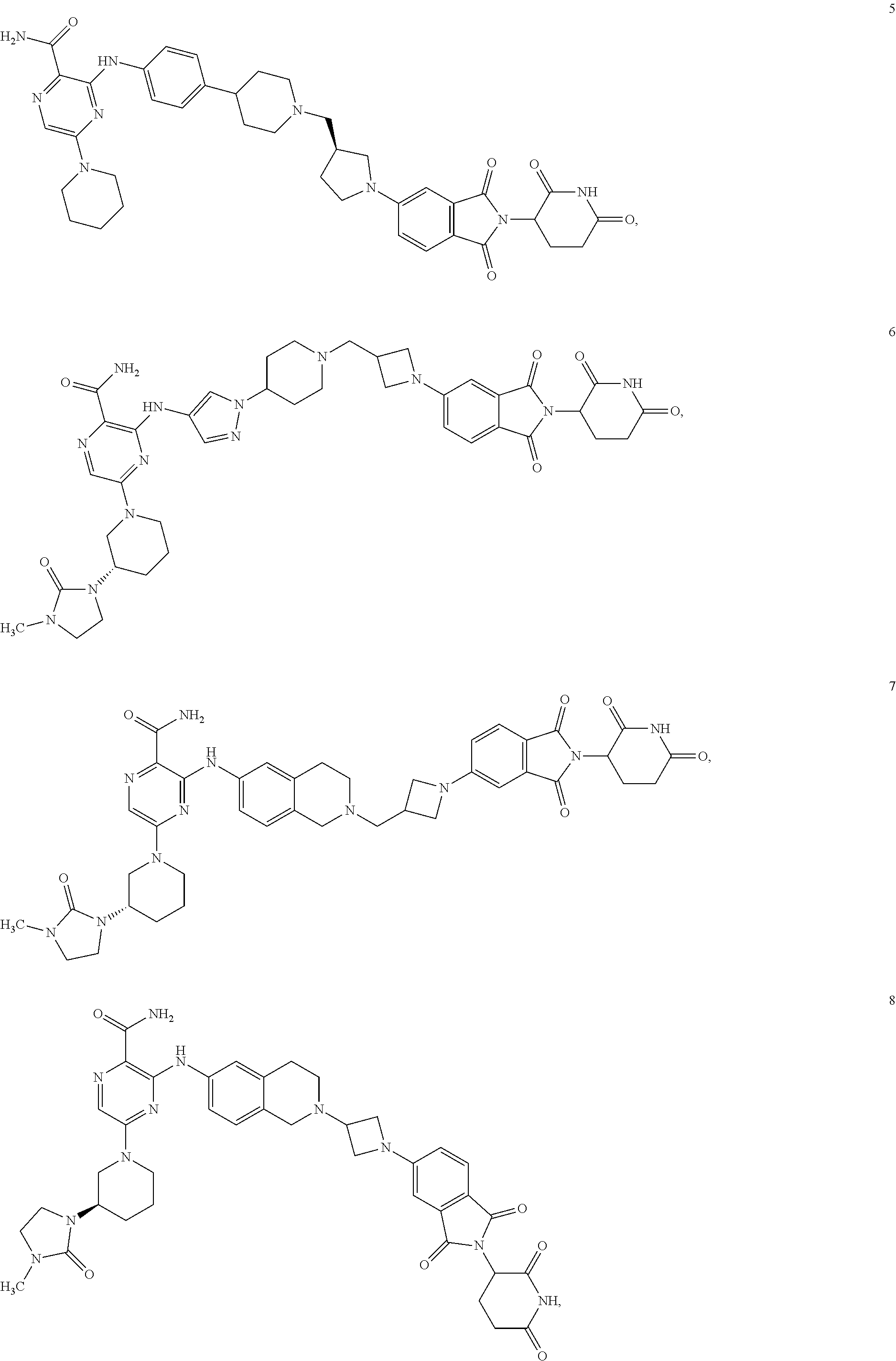 [SMILES: NC(=O)c1ncc(N2CCCCC2)nc1Nc1ccc(C2CCN(C[C@@H]3CCN(c4ccc5c(c4)C(=O)N(C4CCC(=O)NC4=O)C5=O)C3)CC2)cc1 ; CN1CCN([C@H]2CCCN(c3cnc(C(N)=O)c(Nc4cnn(C5CCN(CC6CN(c7ccc8c(c7)C(=O)N(C7CCC(=O)NC7=O)C8=O)C6)CC5)c4)n3)C2)C1=O ; CN1CCN([C@H]2CCCN(c3cnc(C(N)=O)c(Nc4ccc5c(c4)CCN(CC4CN(c6ccc7c(c6)C(=O)N(C6CCC(=O)NC6=O)C7=O)C4)C5)n3)C2)C1=O ; CN1CCN([C@@H]2CCCN(c3cnc(C(N)=O)c(Nc4ccc5c(c4)CCN(C4CN(c6ccc7c(c6)C(=O)N(C6CCC(=O)NC6=O)C7=O)C4)C5)n3)C2)C1=O]

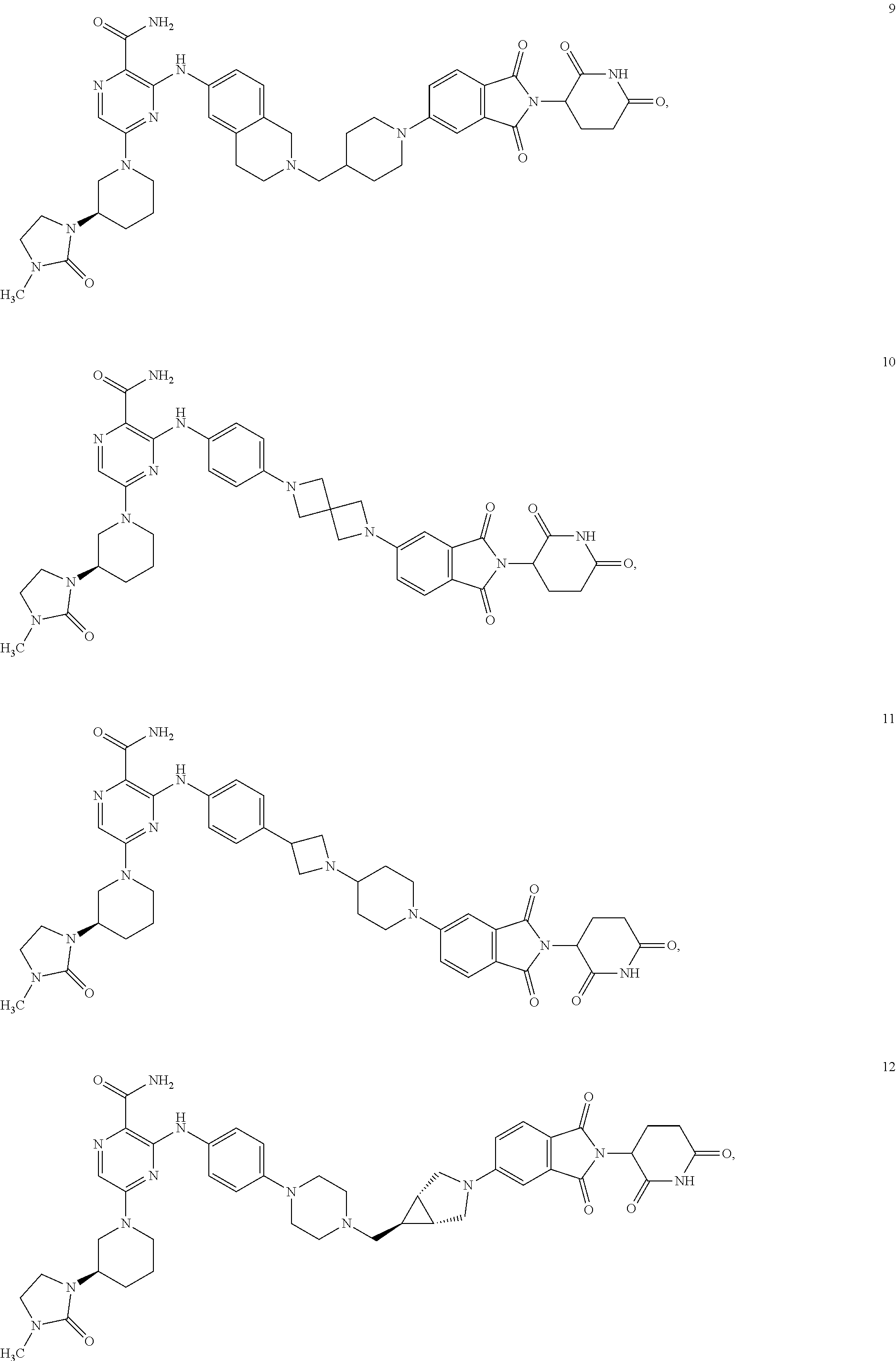 [SMILES: CN1CCN([C@@H]2CCCN(c3cnc(C(N)=O)c(Nc4ccc5c(c4)CCN(CC4CCN(c6ccc7c(c6)C(=O)N(C6CCC(=O)NC6=O)C7=O)CC4)C5)n3)C2)C1=O ; CN1CCN([C@@H]2CCCN(c3cnc(C(N)=O)c(Nc4ccc(N5CC6(C5)CN(c5ccc7c(c5)C(=O)N(C5CCC(=O)NC5=O)C7=O)C6)cc4)n3)C2)C1=O ; CN1CCN([C@@H]2CCCN(c3cnc(C(N)=O)c(Nc4ccc(C5CN(C6CCN(c7ccc8c(c7)C(=O)N(C7CCC(=O)NC7=O)C8=O)CC6)C5)cc4)n3)C2)C1=O ; CN1CCN([C@@H]2CCCN(c3cnc(C(N)=O)c(Nc4ccc(N5CCN(C[C@@H]6[C@H]7CN(c8ccc9c(c8)C(=O)N(C8CCC(=O)NC8=O)C9=O)C[C@@H]67)CC5)cc4)n3)C2)C1=O]

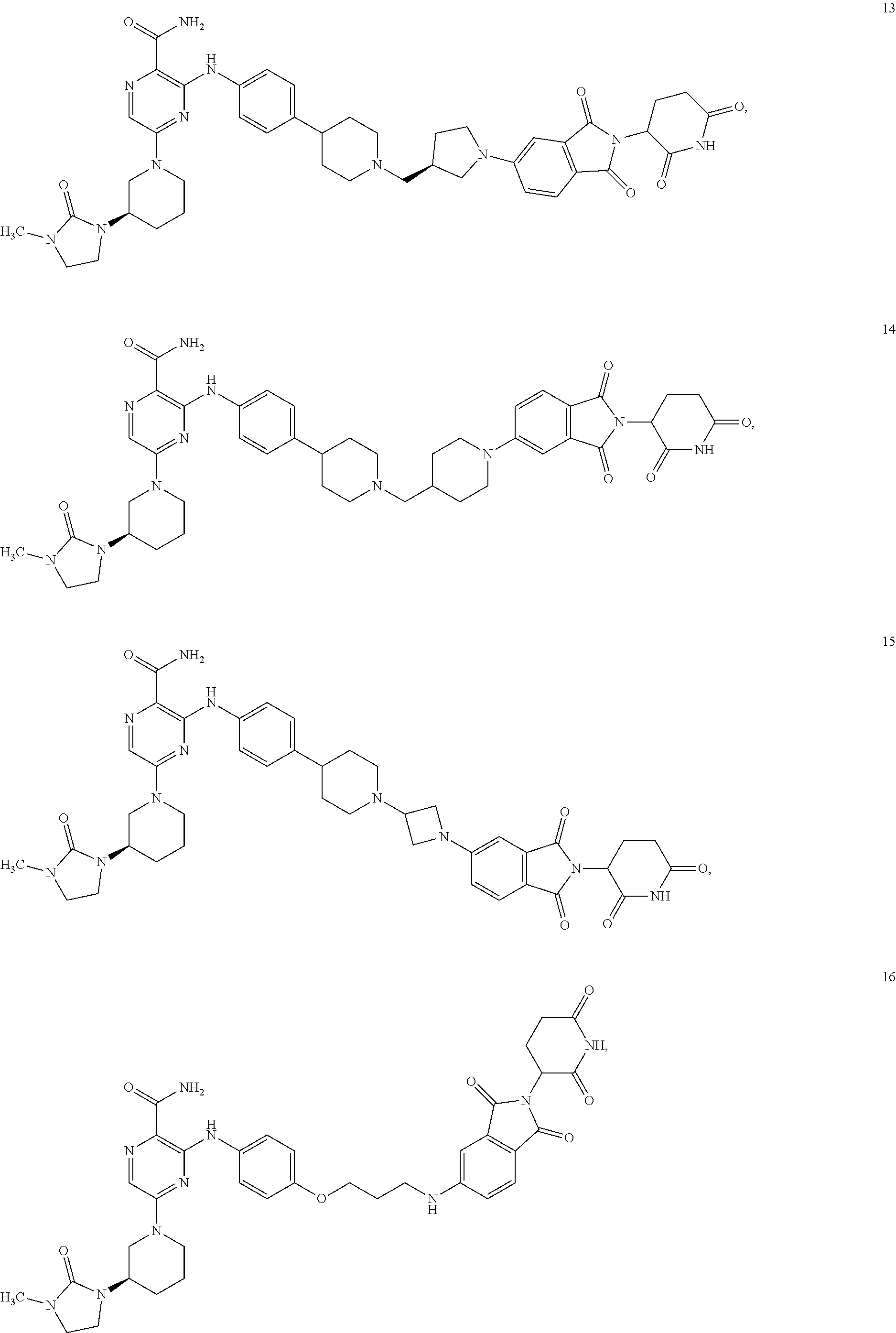 [SMILES: CN1CCN([C@@H]2CCCN(c3cnc(C(N)=O)c(Nc4ccc(C5CCN(C[C@H]6CCN(c7ccc8c(c7)C(=O)N(C7CCC(=O)NC7=O)C8=O)C6)CC5)cc4)n3)C2)C1=O ; CN1CCN([C@@H]2CCCN(c3cnc(C(N)=O)c(Nc4ccc(C5CCN(CC6CCN(c7ccc8c(c7)C(=O)N(C7CCC(=O)NC7=O)C8=O)CC6)CC5)cc4)n3)C2)C1=O ; CN1CCN([C@@H]2CCCN(c3cnc(C(N)=O)c(Nc4ccc(C5CCN(C6CN(c7ccc8c(c7)C(=O)N(C7CCC(=O)NC7=O)C8=O)C6)CC5)cc4)n3)C2)C1=O ; CN1CCN([C@@H]2CCCN(c3cnc(C(N)=O)c(Nc4ccc(OCCCNc5ccc6c(c5)C(=O)N(C5CCC(=O)NC5=O)C6=O)cc4)n3)C2)C1=O]

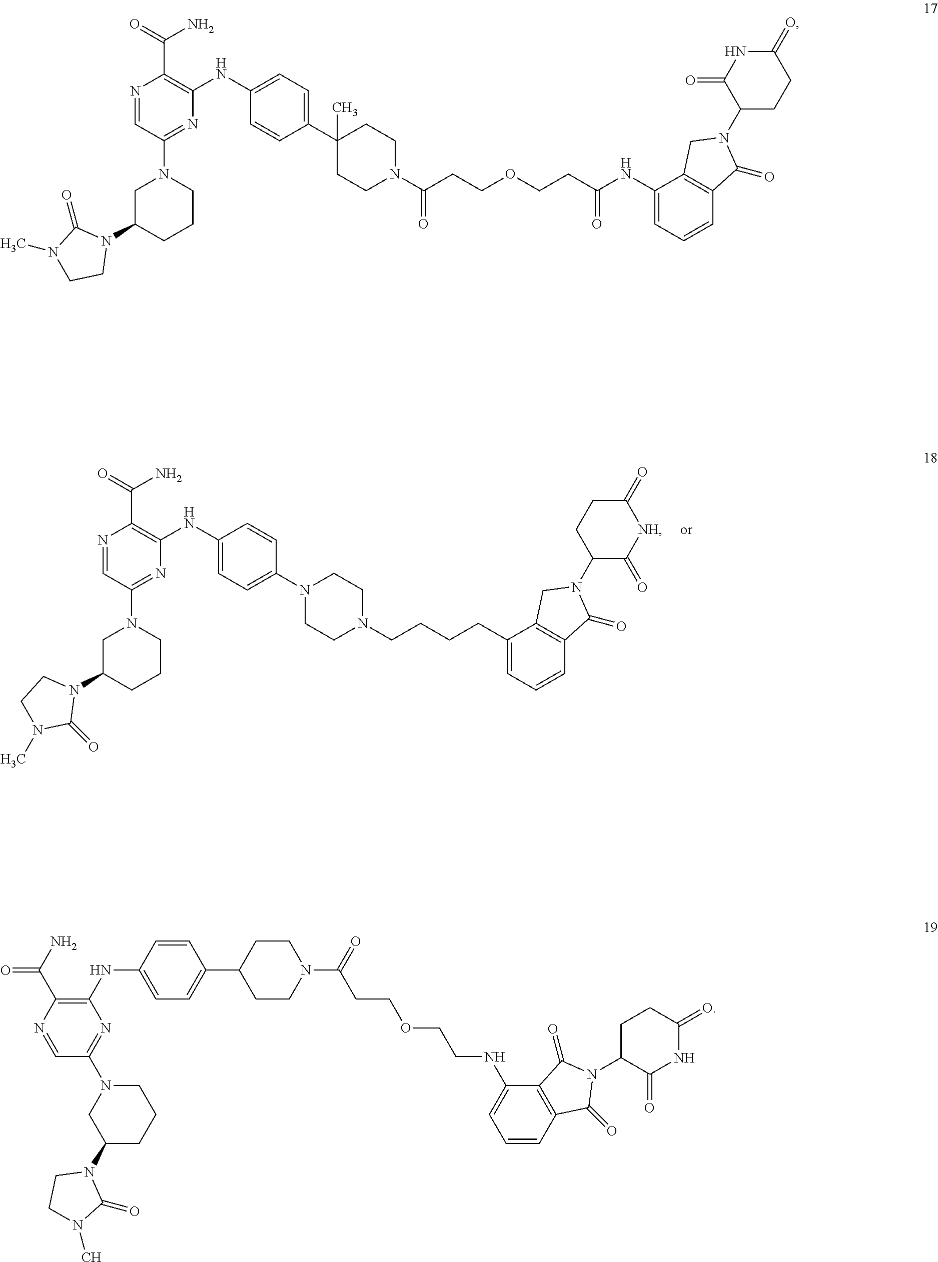 [SMILES: CN1CCN([C@@H]2CCCN(c3cnc(C(N)=O)c(Nc4ccc(C5(C)CCN(C(=O)CCOCCC(=O)Nc6cccc7c6CN(C6CCC(=O)NC6=O)C7=O)CC5)cc4)n3)C2)C1=O ; CN1CCN([C@@H]2CCCN(c3cnc(C(N)=O)c(Nc4ccc(N5CCN(CCCCc6cccc7c6CN(C6CCC(=O)NC6=O)C7=O)CC5)cc4)n3)C2)C1=O ; CN1CCN([C@@H]2CCCN(c3cnc(C(N)=O)c(Nc4ccc(C5CCN(C(=O)CCOCCNc6cccc7c6C(=O)N(C6CCC(=O)NC6=O)C7=O)CC5)cc4)n3)C2)C1=O]

126. The method of claim 1, wherein the compound is administered in the form of a pharmaceutical composition comprising the compound or pharmaceutically acceptable salt thereof and a pharmaceutically acceptable carrier, vehicle, or adjuvant.

127. A compound selected from the following: 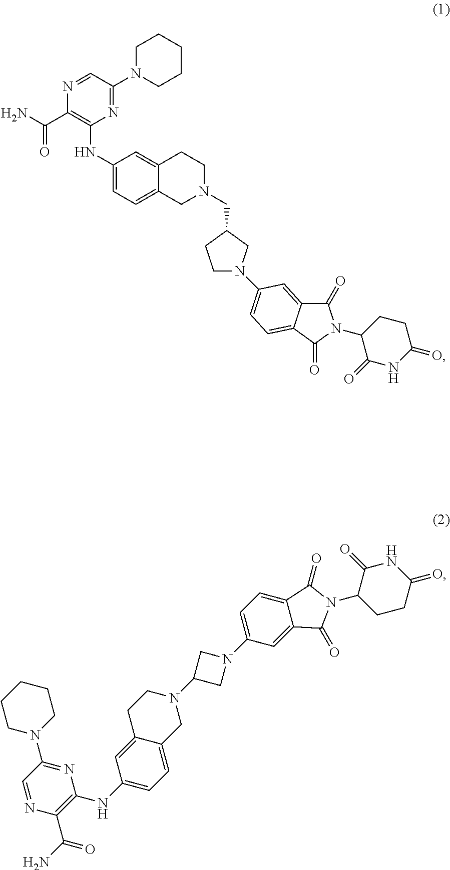 [SMILES: NC(=O)c1ncc(N2CCCCC2)nc1Nc1ccc2c(c1)CCN(C[C@H]1CCN(c3ccc4c(c3)C(=O)N(C3CCC(=O)NC3=O)C4=O)C1)C2 ; NC(=O)c1ncc(N2CCCCC2)nc1Nc1ccc2c(c1)CCN(C1CN(c3ccc4c(c3)C(=O)N(C3CCC(=O)NC3=O)C4=O)C1)C2]
or a pharmaceutically acceptable salt thereof.

128. A pharmaceutical composition comprising a bifunctional compound or pharmaceutically acceptable salt thereof and one or more pharmaceutically acceptable carriers, excipients, or diluents;
wherein the bifunctional compound is a compound of Formula (A) 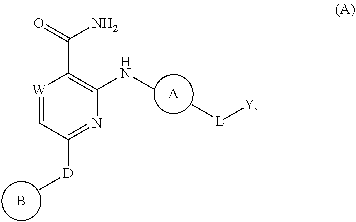
or a pharmaceutically acceptable salt thereof, wherein
W is CH or N;
D is a bond or —NH—;
Ring A is phenyl, a 9-10 membered bicyclic aryl, a 5-6 membered partially or fully unsaturated monocyclic heterocycle, or a 9-10 membered bicyclic heteroaryl, wherein the monocyclic heterocycle and bicyclic heteroaryl of Ring A each possess one to three heteroatoms independently selected from N, O, or S, wherein Ring A is unsubstituted or is independently substituted with up to three substituents selected from halo, —CN, —COOH, NH₂, and substituted or unsubstituted C₁₋₆ alkyl;
Ring B is a phenyl, a 5-6 membered heteroaryl, a 4-6 membered heterocycloalkyl, or a 8-10 membered spiro bicyclic heterocycle, wherein Ring B is substituted or is unsubstituted, and wherein the heteroaryl and heterocycloalkyl of Ring B has one to three heteroatoms independently selected from N, O, or S;
L is —X¹—X²—X³—X⁴—X⁵—;
X¹ is a bond, —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro or fused bicyclic heterocycloalkyl having one to three heteroatoms independently selected from N, O, or S, or 4-6 membered monocyclic heterocycloalkyl having one to two heteroatoms independently selected from N, O, or S, wherein each of the monocyclic and bicyclic heterocycloalkyl of X¹ is substituted with —CH₃ or is unsubstituted;
X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having one to two heteroatoms independently selected from N, O, or S;
X³ is a bond, —C₁₋₈ alkyl-, —C≡C—, 4-6 membered cycloalkyl, —N(R)—, —N(R)—C(O)—, —(O—CH₂—CH₂)ₚ—, —(CH₂—CH₂—O)ₚ—, 4-6 membered heterocycloalkyl having one to two heteroatoms independently selected from N, O, or S, wherein the heterocycloalkyl is substituted with —CH₃ or is unsubstituted;
X⁴ is a bond, —CH₂—CH₂—N(R)—, —N(R)—, —C₁₋₄ alkyl-, —(O—CH₂—CH₂—CH₂)ₘ—, a 5-6 membered saturated, partially unsaturated, or fully unsaturated carbocycle, or a 5-6 membered saturated, partially unsaturated, or fully unsaturated heterocycle having one to three heteroatoms independently selected from N, O, or S;
X⁵ is a bond, —C₁₋₄ alkyl-, —N(R)—, —O—, —C(O)—, or —C(O)—N(R)—;
each R is independently hydrogen or —C₁₋₃ alkyl; and
each of m, n, and p is independently an integer from one to three; and
Y is 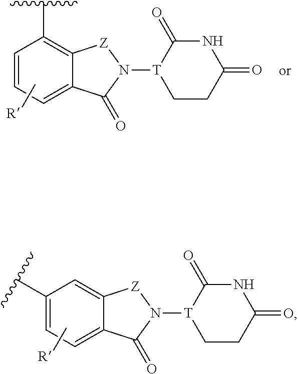
wherein each T is independently CH or N; and each Z is independently —CH₂— or —C(O)—; and each R′ is hydrogen, methyl, or NH₂.

129. The method of claim 1, wherein the step of administering comprises administering to the subject an amount of a pharmaceutical composition comprising the bifunctional compound and one or more pharmaceutically acceptable carriers, excipients, or diluents; and wherein the bifunctional compound is selected from: 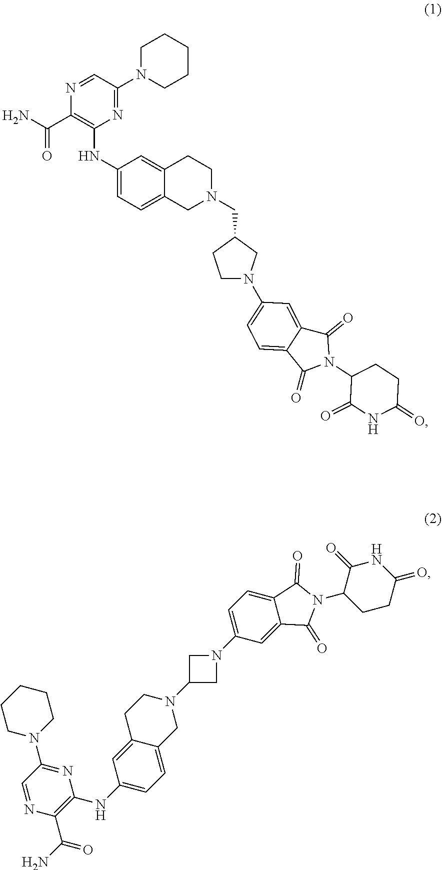 [SMILES: NC(=O)c1ncc(N2CCCCC2)nc1Nc1ccc2c(c1)CCN(C[C@H]1CCN(c3ccc4c(c3)C(=O)N(C3CCC(=O)NC3=O)C4=O)C1)C2 ; NC(=O)c1ncc(N2CCCCC2)nc1Nc1ccc2c(c1)CCN(C1CN(c3ccc4c(c3)C(=O)N(C3CCC(=O)NC3=O)C4=O)C1)C2]
or a pharmaceutically acceptable salt thereof.
